(19) United States
(12) Patent Application Publication
(10) Pub. No.: US 2021/0393693 A1
(43) Pub. Date: Dec. 23, 2021

(54) T-Cell Modulatory Multimeric Polypeptides with Conjugation Sites and Methods of Use Thereof

(71) Applicant: Cue Biopharma, Inc., Cambridge, MA (US)

(72) Inventors: Ronald D. SEIDEL, III, Cambridge, MA (US); Rodolfo CHAPARRO, Cambridge, MA (US); John F. ROSS, Cambridge, MA (US); Chee Meng LOW, Cambridge, MA (US)

(21) Appl. No.: 17/342,518

(22) Filed: Jun. 8, 2021

Related U.S. Application Data
(63) Continuation of application No. PCT/US2019/067679, filed on Dec. 19, 2019.
(60) Provisional application No. 62/782,293, filed on Dec. 19, 2018.
(60) Provisional application No. 62/782,271, filed on Dec. 19, 2018.
(60) Provisional application No. 62/782,261, filed on Dec. 19, 2018.

(51) Int. Cl. A61K 35/17; C07K 14/74; A61K 47/62

(52) U.S. Cl. CPC A61K 35/17; A61K 47/62; C07K 14/70539

(57) ABSTRACT

The present disclosure provides T-cell modulatory multimeric polypeptides (T-Cell-MMP) and their epitope conjugates comprising at least one immunomodulatory polypeptide (“MOD”) that may be selected to exhibit reduced binding affinity to a cognate co-immunomodulatory polypeptide (“Co-MOD”). The epitope may be, for example, a cancer-associated epitope, an infectious disease-associated epitope, or a self-epitope. The T-Cell-MMP-epitope conjugates are useful for modulating the activity of a T-cell by delivering immunomodulatory peptides, such as IL-2 or IL-2 variants that exhibit reduced binding affinity for the IL-2R, to T-cells in an epitope selective/specific manner, and accordingly, for treating individuals with a cancer, infectious disease or autoimmune disorder.

This application claims the benefit of: U.S. Provisional Appln. No. 62/782,271 filed on 19 Dec. 2018, U.S. Provisional Appln. No. 62/782,261 filed on 19 Dec. 2018, and U.S. Provisional Appln. No. 62/782,293 filed on 19 Dec. 2018. This application contains a sequence listing submitted electronically via EFS-web, which serves as both the paper copy and the computer readable form (CRF) and consists of a file entitled “123640-8006WO00_seqlist.txt”, which was created on Dec. 19, 2019, which is 541,609 bytes in size, and which is herein incorporated by reference in its entirety.

### INTRODUCTION

An adaptive immune response involves the engagement of the T-cell receptor (TCR), present on the surface of a T-cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T-cell modulation (activation or inhibition) and effector function. Following epitope-specific cell targeting, the targeted T-cells are activated through engagement of costimulatory proteins found on the APC with counterpart costimulatory proteins on the T-cells. Both signals—epitope/TCR binding and engagement of APC costimulatory proteins with T-cell costimulatory proteins—are required to drive T-cell specificity and activation or inhibition. The TCR is specific for a given epitope; however, the costimulatory protein is not epitope specific and instead is generally expressed on all T-cells or on large T-cell subsets.

### SUMMARY

The present disclosure provides T-cell modulatory multimeric polypeptides (a “T-Cell-MMP” or multiple “T-Cell-MMPs”) that in one embodiment comprise a portion of a MHC receptor and at least one immunomodulatory polypeptide (also referred to herein as a “MOD polypeptide” or, simply, a “MOD”). Any one or more of the MODs present in the T-Cell-MMP may be wild-type (“wt.”) or a variant that exhibits reduced binding affinity to its cellular (e.g., T-cell surface) binding partner/receptor (generally referred to as a “Co-MOD”). The T-Cell-MMPs comprise at least one chemical conjugation site at which a molecule comprising a target epitope (e.g., a peptide or non-peptide such as a carbohydrate) may be covalently bound for presentation to a cell bearing a T-cell receptor. T-Cell-MMPs comprising a chemical conjugation site for linking an epitope are useful for rapidly preparing T-Cell-MMP-epitope conjugates that can modulate the activity of T-cells specific to the epitope presented and, accordingly, for modulating an immune response in an individual involving those T-cells. The T-Cell-MMPs described herein are suitable for production in cell expression systems where most, substantially all (e.g., greater than 85% or 90% of the T-Cell-MMP), or all of the expressed protein is in a soluble non-aggregated state (e.g., in the form of dimers) that is suitably stable at 37° C. for production in tissue culture and use at least up to that temperature. Most, substantially all (e.g., greater than 85% or 90% of the T-Cell-MMP), or all of the expressed protein remains in a soluble non-aggregated state even after conjugation to epitope peptides and is similarly stable. The T-Cell-MMPs and their epitope conjugates may additionally comprise sites for the conjugation of bioactive substances (payloads) such as chemotherapeutic agents for co-delivery with a specific target epitope. As such, T-Cell-MMP-epitope conjugates may be considered a means by which to deliver MODs (e.g., IL-2, 4-1BBL, FasL, TGF-β, CD70, CD80, CD86, OX40L, ICOS-L, ICAM, JAG1, or fragments thereof, or altered (mutated) variants thereof) and/or payloads (e.g., chemotherapeutics) to cells in an epitope specific manner.

In embodiments described herein the T-Cell-MMPs may comprise modifications that assist in the stabilization of the T-Cell-MMP during intracellular trafficking and/or following secretion by cells expressing the multimeric polypeptide even in the absence of an associated epitope peptide. In embodiments described herein the T-Cell-MMPs may include modifications that link the carboxyl end of the MHC-I α₁ helix and the amino end of the MHC-I α₂₋₁ helix. Such modifications include the insertion of cysteine residues that result in the formation of disulfide linkages linking the indicated regions of those helices. For example, the insertion of cysteine residues at amino acid (aa) 84 (Y84C substitution) and 139 (A139C substitution) of MHC-I, or the equivalent positions relative to the sequences forming the helices, may form a disulfide linkage that helps stabilize the T-Cell-MMP. See, e.g., Z. Hein et al. (2014), Journal of Cell Science 127:2885-2897.

### BRIEF DESCRIPTION OF THE DRAWINGS

FIG. 1 depicts preferential activation of an epitope-specific T-cell to an epitope non-specific T-cell by an embodiment of a T-Cell-MMP of the present disclosure bearing an epitope attached by chemical coupling (denoted by “CC”) to a β-2 microglobulin (β2M) polypeptide sequence.

FIGS. 2A-2G provide amino acid sequences of immunoglobulin Fc polypeptides (including SEQ ID NOs. 1-13).

FIGS. 3A, 3B and 3C provide amino acid sequences of human leukocyte antigen (HLA) Class I heavy chain polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO:14; entry 3A.2 is HLA-A*1101, SEQ ID NO:15; entry 3A.3 is HLA-A*2402, SEQ ID NO:16, and entry 3A.4 is HLA-A*3303, SEQ ID NO:17. FIG. 3B provides the sequence for HLA-B*07:02:01 (HLA-B*0702) (NCBI GenBank Accession NP_005505.2 (see, also, GenBank Accession SEQ ID NO:18FIG. 3C provides the sequence for HLA-C*0701 (GenBank Accession NP_001229971.1) (HLA-C*07:01:01:01 or HLA-Cw*070101), (HLA-Cw*07) (see GenBank Accession CAO78194.1), SEQ ID NO:19.

FIG. 3D provides an alignment of eleven mature MHC Class I heavy chain peptide sequences without all, or substantially all, of their leader, transmembrane and intracellular domain regions. The aligned sequences include human HLA-A*0101, SEQ ID NO:20 (see also SEQ ID NO:14); HLA-B*0702, SEQ ID NO:21; HLA-C, SEQ ID NO:22; HLA-A*0201, SEQ ID NO:23; a mouse H2K protein sequence, SEQ ID NO:24; three variants of HLA-A (var. 2, var. 2C [having Y84C and A139C substitutions], and var. 2CP), SEQ ID NOs:25-27; 3 human HLA-A molecules (HLA-A*1101 (HLA-A11), SEQ ID NO:28; HLA-A*2402 (HLA-A24), SEQ ID NO:29; and HLA-A*3303 (HLA-A33), SEQ ID NO:30). HLA-A*0201 is a variant of HLA-A. The Y84A and A236C variant of HLA-A is marked as HLA-A (var. 2). The seventh HLA-A sequence, marked as HLA-A (var. 2C), shows HLA-A substituted with C residues at positions 84, 139 and 236, and the eighth sequence adds one additional proline to the C-terminus of the preceding sequence. The ninth through the eleventh sequences are from HLA-A11 (HLA-A*1101); HLA-A24 (HLA-A*2402); and HLA-A33 (HLA-A*3303), respectively, which are prevalent in certain Asian populations. Indicated in the alignment are the locations (84 and 139 of the mature proteins) where cysteine residues may be inserted in place of the aa at that position for the formation of a disulfide bond to stabilize the MHC-H-β2M complex in the absence of a bound epitope peptide. Also shown in the alignment is position 236 (of the mature polypeptide), which may be replaced by a cysteine residue that can form an interchain disulfide bond with β2M (e.g., at aa 12 of the mature polypeptide). An arrow appears above each of those locations and the residues are bolded. The boxes flanking residues 84, 139 and 236 show the groups of five aas on either side of those six sets of five residues, denoted aa cluster 1, aa cluster 2, aa cluster 3, aa cluster 4, aa cluster 5, and aa cluster 6 (shown in the figure as aac 1 through aac 6, respectively), that may be replaced by 1 to 5 aas selected independently from (i) any naturally occurring aa or (ii) any naturally occurring aa except proline or glycine.

FIGS. 3E-3G provide alignments of the aa sequences of mature HLA-A, -B, and -C class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac 1 to aac 6) that may be replaced by 1 to 5 aas selected independently from (i) any naturally occurring aa or (ii) any naturally occurring aa except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for each of HLA-E, -F, and -G with the variable aa positions indicated as “X” residues sequentially numbered and the locations of aas 84, 139 and 236 double underlined.

FIG. 3I provides an alignment of the consensus aa sequences for HLA-A, -B, -C, -E, F, and -G, which are given in FIGS. 3E to 3H (SEQ ID NOs: 35, 43, and 53-56). The alignment shows the correspondence of aas between the different sequences. Variable residues in each sequence are listed as “X” with the sequential numbering removed. The permissible aas at each variable residue can be determined by reference to FIGS. 3E-3H. As indicated in FIG. 3D, the locations of aas 84, 139 and 236 with their flanking five-aa clusters that may be replaced by 1 to 5 aas selected independently from (i) any naturally occurring aa or (ii) any naturally occurring aa except proline or glycine are also shown.

FIG. 4 provides a multiple aa sequence alignment of β2M precursors (i.e., including the leader sequence) from Homo sapiens (NP_004039.1; SEQ ID NO:57), Pan troglodytes (NP_001009066.1; SEQ ID NO:58), Macaca mulatta (NP_001040602.1; SEQ ID NO:59), Bos taurus (NP_776318.1; SEQ ID NO:60) and Mus musculus (NP_033865.2; SEQ ID NO:61). Underlined aas 1-20 are the signal peptide (sometime referred to as a leader sequence).

FIG. 5 provides six T-Cell-MMP embodiments (structures) marked as A through F. In each case the T-Cell-MMPs comprise: a first polypeptide having an N-terminus and C-terminus and which comprises a first major histocompatibility complex (MHC) polypeptide (MHC-1); and a second polypeptide having an N-terminus and C-terminus and a second MHC polypeptide (MHC-2), and optionally comprising an immunoglobulin (Fc) polypeptide or a non-Ig polypeptide scaffold. In the embodiments shown the first and second polypeptides are shown linked by a disulfide bond; however, the T-Cell-MMPs do not require a disulfide linkage or any other covalent linkage between the first and second polypeptides. The T-Cell-MMPs may also comprise independently selected linker sequences indicated by the dashed line (- - -). The first polypeptide, the second polypeptide, or both the first and second polypeptides of the T-Cell-MMP comprise at least one chemical conjugation site. Some potential locations for the first polypeptide chemical conjugation sites (CC-1) and second polypeptide chemical conjugation sites (CC-2) are shown by arrows. Locations for one or more MODs that are selected independently (e.g., a sequence comprising one, two, three or more MODs connected in sequence with optional aa linkers between the MODs) are shown by “MOD” in the stippled box. The MODs may be variant MODs as described within this disclosure. In A the MOD(s) are located at the C-terminus of the first polypeptide, in B the MOD(s) are located at the N-terminus of the second polypeptide, in C the MOD(s) are located at the C-terminus of the second polypeptide, in D the MODs are located at the C-terminus of the first peptide and the N-terminus of the second peptide, in E the MOD(s) are added with the epitope peptide, and in F the MOD(s) are between the MHC-2 and Fc peptide. Where more than one MOD is present they may be the same (e.g., two IL-2 MODs) or different, and may be placed adjacent to each other.

FIG. 6 provides twelve embodiments of T-Cell-MMP-epitope conjugates, marked as A through L, that parallel the embodiments in FIG. 5. As in FIG. 5, the first polypeptide has an N-terminus and C-terminus with the first MHC polypeptide given as comprising a β-2-microglobulin polypeptide (β2M capable of interacting with the MHC Class I heavy chain (MHC-H) and presenting the epitope to a T-Cell receptor. The second polypeptide has an N-terminus and C-terminus and a MHC-H polypeptide, and optionally comprises an immunoglobulin (Fc) polypeptide or a non-Ig polypeptide scaffold. The optional disulfide bond joining the first and second polypeptides of the T-Cell-MMP-epitope conjugates is shown connecting the β2M peptide sequence and MHC-H peptide sequence in A to F, and the independently selected optional linker sequences, indicated by the dashed line (- - -), are not required. In G to L, the complexes in A to F are repeated; however, a disulfide bond joining the first and second polypeptides is shown joining the MHC-H peptide sequence to a linker sequence interposed between the epitope and β2M peptide sequence (e.g., a bond from a Cys residue at position 84 of a MHC-H chain sequence as indicated in FIG. 3 to the interposed linker). The first polypeptide, the second polypeptide, or both the first and second polypeptides of the T-Cell-MMP may also comprise one or more chemical conjugation sites in addition to the site employed for the conjugation of the epitope. The potential locations for such sites (CC-1 and CC-2) are shown by arrows. The one or more immunomodulatory polypeptides (either MODs or variant MODs) are as described in FIG. 5. The MODs (e.g., tandem IL-2 polypeptides) may be placed on the N-terminus of the MHC-H polypeptide (Position 1 as in B and H), between the MHC-H and Fc (Position 2 as in F and L), on the C-terminus of the MHC-H (Position 3 as in C and I); N-terminal to the peptide (Position 4 as in E and K); or C-terminal to the β2M (Position 5 as in A and G).

FIG. 7 provides examples of two dimers formed from T-Cell-MMPs. The dimer labeled “A” is the result of dimerizing two of the T-Cell-MMPs labeled “A” in FIG. 6. The dimer labeled “B” is the result of dimerizing two of the T-Cell-MMPs labeled “B” in FIG. 6. The embodiment as shown includes one or more disulfide bonds between the polypeptides, each of which is optional. In addition, only a subset of CC-2 sites in the Fc region or the attached optional linker are shown.

FIG. 8 shows some schematics of epitopes having a maleimide group appended for conjugation to a free nucleophile (e.g., cysteine) present in a T-Cell-MMP to form an epitope conjugate. In “a” the maleimide group is attached by an optional linker (e.g., a peptide linker sequence) to the epitope. In “b” through “e,” the linker is a glycine serine polypeptide (GGGGS) repeated n times, where n is 1-5 when present, and n is 0 when the linker is absent. In “c”-“e” the attachment of a maleimide group is through a lysine (K), such as through the epsilon amino group of the lysine. In “d” and “e” the maleimide group is linked to the peptide through an alkyl amide formed with the epsilon amino group of a lysine residue, where m is 1-7.

FIG. 9 shows in part A a map of a T-Cell-MMP with the first polypeptide having a sulfatase motif (aas 26-31 bolded) between two linker sequences as the location for developing a chemical conjugation site (an fGly residue) through the action of an FGE enzyme. At B, FIG. 9 shows a second polypeptide of a T-Cell-MMP having tandem IL-2 MODs attached to the amino end of a human MHC Class I HLA-A heavy chain polypeptide followed by a human IgG1 Fc polypeptide. Linkers are bolded, italicized and underlined.

FIG. 10A to FIG. 10 D show a series of HLA A*1101 heavy chain constructs having, from N-terminus to C-terminus, a human IL-2 signal sequence, shown in underline and bold. The signal (leader) sequence is followed by a MOD, which is indicated as a human IL-2 or an “optional peptide linker-immunomodulatory polypeptide-optional peptide linker.” Where the MOD is not specified, it may be any desired MOD. The remainder of the sequence is HLA A*1101 H chain sequence with three cysteine substitutions (Y84C; A139C; A236C); a linker; and a hIgG1 Fc with two aa substitutions (L234A; L235A). The asterisks indicate stops to the sequences.

FIG. 11A to FIG. 11G provide aa sequences of hepatitis B virus polypeptides.

FIG. 12A provides aa sequences of an HPV E6 polypeptide and FIG. 12B provides aa sequences of an HPV E7 polypeptide (FIG. 10B).

FIG. 13 shows a comparison of two immunomodulatory proteins each having a first polypeptide (comprising a β2M polypeptide sequence) and a second polypeptide (comprising an MHC-H chain α1-α3 segments and an IgFc). The immunomodulatory proteins appear as dimers comprising two copies of both the first and second polypeptides that are associated by a disulfide bond between the β2M and MHC-H sequences and disulfide bonds between the Fc regions. Structure A is a control immunomodulatory protein that has a 9 aa cytomegalovirus (CMV) epitope at the N-terminus of a β2M polypeptide sequence of the first polypeptide. Structure B is a T-Cell-MMP of the present disclosure having a chemical conjugation site indicated with an “*” in the β2M polypeptide sequence; in this instance the chemical conjugation site is within a linker (not shown) at the N-terminus of a β2M polypeptide sequence. An SDS PAGE gel of the expressed and purified proteins under non-reducing and reducing conditions is shown at C with molecular weight markers (left lane), the non-reduced samples conjugated to MART1 and CMV peptides are in the 2nd and 3ird lanes from the left, reduced samples conjugated to MART1 and CMV peptides are in the 4th and 5th lanes from the left. The first polypeptides are labeled as “light chain” and the second polypeptides are labeled as “heavy chain.” See Example 2 for more details.

FIG. 14 shows size exclusion chromatography of T-Cell-MMPs conjugated to CMV (CMV+T-Cell-MMP) and MART-1 (MART+T-Cell-MMP) polypeptides plotted in mAU (milli-absorbance units) vs time in minutes. See example 3.

FIG. 15. shows the response of Ficoll-Paque® samples of leukocytes from CMV responsive donors simulated with various concentrations of MOD constructs and control treatments measured as the number of CMV or MART-1 responsive CD8+ T-cells. See Example 4 for details.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

A polynucleotide or polypeptide has a certain percent “sequence identity” to another polynucleotide or polypeptide, meaning that, when aligned, that percentage of bases or aas is the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.) available over the world wide web at sites including ncbi.nlm.nili.gov/BLAST, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless stated otherwise, sequence alignments are prepared using BLAST.

The terms “amino acid” and “amino acids” are abbreviated as “aa” and “aas,” respectively. Naturally occurring aa or naturally occurring aas, unless stated otherwise, means: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan); all of the L-configuration. Both selenocysteine and hydroxyproline are naturally occurring aas that are specifically referred to in any instance where they are intended to be encompassed.

Non-natural aas are any aa other than the naturally occurring aas recited above, selenocysteine, and hydroxyproline.

“Chemical conjugation” as used herein means formation of a covalent bond. “Chemical conjugation site” as used herein means a location in a polypeptide at which a covalent bond can be formed, including any contextual elements (e.g., surrounding aa sequences) that are required or assist in the formation of a covalent bond to the polypeptide. Accordingly, a site comprising a group of aas that direct enzymatic modification, and ultimately covalent bond formation at an aa within the group, may also be referred to as a chemical conjugation site. In some instances, as will be clear from the context, the term chemical conjugation site may be used to refer to a location where covalent bond formation or chemical modification has already occurred.

The term “conservative aa substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The terms “immunological synapse” or “immune synapse” as used herein generally refer to the natural interface between two interacting immune cells of an adaptive immune response including, e.g., the interface between an APC, or target T-cell, and an effector cell, e.g., a lymphocyte, an effector T-cell, a natural killer cell, or the like. An immunological synapse between an APC and a T-cell is generally initiated by the interaction of a T-cell antigen receptor and one or more MHC molecules, e.g., as described in Bromley et al., Ann Rev Immunol. 2001; 19:375-96; the disclosure of which is incorporated herein by reference in its entirety.

“T-cell” includes all types of immune cells expressing CD3, including T-helper cells (CD4⁺ cells), cytotoxic T-cells (CD8⁺ cells), T-regulatory cells (Treg), and NK-T-cells.

Unless stated otherwise, as used herein, the terms “first major histocompatibility complex (MHC) polypeptide” or “first MHC polypeptide”, and the terms “second MHC polypeptide”, “MHC heavy chain”, and “MHC-H”, refer to MHC Class I receptor elements.

A “MOD” (also termed a co-immunomodulatory or co-stimulatory polypeptide), as the term is used herein, includes a polypeptide on an APC (e.g., a dendritic cell, a B cell, and the like), or a portion of the polypeptide on an APC, that specifically binds a “Co-MOD” (also termed a cognate co-immunomodulatory polypeptide or a cognate co-stimulatory polypeptide) on a T-cell, thereby providing a signal which, in addition to the primary signal provided by, for instance, binding of a TCR/CD3 complex with a MHC polypeptide loaded with peptide, mediates a T-cell response including, but not limited to, proliferation, activation, differentiation, and the like. MODs include, but are not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), transforming growth factor beta (TGF-β), CD30, CD40, CD70, CD83, HVEM, lymphotoxin beta receptor, 3/TR6, ILT3, ILT4, HVEM, an agonist or antibody that binds the Toll ligand receptor, and a ligand that specifically binds with B7-H3. A MOD also encompasses, inter alia, an antibody (or an antigen binding portion thereof, such as a Fab) that specifically binds with a Co MOD present on a T-cell, such as, but not limited to, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

An “immunomodulatory domain” (“MOD”) of a T-Cell-MMP is a polypeptide of the T-Cell-MMP or part thereof that acts as a MOD.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments, or from a series of synthetic oligonucleotides, to provide a synthetic nucleic acid which is capable of being expressed from a recombinant transcriptional unit contained in a cell or in a cell-free transcription and translation system.

The terms “recombinant expression vector” and “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated aa sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

“Binding” as used herein (e.g., with reference to binding of a molecule such as a T-cell-MMP comprising one or more MODs or its epitope conjugate to one or more polypeptides (e.g., a T-cell receptor and a Co-MOD on a T-cell) refers to a non-covalent interaction(s) between the molecules. Non-covalent binding refers to a direct association between two molecules due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, or less than 10⁻¹² M. “Affinity” refers to the strength of non-covalent binding, increased binding affinity being correlated with a lower K_{D}. “Specific binding” generally refers to, e.g., binding between a ligand molecule and its binding site or “receptor” with an affinity of at least about 10⁻⁷ M or greater (e.g., less than 5×10⁻⁷ M, less than 10⁻⁸ M, less than 5×10⁻⁸ M, less than 10⁻⁹ M, less than 10⁻¹⁰ M, less than 10⁻¹¹ M, or less than 10⁻¹² M and greater affinity, or in a range from 10⁻⁷ to 10⁻⁹ or from 10⁻⁹ to 10⁻¹²). “Non-specific binding” generally refers to the binding of a ligand to something other than its designated binding site or “receptor,” typically with an affinity of less than about 10⁻⁷ M (e.g., binding with an affinity of less than about 10⁻⁶ M, less than about 10⁻⁵M, less than about 10⁻⁴ M). However, in some contexts, e.g., binding between a TCR and a peptide/MHC complex, “specific binding” can be in the range of from 1 μM to 100 μM, or from 100 μM to 1 mM. “Covalent binding” as used herein means the formation of one or more covalent chemical bonds between two different molecules

The terms “treatment,” “treating” and the like are used herein to generally mean obtaining a desired pharmacologic and/or physiologic effect. The effect may be prophylactic in terms of completely or partially preventing a disease or symptom thereof, and/or may be therapeutic in terms of a partial or complete cure for a disease and/or adverse effect attributable to the disease. “Treatment” as used herein covers any treatment of a disease or symptom in a mammal and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), canines, felines, etc.

Before the present invention is further described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that the range includes each intervening value, to the tenth of the lower limit, unless the context clearly dictates otherwise, between the upper and lower limit of that range and any other stated or intervening value in that stated range, is encompassed within the invention. The upper and lower limits of smaller ranges may independently be included in the smaller ranges, and are also encompassed within the invention, subject to any specifically excluded limit in the stated range. Where a range includes upper and/or lower limits, ranges excluding either or both of those limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that, as used herein and in the appended claims, the singular forms “a,” “an,” and “the” include plural referents unless the context clearly dictates otherwise. Thus, for example, reference to “multimeric T-cell modulatory polypeptide” includes a plurality of such polypeptides and reference to “the immunomodulatory polypeptide” or “the MOD” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

### I. T-Cell Modulatory Multimeric Polypeptides (T-Cell-MMPs) with Chemical Conjugation Sites for Epitope Binding

The present disclosure provides T-Cell-MMP-epitope conjugates that comprise an epitope-presenting peptide that presents, for example, a cancer-associated epitope, an infectious disease-associated epitope (such as a virus or bacterial epitope), or a self-epitope (e.g., the peptide is a cancer-associated peptide, an infectious disease-associated peptide (e.g., a virus- or bacteria-encoded peptide or a peptide of a self-antigen). Such T-Cell-MMP-epitope conjugates are useful for modulating the activity of T cells, and for modulating an immune response in an individual.

The present disclosure provides T-Cell-MMPs and their epitope conjugates that are useful for modulating the activity of a T-cell, and methods of their preparation and use in modulating an immune response in an individual. The T-Cell-MMPs may comprise one or more independently selected wild-type and/or variant MOD polypeptides that exhibit reduced binding affinity to their Co-MODs and chemical conjugation sites for coupling epitopes and payloads. Included in this disclosure are T-Cell-MMPs that are heterodimeric, comprising two types of polypeptides (a first polypeptide and a second polypeptide), wherein at least one of those polypeptides comprises a chemical conjugation site for the attachment (e.g., covalent attachment) of payloads such as chemotherapeutic agents and/or materials (e.g., epitope peptides and null peptides) that can bind a TCR. Also included in this disclosure are T-Cell-MMPs which have been chemically conjugated to an epitope and/or a payload (e.g., a chemotherapeutic). Depending on the type of MOD(s) present in the T-Cell-MMP, when an epitope specific to a TCR is present on a T-Cell-MMP, the T-cell can respond by undergoing activation including, for example, clonal expansion (e.g., when activating MODs such as IL-2, 4-1BBL and/or CD80 are incorporated into the T-Cell-MMP). Alternatively, the T-cell may undergo inhibition that down regulates T-cell activity (e.g., blocking autoimmune reactions) when MODs such as CD86 and/or PD-L1 are incorporated into the T-Cell-MMPs. Because MODs are not specific to any epitope, activation or inhibition of T-cells can be biased toward epitope-specific interactions by incorporating variant MODs having reduced affinity for their Co-MOD into the T-Cell-MMPs such that the binding of a T-Cell-MMP to a T-cell is strongly affected by, or even dominated by, the MHC-epitope-TCR interaction.

A T-Cell-MMP-epitope conjugate may be considered to function as a surrogate APC, and mimics the adaptive immune response. The T-Cell-MMP-epitope conjugate does so by engaging a TCR present on the surface of a T-cell with a covalently bound epitope presented in the T-Cell-MMP-epitope conjugate complex. This engagement provides the T-Cell-MMP-epitope conjugate with the ability to achieve epitope-specific cell targeting. In embodiments described herein, T-Cell-MMP-epitope conjugates also possess at least one MOD that engages a counterpart costimulatory protein (Co-MOD) on the T-cell. Both signals—epitope/MHC binding to a TCR and MOD binding to a Co-MOD—then drive both the desired T-cell specificity and either inhibition or activation/proliferation.

The T-Cell-MMPs having chemical conjugation sites find use as a platform into which different epitopes and/or payloads may be inserted to prepare materials for therapeutic, diagnostic and research applications. Such T-Cell-MMPs comprising a chemical conjugation site permit the rapid preparation of diagnostics and therapeutics as they permit the epitope containing material (e.g., a peptide) to be rapidly inserted into the T-Cell-MMP and tested for activation or inhibition of T-cells bearing TCRs specific to the epitope.

In an embodiment, a chemical conjugation site of such a T-Cell-MMP may be utilized to attach a payload such as a chemotherapeutic agent or enzyme to the T-Cell-MMP. In the absence of an added epitope, the resulting complex may be used in a fashion similar to an antibody to deliver the payload, particularly when the T-Cell-MMPs form multimers (e.g., dimers or higher order structures) due to the incorporation of an Fc scaffold. Due to the lack of an epitope, the MODs of T-Cell-MMP-payload conjugates will dictate the cells that will receive the payload by their binding specificity and the avidity of the complex for different cells.

In an embodiment, where variant MODs that stimulate T-cell proliferation and an epitope are incorporated into a T-Cell-MMP, contacting the T-cells with at least one concentration of the T-Cell-MMP induces at least a twofold (e.g., at least a 2, 3, 4, 5, 10, 20, 30, 50, 75, or 100 fold) difference in the activation of T-cells (as measured by T-cell proliferation or ZAP-70 activity, see e.g., Wang, et al., Cold Spring Harbor perspectives in biology 2.5 (2010): a002279) having a TCR specific to the epitope, as compared to T-cells contacted with the same concentration of the T-Cell-MMP that do not have a TCR specific to the epitope.

In an embodiment where variant MODs that inhibit T-cell activation and an epitope are incorporated into a T-Cell-MMP, contacting the T-cells with at least one concentration of the T-Cell-MMP prevents activation of T-cells in an epitope specific manner as measured by T-cell proliferation.

The specificity of T-Cell-MMPs into which an epitope has been incorporated will depend on the relative contributions of the epitope and MODs to the binding. Where the MODs dominate the T-Cell-MMPs the binding interactions, the specificity of the T-Cell-MMP of T-cells specific to the epitope will be reduced relative to T-Cell-MMP complexes where the epitope dominates the binding interactions by contributing more to the overall binding energy than the MODs. The greater the contribution of the epitope to a TCR specific to the epitope, the greater the specificity of the T-Cell-MMP will be for that T-cell type. Where an epitope has strong affinity for its TCR, the use of variant MODs with reduced affinity for their Co-MODs will favor epitope selective interactions of the T-Cell-MMP-epitope conjugates, and also facilitate selective delivery of any payload that may be conjugated to the T-Cell-MMP-epitope conjugate.

In addition to being useful as a structure into which to incorporate epitopes and prepare T-Cell-MMPs that are epitope specific, the T-Cell-MMPs described as either lacking an epitope or containing a null peptide may be employed to deliver a payload to target cells bearing receptors for the MODs and/or variant MODs present in the T-Cell-MMPs.

In an embodiment, T-Cell-MMPs bearing MODs inhibitory to T-cell activation and/or proliferation that lack an epitope (or contain a null peptide) may be used as stimulators of T-cells that contain one or more receptors for the MOD or variant MODs present in the T-Cell-MMP. Such stimulatory T-Cell-MMPs may be used to simultaneously deliver a payload (e.g., a chemically conjugated chemotherapeutic) to the T-cells to which the T-Cell-MMPs binds.

In an embodiment, T-Cell-MMPs bearing MODs inhibitory to T-cell activation and/or proliferation that lack an epitope (or that contain a null peptide) may be used as an immunosuppressant alone or in conjunction with other immunosuppressants such as cyclosporine to suppress immune reactions (e.g., prevent graft-v-host or host-v-graft rejection). Such inhibitory T-Cell-MMPs may be used to simultaneously deliver a payload (e.g., a chemically conjugated chemotherapeutic) to the T-cells to which the T-Cell-MMPs binds.

The present disclosure provides T-Cell-MMPs that are useful for modulating the activity of a T-cell and, accordingly, for modulating an immune response in an individual. The T-Cell-MMPs comprise a MOD that exhibits reduced binding affinity to a Co-MOD.

A. T-Cell-MMPs and T-Cell-MMP Epitope Conjugates

The T-Cell-MMP frameworks described herein comprise at least one chemical conjugation site on either the first polypeptide chain or the second polypeptide chain.

In an embodiment, the present disclosure provides a T-Cell-MMP comprising a heterodimer comprising: a) a first polypeptide comprising: a first MHC polypeptide; b) a second polypeptide comprising a second MHC polypeptide; c) at least one of first or second polypeptides comprises a chemical conjugation site, and d) at least one MOD, where the first and/or the second polypeptide comprises the at least one MOD (e.g., one, two, three, or more). Optionally, the first or the second polypeptide comprises an Ig Fc polypeptide or a non-Ig scaffold. One or more of the MODs, which are selected independently, may be a variant MOD that exhibits reduced affinity to a Co-MOD compared to the affinity of a corresponding wild-type MOD for the Co-MOD. The disclosure also provides T-Cell-MMPs in which an epitope (e.g., a peptide bearing an epitope) is covalently bound (directly or indirectly) to the chemical conjugation site forming a T-Cell-MMP-epitope conjugate. In such an embodiment, the epitope (e.g., epitope peptide) present in a T-Cell-MMP-epitope conjugate of the present disclosure may bind to a T-cell receptor (TCR) on a T-cell with an affinity of at least 100 micro molar (μM) (e.g., at least 10 μM, at least 1 μM, at least 100 nM, at least 10 nM, or at least 1 nM). A T-Cell-MMP-epitope conjugate may bind to a first T-cell with an affinity that is at least 25% higher than the affinity with which the T-Cell-MMP-epitope conjugate binds to a second T-cell, where the first T-cell expresses on its surface the Co-MOD and a TCR that binds the epitope with an affinity of at least 100 μM, and where the second T-cell expresses on its surface the Co-MOD but does not express on its surface a TCR that binds the epitope with an affinity of at least 100 μM (e.g., at least 10 μM, at least 1 μM, at least 100 nM, at least 10 nM, or at least 1 nM).

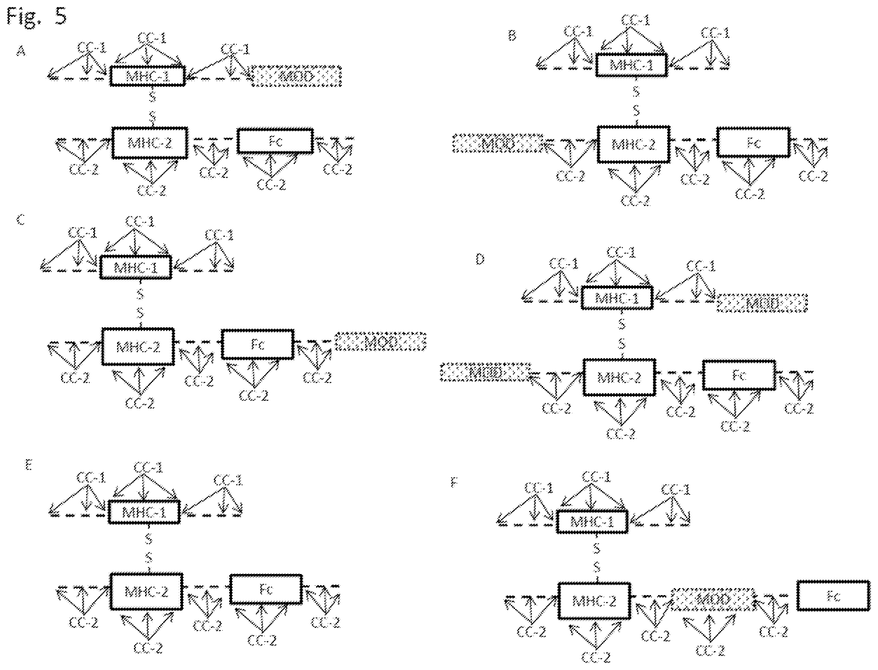

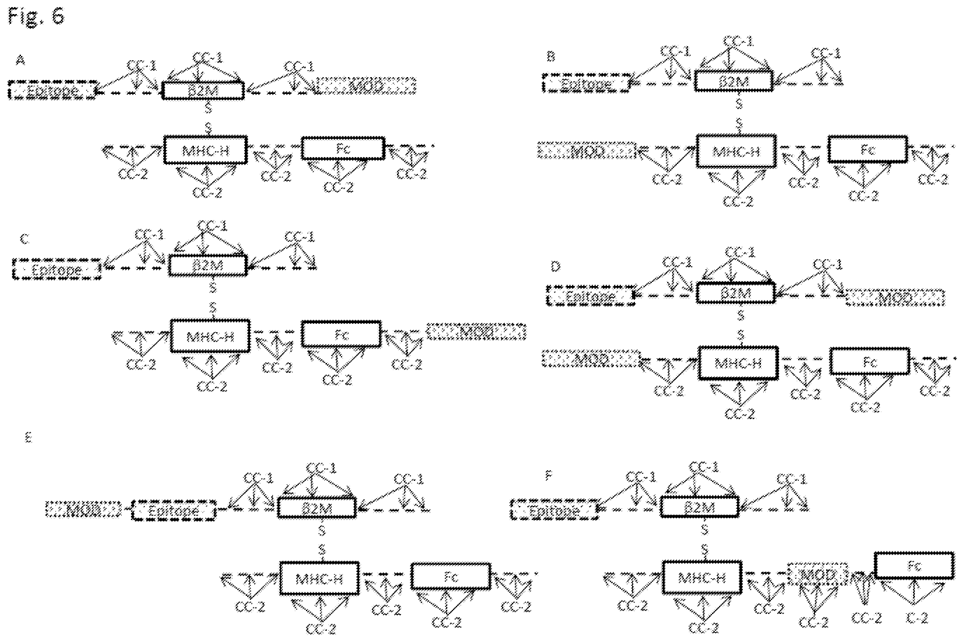

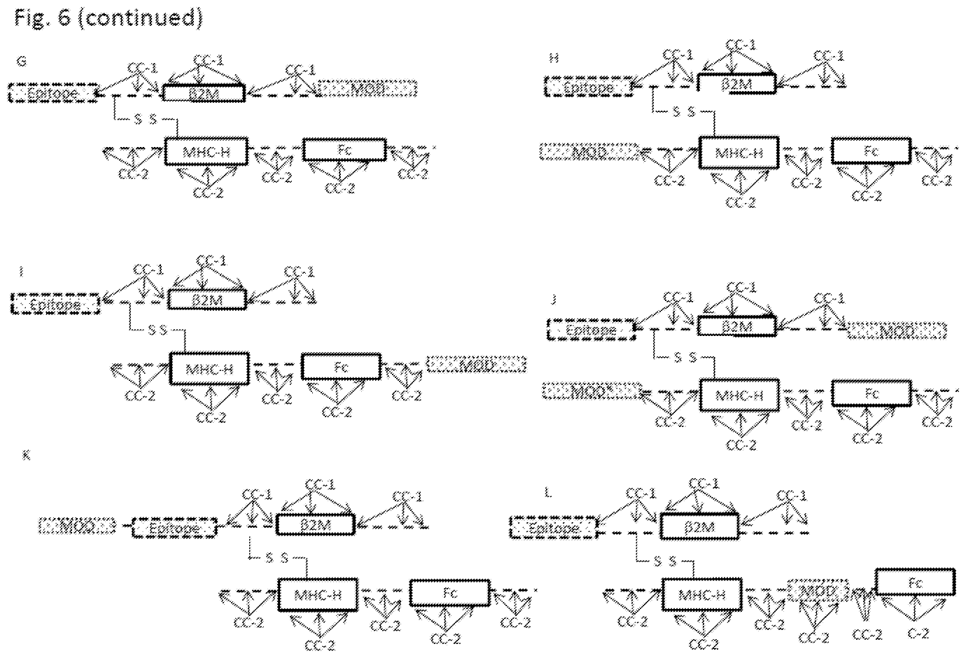

In an embodiment, the present disclosure provides a heterodimeric T-Cell-MMP (which may form higher level multimers, dimers, trimers, etc. of the heterodimers) comprising:
a) a first polypeptide comprising a first MHC polypeptide;
b) a second polypeptide comprising, in order from N-terminus to C-terminus: i) a second MHC polypeptide and ii) an optional immunoglobulin (Ig) Fc polypeptide scaffold or a non-Ig polypeptide scaffold;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
   A) at the C-terminus of the first polypeptide (see, e.g., A in FIG. 5 or 6),
   B) at the N-terminus of the second polypeptide (see, e.g., B in FIG. 5 or 6),
   C) at the C-terminus of the second polypeptide (see, e.g., C in FIG. 5 or 6), or
   D) at the C-terminus of the first polypeptide and at the N-terminus of the second polypeptide (see, e.g., D in FIG. 5 or 6);
wherein each of the one or more MODs is an independently selected wild-type or variant MOD.

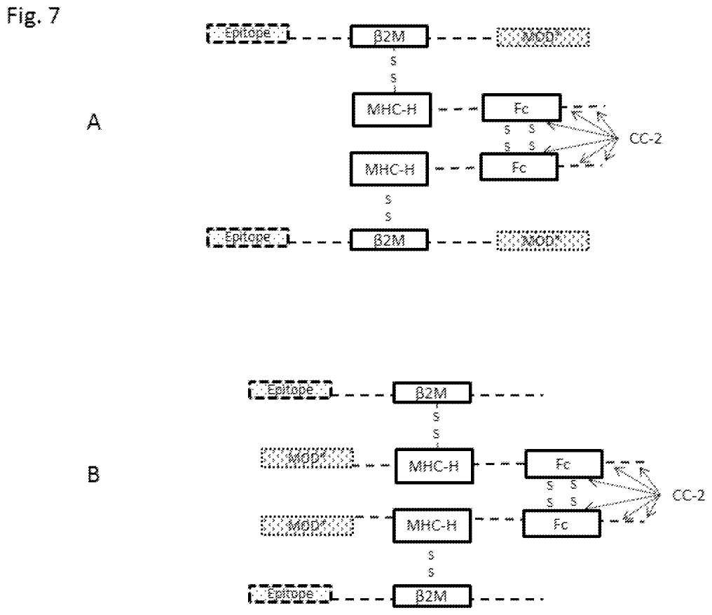

Such T-Cell-MMP frameworks act as a platform on which epitopes (e.g., polypeptide epitopes such as a cancer-associated epitope, an infectious disease-associated epitope, or a self-epitope) can be covalently attached through a linkage to one of the first or second chemical conjugation sites bound to at least one of the first and second MHC polypeptides forming a T-Cell-MMP-epitope conjugate. This permits facile introduction of different epitopes into the framework for presentation in the context of the T-Cell-MMP to a T-cell receptor (TCR) on a T-cell. Payload (e.g., chemotherapeutics) can similarly be attached to a T-Cell-MMP by covalent attachment to one of the first or second chemical conjugation sites (e.g., a site not employed for attachment of an epitope).

Where an immunoglobulin (Ig) Fc polypeptide or a non-Ig polypeptide scaffold that can multimerize is employed, the T-Cell-MMPs may multimerize. The complexes may be in the form of dimers (see, e.g., FIG. 7), trimers, tetramers, or pentamers. Compositions comprising multimers of T-Cell-MMPs may also comprise monomers and, accordingly, may comprise monomers, dimers, trimers, tetramers, pentamers, or combinations of any thereof (e.g., a mixture of monomers and dimers).

In an embodiment, the MODs are independently selected wild-type MODs and/or variant MODs presented in a T-Cell-MMP that optionally comprises an epitope. In an embodiment, the MODs are one or more wt MODs and/or variant MODs capable of stimulating epitope-specific T-cell activation/proliferation (e.g., IL-2, 4-1BBL and/or CD80). In another embodiment, the MODs are one or more wt MODs and/or variant MODs capable of inhibiting T-cell activation/proliferation (e.g., FAS-L and/or PD-L1). When used in conjunction with a T-Cell-MMP bearing a suitable epitope, such activating or inhibitory MODs are capable of epitope-specific T-cell action, particularly where the MODs are variant MODs and the MHC-epitope-TCR interaction is sufficiently strong to dominate the interaction of the T-Cell-MMP with the T-cells.

1 Locations of the First and Second Chemical Conjugation Sites in T-Cell-MMPs

Prior to being subject to chemical conjugation reactions that incorporate an epitope (e.g., an epitope containing peptide) and/or payload, the T-Cell-MMPs described herein comprise at least one chemical conjugation site. Where the T-Cell-MMPs comprise more than one chemical conjugation site, there may be two or more conjugation sites on the first polypeptide (first polypeptide chemical conjugation sites), two or more conjugation sites on the second polypeptide (second polypeptide chemical conjugation sites), or at least one first polypeptide chemical conjugation site and at least one second polypeptide chemical conjugation site. In each instance where more than one chemical conjugation site is present in a T-Cell-MMP molecule, the sites are independently selected and may employ the same or different chemistries, amino acid sequences, or chemical groups for conjugation. Some examples of the locations for first polypeptide chemical conjugation sites (indicated as CC-1) and second polypeptide chemical conjugation sites (indicated as CC-1) are shown in FIGS. 5-7.

In embodiments, the first polypeptide of the T-Cell-MMPs comprise: a first MHC polypeptide without a linker on its N-terminus and C-terminus; a first MHC polypeptide bearing a linker on its N-terminus; a first MHC polypeptide bearing a linker on its C-terminus, or a first MHC polypeptide bearing a linker on its N-terminus and C-terminus. At least one of the one or more first polypeptide chemical conjugation sites is: a) attached to (e.g., at the N- or C-terminus), or within, the sequence of the first MHC polypeptide when the first MHC polypeptide is without a linker on its N- and C-termini; b) attached to, or within, the sequence of the first MHC polypeptide, where the first MHC polypeptide comprises a linker on its N- and C-termini; c) attached to, or within, the sequence of a linker on the N-terminus of the first MHC polypeptide; and/or d) attached to, or within, the sequence of a linker on the C-terminus of the first MHC polypeptide. Additional first polypeptide chemical conjugation sites of a T-Cell-MMP may be present at (attached to or within) any location on the first polypeptide (e.g., more than one enzyme modification sequence serving as a site for chemical conjugation), including the first MHC polypeptide, or in any linker attached to the MHC peptide. In such embodiments, the first MHC polypeptide may comprise a β2M polypeptide sequence as described below.

In embodiments, the second polypeptide of the T-Cell-MMPs comprise: a second MHC polypeptide without a linker on its N-terminus and C-terminus; a second MHC polypeptide bearing a linker on its N-terminus; a second MHC polypeptide bearing a linker on its C-terminus, or a second MHC polypeptide bearing a linker on its N-terminus and C-terminus. At least one of the one or more second polypeptide chemical conjugation sites is: a) attached to (e.g., at the N- or C-terminus), or within, the sequence of the second MHC polypeptide when the second MHC polypeptide is without a linker on its N- and C-termini; b) attached to, or within, the sequence of the second MHC polypeptide where the second MHC polypeptide comprises a linker on its N- and C-termini; c) attached to, or within, the sequence of the linker on the N-terminus of the second MHC polypeptide; and/or d) attached to, or within, the sequence of the linker on the C-terminus of the second MHC polypeptide. In addition, when the second polypeptide contains an immunoglobulin (Fc) polypeptide aa sequence or a non-Ig polypeptide scaffold, along with an additional linker attached thereto, the second polypeptide chemical conjugation sites may be attached to or within the second MHC polypeptide, the immunoglobulin polypeptide, the polypeptide scaffold, or the attached linker. Additional second polypeptide chemical conjugation sites of a T-Cell-MMP may be present at (attached to or within) any location on the second polypeptide (e.g., more than one enzyme modification sequence serving as a site for chemical conjugation), including the second MHC polypeptide, or in any linker attached to it. In such embodiments, the second MHC polypeptide may comprise a MHC heavy chain (MHC-H) polypeptide sequence as described below.

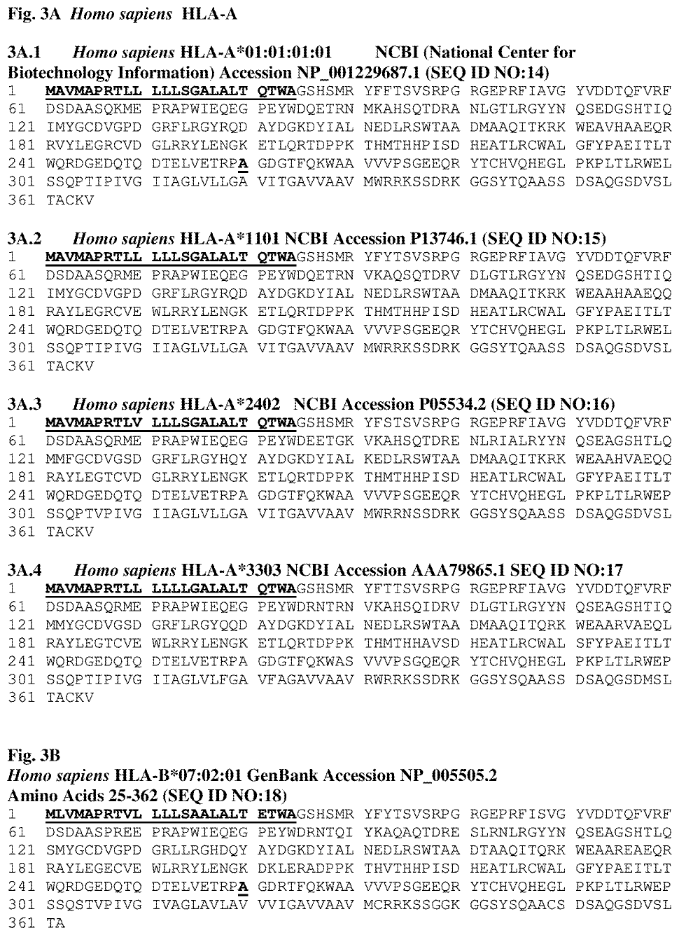

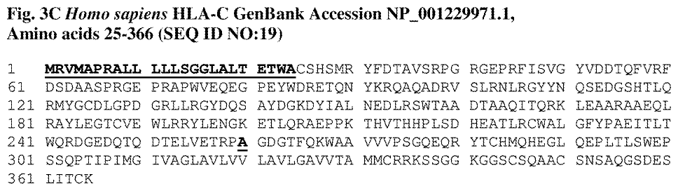

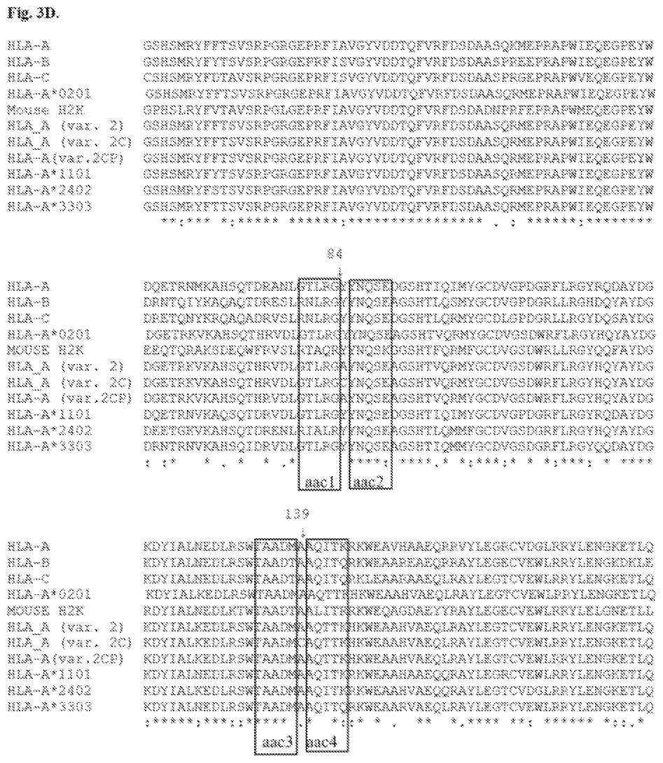

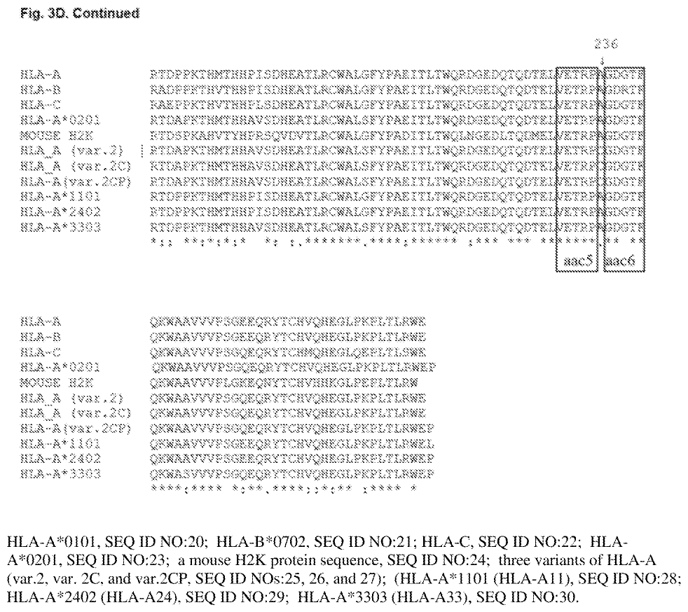

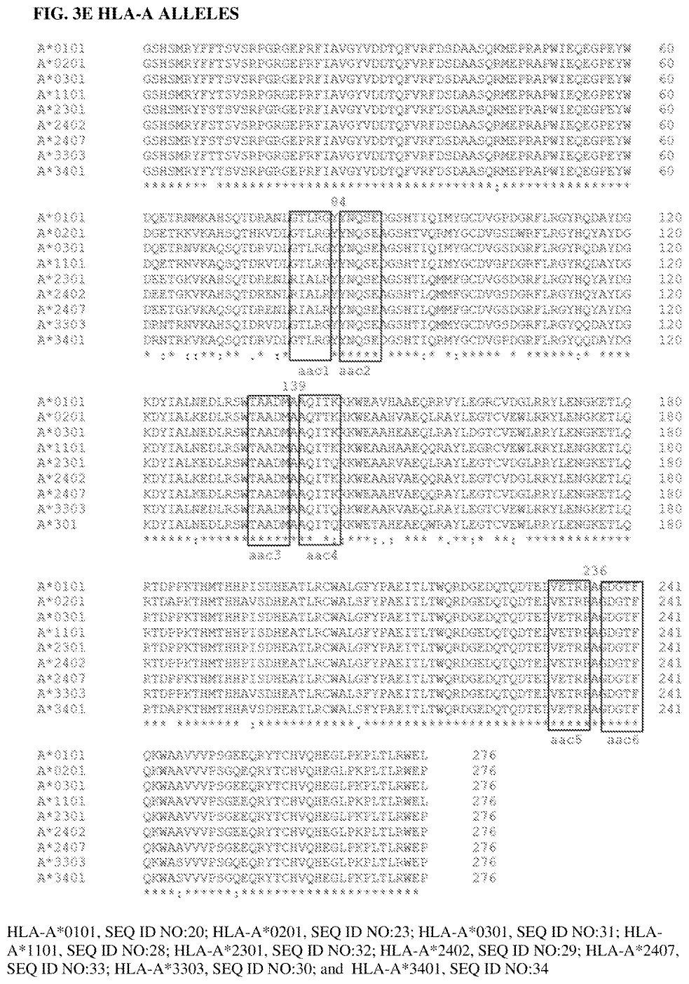

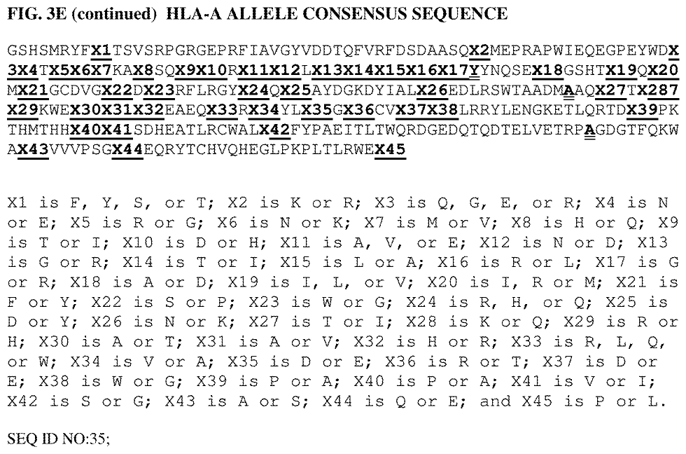

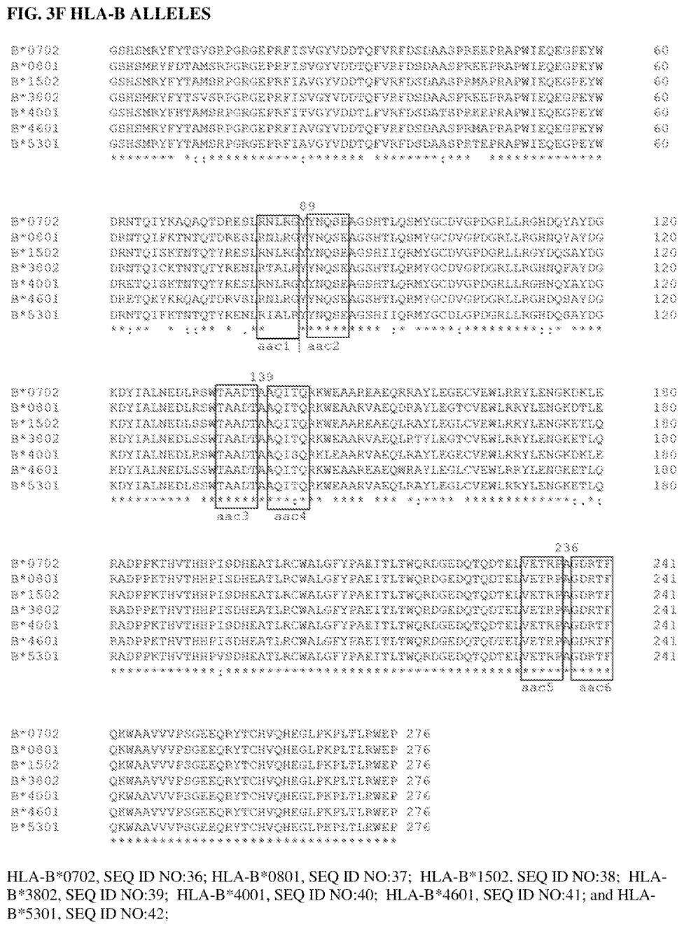

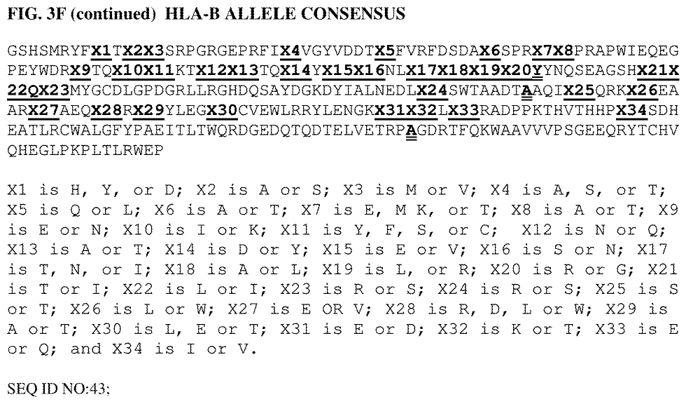

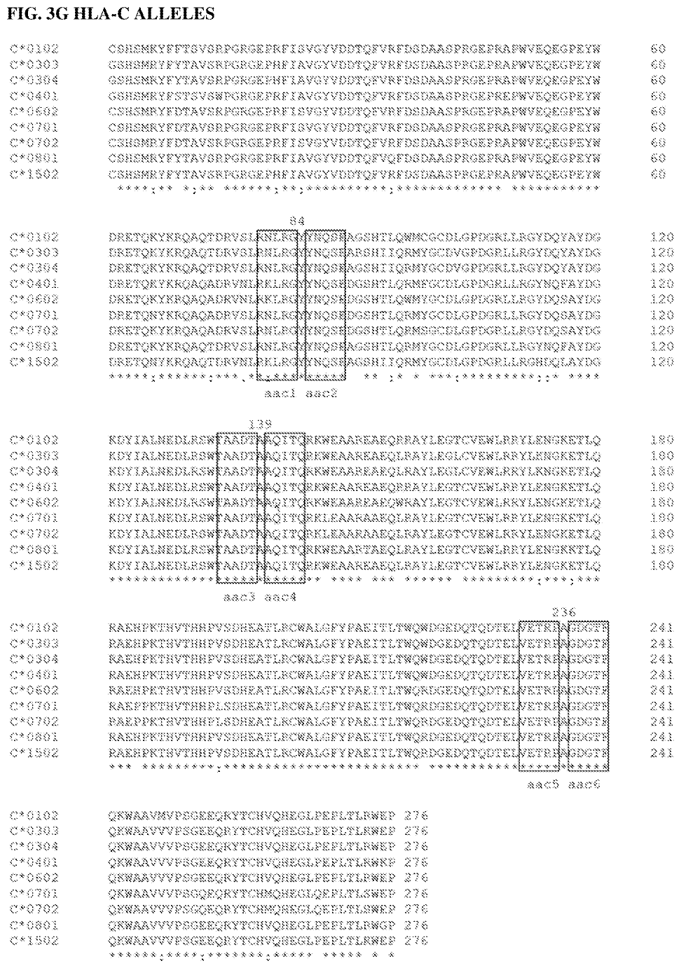

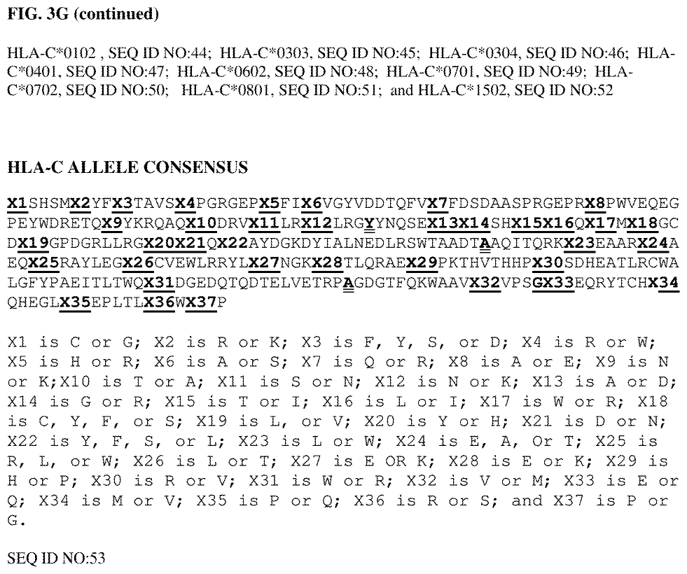

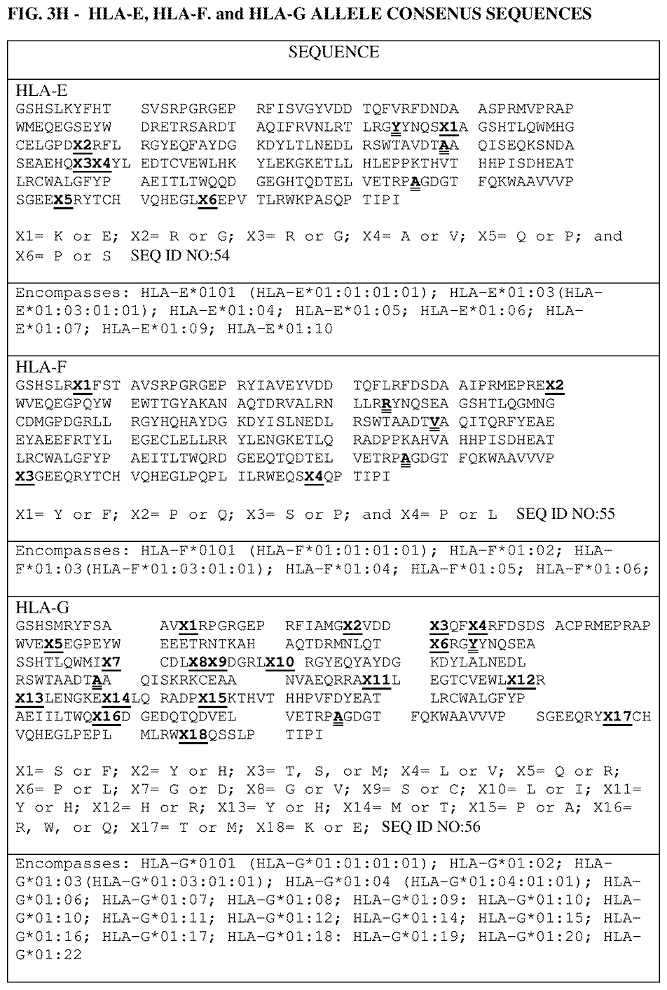

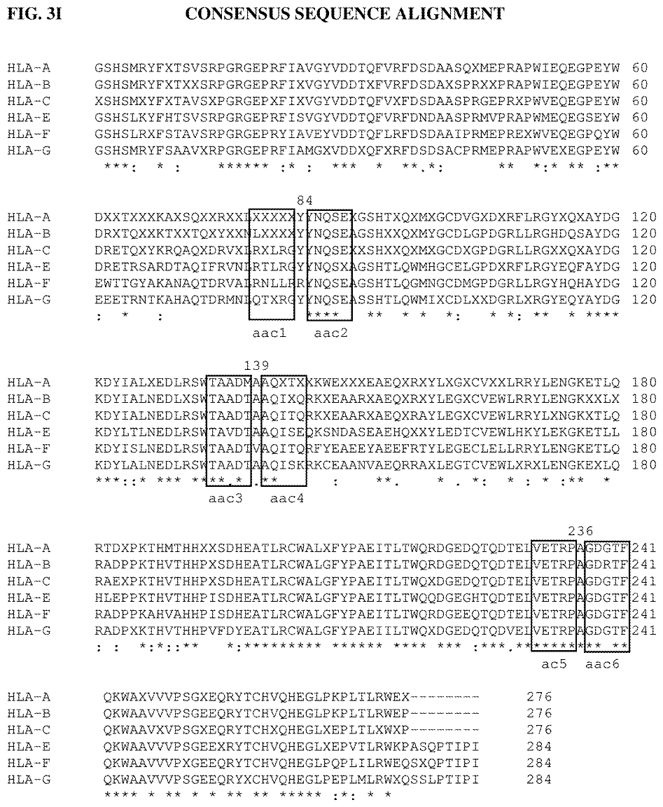

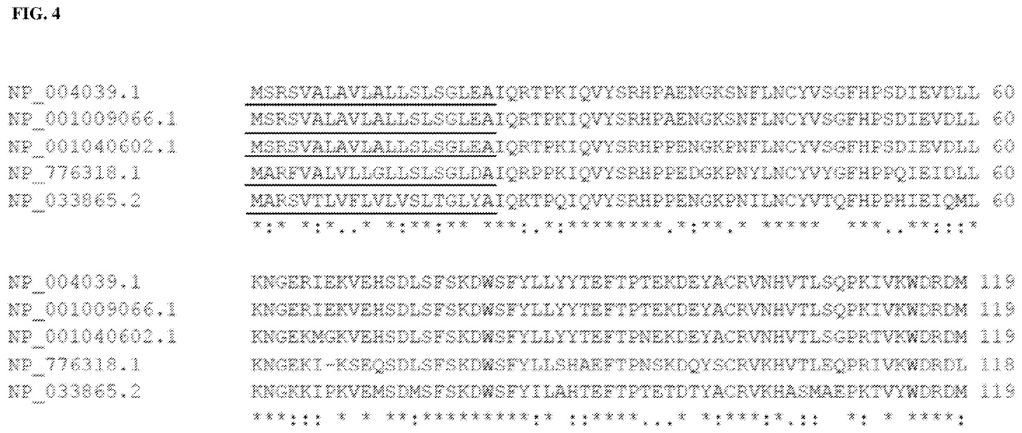

In an embodiment, the first and second MHC polypeptides may be selected to be Class I MHC polypeptides, with the first MHC polypeptide comprising a β2M polypeptide sequence and the second polypeptide comprising a MHC heavy chain sequence, wherein there is at least one chemical conjugation site on the first or second polypeptide. In an embodiment, at least one of the one or more first chemical conjugation sites in the T-Cell-MMP may be attached to (including at the N- or C-terminus) or within either the β2M polypeptide or the linker attached to its N-terminus or C-terminus. In an embodiment, at least one of the one or more second polypeptide chemical conjugation sites in the T-Cell-MMP may be attached to (including at the N- or C-terminus) or within: the MHC-H polypeptide; a linker attached to the N-terminus or C-terminus of the MHC-H polypeptide; or, when present, attached to or within an immunoglobulin (Fc) polypeptide (or a non-Ig polypeptide scaffold) or a linker attached thereto. In another embodiment of such a Class I MHC polypeptide construct, both the first and second polypeptides comprise at least one chemical conjugation site.

Where the T-Cell-MMP comprises a β2M polypeptide sequence, the sequence may have at least 85% aa sequence identity (e.g., at least 90%, 95%, 98% or 99% identity, or even 100% identity) to one of the aa sequences set forth in FIG. 4. The β2M polypeptide may comprise an aa sequence having at least 20, 30, 40, 50, 80, 100, or 110 contiguous aas with identity to a portion of an aa sequence set forth in FIG. 4. The chemical conjugation sequences can be attached to the β2M polypeptide (e.g., at the N- and/or C-termini or linkers attached thereto) or within the β2M polypeptide.

Where the T-Cell-MMP comprises a MHC-H polypeptide, it may be a HLA-A, -B, -C, -E, -F, or -G heavy chain. In an embodiment, the MHC-H polypeptide may comprise an aa sequence having at least 85% aa sequence identity (e.g., at least 90%, 95%, 98% or 99% identity, or even 100% identity) to the aa sequence set forth in one of FIGS. 3A-3H. The MHC Class I heavy chain polypeptides may comprise an aa sequence having at least 20, 30, 40, 50, 80, 100, 150, 200, 250, 300, or 330 contiguous aas with identity to a portion of an aa sequence set forth in FIGS. 3A-3H. The chemical conjugation sequences can be attached (e.g., at the N- and/or C-termini or linkers attached thereto) or within the MHC-H polypeptides.

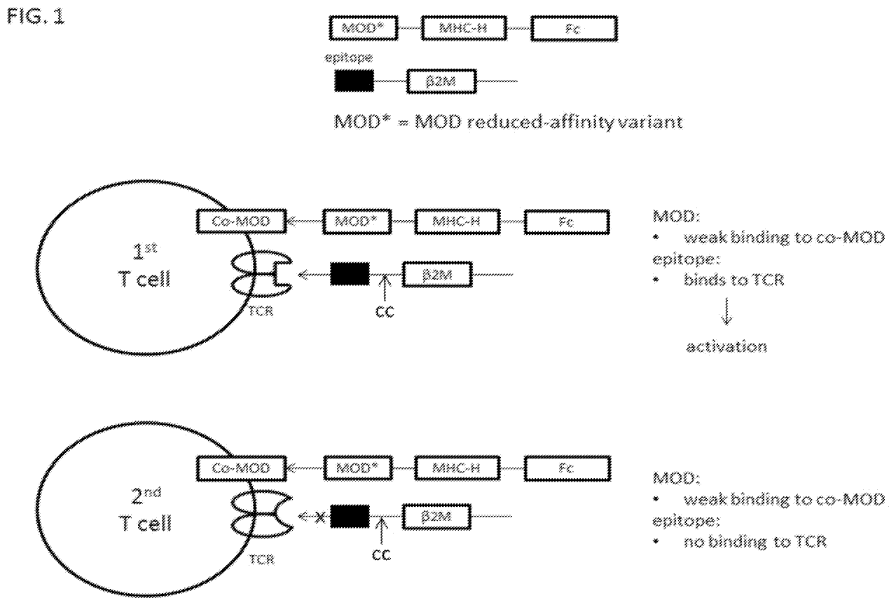

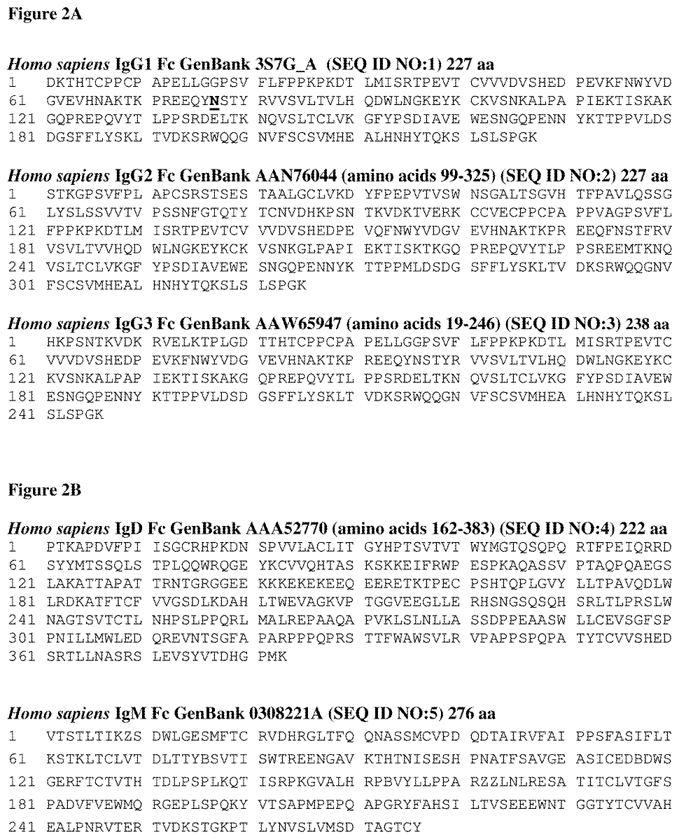

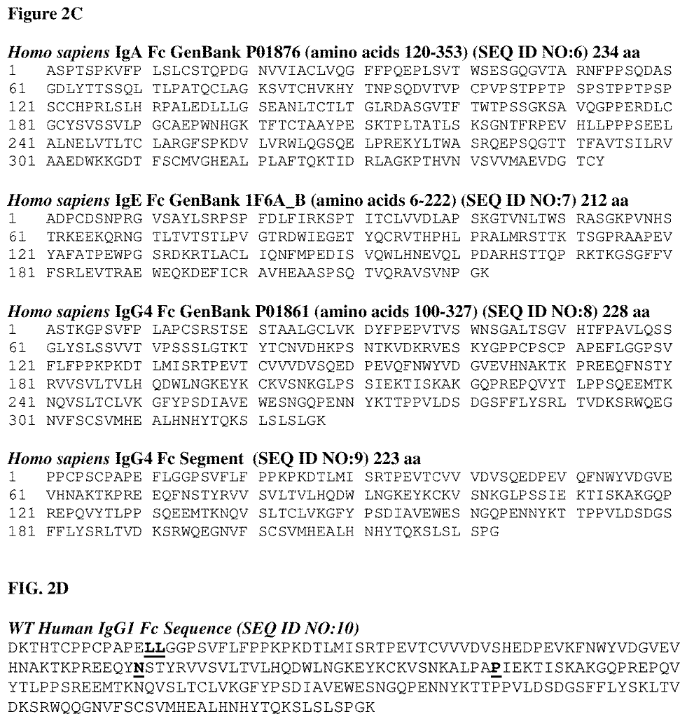

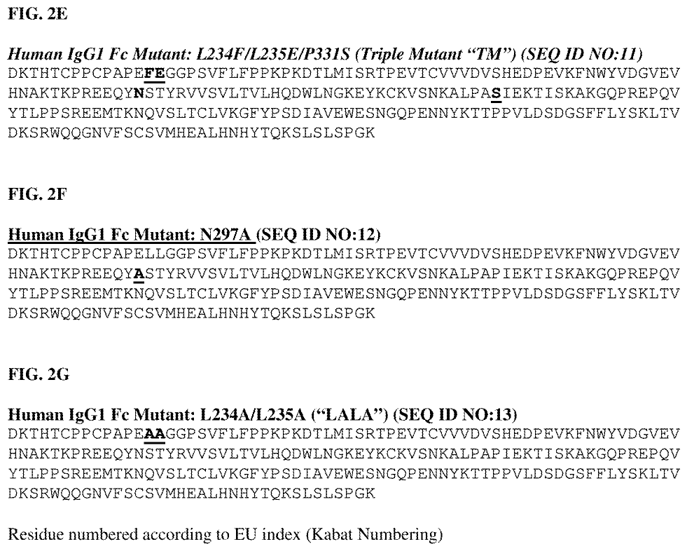

The second polypeptide of the T-Cell-MMP may comprise an Ig Fc polypeptide sequence that can act as part of a molecule scaffold providing structure and the ability to multimerize to the T-Cell-MMP (or its epitope conjugate) and, in addition, potential locations for chemical conjugation. In some embodiments the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In such embodiments the Ig Fc polypeptide may comprise an aa sequence that has at least 85%, 90%, 95%, 98, or 99%, or even 100%, aa sequence identity to an aa sequence depicted in one of FIGS. 2A-2G. Ig Fc polypeptides may comprise a sequence having at least 20, 30, 40, 50, 60, 80, 100, 120, 140, 160, 180, 200, or 220 contiguous aas with identity to a portion of an aa sequence in any of FIGS. 2A-2G. In an embodiment where the second polypeptide comprises an IgG1 Fc polypeptide, the polypeptide may also comprise one or more aa substitutions selected from N297A, L234A, L235A, L234F, L235E, and P331S. In one such embodiment, the IgG1 Fc polypeptide comprises L234A and L235A substitutions either alone or in combination with a second polypeptide chemical conjugation site. The chemical conjugation sites can be located/attached at the N- and/or C-termini or to linkers attached thereto, or within the Ig Fc polypeptides.

2 Chemical Conjugation Sites of T-Cell-MMPs

The first and second polypeptide chemical conjugation sites of the T-Cell-MMPs may be any suitable site that can be modified upon treatment with a reagent and/or catalyst such as an enzyme that permits the formation of a covalent linkage to either one or both of the T-Cell-MMP polypeptides. In an embodiment, there is only one chemical conjugation site that has been introduced into either the first or second polypeptide of a T-Cell-MMP. In an embodiment, each first and second polypeptide chemical conjugations sites are selected to be either the same or different types of chemical conjugation sites, thereby permitting the same or different molecules to be selectively conjugated to each of the polypeptides. In another embodiment, each first and second polypeptide chemical conjugation site is selected such that they are different types of conjugation site on the respective polypeptides, permitting different molecules to be selectively conjugated to each of the polypeptides. In other embodiments, such as where both an epitope molecule and one or more payload molecules are to be incorporated into a T-Cell-MMP, more than one copy of a first and/or a second polypeptide chemical conjugation may be introduced into the T-Cell-MMP. For example, a T-Cell-MMP may have one first polypeptide chemical conjugation site (e.g., for conjugating an epitope) and multiple second polypeptide chemical conjugation sites for delivering molecules of payload (or vice versa).

In embodiments, the first and second chemical conjugation sites may be selected independently from:
a) peptide sequence attached to or within the first or second polypeptide that acts as an enzyme modification sequence (e.g., sulfatase, sortase, and/or transglutaminase sequences);
b) non-natural aas and/or selenocysteines attached to or within the first or second polypeptide;
c) engineered aa chemical conjugation sites;
d) carbohydrate or oligosaccharide moieties attached to the first or second polypeptide; and
e) IgG nucleotide binding sites attached to or within the first or second polypeptide.

a. Sulfatase Motifs

In those embodiments where enzymatic modification is chosen as the means of chemical conjugation, at least one of the one or more first and second chemical conjugation sites may comprise a sulfatase motif. Sulfatase motifs are usually 5 or 6 aas in length, and are described, for example, in U.S. Pat. No. 9,540,438 and U.S. Pat. Pub. No. 2017/0166639 A1, which are incorporated by reference. Insertion of the motif results in the formation of a protein or polypeptide that is sometimes referred to as aldehyde tagged or having an aldehyde tag. The motif may be acted on by formylglycine generating enzyme(s) (“FGE” or “FGEs”) to convert a cysteine or serine in the motif to a formylglycine residue (“fGly” although sometimes denoted “FGly”), which is an aldehyde containing aa that may be utilized for selective (e.g., site specific) chemical conjugation reactions. Accordingly, as used herein, “aldehyde tag” or “aldehyde tagged” polypeptides refer to an aa sequence comprising an unconverted sulfatase motif, as well as to an aa sequence comprising a sulfatase motif in which the cysteine or the serine residue of the motif has been converted to fGly by action of an FGE. In addition, where a sulfatase motif is provided in the context of an aa sequence, both the aa sequence (e.g., polypeptide) containing the unconverted motif as well as its fGly containing counterpart are disclosed. Once incorporated into a polypeptide (e.g., of a T-Cell-MMP), a fGly residue may be reacted with molecules (e.g., epitope peptides) comprising a variety of reactive groups including, but not limited to, thiosemicarbazide, aminooxy, hydrazide, and hydrazino groups to form a conjugate (e.g., a T-Cell-MMP-epitope conjugate) having a covalent bond between the peptide and the molecule via the fGly residue. Sulfatase motifs may be used to incorporate not only epitopes (e.g., epitope presenting peptides), but also to incorporate payloads (e.g., in the formation of conjugates with drugs and diagnostic molecules).

In embodiments, the sulfatase motif is at least 5 or 6 aa residues, but can be, for example, from 5 to 16 (e.g., 6-16, 5-14, 6-14, 5-12, 6-12, 5-10, 6-10, 5-8, or 6-8) aas in length. The sulfatase motif may be limited to a length less than 16, 14, 12, 10, or 8 aa residues.

In an embodiment, the sulfatase motif contains the sequence shown in Formula (I): X1Z1X2Z2X3Z3 (SEQ ID NO:62), where
Z1 is cysteine or serine;
Z2 is either a proline or alanine residue (which can also be represented by “P/A”);
Z3 is a basic aa (arginine, lysine, or histidine, usually lysine), or an aliphatic aa (alanine, glycine, leucine, valine, isoleucine, or proline, usually A, G, L, V, or I);
X1 is present or absent and, when present, can be any aa, though usually an aliphatic aa, a sulfur-containing aa, or a polar uncharged aa (e.g., other than an aromatic aa or a charged aa), usually L, M, V, S or T, more usually L, M, S or V, with the proviso that, when the sulfatase motif is at the N-terminus of the target polypeptide, X1 is present; and
X2 and X3 independently can be any aa, though usually an aliphatic aa, a polar, uncharged aa, or a sulfur containing aa (e.g., other than an aromatic aa or a charged aa), usually S, T, A, V, G or C, more usually S, T, A, V or G.

As indicated above, a sulfatase motif of an aldehyde tag is at least 5 or 6 aa residues, but can be, for example, from 5 to 16 aas in length. The motif can contain additional residues at one or both of the N- and C-termini, such that the aldehyde tag includes both a sulfatase motif and an “auxiliary motif.” In an embodiment, the sulfatase motif includes a C-terminal auxiliary motif (i.e., following the Z3 position of the motif).

A variety of FGEs may be employed for the conversion (oxidation) of cysteine or serine in a sulfatase motif to fGly. As used herein, the term formylglycine generating enzyme, or FGE, refers to fGly-generating enzymes that catalyze the conversion of a cysteine or serine of a sulfatase motif to fGly. As discussed in U.S. Pat. No. 9,540,438, the literature often uses the term formylglycine-generating enzymes for those enzymes that convert a cysteine of the motif to fGly, whereas enzymes that convert a serine in a sulfatase motif to fGly are referred to as Ats-B-like.

Sulfatase motifs of Formula (I) amenable to conversion by a prokaryotic FGE often contain a cysteine or serine at Z1 and a proline at Z2 that may be modified either by the “SUMP I-type” FGE or the “AtsB-type” FGE, respectively. Prokaryotic FGE enzymes that may be employed include the enzymes from Clostridium perfringens (a cysteine type enzyme), Klebsiella pneumoniae (a Serine-type enzyme) or the FGE of Mycobacterium tuberculosis. Where peptides containing a sulfatase motif are being prepared for conversion into fGly-containing peptides by a eukaryotic FGE, for example by expression and conversion of the peptide in a eukaryotic cell or cell free system using a eukaryotic FGE, sulfatase motifs amenable to conversion by a eukaryotic FGE may advantageously be employed.

Host cells for production of polypeptides with unconverted sulfatase motifs, or where the cell expresses a suitable FGE for converting fGly-containing polypeptide sequences, include those of a prokaryotic and eukaryotic organism. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism such as insects and vertebrates, particularly mammals, include, but are not limited to, CHO cells, HEK cells, and the like (e.g., American Type Culture Collection (ATCC) No. CCL-2), CHO cells (e.g., ATCC Nos. CRL9618 and CRL9096), CHO DG44 cells, CHO-Kl cells (ATCC CCL-61), 293 cells (e.g., ATCC No. CRL-1573), Vero cells, NIH 3T3 cells (e.g., ATCC No. CRL-1658), Hnh-7 cells, BHK cells (e.g., ATCC No. CCLlO), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Sulfatase motifs may be incorporated into any desired location on the first or second polypeptide of the T-Cell-MMP (or its epitope conjugate). In an embodiment, a sulfatase motif may be added at or near the terminus of any element in the first or second polypeptide of the T-Cell-MMP (or its epitope conjugate), including the first and/or second MHC polypeptides (e.g., MHC-H and/or β2M polypeptides), the scaffold or Ig Fc, and the linkers adjoining those elements. Accordingly, the sulfatase motif may be linked to an aa in the N-terminal region of β2M (with or without a linker).

In an embodiment a sulfatase motif is incorporated into, or attached to (e.g., via a peptide linker), a T-Cell-MMP (or its epitope conjugate) in a first or second polypeptide that has a β2M polypeptide with a sequence having at least 85% (e.g., at least 90%, 95%, 98% or 99%, or even 100%) aa sequence identity to a sequence shown in FIG. 4 (e.g., any of the full length sequences shown in FIG. 4, or the sequence of any of the mature β2M polypeptides starting at aa 21 and ending at their C-terminus). For the purposes of this embodiment, sequence identity of the β2M polypeptide is determined relative to the corresponding portion of a β2M polypeptide in FIG. 4 without consideration of the added sulfatase motif and any linker sequences present.

U.S. Pat. No. 9,540,438 discusses the incorporation of sulfatase motifs into the various immunoglobulin sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming fGly residues, as well as the chemistry related to the coupling of molecules such as epitopes and payloads to fGly residues.

The incorporation of a sulfatase motif may be accomplished by incorporating a nucleic acid sequence encoding the motif at the desired location in a nucleic acid encoding the first and/or second polypeptide of the T-Cell-MMP. As discussed below, the nucleic acid sequence may be placed under the control of a transcriptional regulatory sequence(s) (a promoter) and provided with regulatory elements that direct its expression. The expressed protein may be treated with one or more FGEs after expression and partial or complete purification. Alternatively, expression of the nucleic acid in cells that express a FGE that recognizes the sulfatase motif results in the conversion of the cysteine or serine of the motif to fGly, which is sometimes called oxoalanine.

In view of the foregoing, this disclosure provides for T-Cell-MMPs comprising one or more fGly residues incorporated into the sequence of the first or second polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above.

After chemical conjugation the T-Cell-MMPs comprise one or more fGly′ residues incorporated into the sequence of the first or second polypeptide chain in the context of the sequence X1(fGly′)X2Z2X3Z3, where the fGly′ residue is formylglycine that has undergone a chemical reaction and now has a covalently attached moiety (e.g., epitope or payload).

A number of chemistries and commercially available reagents can be utilized to conjugate a molecule (e.g., an epitope or payload) to a fGly residue, including, but not limited to, the use of thiosemicarbazide, aminooxy, hydrazide, or hydrazino derivatives of the molecules to be coupled at a fGly-containing chemical conjugation site. For example, epitopes (e.g., epitope peptides) and/or payloads bearing thiosemicarbazide, aminooxy, hydrazide, hydrazino or hydrazinyl functional groups (e.g., attached directly to an aa of a peptide or via a linker such as a PEG) can be reacted with fGly-containing first or second polypeptides of the T-Cell-MMP to form a covalently linked epitope. Similarly, payloads such as drugs and therapeutics can be incorporated using, for example, biotin hydrazide as a linking agent.

An epitope (e.g., an epitope presenting peptide, phosphopeptide, lipopeptide, or glycopeptide) such as an epitope having a length from about 4 aa to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length and/or one or more payloads may be conjugated to a fGly containing polypeptide.

The disclosure provides for methods of preparing T-Cell-MMP-epitope conjugates and/or T-Cell-MMP-payload conjugates comprising:
a) incorporating a sequence encoding a sulfatase motif including a serine or cysteine (e.g., a sulfatase motif of Formula (I) or (II) such as X1CX2PX3Z3 (SEQ ID NO:63); CX1PX2Z3 (SEQ ID NO:64) discussed above) into a nucleic acid encoding a first polypeptide and/or second polypeptide of a T-Cell-MMP;
b) expressing the sulfatase motif-containing first polypeptide and/or second polypeptide in a cell that
   i) expresses a FGE and converts the serine or cysteine of the sulfatase motif to a fGly and partially or completely purifying the fGly-containing first polypeptide and/or second polypeptide separately or as the T-Cell-MMP, or
   ii) does not express a FGE that converts a serine or cysteine of the sulfatase motif to a fGly, purifying or partially purifying the T-Cell-MMP containing the fGly residue and contacting the purified or partially purified T-Cell-MMP with a FGE that converts the serine or cysteine of the sulfatase motif into a fGly residue; and
c) contacting the fGly-containing first and/or second polypeptides separately, or as part of a T-Cell-MMP, with an epitope and/or payload that has been functionalized with a group that forms a covalent bond between the aldehyde of the fGly and epitope and/or payload;
thereby forming aT-Cell-MMP-epitope conjugate and/or T-Cell-MMP payload conjugate.
   In such methods the epitope (epitope containing molecule) and/or payload may be functionalized by any suitable function group that reacts selectively with an aldehyde group. Such groups may, for example, be selected from the group consisting of thiosemicarbazide, aminooxy, hydrazide, and hydrazino. In an embodiment a sulfatase motif is incorporated into a first or second polypeptide comprising a β2M aa sequence with at least 85% (e.g., at least 90%, 95%, 98% or 99%, or even 100%) sequence identity to at least 60, 70, 80 or 90 contiguous aas of a β2M sequence shown in FIG. 4 (e.g., with identity calculated without including or before the addition of the sulfatase motif sequence). For example, the sulfatase motif may be placed between the signal sequence and the sequence of the mature peptide, or at the N-terminus of the mature peptide, and the motif may be separated from the β2M sequence(s) by peptide linkers.

In an embodiment, the method of preparing a T-Cell-MMP-epitope conjugate and/or T-Cell-MMP payload conjugate, a sulfatase motif is incorporated into a polypeptide comprising a sequence having at least 85% (e.g., at least 90%, 95%, 98% or 99%, or even 100%) aa sequence identity to at least 150, 175, 200, or 225 contiguous aas of a sequence shown in FIG. 3 (e.g., 3A-3I, with sequence identity calculated without including the addition of the sulfatase motif sequence). In one such embodiment, the sulfatase motifs may be utilized as sites for the conjugation of, for example, epitopes and/or payloads either directly or indirectly through a peptide or chemical linker.

b. Sortase A Enzyme Sites

Epitopes (e.g., peptides comprising the sequence of an epitope) and payloads may be attached at the N- and/or C-termini of the first and/or second polypeptides of a T-Cell-MMP by incorporating sites for Sortase A conjugation at those locations.

Sortase A recognizes a C-terminal pentapeptide sequence LP(X5)TG/A (SEQ ID NO 65, with X5 being any single amino acid, and G/A being a glycine or alanine), and creates an amide bond between the threonine within the sequence and glycine or alanine in the N-terminus of the conjugation partner.

For attachment of epitopes or payloads to the C-terminus of the first or second polypeptide of the T-Cell-MMP, an LP(X5)TG/A is engineered into the carboxy terminal portion of the desired polypeptide(s). An exposed stretch of glycines or alanines (e.g., (G)₃₋₅ (SEQ ID NOs:66 and 67 when using Sortase A from Staphylococcus aureus or alanines (A)₃₋₅, SEQ ID NOs:68 and 69 when using Sortase A from Streptococcus pyogenes) is engineered into the N-terminus of a peptide that comprises an epitope (or a linker attached thereto), a peptide payload (or a linker attached thereto), or a peptide covalently attached to a non-peptide epitope or payload.

For attachment of epitopes or payloads to the amino terminus of the first or second polypeptide of the T-Cell-MMP, an aa sequence comprising an exposed stretch of glycines (e.g., (G)_{2, 3, 4, or 5}) or alanines (e.g., (A)_{2, 3, 4, or 5}) is engineered to appear at the N-terminus of the desired polypeptide(s), and a LP(X5)TG/A is engineered into the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy engineered peptides results in a cleavage between the Thr and Gly/Ala residues in the LP(X5)TG/A sequence, forming a thioester intermediate with the carboxy labeled peptide. Nucleophilic attack by the N-terminal modified polypeptide results in the formation of a covalently coupled complex of the form: carboxy-modified polypeptide-LP(X5)T*G/A-amino-modified polypeptide, where the “*” represents the bond formed between the threonine of the LP(X5)TG/A motif and the glycine or alanine of the N-terminal modified peptide.

In place of LP(X5)TG/A, a LPETGG (SEQ ID NO:70) peptide may be used for S. aureus Sortase A coupling, or a LPETAA (SEQ ID NO:71) peptide may be used for S. pyogenes Sortase A coupling. The conjugation reaction is still between the threonine and the amino terminal oligoglycine or oligoalanine peptide to yield a carboxy-modified polypeptide-LP(X5)T*G/A-amino-modified polypeptide, where the “*” represents the bond formed between the threonine and the glycine or alanine of the N-terminal modified peptide.

In an embodiment, a A₂₋₅ or a G₂₋₅ motif is incorporated into a polypeptide comprising a sequence having at least 85% (e.g., at least 90%, 95%, 98% or 99%, or even 100%) aa sequence identity to at least 60, 70, 80 or 90 contiguous aas of a sequence shown in FIG. 4 (e.g., either the entire sequences shown in FIF. 4, or the sequence of the mature polypeptides starting at aa 21 and ending at their C-terminus), with sequence identity assessed without consideration of the added A₂₋₅ or G₂₋₅ motif and any linker sequences present.

In an embodiment, an A₂₋₅ or a G₂₋₅ motif is incorporated into a polypeptide comprising a β2M sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (e.g., any of the full length sequences shown in FIG. 4, or any of the mature polypeptide sequences starting at aa 21 and ending at their C-terminus), with aa deletions, insertions and/or changes assessed without consideration of the added A₂₋₅ or G₂₋₅ motif and any linker sequences present. In one such embodiment an A₂₋₅ or a G₂₋₅ motif may either replace and/or be inserted between any of the amino terminal 15 (e.g., 1-5, 5-10 or 10-15) aas of a mature β2M sequence, such as those shown in FIG. 4.

c. Transglutaminase Enzyme Sites

Transglutaminases (mTGs) catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and payloads to T-Cell-MMPs, either directly or indirectly via a linker comprising a free primary amine. As such, glutamine residues present in the first and/or second polypeptides of the T-Cell-MMP may be considered as chemical conjugation sites when they can be accessed by enzymes such as Streptoverticillium mobaraense transglutaminase. That enzyme (EC 2.3.2.13) is a stable, calcium-independent enzyme catalyzing the γ-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial mTGs are generally unable to modify glutamine residues in native IgG1s; however, Schibli and co-workers (Jeger, S., et al. Angew Chem (Int Engl). 2010; 49:99957 and Dennler P, et al. Bioconjug Chem. 2014; 25(3):569-78) found that deglycosylating IgG1s at N297 rendered glutamine residue Q295 accessible and permitted enzymatic ligation to create an antibody drug conjugate. Further, by producing a N297 to Q297 IgG1 mutant, they introduce two sites for enzymatic labeling by transglutaminase.

Where a first and/or second polypeptide of the T-Cell-MMP does not contain a glutamine that may be employed as a chemical conjugation site (e.g., it is not accessible to a transglutaminase or not placed in the desired location), a glutamine residue, or a sequence comprising an accessible glutamine that can act as a substrate of a transglutaminase (sometimes referred to as a “glutamine tag” or a “Q-tag”), may be incorporated into the polypeptide. The added glutamine or Q-tag may act as a first polypeptide chemical conjugation site or a second polypeptide chemical conjugation site. US Pat. Pub. No. 2017/0043033 A1 describes the incorporation of glutamine residues and Q-tags and the use of transglutaminase for modifying polypeptides and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell free system. In embodiments, the glutamine-containing Q-tag comprises an aa sequence selected from the group consisting of LQG, LLQGG (SEQ ID NO:72), LLQG (SEQ ID NO:73), LSLSQG (SEQ ID NO:74), and LLQLQG (SEQ ID NO:75) (numerous others are available).

In an embodiment, the added glutamine residue or Q-tag is attached to (e.g., at the N- or C-terminus), or within, the sequence of the first MHC polypeptide, or, if present, a linker attached to the first MHC polypeptide. In one such embodiment, the first MHC polypeptide of a T-Cell-MMP is a β2M polypeptide, and an added glutamine or Q-tag is incorporated within 20, 15, or 10 aas of the N-terminus of a mature β2M polypeptide sequence, which exclude the 20 base pair signal sequence, provided in FIG. 4 (or a peptide having at least 85%, 90%, 95%, 98%, 99, or even 100% sequence identity to a mature β2M polypeptide in FIG. 4). In another embodiment, the glutamine or Q-tag is present in a polypeptide linker attached to the N-terminus of one of the mature β2M polypeptides provided in FIG. 4.

In an embodiment the added glutamine residue or Q-tag is attached to (e.g., at the N- or C-terminus), or within, the sequence of the second polypeptide of a T-Cell-MMP, for example at a terminus or within a second MHC polypeptide (e.g., a MHC-H peptide), or, if present, a Fc, scaffold peptide or linker attached directly or indirectly to the second MHC polypeptide. In one embodiment, the second MHC polypeptide is a MHC-H polypeptide, the second polypeptide comprises a Fc polypeptide, and an added glutamine or Q-tag is incorporated within the MHC-H or the Fc polypeptide sequence. In another embodiment, the glutamine or Q-tag is present within a polypeptide linker between the MHC-H and Fc polypeptides, or within a linker attached to the carboxyl terminus of the Fc polypeptide.

Payloads and epitopes that contain, or have been modified to contain, a primary amine group may be used as the amine donor in a transglutaminase catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine or another primary amine that a transglutaminase can act on may be incorporated into the peptide. Other amine containing compounds that may provide a primary amine group and that may be incorporated into, or at the end of, an alpha amino acid chain include, but are not limited to, homolysine, 2,7-diaminoheptanoic acid, and aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, including enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria including a variety of Streptoverticillium, Streptomyces, Actinomadura sp., Bacillus, and the like.

As discussed above for other first polypeptide chemical conjugation sites and second polypeptide chemical conjugation sites, a glutamine or Q-tag may be incorporated into any desired location on the first or second polypeptide of the T-Cell-MMP. In an embodiment, a glutamine or Q-tag may be added at or near the terminus of any element in the first or second polypeptide of the T-Cell-MMP, including the first and second MHC polypeptides (e.g., MHC-H and β2M polypeptides), the scaffold or Ig Fc, and the linkers adjoining those elements.

In one embodiment, where the first polypeptide of the T-Cell-MMP comprises a β2M polypeptide sequence, the first polypeptide contains a glutamine or Q-tag at the N-terminus of the polypeptide, or at the N-terminus of a polypeptide linker attached to the first polypeptide (e.g., the linker is attached to the N-terminus of the first polypeptide).

In an embodiment a Q-tag motif is incorporated into a polypeptide comprising a β2M sequence having at least 85% (e.g., at least 90%, 95%, 98% or 99%, or even 100%) aa sequence identity to at least 60, 70, 80 or 90 contiguous aas of a sequence shown in FIG. 4 (e.g., any of the full-length sequences shown in FIG. 4, or the sequence of any of the mature β2M polypeptide starting at aa 21 and ending at their C-terminus), with identity assessed without consideration of the added Q-tag motif and any linker sequences present.

In an embodiment a Q-tag motif is incorporated into a sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either the entire sequences shown in FIG. 4, or the sequence of the mature polypeptides starting at aa 21 and ending at their C-terminus). Changes are assessed without consideration of the aas of the Q-tag motif and any linker sequences present. In one such embodiment a Q-tag motif may replace and/or be inserted between any of the amino terminal 15 (e.g., 1-5, 5-10, or 10-15) aas of a mature β2M sequence, such as those shown in FIG. 4.

Q-tags may be created by modifying the aa sequence around any one, two, or three of the glutamine residues appearing in a β2M and/or MHC-H chain sequence appearing in a T-Cell-MMP and used as a chemical conjugation site for addition of an epitope or payload. Similarly, Q-tags may be incorporated into the IgFc region as second polypeptide chemical conjugation sites and used for the conjugation of, for example, epitopes and/or payloads either directly or indirectly through a peptide or chemical linker bearing primary amine.

d. Selenocysteine and Non-Natural Amino Acids as Chemical Conjugation Sites

One strategy for providing site-specific chemical conjugation sites in the first and/or second polypeptides of a T-Cell-MMP employs the insertion of aas with reactivity distinct from the other aas present in the polypeptide. Such aas include, but are not limited to, the non-natural aas, acetylphenylalanine (p-acetyl-L-phenylalanine, pAcPhe), parazido phenylalanine, and propynyl-tyrosine, and the naturally occurring aa, selenocysteine (Sec).

Thanos et al. in US Pat. Publication No. 20140051836 A1 discuss some other non-natural aas including O-methyl-L-tyrosine, L-3-(2-naphthyl)alanine, a 3-methyl-phenylalanine, an O-4-allyl-L-tyrosine, a 4-propyl-L-tyrosine, a tri-O-acetyl-GlcNAcβ-serine, L-Dopa, a fluorinated phenylalanine, an isopropyl-L-phenylalanine, a p-acyl-L-phenylalanine, a p-benzoyl-L-phenylalanine, L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine. Other non-natural aas include reactive groups including amino, carboxy, acetyl, hydrazino, hydrazido, semicarbazido, sulfanyl, azido and alkynyl. See, e.g., US Pat. Publication No. 20140046030 A1.

In addition to directly synthesizing polypeptides in the laboratory, two methods utilizing stop codons have been developed to incorporate non-natural aas into proteins and polypeptides utilizing transcription-translation systems. The first incorporates selenocysteine (Sec) by pairing the opal stop codon, UGA, with a Sec insertion sequence. The second incorporates non-natural aas into a polypeptide generally through the use of amber, ochre, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655(2006). By way of example, the non-natural amino acid acetylphenylalanine may be incorporated at an amber codon using a tRNA/aminoacyl tRNA synthetase pair in an in vivo or cell free transcription-translation system.

Incorporation of both selenocysteine and non-natural aas requires engineering the necessary stop codon(s) into the nucleic acid coding sequence of the first and/or second polypeptide of the T-Cell-MMP at the desired location(s), after which the coding sequence is used to express the first or second polypeptide strand of the T-Cell-MMP in an in vivo or cell free transcription-translation system.

In vivo systems generally rely on engineered cell-lines to incorporate non-natural aas that act as bio-orthogonal chemical conjugation sites into polypeptides and proteins. See, e.g., International Published Application No. 2002/085923 entitled “In vivo incorporation of unnatural amino acids.” In vivo non-natural aa incorporation relies on a tRNA and an aminoacyl tRNA synthetase (aaRS) pair that is orthogonal to all the endogenous tRNAs and synthetases in the host cell. The non-natural aa of choice is supplemented to the media during cell culture or fermentation, making cell-permeability and stability important considerations.

Various cell-free synthesis systems provided with the charged tRNA may also be utilized to incorporate non-natural aas. Such systems include those described in US Pat. Publication No. 20160115487A1; Gubens et al., RNA. 2010 August; 16(8): 1660-1672; Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once incorporated into the first or second polypeptide of the T-Cell-MMP, epitopes and/or payload bearing groups reactive with the incorporated selenocysteine or non-natural aa are brought into contact with the T-Cell-MMP under suitable conditions to form a covalent bond. By way of example, the keto group of the pAcPhe is reactive towards alkoxy-amines, via oxime coupling, and can be conjugated directly to alkoxyamine containing epitopes and/or payloads or indirectly to epitopes and payloads via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or payloads bearing those groups or bound to linkers bearing those groups can be covalently bound to polypeptide chains bearing selenocysteines.

As discussed above for other first polypeptide chemical conjugation sites and second polypeptide chemical conjugation sites, selenocysteines and/or non-natural aas may be incorporated into any desired location in the first or second polypeptide of the T-Cell-MMP. In an embodiment, selenocysteines and/or non-natural aas may be added at or near the terminus of any element in the first or second polypeptide of the T-Cell-MMP, including the first and second MHC polypeptides (e.g., MHC-H and β2M polypeptides), the scaffold or Ig Fc, and the linkers adjoining those elements. In embodiments selenocysteines and/or non-natural aas may be incorporated into a β2M, class I MHC heavy chain, and/or a Fc Ig polypeptide. In an embodiment, selenocysteines and/or non-natural aas may be incorporated into the first polypeptide near or at the amino terminal end of the first MHC polypeptide (e.g., the β2M polypeptide) or a linker attached to it. For example, where the first polypeptide comprises a β2M sequence, selenocysteines and/or non-natural aas may be incorporated at or near the N-terminus of a β2M sequence, permitting the chemical conjugation of, for example, an epitope either directly or through a linker. By way of example, the sequences of β2M as shown in FIG. 4 begin with a 20 aa leader sequence, and the mature polypeptide begins with the initial sequence IQRTP(K/Q)IQVYS . . . and continues through the remainder of the polypeptide (see SEQ ID NOs:57-61).

In an embodiment selenocysteines and/or non-natural aas are incorporated into a polypeptide comprising a β2M sequence having at least 85% (e.g., at least 90%, 95%, 98% or 99%, or even 100%) aa sequence identity to a β2M sequence shown in FIG. 4 (e.g., any of the full length sequences shown in FIG. 4, or the sequence of any of the mature β2M polypeptides starting at aa 21 and ending at their C-terminus), with sequence identity assessed without consideration of the added selenocysteines and/or non-natural aas and any linker sequences present.

In an embodiment selenocysteines and/or non-natural aas are incorporated into a polypeptide comprising a β2M sequence having 1 to 15 (e.g., 1, 2, 3, 4, 5, 6, 7, 8, 9, 10, 11, 12, 13, 14, or 15) aa deletions, insertions and/or changes compared with a β2M sequence shown in FIG. 4 (e.g., any of the full-length sequences shown in FIG. 4, or the sequence of any of the mature β2M polypeptides starting at aa 21 and ending at their C-terminus). Changes are assessed without consideration of the aas of the selenocysteines and/or non-natural aas and any linker sequences present. In one such embodiment a selenocysteine and/or non-natural aa may replace and/or be inserted between any of the amino terminal 15 aas of a mature β2M sequence, such as those shown in FIG. 4.

In other embodiments, selenocysteines and/or non-natural aas may be incorporated into polypeptides comprising a MHC-H chain or IgFc polypeptide sequences (including linkers attached thereto) as chemical conjugation sites. In one such embodiment they may be utilized as sites for the conjugation of, for example, epitopes and/or payloads conjugated to the T-Cell-MMP either directly or indirectly through a peptide or chemical linker.

e. Engineered Amino Acid Chemical Conjugation Sites

Any of the variety of functionalities (e.g., —SH, —NH₃, —OH, —COOH and the like) present in the side chains of naturally occurring amino acids, or at the termini of polypeptides, can be used as chemical conjugation sites. This includes the side chains of lysine and cysteine, which are readily modifiable by reagents including N-hydroxysuccinimide and maleimide functionalities, respectively. The main disadvantages of utilizing such amino acid residues is the potential variability and heterogeneity of the products. For example, an IgG has over 80 lysines, with over 20 at solvent-accessible sites. See, e.g., McComb and Owen, AAPS J. 117(2): 339-351. Cysteines tend to be less widely distributed; they tend to be engaged in disulfide bonds and may be inaccessible and not located where it is desirable to place a chemical conjugation site. Accordingly, it is possible to engineer the first and/or second polypeptide to incorporate non-naturally occurring amino acids at the desired locations for selective modification of the T-Cell-MMP first and/or second polypeptides. Engineering may take the form of direct chemical synthesis of the polypeptides (e.g., by coupling appropriately blocked amino acids) and/or by modifying the sequence of a nucleic acid encoding the polypeptide followed expression in a cell or cell free system. Accordingly, the specification includes and provides for the preparation of the first and/or second polypeptide of a T-Cell-MMP by transcription/translation bearing a non-natural or natural (including selenocysteine) amino acid to be used as a chemical conjugation site (e.g., for epitopes or peptides).

The specification also includes and provides for the preparation of all or part of the first and/or second polypeptide of a T-Cell-MMP by transcription/translation, and joining to the C- or N-terminus of the translated portion of the first and/or second polypeptide an engineered polypeptide bearing a non-natural or natural (including selenocysteine) amino acid to be used as a chemical conjugation site (e.g., for epitopes or peptides). The engineered peptide may be joined by any suitable method, including the use of a sortase as described for epitope peptides above and may include a linker peptide sequence. In an embodiment the engineered peptide may comprise a sequence of 2, 3, 4, or 5 alanines or glycines that may serve for sortase conjugation and/or as part of a linker sequence.

In one embodiment, a first or second polypeptide of a T-Cell-MMP contains at least one naturally occurring aa (e.g., a cysteine) to be used as a chemical conjugation site engineered into a β2M sequence as shown in FIG. 4, an IgFc sequence as shown in any of FIGS. 2A-G, or a MHC Class I heavy chain polypeptide as shown in FIGS. 3A-3I. In an embodiment, at least one naturally occurring aa to be used as a chemical conjugation site is engineered into a polypeptide having at least 85% (e.g., at least 90%, 95%, 98% or 99%, or even 100%) aa sequence identity to a β2M sequence as shown in FIG. 4, an IgFc sequence as shown in FIG. 2, or a MHC Class I heavy chain polypeptide as shown in any of FIGS. 3A-3I. At least one naturally occurring aa (e.g., a cysteine) may be engineered as a chemical conjugation site in a T-Cell-MMP first or second polypeptide comprising a β2M aa sequence having at least 90% (e.g., at least 93%, 95%, 98% or 99%, or even 100%) aa sequence identity with at least the amino terminal 10, 20, 30, 40, 50, 60 or 70 aas of a mature β2M sequence as shown in FIG. 4. At least one naturally occurring aa (e.g., a cysteine) may be engineered as a chemical conjugation site in a T-Cell-MMP first or second polypeptide comprising an IgFc sequence as shown in any of FIGS. 2A-2G. At least one naturally occurring aa (e.g., a cysteine) may be engineered as a chemical conjugation site in a T-Cell-MMP first or second polypeptide comprising a MHC Class I heavy chain polypeptide as shown in any of FIGS. 3A to 3I. In another embodiment, at least one naturally occurring aa to be used as a chemical conjugation site is engineered into a first or second polypeptide comprising a contiguous sequence of at least 30, 40, 50, 60, 70, 80, 90, or 100 aas having 100% aa sequence identity to a MHC Class I heavy chain sequence as shown in any of FIGS. 3A to 3I. In any of the embodiments mentioned above where a naturally occurring aa is engineered into a polypeptide, the aa may be selected from the group consisting of arginine, lysine, cysteine, serine, threonine, glutamic acid, glutamine, aspartic acid, and asparagine. Alternatively, the aa engineered as a conjugation site is selected from the group consisting of lysine, cysteine, serine, threonine, and glutamine. The aa engineered as a conjugation site may also be selected from the group consisting of lysine, glutamine, and cysteine. In an embodiment, the engineered aa is cysteine. In an embodiment, the engineered aa is lysine. In another embodiment, the engineered aa is glutamine.

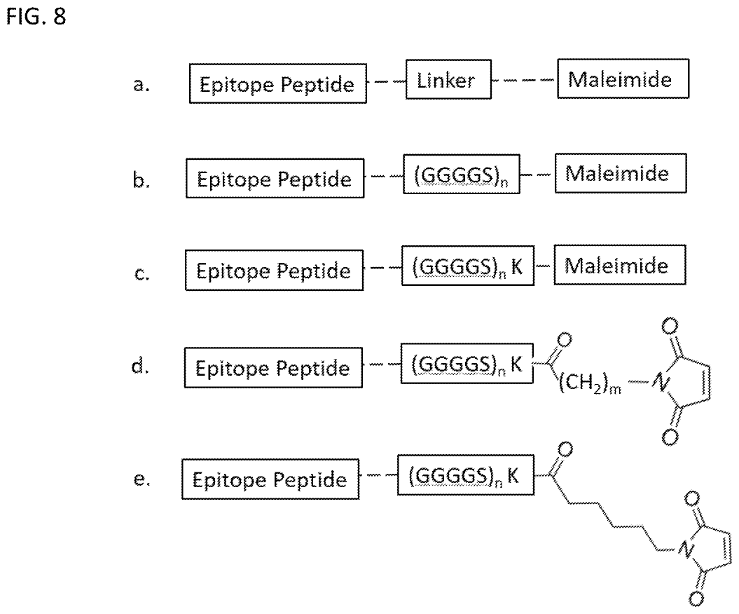

Any method known in the art may be used to couple payloads or epitopes to amino acids engineered into the first or second polypeptides of the T-Cell-MMP. By way of example, maleimides may be utilized to couple to sulfhydryls, N-hydroxysuccinimide may be utilized to couple to amine groups, acid anhydrides or chlorides may be used to couple to alcohols or amines, and dehydrating agents may be used to couple alcohols or amines to carboxylic acid groups. Accordingly, using such chemistry an epitope or payload may be coupled directly, or indirectly through a linker (e.g., a homo- or hetero-bifunctional crosslinker), to a location on a first and/or second polypeptide. A number of bifunctional crosslinkers may be utilized, including, but not limited to, those described for linking a payload to the T-Cell-MMP described herein below. For example, an epitope peptide (or a peptide-containing payload) including a maleimide group attached by way of a homo- or hetero-bifunctional linker (see, e.g., FIG. 8) or a maleimide amino acid can be conjugated to a sulfhydryl of a chemical conjugation site (e.g., a cysteine residue) that is naturally occurring or engineered into a T-Cell-MMP.

Maleimido amino acids can be incorporated directly into peptides (e.g., epitope peptides) using a Diels-Alder/retro-Diels-Alder protecting scheme as part of a solid phase peptide synthesis. See, e.g., Koehler, Kenneth Christopher (2012), “Development and Implementation of Clickable Amino Acids,” Chemical & Biological Engineering Graduate Theses & Dissertations, 31, https://scholar.colorado.edu/chbe_gradetds/31.

A maleimide group may also be appended to an epitope peptide using a homo- or hetero-bifunctional linker (sometimes referred to as a crosslinker) that attaches a maleimide directly (or indirectly, e.g., through an intervening linker that may comprise additional aas bound to the peptide presenting the epitope) to the epitope peptide. For example, a heterobifunctional N-hydroxysuccinimide-maleimide crosslinker can attach maleimide to an amine group of, a peptide lysine. Some specific cross linkers include molecules with a maleimide functionality and either a N-hydroxysuccinimide ester (NHS) or N-succinimidyl group that can attach a maleimide to an amine (e.g., an epsilon amino group of lysine). Examples of such crosslinkers include, but are not limited to, NHS-PEG4-maleimide, γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); and N-(α-maleimidoacetoxy)-succinimide ester (AMAS), which offer different lengths and properties for peptide immobilization. Other amine reactive crosslinkers that incorporate a maleimide group include N-succinimidyl 4-(2-pyridyldithio)butanoate (SPDB). Additional crosslinkers (bifunctional agents) are recited below. In an embodiment the epitopes coupled to the T-Cell-MMP have a maleimido alkyl carboxylic acid coupled to the peptide by an optional linker (see, e.g., FIG. 8), for example by an amide formed with the epsilon amino group of a lysine. The maleimido carboxylic acid can be, for example, a maleimido ethanoic, propanoic, butanoic, pentanoic, hexanoic, heptanoic, or octanoic acid.

Accordingly, an epitope peptide may be coupled to a cysteine present (e.g., engineered into), for example, in the binding pocket of a T-Cell-MMP through a bifunctional linker comprising a maleimide or a maleimide amino acid incorporated into the peptide. An epitope peptide may be conjugated (e.g., by one or two maleimide amino acids or at least one maleimide containing bifunctional linker) to a MHC heavy chain having cysteine residues at any one or more (e.g., 1 or 2) aa positions selected from positions 5, 7, 59, 84, 116, 139, 167, 168, 170, and/or 171 (e.g., Y7C, Y59C, Y84C, Y116C, A139C, W167C, L168C, R170C, and Y171C substitutions) with the numbering as in FIGS. 3D-3I. An epitope peptide may be conjugated (e.g., by one or two maleimide amino acids or at least one maleimide containing bifunctional linker) to a MHC heavy chain having cysteine residues at any one or more (e.g., 1 or 2) aa positions selected from positions 7, 84 and/or 116, (e.g., Y7C, Y84C, and Y116C substitutions) with the numbering as in FIGS. 3D-3H. An epitope peptide may be conjugated (e.g., by one or two maleimide amino acids or at least one maleimide containing bifunctional linker) to a MHC heavy chain having cysteine residues at any one or more (e.g., 1 or 2) aa positions selected from positions 84 and/or 116 (e.g., Y84C and/or Y116C substitutions) with the numbering as in FIGS. 3D-3H.

Epitope peptides may also be coupled to a cysteine present (e.g., engineered into), a β2M polypeptide sequence having at least 85% (e.g., at least 90%, 95% 97% or 100%) sequence identity to at least 60 contiguous amino acids (e.g., at least 70, 80, 90 or all contiguous aas) of a mature β2M polypeptide sequence set forth in FIG. 4. Epitopes may be conjugated to cysteines at positions 2, 44, 50, 77, 85, 88, 91, or 98 of the mature polypeptides (aas 22, 64, 70, 97, 85, 108, 111, or 118 of the β2M sequences as shown in FIG. 4). Accordingly, the β2M sequences of a T-Cell-MMP or its epitope conjugate may contain cysteine chemical conjugation site engineered into the mature β2M sequence selected from Q2C, E44C, E50C, E77C, V85V, S88C, K91C, and D98C. Any of those substitutions may be accompanied by an R12C substitution which that forms a disulfide bond with a cysteine engineered into position 236 (e.g., A236C) of the MHC-H chain to form a interchain disulfide bond between the β2M sequence and the MHC H sequence as described, for example, in Section I.A.4. The cysteine chemical conjugation sites in β2M sequences may also be combined with Y84C and A139C substitutions made to stabilize the MHC H.

A T-Cell-MMP or its epitope conjugate may comprise a Q2C substitution in its β2M sequence for conjugation of epitope peptides (e.g., peptides, glycopeptides, lipopeptides or phosphopeptides) to β2M the sequence directly or indirectly via a linker). The β2M sequence with a Q2C substitution may also include a R12C substation. In a T-cell-MMP, a β2M sequence comprising a Q2C substitution may be combined with an MHC-H chain comprising a Y84C and A139C. In a T-cell-MMP, a β2M sequence comprising a Q2C substitution and an R12C substation may be combined with an MHC-H chain comprising a Y84C, A139C, and A236C substitutions. Any of the foregoing may be used to prepare a T-Cell-MMP-epitope conjugate.

A T-Cell-MMP or its epitope conjugate may comprise an E44C substitution in its β2M sequence for conjugation of epitope peptides (e.g., peptides, glycopeptides, lipopeptides or phosphopeptides) to β2M the sequence directly or indirectly via a linker). The β2M sequence with an E44C substitution may also include a R12C substation. In a T-cell-MMP, a β2M sequence comprising a E44C substitution may be combined with an MHC-H chain comprising a Y84C and A139C. In a T-cell-MMP, a β2M sequence comprising a E44C substitution and an R12C substation may be combined with an MHC-H chain comprising a Y84C, A139C, and A236C substitutions. Any of the foregoing may be used to prepare a T-Cell-MMP-epitope conjugate.

A pair of sulfhydryl groups may be employed simultaneously to create a chemical conjugate to a T-Cell-MMP. In such an embodiment a T-Cell-MMP that has a disulfide bond, or has two cysteines (or selenocysteines) engineered into locations proximate to each other, may be utilized as a chemical conjugation site by incorporation of bis-thiol linkers. Bis-thiol linkers, described by Godwin and co-workers, avoid the instability associated with reducing a disulfide bond by forming a bridging group in its place and at the same time permit the incorporation of another molecule, which can be an epitope or payload. See, e.g., Badescu G, et al., (2014), Bioconjug Chem., 25(6):1124-36, entitled Bridging disulfides for stable and defined antibody drug conjugates, describing the use of bis-sulfone reagents, which incorporate a hydrophilic linker (e.g., PEG (polyethyleneglycol) linker).

Where a T-Cell-MMP comprises a disulfide bond, the bis-thiol linker may be used to incorporate an epitope or payload by reducing the bond, generally with stoichiometric or near stoichiometric amounts of dithiol reducing agents (e.g., dithiothreitol) and allowing the linker to react with both cysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60:3-12. Where the first and/or second polypeptides of the T-Cell-MMP do not comprise a pair of cysteines and/or selenocysteines (e.g., a selenocysteine and a cysteine), they may be engineered into the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be present on the first and/or second polypeptide of a T-Cell-MMP. Alternatively, the cysteines and/or selenocysteines may be present on the first polypeptide and their counterparts for bis-thiol linker reaction present on the second polypeptide of a T-Cell-MMP.

In an embodiment, a pair of cysteines and/or selenocysteines is incorporated into a first or second polypeptide of a T-Cell-MMP comprising a β2M sequence having at least 85% (e.g., at least 90%, 95%, 98% or 99%, or even 100%) aa sequence identity to a sequence shown in FIG. 4 before the addition of the pair of cysteines and/or selenocysteines, or into a peptide linker attached to one of those sequences. In one such embodiment the pair of cysteines and/or selenocysteines may be utilized as a bis-thiol linker coupling site for the conjugation of, for example, epitopes and/or payloads either directly or indirectly through a peptide or chemical linker. In one embodiment, the pair of cysteines and/or selenocysteines is located within 10, 20, 30, 40 or 50 aas of the amino terminus of the first polypeptide of the T-Cell-MMP.

In another embodiment, a pair of cysteines and/or selenocysteines is incorporated into an IgFc sequence incorporated into a second polypeptide to provide a chemical conjugation site. In an embodiment a pair of cysteines and/or selenocysteines is incorporated into a polypeptide comprising an IgFc sequence having at least 85% (e.g., at least 90%, 95%, 98% or 99%, or even 100%) aa sequence identity to a sequence shown in any of FIGS. 2A-2G before the addition of the pair of cysteines or selenocysteines, or into a peptide linker attached to one of those sequences. In one such embodiment the pair of cysteines and/or selenocysteines may be utilized as a bis-thiol linker coupling site for the conjugation of, for example, epitopes and/or payloads either directly or indirectly through a peptide or chemical linker.

In another embodiment, a pair of cysteines and/or selenocysteines is incorporated into a polypeptide comprising a MHC Class I heavy chain polypeptide sequence as a chemical conjugation site. In an embodiment, a pair of cysteines and/or selenocysteines is incorporated into a polypeptide comprising a sequence having at least 85% (e.g., at least 90%, 95%, 98% or 99%, or even 100%) aa sequence identity to a sequence shown in any of FIGS. 3A-3I before the addition of a pair of cysteines or selenocysteines, or into a peptide linker attached to one of those sequences. In one such embodiment the pair of cysteines and/or selenocysteines may be utilized as a bis-thiol linker coupling site for the conjugation of, for example, epitopes and/or payloads either directly or indirectly through a peptide or chemical linker.

f. Other Chemical Conjugation Sites

(i) Carbohydrate Chemical Conjugation Sites

Many proteins prepared by cellular expression contain added carbohydrates (e.g., oligosaccharides of the type added to antibodies expressed in mammalian cells). Accordingly, where first and/or second polypeptides of a T-Cell-MMP are prepared by cellular expression, carbohydrates may be present and available as site selective chemical conjugation sites in glycol-conjugation reactions. McCombs and Owen, AAPS Journal, (2015) 17(2): 339-351, and references cited therein, describe the use of carbohydrate residues for glycol-conjugation of molecules to antibodies.

The addition and modification of carbohydrate residues may also be conducted ex vivo, through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites.

In an embodiment, the incorporation of an IgFc scaffold with known glycosylation sites may be used to introduce site specific chemical conjugation sites.

This disclosure includes and provides for T-Cell-MMPs and their epitope conjugates having carbohydrates as chemical conjugation (glycol-conjugation) sites. The disclosure also includes and provides for the use of such molecules in forming conjugates with epitopes and with other molecules such as drugs and diagnostic agents, and the use of those molecules in methods of medical treatment and diagnosis.

(ii) Nucleotide Binding Sites

Nucleotide binding sites offer site-specific functionalization through the use of a UV-reactive moiety that can covalently link to the binding site. Bilgicer et al., Bioconjug Chem. 2014; 25(7):1198-202, reported the use of an indole-3-butyric acid (IBA) moiety that can be covalently linked to an IgG at a nucleotide binding site. By incorporation of the sequences required to form a nucleotide binding site, chemical conjugates of T-Cell-MMP with suitably modified epitopes and/or other molecules (e.g., drugs or diagnostic agents) bearing a reactive nucleotide may be employed to prepare T-Cell-MMP-epitope conjugates.

This disclosure includes and provides for T-Cell-MMPs having nucleotide binding sites as chemical conjugation sites. The disclosure also includes and provides for the use of such molecules in forming conjugates with epitopes and with other molecules such as drugs and diagnostic agents, and the use of those molecules in methods of treatment and diagnosis.

3 Binding and Properties of T-Cell-MMPs, Epitopes and MOD

The present disclosure provides T-Cell-MMP-epitope conjugates. In one embodiment the disclosure provides for a T-Cell-MMP-epitope conjugate comprising: a) a first polypeptide; and b) a second polypeptide, wherein the first and second polypeptides of the multimeric polypeptide comprise an epitope (e.g., a cancer-associated epitope, an infectious disease-associated epitope, or a self-epitope); a first MHC polypeptide; a second MHC polypeptide; and optionally an immunoglobulin (Ig) Fc polypeptide or a non-Ig scaffold. In another embodiment, the present disclosure also provides a T-Cell-MMP-epitope conjugate comprising: a) a first polypeptide comprising, in order from N-terminus to C-terminus: i) an epitope (e.g., a cancer-associated epitope, an infectious disease-associated epitope, or a self-epitope); and ii) a first MHC polypeptide; and b) a second polypeptide comprising, in order from N-terminus to C-terminus: i) a second MHC polypeptide; and ii) optionally an Ig Fc polypeptide or a non-Ig scaffold. In addition to those components recited above, at least one of the first and second polypeptides of the T-Cell-MMP-epitope conjugates of the present disclosure comprise one or more (e.g., at least one or at least two) MODs. The one or more MODs are located: A) at the C-terminus of the first polypeptide; B) at the N-terminus of the second polypeptide; C) at the C-terminus of the second polypeptide; D) at the C-terminus of the first polypeptide and at the N-terminus of the second polypeptide; and/or E) between the MHC polypeptide and an Ig Fc polypeptide of the second polypeptide. In an embodiment, at least one (e.g., at least two, or at least three) of the one or more MODs is a variant MOD that exhibits reduced affinity to a Co-MOD compared to the affinity of a corresponding wild-type MOD for the Co-MOD.

In an embodiment, the epitope present in a T-Cell-MMP-epitope conjugate of the present disclosure (see, e.g., FIG. 6) binds to a T-cell receptor (TCR) on a T-cell with an affinity of at least 100 μM (e.g., at least 10 μM, at least 1 μM, at least 100 nM, at least 10 nM or at least 1 nM). In an embodiment, a T-Cell-MMP-epitope conjugate of the present disclosure binds to a first T-cell with an affinity that is at least 25% higher than the affinity with which the T-Cell-MMP-epitope conjugate binds to a second T-cell, where the first T-cell expresses on its surface the Co-MOD and a TCR that binds the epitope with an affinity of at least 100 μM, and where the second T-cell expresses on its surface the Co-MOD but does not express on its surface a TCR that binds the epitope with an affinity of at least 100 μM (e.g., at least 10 μM, at least 1 μM, at least 100 nM, at least 10 nM, or at least 1 nM). In some cases, the peptide presenting an epitope present in a T-Cell-MMP-epitope conjugate of the present disclosure presents a cancer-associated epitope, an infectious disease-associated epitope (such as a virus or bacterial epitope), or a self-epitope (e.g., the peptide is a cancer-associated peptide, an infectious disease-associated peptide (e.g., a virus- or bacteria-encoded peptide) or a peptide of a self-antigen.

In some cases, the epitope present in a T-Cell-MMP-epitope conjugate of the present disclosure binds to a TCR on a T-cell with an affinity of from about 10⁻⁴ M to about 5×10⁻⁴ M, from about 5×10⁻⁴ M to about 10⁻⁵ M, from about 10⁻⁵ M to about 5×10⁻⁵ M, from about 5×10⁻⁵ M to about 10⁻⁶ M, from about 10⁻⁶ M to about 5×10⁻⁶ M, from about 5×10⁻⁶ M to about 10⁻⁷ M, from about 10⁻⁷ M to about 10⁻⁸ M or from about 10⁻⁸ M to about 10⁻⁹ M. Expressed another way, in some cases, the epitope present in a T-Cell-MMP-epitope conjugate of the present disclosure binds to a TCR on a T-cell with an affinity of from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, or from about 75 μM to about 100 μM.

In an embodiment, a variant MOD present in a T-Cell-MMP-epitope conjugate of the present disclosure binds to its Co-MOD with an affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the Co-MOD.

In some cases, a variant MOD present in a T-Cell-MMP-epitope conjugate of the present disclosure has a binding affinity for a Co-MOD that is from 1 nM to 100 nM, or from 100 nM to 100 μM. For example, in some cases, a variant MOD present in a T-Cell-MMP-epitope conjugate of the present disclosure has a binding affinity for a Co-MOD that is from about 1 nM to about 5 nM, from about 5 nM to about 10 nM, from about 10 nM to about 50 nM, from about 50 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant MOD present in a T-Cell-MMP of the present disclosure has a binding affinity for a Co-MOD that is from about 1 nM to about 5 nM, from about 5 nM to about 10 nM, from about 10 nM to about 50 nM, or from about 50 nM to about 100 nM.

The combination of the reduced affinity of the MOD for its Co-MOD and the affinity of the epitope for a TCR provides for enhanced selectivity of a T-Cell-MMP-epitope conjugate of the present disclosure, while still allowing for activity of the MOD. For example, a T-Cell-MMP-epitope conjugate of the present disclosure binds selectively to a first T-cell that displays both: i) a TCR specific for the epitope present in the T-Cell-MMP-epitope conjugate; and ii) a Co-MOD that binds to the MOD present in the T-Cell-MMP-epitope conjugate, compared to binding to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate. For example, a T-Cell-MMP-epitope conjugate of the present disclosure binds to the first T-cell with an affinity that is at least 10%, at least 15%, at least 20%, at least 25%, at least 30%, at least 40%, at least 50%, at least 60%, at least 70%, at least 80%, at least 90%, at least 200% (2-fold), at least 250% (2.5-fold), at least 500% (5-fold), at least 1,000% (10-fold), at least 1,500% (15-fold), at least 2,000% (20-fold), at least 2,500% (25-fold), at least 5,000% (50-fold), at least 10,000% (100-fold), or more than 100-fold, higher than the affinity to which it binds the second T-cell.

In some cases, a T-Cell-MMP-epitope conjugate of the present disclosure, when administered to an individual in need thereof, induces both an epitope-specific T-cell response and an epitope non-specific T-cell response. In other words, in some cases, the T-Cell-MMP-epitope conjugate of the present disclosure, when administered to an individual in need thereof, induces an epitope-specific T-cell response by modulating the activity of a first T-cell that displays both: i) a TCR specific for the epitope present in the T-Cell-MMP-epitope conjugate; and ii) a Co-MOD that binds to the MOD present in the T-Cell-MMP-epitope conjugate. The T-Cell-MMP-epitope conjugate also induces an epitope non-specific T-cell response by modulating the activity of a second T-cell that displays: i) a TCR specific for an epitope other than the epitope present in the T-Cell-MMP-epitope conjugate; and ii) a Co-MOD that binds to the MOD present in the T-Cell-MMP-epitope conjugate. The ratio of the epitope-specific T-cell response to the epitope-non-specific T-cell response is at least 2:1, at least 5:1, at least 10:1, at least 15:1, at least 20:1, at least 25:1, at least 50:1, or at least 100:1. The ratio of the epitope-specific T-cell response to the epitope-non-specific T-cell response is from about 2:1 to about 5:1, from about 5:1 to about 10:1, from about 10:1 to about 15:1, from about 15:1 to about 20:1, from about 20:1 to about 25:1, from about 25:1 to about 50:1, from about 50:1 to about 100:1, or more than 100:1. “Modulating the activity” of a T-cell can include, but is not limited to, one or more of: i) activating a cytotoxic (e.g., CD8⁺) T-cell; ii) inducing cytotoxic activity of a cytotoxic (e.g., CD8⁺) T-cell; iii) inducing production and release of a cytotoxin (e.g., a perforin; a granzyme; a granulysin) by a cytotoxic (e.g., CD8⁺) T-cell; and iv) inhibiting activity of an autoreactive T-cell; and the like.

The combination of the reduced affinity of the MOD for its Co-MOD and the affinity of the epitope for a TCR provides for enhanced selectivity of a T-Cell-MMP-epitope conjugate of the present disclosure. Thus, for example, a T-Cell-MMP-epitope conjugate of the present disclosure binds with higher avidity to a first T-cell that displays both: i) a TCR specific for the epitope present in the T-Cell-MMP-epitope conjugate; and ii) a Co-MOD that binds to the MOD present in the T-Cell-MMP-epitope conjugate, compared to the avidity with which it binds to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate.

a. Determining Binding Affinity

Binding affinity between a MOD and its Co-MOD can be determined by bio-layer interferometry (BLI) using purified MOD and purified Co-MOD. Binding affinity between a T-Cell-MMP-epitope conjugate and its Co-MOD can be determined by BLI using purified T-Cell-MMP-epitope conjugate and the Co-MOD. BLI methods are well known to those skilled in the art. See, e.g., Lad et al. (2015) J. Biomol. Screen., 20(4):498-507; and Shah and Duncan (2014) J. Vis. Exp. 18:e51383. The specific and relative binding affinities described in this disclosure between a Co-MOD and a MOD, or between a Co-MOD and a T-Cell-MMP (or its epitope conjugate), can be determined using the following procedures.

A BLI assay can be carried out using an Octet RED 96 (Pal FortéBio) instrument, or a similar instrument, as follows. For example, to determine binding affinity of a Co-MOD for a T-Cell-MMP (or its epitope conjugate) (e.g., a T-Cell-MMP-epitope conjugate of the present disclosure with a variant MOD, or a control T-Cell-MMP-epitope conjugate comprising a wild-type MOD), the T-Cell-MMP (or its epitope conjugate) is immobilized onto an insoluble support (a “biosensor”). The immobilized T-Cell-MMP (or its epitope conjugate) is the “target” Immobilization can be effected by immobilizing a capture antibody onto the insoluble support, where the capture antibody immobilizes the T-Cell-MMP (or its epitope conjugate). For example, where the T-Cell-MMP comprises an IgFc polypeptide, immobilization can be effected by immobilizing anti-Fc (e.g., anti-human IgG Fc) antibodies onto the insoluble support, and contacting the T-Cell-MMP-epitope conjugate with the immobilized anti-Fc antibodies which will bind to and immobilize it. A Co-MOD is applied, at several different concentrations, to the immobilized T-Cell-MMP (or its immobilized epitope conjugate), and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the Co-MOD to the immobilized T-Cell-MMP (or its epitope conjugate) is conducted at 30° C. As a positive control for binding affinity, an anti-MHC Class I monoclonal antibody can be used. For example, anti-HLA Class I monoclonal antibody (mAb) W6/32 (American Type Culture Collection No. HB-95; Parham et al. (1979) J. Immunol. 123:342), which has a K_{D} of 7 nM, can be used. A standard curve can be generated using serial dilutions of the anti-MHC Class I monoclonal antibody. The Co-MOD, or the anti-MHC Class I mAb, is the “analyte.” BLI analyzes the interference pattern of white light reflected from two surfaces: i) the immobilized polypeptide (“target”); and ii) an internal reference layer. A change in the number of molecules (“analyte”; e.g., Co-MOD; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/kₐ) gives rise to the affinity constant K_{D}. The assay can also be conducted with purified wild-type or its variant MOD immobilized on the biosensor while the Co-MOD is applied, at several different concentrations, to determine the binding parameters between a MOD and its Co-MOD.

Determining the binding affinity of a Co-MOD (e.g., IL-2R) with both a wt. MOD (e.g., IL-2) and a variant MOD (e.g., an IL-2 variant as disclosed herein), or with a T-Cell-MMP (or its epitope conjugate) containing wt. or variant MODs, thus allows one to determine the relative binding affinity of the wt. and variant molecules. That is, one can determine whether the binding affinity of a variant MOD for its receptor (its Co-MOD) is reduced as compared to the binding affinity of the wt. MOD for the same Co-MOD, and, if so, what is the percentage reduction from the binding affinity of the wt. Co-MOD.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008), J. Anal. Biochem., 377:209.

Unless otherwise stated herein, the affinity of a T-Cell-MMP-epitope conjugate of the present disclosure for a Co-MOD, or the affinity of a control T-Cell-MMP-epitope conjugate (where a control T-Cell-MMP-epitope conjugate comprises a wild-type MOD) for a Co-MOD, is determined using BLI, as described above. Likewise, the affinity of a MOD and its Co-MOD polypeptide can be determined using BLI as described above.

In some cases, the ratio of: i) the binding affinity of a control T-Cell-MMP-epitope conjugate (where the control comprises a wt. MOD) to a Co-MOD to ii) the binding affinity of a T-Cell-MMP-epitope conjugate of the present disclosure comprising a variant of the wt. MOD to the Co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control T-Cell-MMP-epitope conjugate (where the control comprises a wt. MOD) to a Co-MOD to ii) the binding affinity of a T-Cell-MMP-epitope conjugate of the present disclosure comprising a variant of the wt. MOD to the Co-MOD, when measured by BLI, is in a range of from 1.5:1 to 10⁶:1, e.g., from 1.5:1 to 10:1, from 2.0:1 to 5:1, from 10:1 to 15:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As an example, where a control T-Cell-MMP-epitope conjugate comprises a wt. IL-2 polypeptide, and where a T-Cell-MMP-epitope conjugate of the present disclosure comprises a variant IL-2 polypeptide (comprising from 1 to 10 aa substitutions relative to the aa sequence of the wt. IL-2 polypeptide) as the MOD, the ratio of: i) the binding affinity of the control T-Cell-MMP-epitope conjugate to an IL-2 receptor (the Co-MOD) to ii) the binding affinity of the T-Cell-MMP-epitope conjugate of the present disclosure to the IL-2 receptor (the Co-MOD), when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, where a control T-Cell-MMP-epitope conjugate comprises a wt. IL-2 polypeptide, and where a T-Cell-MMP-epitope conjugate of the present disclosure comprises a variant IL-2 polypeptide (comprising from 1 to 10 aa substitutions relative to the aa sequence of the wt. IL-2 polypeptide) as the MOD, the ratio of: i) the binding affinity of the control T-Cell-MMP-epitope conjugate to the IL-2 receptor (the Co-MOD) to ii) the binding affinity of the T-Cell-MMP-epitope conjugate of the present disclosure to the IL-2 receptor, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As another example, where a control T-Cell-MMP-epitope conjugate comprises a wt. PD-L1 polypeptide, and where a T-Cell-MMP-epitope conjugate of the present disclosure comprises a variant PD-L1 polypeptide (comprising from 1 to 10 aa substitutions relative to the aa sequence of the wild-type PD-L1 polypeptide) as the MOD, the ratio of: i) the binding affinity of the control T-Cell-MMP-epitope conjugate to a PD-1 polypeptide (i.e., the Co-MOD) to ii) the binding affinity of the T-Cell-MMP-epitope conjugate of the present disclosure to the PD-1 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control T-Cell-MMP-epitope conjugate comprises a wt. CD80 polypeptide, and where a T-Cell-MMP-epitope conjugate of the present disclosure comprises a variant CD80 polypeptide (comprising from 1 to 10 aa substitutions relative to the aa sequence of the wt. CD80 polypeptide) as the MOD, the ratio of: i) the binding affinity of the control T-Cell-MMP-epitope conjugate to CTLA4 polypeptide (i.e., the Co-MOD) to ii) the binding affinity of the T-Cell-MMP-epitope conjugate of the present disclosure to the CTLA4 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control T-Cell-MMP-epitope conjugate comprises a wt. CD80 polypeptide, and where a T-Cell-MMP-epitope conjugate of the present disclosure comprises a variant CD80 polypeptide (comprising from 1 to 10 aa substitutions relative to the aa sequence of the wt. CD80 polypeptide) as the MOD, the ratio of: i) the binding affinity of the control T-Cell-MMP-epitope conjugate to a CD28 polypeptide (i.e., the Co-MOD) to ii) the binding affinity of the T-Cell-MMP-epitope conjugate of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control T-Cell-MMP-epitope conjugate comprises a wt. 4-1BBL polypeptide, and where a T-Cell-MMP-epitope conjugate of the present disclosure comprises a variant 4-1BBL polypeptide (comprising from 1 to 10 aa substitutions relative to the aa sequence of the wt. 4-1BBL polypeptide) as the MOD, the ratio of: i) the binding affinity of the control T-Cell-MMP-epitope conjugate to a 4-1BB polypeptide (i.e., the Co-MOD) to ii) the binding affinity of the T-Cell-MMP-epitope conjugate of the present disclosure to the 4-1BB polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control T-Cell-MMP-epitope conjugate comprises a wt. CD86 polypeptide, and where a T-Cell-MMP-epitope conjugate of the present disclosure comprises a variant CD86 polypeptide (comprising from 1 to 10 aa substitutions relative to the aa sequence of the wild-type CD86 polypeptide) as the MOD, the ratio of: i) the binding affinity of the control T-Cell-MMP-epitope conjugate to a CD28 polypeptide (i.e., the Co-MOD) to ii) the binding affinity of the T-Cell-MMP-epitope conjugate of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

Binding affinity of a T-Cell-MMP-epitope conjugate of the present disclosure to a target T-cell can be measured in the following manner: A) contacting a T-Cell-MMP-epitope conjugate of the present disclosure with a target T-cell expressing on its surface with: i) a Co-MOD that binds to the parental wt. MOD; and ii) a TCR that binds to the epitope, where the T-Cell-MMP-epitope conjugate comprises an epitope tag or fluorescent label, such that the T-Cell-MMP-epitope conjugate binds to the target T-cell; B) if the T-Cell-MMP-epitope conjugate is unlabeled, contacting the target T-cell-bound T-Cell-MMP-epitope conjugate with a fluorescently labeled binding agent (e.g., a fluorescently labeled antibody) that binds to the epitope tag, generating a T-Cell-MMP-epitope conjugate/target T-cell/binding agent complex; and C) measuring the mean fluorescence intensity (MFI) of the T-Cell-MMP-epitope conjugate/target T-cell/binding agent complex using flow cytometry. The epitope tag can be, e.g., a FLAG tag, a hemagglutinin tag, a c-myc tag, a poly(histidine) tag, etc. The MFI measured over a range of concentrations of the T-Cell-MMP-epitope conjugate (library member) provides a measure of the affinity. The MFI measured over a range of concentrations of the T-Cell-MMP-epitope conjugate (library member) provides a half maximal effective concentration (EC₅₀) of the T-Cell-MMP-epitope conjugate. In some cases, the EC₅₀ of a T-Cell-MMP-epitope conjugate of the present disclosure for a target T-cell is in the nM range; and the EC₅₀ of the T-Cell-MMP-epitope conjugate for a control T-cell (where a control T-cell expresses on its surface: i) a Co-MOD that binds the parental wt. MOD; and ii) a T-cell receptor that does not bind to the epitope present in the T-Cell-MMP-epitope conjugate) is in the μM range. In some cases, the ratio of the EC₅₀ of a T-Cell-MMP-epitope conjugate of the present disclosure for a control T-cell to the EC₅₀ of the T-Cell-MMP-epitope conjugate for a target T-cell is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at lease 10⁵:1, or at least 10⁶:1. The ratio of the EC₅₀ of a T-Cell-MMP-epitope conjugate of the present disclosure for a control T-cell to the EC₅₀ of the T-Cell-MMP-epitope conjugate for a target T-cell is an expression of the selectivity of the T-Cell-MMP-epitope conjugate.

In some cases, when measured as described in the preceding paragraph, a T-Cell-MMP-epitope conjugate of the present disclosure exhibits selective binding to a target T-cell, compared to binding of the T-Cell-MMP-epitope conjugate (library member) to a control T-cell that comprises: i) the Co-MOD that binds the parental wt. MOD; and ii) a TCR that binds to an epitope other than the epitope present in the T-Cell-MMP-epitope conjugate (library member).

b. Dimerized Multimeric T-Cell Modulatory Polypeptides

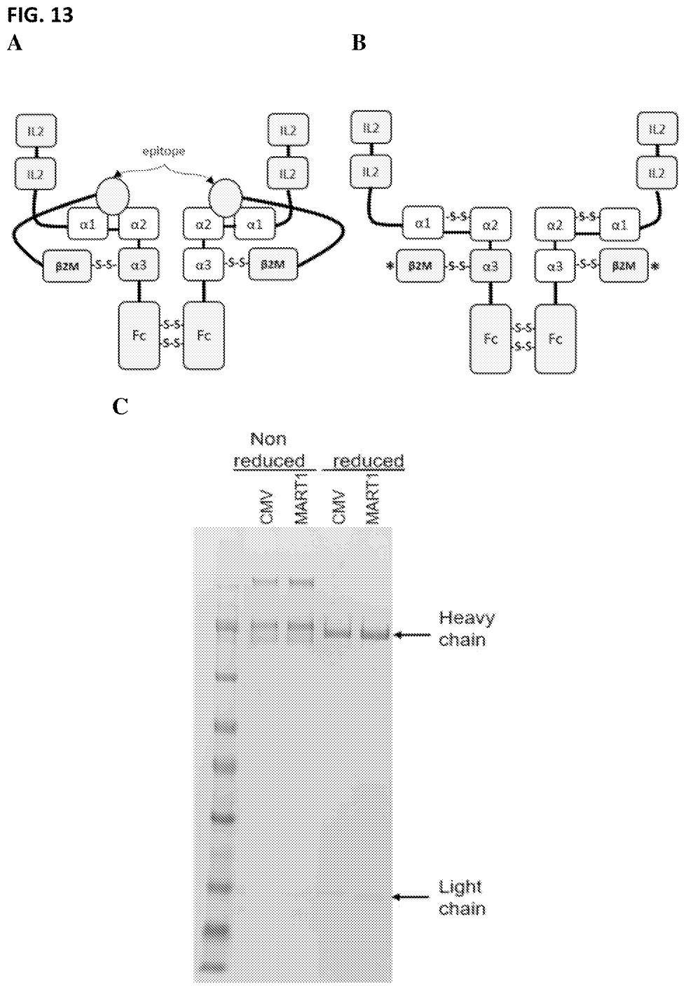

T-Cell-MMPs and T-Cell-MMP-epitope conjugates of the present disclosure can be in the form of dimers, i.e., the present disclosure provides a multimeric polypeptide comprising a dimer of a multimeric T-Cell-MMP of the present disclosure. An example of a dimerized T-Cell-MMP is shown in FIG. 13 structure B, and examples of dimerized T-Cell-MMP-epitope conjugates are shown in FIG. 7. Thus, the present disclosure provides a multimeric T-Cell-MMP comprising: A) a first heterodimer comprising: a) a first polypeptide comprising: i) a peptide epitope; and ii) a first MHC polypeptide; and b) a second polypeptide comprising a second MHC polypeptide, wherein the first heterodimer comprises one or more MODs; and B) a second heterodimer comprising: a) a first polypeptide comprising: i) a peptide epitope; and ii) a first MHC polypeptide; and b) a second polypeptide comprising a second MHC polypeptide, wherein the second heterodimer comprises one or more MODs, and wherein the first heterodimer and the second heterodimer are covalently linked to one another. In some cases, the two heterodimers that form the dimerized T-Cell-MMPs are identical to one another in aa sequence. In some cases, the first heterodimer and the second heterodimer are covalently linked to one another via a C-terminal region of the second polypeptide of the first heterodimer and a C-terminal region of the second polypeptide of the second heterodimer (see, e.g., the disulfide bonds between the IgFc regions (“Fc”) in FIG. 7 and FIG. 13 structure B). In some cases, the first heterodimer and the second heterodimer are covalently linked to one another via an aa in the C-terminal region of the second polypeptide of the first heterodimer and an aa in the C-terminal region of the second polypeptide of the second heterodimer; for example, in some cases, the C-terminal aa of the second polypeptide of the first heterodimer and the C-terminal aa of the second polypeptide of the second heterodimer are linked to one another, either directly or via a linker. The linker can be a peptide linker. The peptide linker can have a length of from 1 aa to 200 aa (e.g., from 1 aa to 5 aa, from 5 aa to 10 aa, from 10 aa to 25 aa, from 25 aa to 50 aa, from 50 aa to 100 aa, from 100 aa to 150 aa, or from 150 aa to 200 aa).

The first MHC polypeptides of the first and second heterodimers may be β2M polypeptides, and the second MHC polypeptides of the first and second heterodimers may be MHC Class I heavy chain polypeptides. In some cases, the MOD of the first heterodimer and the MOD of the second heterodimer comprise the same aa sequence. In some cases, the MOD of the first heterodimer and the MOD of the second heterodimer are variant MODs that comprise from 1 to 10 aa substitutions compared to a corresponding parental wt. MOD, wherein from 1 to 10 aa substitutions result in reduced affinity binding of the variant MOD to a Co-MOD. In some cases, the MOD(s) of the first heterodimer and the MOD(s) of the second heterodimer are each independently selected from the group consisting of IL-2, 4-1BBL, PD-L1, CD70, CD80, CD86, ICOS-L, OX-40L, FasL, JAG1(CD339), TGF-β, ICAM, and variant MODs thereof (e.g., variant MODs having 1 to 10 aa substitutions compared to a corresponding parental wt. MOD). Examples of suitable MHC polypeptides, MODs, and peptide epitopes are described below.

In some cases, the peptide epitope of the first heterodimer and the peptide epitope of the second heterodimer comprise the same amino acid sequence.

In addition to dimers, the T-Cell-MMPs and T-Cell-MMP-epitope conjugates of the present disclosure may form higher order complexes including trimers, tetramers, or pentamers. Compositions comprising multimers of T-Cell-MMPs may also comprise lower order complexes such as monomers and, accordingly, may comprise monomers, dimers, trimers, tetramers, pentamers, or combinations of any thereof (e.g., a mixture of monomers and dimers).

4 MHC Polypeptides of T-Cell-MMPs

As noted above, T-Cell-MMPs and T-Cell-MMP-epitope conjugates include MHC polypeptides. For the purposes of the instant disclosure, the term “major histocompatibility complex (MHC) polypeptides” is meant to include MHC Class I polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC Class I heavy chain and/or portions thereof). In some cases, the first MHC polypeptide is a MHC Class I β2M (β2M) polypeptide, and the second MHC polypeptide is a MHC Class I heavy chain (MHC-H). In an embodiment, both the β2M and MHC-H chain sequences in a T-Cell-MMP (or its epitope conjugate) are of human origin. Unless expressly stated otherwise, the T-Cell-MMPs and the T-Cell-MMP-epitope conjugates described herein are not intended to include membrane anchoring domains (transmembrane regions) of a MHC Class I heavy chain, or a part of that molecule sufficient to anchor the resulting T-Cell-MMP, or a peptide thereof, to a cell (e.g., eukaryotic cell such as a mammalian cell) in which it is expressed. In some cases, the MHC Class I heavy chain present in T-Cell-MMPs and T-Cell-MMP-epitope conjugates does not include a signal peptide, a transmembrane domain, or an intracellular domain (cytoplasmic tail) associated with a native MHC Class I heavy chain. Thus, e.g., in some cases, the MHC Class I heavy chain present in a T-Cell-MMP of the present disclosure includes only the α1, α2, and α3 domains of an MHC Class I heavy chain. In some cases, the MHC Class I heavy chain present in a T-Cell-MMP of the present disclosure has a length of from about 270 amino acids (aa) to about 290 aa. In some cases, the MHC Class I heavy chain present in a T-Cell-MMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, a MHC polypeptide of a T-Cell-MMP, or a T-Cell-MMP-epitope conjugate is a human MHC polypeptide, where human MHC polypeptides are also referred to as “human leukocyte antigen” (“HLA”) polypeptides, more specifically, a Class I HLA polypeptide, e.g., a β2M polypeptide, or a Class I HLA heavy chain polypeptide. Class I HLA heavy chain polypeptides that can be included in T-Cell-MMPs or their epitope conjugates include HLA-A, -B, -C, -E, -F, and/or -G heavy chain polypeptides. In an embodiment, the Class I HLA heavy chain polypeptides of T-Cell-MMPs or their epitope conjugates comprise polypeptides having a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aas) of the aa sequence of any of the human HLA heavy chain polypeptides depicted in FIGS. 3A to 3I. For example, they may comprise 1-30, 1-5, 5-10, 10-15, 15-20, 20-25 or 25-30 aa insertions, deletions, and/or substitutions (in addition to those locations indicated as being variable in the heavy chain consensus sequences of FIGS. 3E to 3I).

As an example, a MHC Class I heavy chain polypeptide of a multimeric polypeptide can comprise an aa sequence having at least 75%, at least 80%, at least 85%, at least 90%, at least 95%, at least 98%, at least 99%, or 100% aa sequence identity to aas 25-300 (lacking all, or substantially all, of the leader, transmembrane and cytoplasmic sequences) or 25-365 (lacking the leader) of the human HLA-A heavy chain polypeptides depicted in FIGS. 3A, 3B and/or 3C.

a. MHC Class I Heavy Chains

Class I human MHC polypeptides may be drawn from the classical HLS alleles (HLA-A, B, and C), or the non-classical HLA alleles (e.g., HLA-E, F and G). The following are non-limiting examples of MHC-H alleles and variants of those alleles that may be incorporated into T-Cell-MMPs and their epitope conjugates.

(i) HLA-A Heavy Chains

The HLA-A heavy chain peptide sequences, or portions thereof, that may be incorporated into a T-Cell-MMP or its epitope conjugate include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 3E. Any of those alleles may comprise a substitution at one or more of positions 84, 139 and/or 236 (as shown in FIG. 3E) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, a HLA-A sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of those HLA-A alleles may also be incorporated into a T-Cell-MMP (e.g., it may comprise 1-30, 1-5, 5-10, 10-15, 15-20, 20-25, or 25-30 aa insertions, deletions, and/or substitutions).

(a) HLA-A*0101 (HLA-A*01:01:01:01)

An MHC Class I heavy chain polypeptide of a T-Cell-MMP or a T-Cell-MMP-epitope conjugate may comprise aa sequence of HLA-A*01:01:01:01 (HLA-A in FIG. 3D (SEQ ID NO:20) or FIG. 3E), or a sequence having at least 75% (at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A heavy chain polypeptide of a T-Cell-MMP or its epitope conjugate has less than 100% identity to the sequence labeled HLA-A in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C).

(b) HLA-A*0201

An MHC Class I heavy chain polypeptide of a T-Cell-MMP or a T-Cell-MMP-epitope conjugate may comprise an aa sequence of HLA-A*0201 (SEQ ID NO:23) provided in FIG. 3D or FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A*0201 heavy chain polypeptide of a T-Cell-MMP or its epitope conjugate has less than 100% identity to the sequence labeled HLA-A*0201 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*0201 heavy chain polypeptide of a T-Cell-MMP or its epitope conjugate comprises the Y84A and A236C substitutions. In an embodiment, the HLA-A*0201 heavy chain polypeptide of a T-Cell-MMP or its epitope conjugate comprises the Y84C and A139C substitutions. In an embodiment, the HLA-A*0201 heavy chain polypeptide of a T-Cell-MMP or its epitope conjugate comprises the Y84C, A139C and A236C substitutions.

(c) HLA-A*1101

An MHC Class I heavy chain polypeptide of a T-Cell-MMP or a T-Cell-MMP-epitope conjugate may comprise an aa sequence of HLA-A*1101 (SEQ ID NO:28) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a T-Cell-MMP or its epitope conjugate has less than 100% identity to the sequence labeled HLA-A*1101 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*1101 heavy chain polypeptide of a T-Cell-MMP or its epitope conjugate comprises the Y84A and A236C substitutions. In an embodiment, the HLA-A*1101 heavy chain polypeptide of a T-Cell-MMP or its epitope conjugate comprises the Y84C and A139C substitutions. In an embodiment, the HLA-A*1101 heavy chain polypeptide of a T-Cell-MMP or its epitope conjugate comprises the Y84C, A139C and A236C substitutions.

(d) HLA-A*2402

An MHC Class I heavy chain polypeptide of a T-Cell-MMP or a T-Cell-MMP-epitope conjugate may comprise an aa sequence of HLA-A*2402 (SEQ ID NO:29) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a T-Cell-MMP or its epitope conjugate has less than 100% identity to the sequence labeled HLA-A*2402 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*2402 heavy chain polypeptide of a T-Cell-MMP or its epitope conjugate comprises the Y84A and A236C substitutions. In an embodiment, the HLA-A*2402 heavy chain polypeptide of a T-Cell-MMP or its epitope conjugate comprises the Y84C and A139C substitutions. In an embodiment, the HLA-A*2402 heavy chain polypeptide of a T-Cell-MMP or its epitope conjugate comprises the Y84C, A139C and A236C substitutions.

(e) HLA-A*3303

An MHC Class I heavy chain polypeptide of a T-Cell-MMP or a T-Cell-MMP-epitope conjugate may comprise an aa sequence of HLA-A*3303 (SEQ ID NO:30) provided in FIG. 3D or 3E, or a sequence having at least 75% (e.g., at least 80%, at least 85%, at least 90%, at least 95%, at least 98%, or at least 99%) or 100% aa sequence identity to all or part (e.g., 50, 75, 100, 150, 200, or 250 contiguous aas) of that sequence (e.g., it may comprise 1-25, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 heavy chain polypeptide of a T-Cell-MMP or its epitope conjugate has less than 100% identity to the sequence labeled HLA-A*3303 in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*3303 heavy chain polypeptide of a T-Cell-MMP or its epitope conjugate comprises the Y84A and A236C substitutions. In an embodiment, the HLA-A*3303 heavy chain polypeptide of a T-Cell-MMP or its epitope conjugate comprises the Y84C and A139C substitutions. In an embodiment, the HLA-A*3303 heavy chain polypeptide of a T-Cell-MMP or its epitope conjugate comprises the Y84C, A139C and A236C substitutions.

(ii) HLA-B Heavy Chains.

The HLA-B heavy chain peptide sequences, or portions thereof, that may be incorporated into a T-Cell-MMP or its epitope conjugate include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 3F. Any of those alleles may comprise a substitution at one or more of positions 84, 139 and/or 236 (as shown in FIG. 3F) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, a HLA-B sequence having at least 75% (e.g., at least 80%, at least 85%, at least 90%, at least 95%, at least 98%, at least 99%) or 100% aa sequence identity to all or part (e.g., 50, 75, 100, 150, 200, or 250 contiguous aas) of the sequence of those HLA-B alleles may also be incorporated into a T-Cell-MMP (e.g., it may comprise 1-25, 1-5, 5-10, 10-15, 15-20, 20-25, or 25-30 aa insertions, deletions, and/or substitutions).

(a) HLA-B*0702

An MHC Class I heavy chain polypeptide of a T-Cell-MMP or a T-Cell-MMP-epitope conjugate may comprise an aa sequence of HLA-B*0702 (SEQ ID NO:21) in FIG. 3D (labeled HLA-B in FIG. 3D), or a sequence having at least 75% (e.g., at least 80%, at least 85%, at least 90%, at least 95%, at least 98%, at least 99%) or 100% aa sequence identity to all or part (e.g., 50, 75, 100, 150, 200, or 250 contiguous aas) of that sequence (e.g., it may comprise 1-25, 1-5, 5-10, 10-15, 15-20, 20-25, or 25-30 aa insertions, deletions, and/or substitutions). In an embodiment, where the HLA-B heavy chain polypeptide of a T-Cell-MMP or its epitope conjugate has less than 100% identity to the sequence labeled HLA-B in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-B heavy chain polypeptide of a T-Cell-MMP or its epitope conjugate comprises the Y84A and A236C substitutions. In an embodiment, the HLA-B*0702 heavy chain polypeptide of a T-Cell-MMP or its epitope conjugate comprises the Y84C and A139C substitutions. In an embodiment, the HLA-B heavy chain polypeptide of a T-Cell-MMP or its epitope conjugate comprises the Y84C, A139C and A236C substitutions.

(iii) HLA-C Heavy Chains

The HLA-C heavy chain peptide sequences, or portions thereof, that may be incorporated into a T-Cell-MMP or its epitope conjugate include, but are not limited to, the alleles: C*0102, C*0303, C*0304, C*0401, C*0602, C*0701, C*0702, C*0801, and C*1502, which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-C sequence having at least 75% (e.g., at least 80%, at least 85%, at least 90%, at least 95%, at least 98%, at least 99%) or 100% aa sequence identity to all or part (e.g., 50, 75, 100, 150, 200, or 250 contiguous aas) of the sequence of those HLA-C alleles may also be incorporated into a T-Cell-MMP (e.g., it may comprise 1-25, 1-5, 5-10, 10-15, 15-20, 20-25, or 25-30 aa insertions, deletions, and/or substitutions).

(a) HLA-C*701 and HLA-C*702

An MHC Class I heavy chain polypeptide of a T-Cell-MMP or a T-Cell-MMP-epitope conjugate may comprise an aa sequence of HLA-C*701 (SEQ ID NO:49) or HLA-C*702 (SEQ ID NO:50) in FIG. 3G (labeled HLA-C in FIG. 3D), or a sequence having at least 75% (e.g., at least 80%, at least 85%, at least 90%, at least 95%, at least 98%, at least 99%) or 100% aa sequence identity to all or part (e.g., 50, 75, 100, 150, 200, or 250 contiguous aas) of those sequences (e.g., it may comprise 1-25, 1-5, 5-10, 10-15, 15-20, 20-25, or 25-30 aa insertions, deletions, and/or substitutions relative to those sequences). In an embodiment, where the HLA-C heavy chain polypeptide of a T-Cell-MMP or its epitope conjugate has less than 100% identity to the sequence labeled HLA-C in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-C heavy chain polypeptide of a T-Cell-MMP or its epitope conjugate comprises the Y84A and A236C substitutions. In an embodiment, the HLA-C*701 heavy chain polypeptide of a T-Cell-MMP or its epitope conjugate comprises the Y84C and A139C substitutions. In an embodiment, the HLA-C heavy chain polypeptide of a T-Cell-MMP or its epitope conjugate comprises the Y84C, A139C and A236C substitutions.

(iv) Non-Classical HLA-E, F and G Heavy Chains

The non-classical HLA heavy chain peptide sequences, or portions thereof, that may be incorporated into a T-Cell-MMP or its epitope conjugate include, but are not limited to, those of the HLA-E, F, and/or G alleles. Sequences for those alleles, (and the HLA-A, B and C alleles) may be found on the world wide web at, for example, hla.alleles.org/nomenclature/index.html, the European Bioinformatics Institute (www.ebi.ac.uk), which is part of the European Molecular Biology Laboratory (EMBL), and the National Center for Bioecology Information (www.ncbi.nlm.nih.gov).

Some suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H, and aligned with consensus sequences of the above-mentioned HLA-A, -B, and -C alleles provided in FIGS. 3E-3G and in FIG. 3I.

Any of the above-mentioned HLA-E, F and/or G alleles may comprise a substitution at one or more of positions 84, 139 and/or 236 as shown in FIG. 3I for the consensus sequences. In an embodiment, the substitutions may be selected from: a position 84 tyrosine to alanine (Y84A) or cysteine (Y84C), or in the case of HLA-F a R84A or R84C substitution; a position 139 alanine to cysteine (A139C), or in the case of HLA-F a V139C substitution; and an alanine to cysteine at position 236 (A236C). In addition, HLA-E, -F, and/or -G sequences having at least 75% (e.g., at least 80%, at least 85%, at least 90%, at least 95%, at least 98%, at least 99%) or 100% aa sequence identity to all or part (e.g., 50, 75, 100, 150, 200, or 250 contiguous aas) of any of the consensus sequences set forth in FIG. 3I may also be employed (e.g., the sequences may comprise 1-25, 1-5, 5-10, 10-15, 15-20, 20-25, or 25-30 aa insertions, deletions, and/or substitutions in addition to changes at variable residues listed therein).

(v) Mouse H2K

An MHC Class I heavy chain polypeptide of a T-Cell-MMP or a T-Cell-MMP-epitope conjugate may comprise an aa sequence of MOUSE H2K (SEQ ID NO:24) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a T-Cell-MMP or its epitope conjugate has less than 100% identity to the sequence labeled MOUSE H2K in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the MOUSE H2K heavy chain polypeptide of a T-Cell-MMP or its epitope conjugate comprises the Y84A and A236C substitutions. In an embodiment, the MOUSE H2K heavy chain polypeptide of a T-Cell-MMP or its epitope conjugate comprises the Y84C and A139C substitutions. In an embodiment, the MOUSE H2K heavy chain polypeptide of a T-Cell-MMP or its epitope conjugate comprises the Y84C, A139C and A236C substitutions.

(vi) The Effect of Amino Acid Substitutions in MHC Polypeptides on T-Cell-MMPs

(a) Substitutions at Positions 84, 139 and 236

Substitution of position 84 of the MHC H chain (see FIG. 3I), particularly when it is a tyrosine residue, with a small amino acid such as alanine (Y84A) tends to open one end of the MHC binding pocket, allowing a linker (e.g., attached to an epitope peptide) to “thread” through the end of the pocket, and accordingly, permits a greater variation in the size of the epitope (e.g., longer peptides bearing epitope sequences) that can fit into the MHC pocket and be presented by the T-Cell-MMP. In an embodiment, the HLA-A heavy chain polypeptide of a T-Cell-MMP or its epitope conjugate comprises the Y84A and A236C substitutions. In an embodiment, the HLA-A heavy chain polypeptide of a T-Cell-MMP or its epitope conjugate comprises the Y84C and A139C substitutions. When amino acids 84 and 139 are both cysteines they may form an intrachain disulfide bond which can stabilize the MHC Class 1 protein and permit translation and excretion of the T-Cell-MMP by eukaryotic cells, even when not loaded with an epitope peptide. When position 84 is a C residue, it can also form an interchain disulfide bond with a linker attached to the N-terminus of a β2M polypeptide (e.g., epitope-linker sequence-mature β2M polypeptide, such as epitope-GCGGS(G₄S) linker sequence (SEQ ID NO:93)-mature β2M polypeptide, see SEQ ID NOs:57 to 61). When amino acid 236 is a cysteine it can form an interchain disulfide bond with a cysteine at amino acid 12 of a variant β2M polypeptide that comprises R12C substitution at that position. Some possible combinations of MHC Class 1 heavy chain sequence modifications that may be incorporated into a T-Cell-MMP or its epitope conjugate are shown in the Table that follows. Any combination of substitutions provided in the table at residues 84, 139 and 236 may be combined with any combination of substitutions in the epitope binding cleft, such as those described at positions 116 and 167.

(b) Substitutions at Positions 116 and 167

Any MHC Class I heavy chain sequences (including those disclosed above for: the HLA-A*0101 (HLA-A*01:01:01:01); HLA-A*0201; HLA-A*1101; HLA-A*2402; HLA-A*3303; HLA-B; HLA-C; and Mouse H2K, or the HLA-A, B, C, E, F, and/or G) may further comprise a cysteine substitution at position 116 (e.g., Y116C), providing thiol for anchoring an epitope peptide such as by reaction with a maleimide peptide and/or one of an alanine (W167A) or cysteine (W167C) at position 167. As with substitutions that open one end of the MHC-H binding pocket (e.g., at position 84 or its equivalent such as Y84A), substitution of an alanine or glycine at position 167 or its equivalent (e.g., a W167A substitution) opens the other end of the MHC binding pocket, creating a groove that permits greater variation (e.g., longer length) of the epitope peptides that may be presented by the T-Cell-MMP-epitope conjugates. Substitutions at positions 84 and 167 or their equivalent (e.g., Y84A in combination with W167A or W167G) may be used in combination to modify the binding pocket of MHC-H chains. The placement of a cysteine at position 167 (e.g., a W167C substitution) or its equivalent provides a thiol residue for anchoring an epitope peptide. Cysteine substitutions at positions 116 and 167 may be used separately to anchor epitopes (e.g., epitope peptides), or in combination to anchor the epitope in two locations (e.g., the ends of the epitope containing peptide. Substitutions at positions 116 and/or 167 may be combined with any one or more substitutions at positions 84, 139 and/or 236 described above.

SOME COMBINATIONS OF MHC CLASS 1 HEAVY CHAIN SEQUENCE MODIFICATIONS
THAT MAY BE INCORPORATED INTO A T-CELL-MMP OR ITS EPITOPE CONJUGATE

| | | | Specific | Substitutions |
|---|---|---|---|---|
| | HLA Heavy | | Substitutions | at positions |
| | Chain Sequence | | at aa positions | 116 and/or |
| Entry | From FIGs. 3D-H | Sequence Identity Ranges [symbol] | 84, 139 and/or 236 | 167 |
| 1 | HLA-A | 75%-99.8%, 80%-99.8%, 85%-99.8%, | None; Y84C; Y84A; | None; |
| | Consensus | 90%-99.8%, 95%-99.8%, 98%-99.8%, | A139C;A236C; | Y116C; |
| | FIG. 3E | or 99%-99.8%; or 1-25, 1-5, 5-10, | (Y84A & A236C); | W167A; |
| | | 10-15, 15-20, or 20-25 aa insertions, | (Y84C & A139C); or | W167C; or |
| | | deletions, and/or substitutions | (Y84C, A139C & | (Y116C & |
| | | (not counting variable residues) | A236C) | W167C) |
| 2 | A*0101, A*0201, | 75%-99.8%, 80%-99.8%, 85%-99.8%, | None; Y84C; Y84A; | None; |
| | A*0301, A*1101, | 90%-99.8%, 95%-99.8%, 98%-99.8%, | A139C; A236C; | Y116C; |
| | A*2402, A*2301, | or 99%-99.8%; or 1-25, 1-5, 5-10, | (Y84A & A236C); | W167A; |
| | A*2402, A*2407, | 10-15, 15-20, or 20-25 aa insertions, | (Y84C & A139C); or | W167C; or |
| | A*3303, or A*3401 | deletions, and/or substitutions | (Y84C, A139C & | (Y116C & |
| | | | A236C) | W167C) |
| 3 | HLA-B | 75%-99.8%, 80%-99.8%, 85%-99.8%, | None; Y84C; Y84A; | None; |
| | Consensus | 90%-99.8%, 95%-99.8%, 98%-99.8%, | A139C; A236C; | Y116C; |
| | FIG. 3F | or 99%-99.8%; or 1-25, 1-5, 5-10, | (Y84A & A236C); | W167A; |
| | | 10-15, 15-20, or 20-25 aa insertions, | (Y84C & A139C); or | W167C; or |
| | | deletions, and/or substitutions | (Y84C, A139C & | (Y116C & |
| | | (not counting variable residues) | A236C) | W167C) |
| 4 | B*0702, B*0801, | 75%-99.8%, 80%-99.8%, 85%-99.8%, | None; Y84C; Y84A; | None; |
| | B*1502, B*3802, | 90%-99.8%, 95%-99.8%, 98%-99.8%, | A139C; A236C; | Y116C; |
| | B*4001, B*4601, | or 99%-99.8%; or 1-25, 1-5, 5-10, | (Y84A & A236C); | W167A; |
| | or B*5301 | 10-15, 15-20, or 20-25 aa insertions, | (Y84C & A139C); or | W167C; or |
| | | deletions, and/or substitutions | (Y84C, A139C & | (Y116C & |
| | | | A236C) | W167C) |
| 5 | HLA-C | 75%-99.8%, 80%-99.8%, 85%-99.8%, | None; Y84C; Y84A; | None; |
| | Consensus | 90%-99.8%, 95%-99.8%, 98%-99.8%, | A139C; A236C; | Y116C; |
| | FIG. 3G | or 99%-99.8%; or 1-25, 1-5, 5-10, | (Y84A & A236C); | W167A; |
| | | 10-15, 15-20, or 20-25 aa insertions, | (Y84C & A139C); or | W167C; or |
| | | deletions, and/or substitutions | (Y84C, A139C & | (Y116C & |
| | | (not counting variable residues) | A236C) | W167C) |
| 6 | C*0102, C*0303, | 75%-99.8%, 80%-99.8%, 85%-99.8%, | None; Y84C; Y84A; | None; |
| | C*0304, C*0401, | 90%-99.8%, 95%-99.8%, 98%-99.8%, | A139C; A236C; | Y116C; |
| | C*0602, C*0701, | or 99%-99.8%; or 1-25, 1-5, 5-10, | (Y84A & A236C); | W167A; |
| | C*702, C*0801, | 10-15, 15-20, or 20-25 aa insertions, | (Y84C & A139C); or | W167C; or |
| | or C*1502 | deletions, and/or substitutions | (Y84C, A139C & | (Y116C & |
| | | | A236C) | W167C) |
| 7 | HLA-E, F, or G | 75%-99.8%, 80%-99.8%, 85%-99.8%, | None; Y84C; Y84A; | None; |
| | Consensus FIG. | 90%-99.8%, 95%-99.8%, 98%-99.8%, | A139C; A236C; | Y116C; |
| | 3H | or 99%-99.8%; or 1-25, 1-5, 5-10, | (Y84A & A236C); | W167A; |
| | | 10-15, 15-20, or 20-25 aa insertions, | (Y84C & A139C); or | W167C; or |
| | | deletions, and/or substitutions | (Y84C, A139C & | (Y116C & |
| | | (not counting variable residues) | A236C) | W167C) |
| 8 | MOUSE H2K | 75%-99.8%, 80%-99.8%, 85%-99.8%, | None; Y84C; Y84A; | None; |
| | | 90%-99.8%, 95%-99.8%, 98%-99.8%, | A139C; A236C; | Y116C; |
| | | or 99%-99.8%; or 1-25, 1-5, 5-10, | (Y84A & A236C); | W167A; |
| | | 10-15, 15-20, or 20-25 aa insertions, | (Y84C & A139C); or | W167C; or |
| | | deletions, and/or substitutions | (Y84C, A139C & | (Y116C & |
| | | | A236C) | W167C) |

[symbol]

The Sequence Identity Range is the permissible range in sequence identity of a MHC-H polypeptide sequence incorporated into a T-Cell-MMP relative to the corresponding portion of the sequences listed in FIG. 3D-3H not counting the variable residues in the consensus sequences.

b. MHC Class I β2-Microglobins and Combinations with MHC-H Polypeptides

A β2M polypeptide of a T-Cell-MMP or its epitope conjugate can be a human β2M polypeptide, a non-human primate β2M polypeptide, a murine β2M polypeptide, and the like. In some instances, a β2M polypeptide comprises an aa sequence having at least 75%, at least 80%, at least 85%, at least 90%, at least 95%, at least 98%, at least 99%, or 100% aa sequence identity to a β2M aa sequence depicted in FIG. 4. In some instances, a β2M polypeptide comprises an aa sequence having at least 75%, at least 80%, at least 85%, at least 90%, at least 95%, at least 98%, at least 99%, or 100% aa sequence identity to aas 21 to 119 of a β2M aa sequence depicted in FIG. 4.

In some cases, a MHC polypeptide comprises a single aa substitution relative to a reference MHC polypeptide (where a reference MHC polypeptide can be a wt. MHC polypeptide), where the single aa substitution substitutes an aa with a cysteine (Cys) residue. Such cysteine residues, when present in a MHC polypeptide of a first polypeptide of a T-Cell-MMP, or its epitope conjugate, can form a disulfide bond with a cysteine residue present in a second polypeptide chain.

In some cases, a first MHC polypeptide in a first polypeptide of a T-Cell-MMP and/or a second MHC polypeptide in a second polypeptide of a T-Cell-MMP, include a substitution of an aa with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of the following pairs of residues in a HLA β2M (see FIG. 4) and a HLA Class I heavy chain (see FIGS. 3D-3I) is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12)β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A, -B, or -C aa sequence (i.e., residue 260 of the aa sequence depicted in FIGS. 3A-3C respectively) is substituted with a Cys. In some cases, residue 32 (corresponding to Arg-12 of mature β2M) of an aa sequence depicted in FIG. 4 is substituted with a Cys.

Separately, or in addition to, the pairs of cysteine residues in a β2M and HLA Class I heavy chain polypeptide that may be used to form interchain disulfide bonds between the first and second polypeptides of a T-Cell-MMP (discussed above), the HLA-heavy chain of a T-Cell-MMP or its epitope conjugate may be substituted with cysteines to form an intrachain disulfide bond between a cysteine substituted into the carboxyl end portion of the α1 helix and a cysteine in the amino end portion of the α2-1 helix. Such disulfide bonds stabilize the T-Cell-MMP and permit its cellular processing and excretion from eukaryotic cells in the absence of a bound epitope peptide (or null peptide). In one embodiment the carboxyl end portion of the α1 helix is from about aa position 79 to about aa position 89 and the amino end portion of the α2-1 helix is from about aa position 134 to about aa position 144 of the MHC Class I heavy chain (the aa positions are determined based on the sequence of the heavy chains without their leader sequence (see, e.g., FIGS. 3D-3H). In one such embodiment the disulfide bond is between a cysteine located at positions 83, 84, or 85 and a cysteine located at any of positions 138, 139 or 140 of the MHC Class I heavy chain. For example, a disulfide bond may be formed from cysteines incorporated into the MHC Class I heavy chain at aa 83 and a cysteine at an aa located at any of positions 138, 139 or 140. Alternatively, a disulfide bond may be formed between a cysteine inserted at position 84 and a cysteine inserted at any of positions 138, 139 or 140, or between a cysteine inserted at position 85 and a cysteine at any one of positions 138, 139 or 140. In an embodiment, the MHC Class 1 heavy chain intrachain disulfide bond is between cysteines substituted into a heavy chain sequence at positions 84 and 139 (e.g., the heavy chain sequence may be one of the heavy chain sequences set forth in FIGS. 3D-3H). As noted above, any of the MHC Class I intrachain disulfide bonds, including a disulfide bond between cysteines at 84 and 139, may be combined with intrachain disulfide bonds including a bond between MHC Class 1 heavy position 236 and position 12 of a mature β2M polypeptide sequence (lacking its leader) as shown, for example, in FIG. 4.

In another embodiment, an intrachain disulfide bond may be formed in a MHC-H sequence of a T-Cell-MMP, or its epitope conjugate, between a cysteine substituted into the region between aa positions 79 and 89 and a cysteine substituted into the region between aa positions 134 and 144 of the sequences given in FIGS. 3D-3H. In such an embodiment, the MHC Class I heavy chain sequence may have insertions, deletions and/or substitutions of 1 to 5 aas preceding or following the cysteines forming the disulfide bond between the carboxyl end portion of the α1 helix and the amino end portion of the α2-1 helix. Any inserted aas may be selected from the naturally occurring aas or the naturally occurring aas except proline and alanine.

In an embodiment, the β2M polypeptide of a T-Cell-MMP or its epitope conjugate comprises a mature β2M polypeptide sequence (aas 21-119) of any one of NP_004039.1, NP_ 001009066.1, NP_001040602.1, NP_776318.1, or NP_033865.2 (SEQ ID NOs:57 to 61).

In some cases, a HLA Class I heavy chain polypeptide of a T-Cell-MMP or its epitope conjugate comprises any one of the HLA-A, -B, -C, -E, -F, or -G sequences in FIGS. 3D-3H. Any of the heavy chain sequences may further comprise cysteine substitutions at positions 84 and 139, which may form an intrachain disulfide bond.

In an embodiment, the β2M polypeptide of a T-Cell-MMP, or its epitope conjugate, comprises a mature β2M polypeptide sequence (aas 21-119) of any one of the sequences in FIG. 4, which further comprises a R12C substitution.

In an embodiment, a T-Cell-MMP, or its epitope conjugate, comprises a first polypeptide comprising a mature β2M polypeptide sequence (e.g., aas 21-119 of any one of the sequences in FIG. 4) having a R12C substitution, and a second polypeptide comprising any one of the HLA-A, -B, -C, -E, -F, or -G sequences in FIGS. 3D-3H bearing a cysteine at position 236. In such embodiments an intrachain disulfide bond may form between the cysteines at positions 12 and 236. In addition, any of the heavy chain sequences may further comprise cysteine substitutions at positions 84 and 139, which may form an intrachain disulfide bond.

In some cases, a HLA Class I heavy chain polypeptide of a T-Cell-MMP, or its epitope conjugate, comprises the aa sequence of HLA-A*0201 (FIG. 3D). In some cases, a HLA Class I heavy chain polypeptide of a T-Cell-MMP, or its epitope conjugate, comprises the aa sequence of HLA-A*0201 having an A236C substitution (FIG. 3D). In some cases, a HLA Class I heavy chain polypeptide of a T-Cell-MMP, or its epitope conjugate, comprises the aa sequence of HLA-A*0201 having a Y84A and a A236C substitution (FIG. 3D).

In an embodiment, a T-Cell-MMP, or its epitope conjugate, comprises a first polypeptide comprising aa residues 21-119 of NP_004039.1 with a R12C substitution (see FIG. 4), and a second polypeptide comprising a HLA-A*0201 (HLA-A2) sequence in FIG. 3D. In one such embodiment the HLA-A*0201 sequence has an A236C substitution. In another such embodiment, the HLA-A*0201 sequence has a Y84C and A139C substitution. In another such embodiment, the HLA-A*0201 sequence has a Y84C, A139C, and A236C substitution. As indicated, MHC-H sequences with Y84C and A139C substitutions may form a stabilizing intrachain disulfide bond, and a cysteine at position 236 of the mature MHC-H may bond to a cysteine at position 12 of a mature β2M polypeptide.

In an embodiment, a T-Cell-MMP, or its epitope conjugate, comprises a first polypeptide comprising aa residues 21-119 of NP_004039.1 with a R12C substitution (see FIG. 4), and a second polypeptide, a HLA Class I heavy chain polypeptide, comprises the aa sequence GSHSMRYFFTSVSRPGRGEPRFIAVGYVDDTQFVRFDSDAASQRMEPRAPWIEQEGPEYWDGET RKVKAHSQTHRVDL(aa cluster 1){C}(aa cluster 2)AGSHTVQRMYGCDVGSDWRFLRGYHQYAY DGKDYIALKEDLRSW(aa cluster 3){C}(aa cluster 4)HKWEAAHVAEQLRAYLEGTCVEWLRRYLE NGKETLQRTDAPKTHMTHHAVSDHEATLRCWALSFYPAEITLTWQRDGEDQTQDTELVETRPA GDGTFQKWAAVVVPSGQEQRYTCHVQHEGLPKPLTLRWEP (SEQ ID NO:76); or, the first polypeptide comprises the aa sequence IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:77), and the second polypeptide comprises the aa sequence, GSHSMRYFFTSVSRPGRGEPRFIAVGYVDDTQFVRFDSDAASQRMEPRAPWIEQEGPEYWDGET RKVKAHSQTHRVDL(aa cluster 1){C}(aa cluster 2)AGSHTVQRMYGCDVGSDWRFLRGYHQYAY DGKDYIALKEDLRSW(aa cluster 3){C}(aa cluster 4))HKWEAAHVAEQLRAYLEGTCVEWLRRYL ENGKETLQRTDAPKTHMTHHAVSDHEATLRCWALSFYPAEITLTWQRDGEDQTQDTEL(aa dust er 5)(C)(aa cluster 6)QKWAAVVVPSGQEQRYTCHVQHEGLPKPLTLRWEP (SEQ ID NO:78); where the cysteine residues indicated as {C} form a disulfide bond between the α1 and α2-1 helices and the (C) residue forms a disulfide bond with the mature β2M polypeptide cysteine at position 12.

Each occurrence of aa cluster 1, aa cluster 2, aa cluster 3, aa cluster 4, aa cluster 5, and aa cluster 6 is independently selected to be 1-5 aa residues, wherein the aa residues are each selected independently from i) any naturally occurring (proteogenic) aa or ii) any naturally occurring aa except proline or glycine.

In an embodiment where the MHC Class I heavy chain is an HLA-A chain:
aa cluster 1 may be the amino acid sequence GTLRG or that sequence with one or two aas deleted or substituted with other naturally occurring aas (e.g., L replaced by I, V, A or F);
aa cluster 2 may be the amino acid sequence YNQSE or that sequence with one or two aas deleted or substituted with other naturally occurring aas (e.g., N replaced by Q, Q replaced by N, and/or E replaced by D);
aa cluster 3 may be the amino acid sequence TAADM or that sequence with one or two aas deleted or substituted with other naturally occurring aas (e.g., T replaced by S, A replaced by G, D replaced by E, and/or M replaced by L, V, or I);
aa cluster 4 may be the amino acid sequence AQTTK or that sequence with one or two aas deleted or substituted with other naturally occurring aas (e.g., A replaced by G, Q replaced by N, or T replaced by S, and or K replaced by R or Q);
aa cluster 5 may be the amino acid sequence VETRP or that sequence with one or two aas deleted or substituted with other naturally occurring aas (e.g., V replaced by I or L, E replaced by D, T replaced by S, and/or R replaced by K); and/or
aa cluster 6 may be the amino acid sequence GDGTF or that sequence with one or two aas deleted or substituted with other naturally occurring aas (e.g., D replaced by E, T replaced by S, or F replaced by L, W, or Y).

In some cases, the β2M polypeptide comprises the amino acid sequence:

(SEQ ID NO: 79)
IQRTPKIQVYSCHPAENGKSNFLNCYVSGFHPSDIEVDLLKNGERIEKV
EHSDLSFSKDWSFYLLYYTEFTPTEKDEYACRVNHVTLSQPKIVKWDRD
M.

In some cases, the first polypeptide and the second polypeptide of a T-Cell-MMP of the present disclosure are disulfides linked to one another through: i) a Cys residue present in a linker connecting the peptide epitope and a β2M polypeptide in the first polypeptide chain (e.g., with the epitope placed in the N-terminal to the linker and the β2M sequences); and ii) a Cys residue present in a MHC Class I heavy chain in the second polypeptide chain. In some cases, the Cys residue present in the MHC Class I heavy chain is a Cys introduced as a Y84C substitution. In some cases, the linker connecting the peptide epitope and the β2M polypeptide in the first polypeptide chain is GCGGS(G₄S)n, where n is 1, 2, 3, 4, 5, 6, 7, 8, or 9 (SEQ ID NO:93) (e.g., epitope-GCGGS(G₄S)n-mature β2M polypeptide). For example, in some cases, the linker comprises the aa sequence GCGGSGGGGSGGG GSGGGGS (SEQ ID NO:95). As another example, the linker comprises the aa sequence GCGGSGGG GSGGGGS (SEQ ID NO:96). Examples of such a disulfide-linked first and second polypeptide are depicted schematically in FIGS. 6E-6H.

5 Scaffold Polypeptides

T-Cell-MMPs and T-Cell-MMP-epitope conjugates can comprise a Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, a Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:80), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see, also, Schellenberger et al. (2009) Nat Biotechnol. 27:1186). Suitable albumin polypeptides include, e.g., human serum albumin.

Suitable scaffold polypeptides will in some cases be half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, a Fc polypeptide increases the in vivo half-life (serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

6 Fc polypeptides

In some cases, the first and/or the second polypeptide chains of a T-Cell-MMP or its corresponding T-Cell-MMP-epitope conjugate (multimeric polypeptide(s)) comprise a Fc polypeptide which may be modified to include one or more chemical conjugation sites within or attached (e.g., at a terminus or attached by a linker) to the polypeptide. The Fc polypeptide of a T-Cell-MMP or T-Cell-MMP-epitope conjugate can be, for example, from an IgA, IgD, IgE, IgG, or IgM, which may contain a human polypeptide sequence, a humanized polypeptide sequence, a Fc region polypeptide of a synthetic heavy chain constant region, or a consensus heavy chain constant region. In embodiments, the Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. Unless stated otherwise, the Fc polypeptides used in the T-Cell-MMPs and their epitope conjugates do not comprise a transmembrane anchoring domain or a portion thereof sufficient to anchor the T-Cell-MMP or its epitope conjugate to a cell membrane. In some cases, the Fc polypeptide comprises an aa sequence having at least about 70% (e.g., at least about 75%, 80%, 85%, 90%, 95%, 98%, 99%, or 100%) aa sequence identity to an aa sequence of a Fc region depicted in FIGS. 2A-2G. In some cases, the Fc region comprises an aa sequence having at least about 70% (e.g., at least about 75%, 80%, 85%, 90%, 95%, 98%, 99%, or 100%) aa sequence identity to the human IgG1 Fc polypeptide depicted in FIG. 2A. In some cases, the Fc region comprises an aa sequence having at least about 70%, (e.g., at least about 75%, 80%, 85%, 90%, 95%, 98%, 99%, or 100%) aa sequence identity to the human IgG1 Fc polypeptide depicted in FIG. 2A; and comprises a substitution of N77, which is underlined and bolded; e.g., the Fc polypeptide comprises a N77A substitution. In some cases, the Fc polypeptide comprises an aa sequence having at least about 70% (e.g., at least about 75%, 80%, 85%, 90%, 95%, 98%, 99%, or 100%) aa sequence identity to the human IgG2 Fc polypeptide depicted in FIG. 2A; e.g., the Fc polypeptide comprises an aa sequence having at least about 70% (e.g., at least about 75%, 80%, 85%, 90%, 95%, 98%, 99%, or 100%) aa sequence identity to aas 99-325 of the human IgG2 Fc polypeptide depicted in FIG. 2A. In some cases, the Fc polypeptide comprises an aa sequence having at least about 70% (e.g., at least about 75%, 80%, 85%, 90%, 95%, 98%, 99%, or 100%) aa sequence identity to the human IgG3 Fc polypeptide depicted in FIG. 2A; e.g., the Fc polypeptide comprises an aa sequence having at least about 70% (e.g., at least about 75%, 80%, 85%, 90%, 95%, 98%, 99%, or 100%) aa sequence identity to aas 19-246 of the human IgG3 Fc polypeptide depicted in FIG. 2A. In some cases, the Fc polypeptide comprises an aa sequence having at least about 70% (e.g., at least about 75%, 80%, 85%, 90%, 95%, 98%, 99%, or 100%) aa sequence identity to the human IgM Fc polypeptide depicted in FIG. 2B; e.g., the Fc polypeptide comprises an aa sequence having at least about 70% (e.g., at least about 75%, 80%, 85%, 90%, 95%, 98%, 99%, or 100%) aa sequence identity to aas 1-276 of the human IgM Fc polypeptide depicted in FIG. 2B. In some cases, the Fc polypeptide comprises an aa sequence having at least about 70% (e.g., at least about 75%, 80%, 85%, 90%, 95%, 98%, 99%, or 100%) aa sequence identity to the human IgA Fc polypeptide depicted in FIG. 2C; e.g., the Fc polypeptide comprises an aa sequence having at least about 70% (e.g., at least about 75%, 80%, 85%, 90%, 95%, 98%, 99%, or 100%) aa sequence identity to aas 1-234 of the human IgA Fc polypeptide depicted in FIG. 2C.

In some cases, the Fc polypeptide present in a multimeric polypeptide comprises the aa sequence depicted in FIG. 2A (human IgG1 Fc). In some cases, the Fc polypeptide present in a multimeric polypeptide comprises the aa sequence depicted in FIG. 2A (human IgG1 Fc), except for a substitution of N297 (N77 of the aa sequence depicted in FIG. 2A) with an aa other than asparagine. In some cases, the Fc polypeptide present in a multimeric polypeptide comprises the aa sequence depicted in FIG. 2C (human IgG1 Fc comprising an N297A substitution, which is N77 of the aa sequence depicted in FIG. 2A). In some cases, the Fc polypeptide present in a multimeric polypeptide comprises the aa sequence depicted in FIG. 2A (human IgG1 Fc), except for a substitution of L234 (L14 of the aa sequence depicted in FIG. 2A) with an aa other than leucine. In some cases, the Fc polypeptide present in a multimeric polypeptide comprises the aa sequence depicted in FIG. 2A (human IgG1 Fc), except for a substitution of L235 with an aa other than leucine.

In some cases, the Fc polypeptide present in a multimeric polypeptide comprises the aa sequence depicted in FIG. 2E. In some cases, the Fc polypeptide present in a multimeric polypeptide comprises the aa sequence depicted in FIG. 2F. In some cases, the Fc polypeptide present in a multimeric polypeptide comprises the aa sequence depicted in FIG. 2G (human IgG1 Fc comprising an L234A substitution and an L235A substitution, corresponding to positions 14 and 15 of the aa sequence depicted in FIG. 2G). In some cases, the Fc polypeptide present in a multimeric polypeptide comprises the aa sequence depicted in FIG. 2A (human IgG1 Fc), except for a substitution of P331 (P111 of the aa sequence depicted in FIG. 2A) with an aa other than proline; in some cases, the substitution is a P331S substitution. In some cases, the Fc polypeptide present in a multimeric polypeptide comprises the aa sequence depicted in FIG. 2A (human IgG1 Fc), except for substitutions at L234 and L235 (L14 and L15 of the aa sequence depicted in FIG. 2A) with aas other than leucine. In some cases, the Fc polypeptide present in a multimeric polypeptide comprises the aa sequence depicted in FIG. 2A (human IgG1 Fc), except for substitutions at L234 and L235 (L14 and L15 of the aa sequence depicted in FIG. 2A) with aas other than leucine, and a substitution of P331 (P111 of the aa sequence depicted in FIG. 2A) with an aa other than proline. In some cases, the Fc polypeptide present in a multimeric polypeptide comprises the aa sequence depicted in FIG. 2E (human IgG1 Fc comprising L234F, L235E, and P331S substitutions, corresponding to aa positions 14, 15, and 111 of the aa sequence depicted in FIG. 2E). In some cases, the Fc polypeptide present in a multimeric polypeptide is an IgG1 Fc polypeptide that comprises L234A and L235A substitutions (substitutions of L14 and L15 of the aa sequence depicted in FIG. 2A with Ala), as depicted in FIG. 2G.

In some cases, the Fc polypeptide comprises an aa sequence having at least about 70% (e.g., at least about 75%, 80%, 85%, 90%, 95%, 98%, 99%, or 100%) aa sequence identity to a human IgG4 Fc polypeptide depicted in FIG. 2C. In some cases, the Fc polypeptide comprises an aa sequence having at least about 70% (e.g., at least about 75%, 80%, 85%, 90%, 95%, 98%, 99%, or 100%) aa sequence identity to aas 100 to 327 of the GenBank P01861 human IgG4 Fc polypeptide depicted in FIG. 2C.

7 Linkers

T-Cell-MMPs (and their T-Cell-MMP-epitope conjugates) can include one or more independently selected linker peptides interposed between, for example, any one or more of: i) a MHC polypeptide and an Ig Fc polypeptide, where such a linker is referred to herein as a “L1 linker”; ii) a MHC polypeptide and a MOD, where such a linker is referred to herein as a “L2 linker”; iii) a first MOD and a second MOD, where such a linker is referred to herein as a “L3 linker” (e.g., between a first variant 4-1BBL polypeptide and a second variant 4-1BBL polypeptide; or between a second variant 4-1BBL polypeptide and a third variant 4-1BBL polypeptide); iv) a conjugation site or a peptide antigen (conjugated “epitope” peptide) and a MHC Class I polypeptide (e.g., β2M); v) a MHC Class I polypeptide and a dimerization polypeptide (e.g., a first or a second member of a dimerizing pair); and vi) a dimerization polypeptide (e.g., a first or a second member of a dimerizing pair) and an IgFc polypeptide.

Suitable linkers (also referred to as “spacers”) can be readily selected and can be of any of a number of suitable lengths, such as from 1 aa to 25 aa, from 3 aa to 20 aa, from 2 aa to 15 aa, from 3 aa to 12 aa, from 4 aa to 10 aa, from 5 aa to 9 aa, from 6 aa to 8 aa, or from 7 aa to 8 aa. In embodiments, a suitable linker can be 1, 2, 3, 4, 5, 6, 7, 8, 9, 10, 11, 12, 13, 14, 15, 16, 17, 18, 19, 20, 21, 22, 23, 24, or 25 aa in length. In some cases, a linker has a length of from 25 aa to 50 aa, e.g., from 25 to 30, from 30 to 35, from 35 to 40, from 40 to 45, or from 45 to 50 aa in length.

Exemplary linkers include glycine polymers (G)ₙ; glycine-serine polymers (including, for example, (GS), (GSGGS) (SEQ ID NO:81) and (GGGS) (SEQ ID NO:82), any of which may be repeated from 1 to 10 times (e.g., repeated 1, 2, 3, 4, 5, 6, 7, 8, 9, or 10 times); glycine-alanine polymers; alanine-serine polymers; and other flexible linkers known in the art. Glycine and glycine-serine polymers can both be used; both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers can also comprise aa sequences including, but not limited to, GGSG (SEQ ID NO:83), GGSGG (SEQ ID NO:84), GSGSG (SEQ ID NO:85), GSGGG (SEQ ID NO:86), GGGSG (SEQ ID NO:87), GSSSG (SEQ ID NO:88), which may be repeated from 1 to 10 times (e.g., repeated 1, 2, 3, 4, 5, 6, 7, 8, 9, or 10 times), combinations thereof, and the like. Exemplary linkers can comprise the sequence Gly(Ser)₄ (SEQ ID NO:89) or Gly₄Ser (SEQ ID NO:90), either of which may be repeated from 1 to 10 times (e.g., 1, 2, 3, 4, 5, 6, 7, 8, 9, or 10 times). In one embodiment the linker comprises the aa sequence AAAGG (SEQ ID NO:91), which may be repeated from 1 to 10 times.

In some cases, a linker comprises the aa sequence (GGGGS) (SEQ ID NO:92), which may be repeated from 1 to 10 times (e.g., 1, 2, 3, 4, 5, 6, 7, 8, 9, or 10 times). In some cases, a linker polypeptide, present in a first polypeptide of a T-Cell-MMP or its epitope conjugate, includes a cysteine residue that can form a disulfide bond with a cysteine residue present in an epitope presenting polypeptide or a second polypeptide of a T-Cell-MMP or its epitope conjugate. In some cases, for example, the linker comprises the aa sequence GCGGS(G₄S) (SEQ ID NO:93) where the G₄S unit may be repeated from 1 to 10 times (e.g., repeated 1, 2, 3, 4, 5, 6, 7, 8, 9, or 10 times), GCGASGGGGSGGGGS (SEQ ID NO:94), the sequence GCGGSGGGGSGGGGSGGGGS (SEQ ID NO:95) or the sequence GCGGSGGGGSGGGGS (SEQ ID NO:96).

Linkers, including the polypeptide linkers described above, may be present between a payload coupled to the first or second polypeptide of a T-Cell-MMP (or its epitope conjugate). In addition to the polypeptide linkers recited above, the linkers used to attach a payload or epitope (e.g., peptide) to the first and/or second polypeptide can be non-peptides. Such non-peptide linkers include polymers comprising, for example, polyethylene glycol (PEG). Other linkers, including those resulting from coupling with a bifunctional crosslinking agent, such as those recited below, may also be utilized.

8 Immunomodulatory Polypeptides (MODs)

In some cases, a MOD present in a T-Cell-MMP of the present disclosure is a wt. MOD. In other cases, a MOD present in a T-Cell-MMP of the present disclosure is a variant MOD that has reduced affinity for a Co-MOD, compared to the affinity of a corresponding wt. MOD for the Co-MOD. Some MOD polypeptides that may be incorporated into T-Cell-MMPs exhibit reduced affinity for Co-MODs. The MOD polypeptides can have from 1 aa to 10 aa differences from a wt. immunomodulatory domain. For example, in some cases, a variant MOD polypeptide present in a T-Cell-MMP of the present disclosure may differ in aa sequence by, for example, 1 aa, 2 aa, 3 aa, 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa (e.g., from 1 aa to 5 aa, from 5 aa to 10 aa, or from 10 aa to 20 aa) from a corresponding wild-type MOD. As an example, in some cases, a variant MOD polypeptide present in a T-Cell-MMP of the present disclosure has and/or includes 1, 2, 3, 4, 5, 6, 7, 8, 9, 10, 11, 12, 13, 14, 15, 16, 17, 18, 19, or 20 (e.g., from about 1 to about 20; 1 to 2; 1 to 3; 1 to 5; 2 to 4; 2 to 5; 2 to 6; 2 to 7; 2 to 8; 2 to 9; 2 to 10; 2 to 11; 2 to 12; 2 to 13; 2 to 14; 2 to 15; 2 to 16; 2 to 17; 2 to 18; 2 to 19, 2 to 20; 5 to 10; or 10 to 20) aa substitutions, compared to a corresponding reference (e.g., wt.) MOD. In some cases, variant MOD polypeptides present in a T-Cell-MMP include a single aa substitution compared to a corresponding reference (e.g., wt.) MOD.

As discussed above, variant MODs suitable for inclusion as domains (MOD polypeptides) in T-Cell-MMPs of the present disclosure (and/or their epitope conjugates) include those that exhibit reduced affinity for a Co-MOD, compared to the affinity of a corresponding wt. MOD for the Co-MOD. Suitable variant MODs can be identified by, for example, mutagenesis, such as scanning mutagenesis (e.g., alanine, serine, or glycine scanning mutagenesis).

Exemplary pairs of MODs and Co-MODs include, but are not limited to, entries (a) to (r) listed in the following table:

Exemplary Pairs of MODs and Co-MODs

a) 4-1BBL (MOD) and 4-1BB (Co-MOD);
b) PD-Ll (MOD) and PD1 (Co-MOD);
e) IL-2 (MOD) and IL-2 receptor (Co-MOD);
d) CD80 (MOD) and CD28 (Co-MOD);
e) CD86 (MOD) and CD28 (Co-MOD);
f) OX40L (CD252) (MOD) and OX40 (CD134)(Co-MOD);
g) Fas ligand (MOD) and Fas (Co-MOD);
h) ICOS-L (MOD) and ICOS (Co-MOD);
i) ICAM (MOD) and LFA-1 (Co-MOD);
j) CD30L (MOD) and CD30 (Co-MOD);
k) CD40 (MOD) and CD40L (Co-MOD);
l) CD83 (MOD) and CD83L (Co-MOD);
m) HVEM (CD270) (MOD) and CD160 (Co-MOD);
n) JAG1 (CD339) (MOD) and Notch (Co-MOD);
o) JAG1 (CD339) (MOD) and CD46 (Co-MOD);
p) CD70 (MOD) and CD27 (Co-MOD);
q) CD80 (MOD) and CTLA4 (Co-MOD);
r) CD86 (MOD) and CTLA4 (Co-MOD); and
s) PD-L1(MOD) and CD-80 (Co-MOD)

In some cases, a variant MOD present in a T-Cell-MMP of the present disclosure has a binding affinity for a Co-MOD that is from 100 nM to 100 μM. For example, in some cases, a variant MOD polypeptide present in a T-Cell-MMP of the present disclosure (or its epitope conjugate) has a binding affinity for a Co-MOD that is from about 100 nM to about 150 nM, from about 100 nM to about 500 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 500 nM to about 1 μM, from about 600 nM to about 700 nM, from about 700 nM to about 800 nM, from about 800 nM to about 900 nM, from about 900 nM to about 1 μM, from about 1 μM to about 5 μM, from about 1 μM to about 25 μM from about 5 μM to about 10 μM, from about 10 μM to about 15 μM, from about 15 μM to about 20 μM, from about 20 μM to about 25 μM, from about 25 μM to about 50 μM, from about 25 μM to about 100 μM, from about 50 μM to about 75 μM, or from about 75 μM to about 100 μM.

A variant MOD present in a T-Cell-MMP of the present disclosure exhibits reduced affinity for a cognate Co-MOD. Similarly, a T-Cell-MMP of the present disclosure that comprises a variant MOD exhibits reduced affinity for a cognate Co-MOD. Thus, for example, a T-Cell-MMP of the present disclosure that comprises a variant MOD has a binding affinity for a cognate Co-MOD that is from 100 nM to 100 μM. For example, in some cases, a T-Cell-MMP of the present disclosure that comprises a variant MOD has a binding affinity for a cognate Co-MOD that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

a. Wild-Type and Variant PD-L1 MODs

As one non-limiting example, a MOD or variant MOD present in a masked TGF-β construct or complex is a PD-L1 or variant PD-L1 polypeptide. Wild-type PD-L1 binds to PD1.

A wild-type human PD-L1 polypeptide can comprise the following amino acid sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:97); where aas 1-18 form the signal sequence, aas 19-127 form the Ig-like, V-type, or IgV domain, and 133-225 form the Ig-like C2 type domain.

A wild-type human PD-L1 ectodomain can comprise the following amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:98); where aas 1-109 form the Ig-like, V-type, or “IgV” domain, and aas 115-207 form the Ig-like C2 type domain.

A wild-type PD-L1 IgV domain, suitable for use as a MOD may comprise all or part of the PD-L1 IgV domain (aas 19-127 of SEQ D No. 97), and a carboxyl terminal stabilization sequence, such as for instance the last seven aas (bolded and italicized) of the sequence:

(SEQ ID NO: 99)
AFT VTVPKDLYVV EYGSNMTIEC KFPVEKQLDL AALIVYWEME
DKNIIQFVHG EEDLKTQHSS YRQRARLLKD QLSLGNAA [symbol]
ITDVKLQDAG VYRCMISYGG ADYKRITVKV NAPY [symbol] .

Where the carboxyl stabilizing sequence comprises a histidine (e.g., a histidine approximately 5 residues to the C-terminal side of the tyrosine (Y) appearing as aa 117 of SEQ ID NO:99) at about aa 122, the histidine may form a stabilizing electrostatic bond with the backbone amide at aas 82 and 83 (bolded and italicized in SEQ ID NO:99 (Q107 and L106 of SEQ ID NO:97). As an alternative, a stabilizing disulfide bond may be formed by substituting one of aas 82 or 83) (Q107 and L106 of SEQ ID NO:97) and one of aa residues 121, 122, or 123 (equivalent to aa positions 139-141 of SEQ ID NO:97).

A wild-type PD-1 polypeptide can comprise the following amino acid sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:100).

In some cases, a variant PD-L1 polypeptide (e.g., a variant of SEQ ID NO:98 or PD-L1's IgV domain, SEQ ID NO:99) exhibits reduced binding affinity to PD-1 (e.g., a PD-1 polypeptide comprising the aa sequence set forth in SEQ ID NO:100), compared to the binding affinity of a PD-L1 polypeptide comprising the aa sequence set forth in SEQ ID NO:97 or SEQ ID NO:98. For example, in some cases, a variant PD-L1 polypeptide binds PD-1 (e.g., a PD-1 polypeptide comprising the aa sequence set forth in SEQ ID NO:100) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:97 or SEQ ID NO:98.

In some cases, a variant PD-L1 polypeptide (e.g., a variant of SEQ ID NO:98 or its IgV domain, SEQ ID NO:99) has a binding affinity to PD-1 (e.g., of SEQ ID NO:100) that is from 1 nM to 1 mM (e.g., from 1 nM to 10 nM, from 10 nM to 100 nM, from 100 nM to 1 μM, from 1 μM to 10 μM, from 10 μM to 100 μM, or from 100 μM to 1 mM). As another example, in some cases, a variant PD-L1 polypeptide (e.g., a variant of SEQ ID NO:98) has a binding affinity for PD-1 (e.g., a PD-1 polypeptide comprising the aa sequence set forth in SEQ ID NO:100) that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 20 μM, from about 20 μM to about 30 μM, from about 30 μM to about 50 μM, from about 50 μM to about 75 μM, or from about 75 μM to about 100 μM.

A number of aa substitutions may be made in the PD-L1 ectodomain sequences used as MODs, including substitutions to sequences having greater than 90% (95%, 98% or 99%) sequence identity to at least 85 contiguous aas (e.g., at least 90, at least 95, at least 100, or at least 105 contiguous aas) of any one of SEQ ID NO:97, SEQ ID NO:98, aas 19-127 (the IgV domain) of SEQ ID NO:97, and SEQ ID NO:99. The substitutions may include (a) disulfide bond substitution pairs, D103C and G33C or V104 and S34C; (b) salt bridge forming substitution pairs, Q107D and K62R or Q107D and S80R; and/or (c) Pi stacking substitutions M34Y or M34F. A PD-L1 MOD sequence may comprise a sequence having at least 85 contiguous aas (e.g., at least 90, at least 95, at least 100, or at least 105 contiguous aas) of SEQ ID NO:98, and at least one disulfide, salt bridge, or Pi stacking substitution. A PD-L1 MOD sequence may comprise a sequence having at least 85 contiguous aas (e.g., at least 90, at least 95, at least 100, or at least 105 contiguous aas) of aas 19-127 (the IgV domain) of SEQ ID NO:97, and at least one disulfide, salt bridge, or Pi stacking substitution. A PD-L1 MOD sequence may comprise a sequence having at least 85 contiguous aas (e.g., at least 90, at least 95, at least 100, or at least 105 contiguous aas) of SEQ ID NO:99, and at least one disulfide, salt bridge, or Pi stacking substitution.

In some cases, a variant PD-L1 polypeptide has a single aa substitution compared to the PD-L1 aa sequence set forth in SEQ ID NO:97, SEQ ID NO:98 or PD-L1's IgV domain. In some cases, a variant PD-L1 polypeptide has from 2 aa to 10 aa substitutions compared to the PD-L1 aa sequence set forth in SEQ ID NO:97, SEQ ID NO:98 or PD-L1's IgV domain. In some cases, a variant PD-L1 polypeptide has 2 aa substitutions compared to the PD-L1 aa sequence set forth in SEQ ID NO:97, SEQ ID NO:98 or PD-L1's IgV domain. In some cases, a variant PD-L1 polypeptide has 3 aa or 4 aa substitutions compared to the PD-L1 aa sequence set forth in SEQ ID NO:97, SEQ ID NO:98 or PD-L1's IgV domain. In some cases, a variant PD-L1 polypeptide has 5 aa or 6 aa substitutions compared to the PD-L1 aa sequence set forth in SEQ ID NO:97, SEQ ID NO:98 or PD-L1's IgV domain provided in SEQ ID NO:99. In some cases, a variant PD-L1 polypeptide has 7 aa or 8 aa substitutions compared to the PD-L1 aa sequence set forth in SEQ ID NO:97, SEQ ID NO:98 or PD-L1's IgV domain. In some cases, a variant PD-L1 polypeptide has 9 aa or 10 aa substitutions compared to the PD-L1 aa sequence set forth in SEQ ID NO:97, SEQ ID NO:98 or PD-L1's IgV domain.

Suitable variant PD-L1 polypeptide sequences include polypeptide sequences having at least 80%, at least 85%, at least 90%, at least 95%, at least 98%, or at least 99% aa sequence identity to at least 170 contiguous aas (e.g., at least 180, 190 or 200 contiguous aa) of SEQ ID NO:98 (e.g., which have at least one aa insertion, deletion or substitution). Suitable variant PD-L1 IgV polypeptide sequences include polypeptide sequences having at least 80%, at least 85%, at least 90%, at least 95%, at least 98%, or at least 99% aa sequence identity to at least 70 contiguous aas (e.g., at least 80, 90, 100 or 105 contiguous aas) of aas 1-109 of SEQ ID NO:98 or SEQ ID NO:99 (e.g., which have at least one aa insertion, deletion or substitution).

Variant PD-L1 polypeptide sequences include polypeptide sequences having at least 90% (e.g., at least 95%, 98%, or 99%), or 100%, aa sequence identity to SEQ ID NO:98 or SEQ ID NO:99, wherein the residue at position 8 is an aa other than D; in one such instance that residue is an A, and in another, R. Variant PD-L1 polypeptide sequences include polypeptide sequences having at least 90% (e.g., at least 95%, 98%, or 99%), or 100%, aa sequence identity to SEQ ID NO:98 or SEQ ID NO:99, wherein the residue at position 36 is an aa other than I; in one such instance that residue is an A, and in another, D. Variant PD-L1 polypeptide sequences also include polypeptide sequences having at least 90% (e.g., at least 95%, 98%, or 99%), or 100%, aa sequence identity to SEQ ID NO:98 or SEQ ID NO:99, wherein the residue at position 54 is an aa other than E; in one instance that residue is an A, and in another, R.

b. Wild-Type and Variant CD80 MODs

In some cases, a variant MOD polypeptide present in a T-Cell-MMP of the present disclosure is a variant CD80 polypeptide. Wild-type CD80 binds to CD28.

A wild-type amino acid sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:101).

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:102). In some cases, where a T-Cell-MMP of the present disclosure comprises a variant CD80 polypeptide, a Co-MOD is a CD28 polypeptide comprising the amino acid sequence of SEQ ID NO:102.

A wild-type CD28 amino acid sequence can also be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:103).

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:104).

In some cases, a variant CD80 polypeptide exhibits reduced binding affinity to CD28, compared to the binding affinity of a CD80 polypeptide comprising the amino acid sequence set forth in SEQ ID NO:102 for CD28. For example, in some cases, a variant CD80 polypeptide binds CD28 with a binding affinity that is at least 10% less (e.g., at least: 15% less, 20% less, 25% less, 30% less, 35% less, 40% less, 45% less, 50% less, 55% less, 60% less, 65% less, 70% less, 75% less, 80% less, 85% less, 90% less, 95% less, or more than 95% less) than the binding affinity of a CD80 polypeptide comprising the amino acid sequence set forth in SEQ ID NO:102 for CD28 (e.g., a CD28 polypeptide comprising the amino acid sequence set forth in one of SEQ ID NOs:102, 103, or 104).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the amino acid sequence set forth in SEQ ID NO:102, SEQ ID NO:103, or SEQ ID NO:104) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO:101. In some cases, a variant CD80 polypeptide has from 2 to 10 amino acid substitutions compared to the CD80 amino acid sequence set forth in SEQ ID NO:101. In some cases, a variant CD80 polypeptide has 2, 3, 4, 5, 6, 7, 8. 9, or 10 amino acid substitutions compared to the CD80 amino acid sequence set forth in SEQ ID NO:101.

Some suitable CD80 variants include a polypeptide that comprises an amino acid sequence having a sequence identity of at least 90% (less than 20 substitutions), at least 95% (less than 10 substitutions), at least 97% (less than 6 substitutions), at least 98% (less than 4 substitutions), at least 99% (less than 2 substitutions), or at least 99.5% (one substitution) amino acid sequence identity to any one of the CD80 amino acid sequences that follow.

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:105), where X is any amino acid other than Asn. In some cases, X is Ala.

VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:106), where X is any amino acid other than Asn. In some cases, X is Ala.

VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:107), where X is any amino acid other than Ile. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLX̅ YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:108), where X is any amino acid other than Lys. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO:109), where X is any amino acid other than Gln. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO:110), where X is any amino acid other than Asp. In some cases, X is Ala.

VIHVTK EVKEVATLSC GHNVSVEEXA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:111), where X is any amino acid other than Leu. In some cases, X is Ala.

VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:112), where X is any amino acid other than Tyr. In some cases, X is Ala.

VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:113), where X is any amino acid other than Gln. In some cases, X is Ala.

VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:114), where X is any amino acid other than Met. In some cases, X is Ala.

VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:115), where X is any amino acid other than Val. In some cases, X is Ala.

VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:116), where X is any amino acid other than Ile. In some cases, X is Ala.

VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:117), where X is any amino acid other than Tyr. In some cases, X is Ala.

VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:118), where X is any amino acid other than Asp. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:119), where X is any amino acid other than Phe. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO:120), where X is any amino acid other than Ser. In some cases, X is Ala.

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:121), where X is any amino acid other than Pro. In some cases, X is Ala.

c. Wild-Type and Variant CD86 MODs

In some cases, a variant MOD polypeptide present in a T-Cell-MMP of the present disclosure is a variant CD86 polypeptide. Wild-type CD86 binds to CD28.

The amino acid sequence of the full ectodomain of a wild-type human CD86 can be as follows:

(SEQ ID NO: 122)
APLKIQAYFNETADLPCQFANSQNQSLSELVVFWQDQENLVLNEVYLG
KEKFDSVHSKYMNRTSFDSDSWTLRLHNLQIKDKGLYQCIIHHKKPTGM
IRIHQMNSELSVLANFSQPEIVPISNITENVYINLTCSSIHGYPEPKKM
SVLLRTKNSTIEYDGIMQKSQDNVTELYDVSISLSVSFPDVTSNMTIFC
ILETDKTRLLSSPFSIELEDPQPPPDHIP.

The amino acid sequence of the IgV domain of a wild-type human CD86 can be as follows:

(SEQ ID NO: 123)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVL.

In some cases, a variant CD86 polypeptide exhibits reduced binding affinity to CD28, compared to the binding affinity of a CD86 polypeptide comprising the amino acid sequence set forth in SEQ ID NO:122 or SEQ ID NO:123 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a CD86 polypeptide comprising the amino acid sequence set forth in SEQ ID NO:122 or SEQ ID NO:123 for CD28 (e.g., a CD28 polypeptide comprising the amino acid sequence set forth in one of SEQ ID NOs:102, 103, or 104).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the amino acid sequence set forth in one of SEQ ID NOs: 102, 103, or 104) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO:122. In some cases, a variant CD86 polypeptide has from 2 to 10 amino acid substitutions compared to the CD86 amino acid sequence set forth in SEQ ID NO:122. In some cases, a variant CD86 polypeptide has 2, 3, 4, 5, 6, 7, 8, 9, or 10 amino acid substitutions compared to the CD86 amino acid sequence set forth in SEQ ID NO:122.

In some cases, a variant CD86 polypeptide has a single amino acid substitution compared to the CD86 amino acid sequence set forth in SEQ ID NO:123. In some cases, a variant CD86 polypeptide has from 2 to 10 amino acid substitutions compared to the CD86 amino acid sequence set forth in SEQ ID NO:123. In some cases, a variant CD86 polypeptide has 2, 3, 4, 5, 6, 7, 8, 9, or 10 amino acid substitutions compared to the CD86 amino acid sequence set forth in SEQ ID NO:123.

Suitable CD86 variants include a polypeptide that comprises an amino acid sequence having at least 90%, at least 95%, at least 98%, at least 99%, or 100% amino acid sequence identity to any one of the amino acid sequences that follow.

APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:124), where X is any amino acid other than Asn. In some cases, X is Ala.

APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:125), where X is any amino acid other than Asp. In some cases, X is Ala.

APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:126), where X is any amino acid other than Trp. In some cases, X is Ala.

APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:127), where X is any amino acid other than His. In some cases, X is Ala.

(SEQ ID NO: 128)
APLKIQAYFNETADLPCQFANSQNQSLSELVVFWQDQENLVLNEVYLGKE
KFDSVHSKYMXRTSFDSDSWTLRLHNLQIKDKGLYQCIIHHKKPTGMIRI
HQMNSELSVL,

where X is any amino acid other than Asn. In some cases, X is Ala.

(SEQ ID NO: 129)
APLKIQAYFNETADLPCQFANSQNQSLSELVVFWQDQENLVLNEVYLGK
EKFDSVHSKYMNRTSFXSDSWTLRLHNLQIKDKGLYQCIIHHKKPTGMI
RIHQMNSELSVL,

where X is any amino acid other than Asp. In some cases, X is Ala.

(SEQ ID NO: 130)
APLKIQAYFNETADLPCQFANSQNQSLSELVVFWQDQENLVLNEVYLGK
EKFDSVHSKYMNRTSFDSDSXTLRLHNLQIKDKGLYQCIIHHKKPTGMI
RIHQMNSELSVL,

where X is any amino acid other than Trp. In some cases, X is Ala.

(SEQ ID NO: 131)
APLKIQAYFNETADLPCQFANSQNQSLSELVVFWQDQENLVLNEVYLGK
EKFDSVHSKYMNRTSFDSDSWTLRLHNLQIKDKGLYQCIIHXKKPTGMI
RIHQMNSELSVL,

where X is any amino acid other than His. In some cases, X is Ala.

APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:132), where X is any amino acid other than Val. In some cases, X is Ala.

(SEQ ID NO: 133)
APLKIQAYFNETADLPCQFANSQNQSLSELVVFWQDQENLXLNEVYLGK
EKFDSVHSKYMNRTSFDSDSWTLRLHNLQIKDKGLYQCIIHHKKPTGMI
RIHQMNSELSVL,

where X is any amino acid other than Val. In some cases, X is Ala.

APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:134), where X is any amino acid other than Gln. In some cases, X is Ala.

(SEQ ID NO: 135)
APLKIQAYFNETADLPCQFANSQNQSLSELVVFWXDQENLVLNEVYLGK
EKFDSVHSKYMNRTSFDSDSWTLRLHNLQIKDKGLYQCIIHHKKPTGMI
RIHQMNSELSVL,

where X is any amino acid other than Gln. In some cases, X is Ala.

APLKIQAYFNETADLPCQFANSQNQSLSELVVXWQDQENLVLNEVYLGKEKFDSVHSK YMNRTSFDSDSWTLRLHNLQIKDKGLYQCIIHHKKPTGMIRIHQMNSELSVLANFSQPEIVPISNI TENVYINLTCSSIHGYPEPKKMSVLLRTKNSTIEYDGIMQKSQDNVTELYDVSISLSVSFPDVTSN MTIFCILETDKTRLLSSPFSIELEDPQPPPDHIP (SEQ ID NO:136), where X is any amino acid other than Phe. In some cases, X is Ala.

(SEQ ID NO: 137)
APLKIQAYFNETADLPCQFANSQNQSLSELVVXWQDQENLVLNEVYLGKE
KFDSVHSKYMNRTSFDSDSWTLRLHNLQIKDKGLYQCIIHHKKPTGMIRI
HQMNSELSVL,

where X is any amino acid other than Phe. In some cases, X is Ala.

APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVLANFSQPEIVPISNIT ENVYINLTCSSIHGYPEPKKMSVLLRTKNSTIEYDGIMQKSQDNVTELYDVSISLSVSFPDVTSNM TIFCILETDKTRLLSSPFSIELEDPQPPPDHIP (SEQ ID NO:138), where X is any amino acid other than Leu. In some cases, X is Ala.

(SEQ ID NO: 139)
APLKIQAYFNETADLPCQFANSQNQSLSELVVFWQDQENLVLNEVYLGKE
KFDSVHSKYMNRTSFDSDSWTXRLHNLQIKDKGLYQCIIHHKKPTGMIRI
HQMNSELSVL,

where X is any amino acid other than Leu. In some cases, X is Ala.

APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVLANFSQPEIVPISNITE NVYINLTCSSIHGYPEPKKMSVLLRTKNSTIEYDGIMQKSQDNVTELYDVSISLSVSFPDVTSNMT IFCILETDKTRLLSSPFSIELEDPQPPPDHIP (SEQ ID NO:140), where X is any amino acid other than Tyr. In some cases, X is Ala.

(SEQ ID NO: 141)
APLKIQAYFNETADLPCQFANSQNQSLSELVVFWQDQENLVLNEVYLGKE
KFDSVHSKXMNRTSFDSDSWTLRLHNLQIKDKGLYQCIIHHKKPTGMIRI
HQMNSELSVL,

where X is any amino acid other than Tyr. In some cases, X is Ala.

APLKIQAYFNETADLPCQFANSQNQSLSELVVFWQDQENLVLNEVYLGKEKFDSVHSKY MX₁RTSFDSDSWTLRLHNLQIKDKGLYQCIIHX₂KKPTGMIRIHQMNSELSVLANFSQPEIVPISNIT ENVYINLTCSSIHGYPEPKKMSVLLRTKNSTIEYDGIMQKSQDNVTELYDVSISLSVSFPDVTSNM TIFCILETDKTRLLSSPFSIELEDPQPPPDHIP (SEQ ID NO:142), where X₁ is any amino acid other than Asn and the second X₂ is any amino acid other than His. In some cases, X₁ and X₂ are both Ala.

(SEQ ID NO: 143)
APLKIQAYFNETADLPCQFANSQNQSLSELVVFWQDQENLVLNEVYLGKE
KFDSVHSKYMXRTSFDSDSWTLRLHNLQIKDKGLYQCIIHXKKPTGMIRI
HQMNSELSVL,

where X₁ is any amino acid other than Asn and X₂ is any amino acid other than His. In some cases, X₁ and X₂ are both Ala.

APLKIQAYFNETADLPCQFANSQNQSLSELVVFWQDQENLVLNEVYLGKEKFDSVHSKY MNRTSFX₁SDSWTLRLHNLQIKDKGLYQCIIHX₂KKPTGMIRIHQMNSELSVLANFSQPEIVPISNIT ENVYINLTCSSIHGYPEPKKMSVLLRTKNSTIEYDGIMQKSQDNVTELYDVSISLSVSFPDVTSNM TIFCILETDKTRLLSSPFSIELEDPQPPPDHIP (SEQ ID NO:144), where X₁ is any amino acid other than Asp, and X₂ is any amino acid other than His. In some cases, X₁ is Ala and X₂ is Ala.

(SEQ ID NO: 145)
APLKIQAYFNETADLPCQFANSQNQSLSELVVFWQDQENLVLNEVYLGKE
KFDSVHSKYMNRTSFX₁SDSWTLRLHNLQIKDKGLYQCIIHX₂KKPTGMI
RIHQMNSELSVL,

where X₁ is any amino acid other than Asn and X₂ is any amino acid other than His. In some cases, X₁ and X₂ are both Ala.

APLKIQAYFNETADLPCQFANSQNQSLSELVVFWQDQENLVLNEVYLGKEKFDSVHSKY MX₁RTSF\vec{X}₂SDSWTLRLHNLQIKDKGLYQCIIHX₃KKPTGMIRIHQMNSELSVLANFSQPEIVPISNI TENVYINLTCSSIHGYPEPKKMSVLLRTKNSTIEYDGIMQKSQDNVTELYDVSISLSVSFPDVTSN MTIFCILETDKTRLLSSPFSIELEDPQPPPDHIP (SEQ ID NO:146), where X₁ is any amino acid other than Asn, X₂ is any amino acid other than Asp, and X₃ is any amino acid other than His. In some cases, X₁ is Ala, X₂ is Ala, and X₃ is Ala.

(SEQ ID NO: 147)
APLKIQAYFNETADLPCQFANSQNQSLSELVVFWQDQENLVLNEVYLGKE
KFDSVHSKYMX₁RTSFX₂SDSWTLRLHNLQIKDKGLYQCIIHX₃KKPTGM
IRIHQMNSELSVL,

where X₁ is any amino acid other than Asn, X₂ is any amino acid other than Asp, and X₃ is any amino acid other than His. In some cases, X₁ is Ala, X₂ is Ala, and X₃ is Ala.

d. Wild-Type and Variant 4-1BBL MODs

In some cases, a variant MOD polypeptide present in a T-Cell-MMP of the present disclosure is a variant 4-1BBL polypeptide. Wild-type 4-1BBL binds to 4-1BB (CD137).

A wild-type 4-1BBL amino acid sequence can be as follows:

(SEQ ID NO: 148)

MEYASDASLD PEAPWPPAPR ARACRVLP [symbol] [symbol] [symbol] [symbol] [symbol]
A CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:23-25, as follows:

| (SEQ ID NO: 149) | |
|---|---|
| | PAGLLDLRQG MFAQLVAQNV LLIDGPLSWY SDPGLAGVSL |
| | TGGLSYKEDT KELVVAKAGV YYVFFQLELR RVVAGEGSGS |
| | VSLALHLQPL RSAAGAAALA LTVDLPPASS EARNSAFGFQ |
| | GRLLHLSAGQ RLGVHLHTEA RARHAWQLTQ GATVLGLFRV |
| | TPEIPAGLPS PRSE; |
| | (SEQ ID NO: 150) |
| | D PAGLLDLRQG MFAQLVAQNV LLIDGPLSWY SDPGLAGVSL |
| | TGGLSYKEDT KELVVAKAGV YYVFFQLELR RVVAGEGSGS |
| | VSLALHLQPL RSAAGAAALA LTVDLPPASS EARNSAFGFQ |
| | GRLLHLSAGQ RLGVHLHTEA RARHAWQLTQ GATVLGLFRV |
| | TPEIPAGLPS PRSE; |
| | or |
| | (SEQ ID NO: 151) |
| | D PAGLLDLRQG MFAQLVAQNV LLIDGPLSWY SDPGLAGVSL |
| | TGGLSYKEDT KELVVAKAGV YYVFFQLELR RVVAGEGSGS |
| | VSLALHLQPL RSAAGAAALA LTVDLPPASS EARNSAFGFQ |
| | GRLLHLSAGQ RLGVHLHTEA RARHAWQLTQ GATVLGLFRV |
| | TPEIPA |

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:152). In some cases, where a T-Cell-MMP of the present disclosure comprises a variant 4-1BBL polypeptide, a Co-MOD is a 4-1BB polypeptide comprising the amino acid sequence of SEQ ID NO:152.

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs:148-151. For example, in some cases, a variant 4-1BBL polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a 4-1BBL polypeptide comprising the amino acid sequence set forth in one of SEQ ID NOs:148-151 for a 4-1BB polypeptide (e.g., a 4-1BB polypeptide comprising the amino acid sequence set forth in SEQ ID NO:152), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the amino acid sequence set forth in SEQ ID NO:152) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs:148-151. In some cases, a variant 4-1BBL polypeptide has from 2 to 10 amino acid substitutions compared to the 4-1BBL amino acid sequence set forth in one of SEQ ID NOs:148-151. In some cases, a variant 4-1BBL polypeptide has 2, 3, 4, 5, 6, 7, 8, 9, or 10 amino acid substitutions compared to the 4-1BBL amino acid sequence set forth in one of SEQ ID NOs:148-151.

Suitable 4-1BBL variants include a polypeptide that comprises an amino acid sequence having at least 90%, at least 95%, at least 98%, at least 99%, or 100% amino acid sequence identity to any one of the amino acid sequences that follow.

PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:153), where X is any amino acid other than Lys. In some cases, X is Ala.

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO:154), where X is any amino acid other than Gln. In some cases, X is Ala.

PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:155), where X is any amino acid other than Met. In some cases, X is Ala.

PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:156), where X is any amino acid other than Phe. In some cases, X is Ala.

PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:157), where X is any amino acid other than Gln. In some cases, X is Ala.

PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:158), where X is any amino acid other than Leu. In some cases, X is Ala.

PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:159), where X is any amino acid other than Val. In some cases, X is Ala.

PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:160), where X is any amino acid other than Gln. In some cases, X is Ala.

PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:161), where X is any amino acid other than Asn. In some cases, X is Ala.

PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:162), where X is any amino acid other than Val. In some cases, X is Ala.

PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:163), where X is any amino acid other than Leu. In some cases, X is Ala.

PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:164), where X is any amino acid other than Leu. In some cases, X is Ala.

PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:165), where X is any amino acid other than Ile. In some cases, X is Ala.

PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:166), where X is any amino acid other than Asp. In some cases, X is Ala.

PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:167), where X is any amino acid other than Gly. In some cases, X is Ala.

PAGLLDLRQG MFAQLVAQNV LLIGGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:168), where X is any amino acid other than Pro. In some cases, X is Ala.

PAGLLDLRQG MFAQLVAQNV LLIGGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:169), where X is any amino acid other than Leu. In some cases, X is Ala.

PAGLLDLRQG MFAQLVAQNV LLIGGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:170), where X is any amino acid other than Ser. In some cases, X is Ala.

PAGLLDLRQG MFAQLVAQNV LLIGGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:171), where X is any amino acid other than Trp. In some cases, X is Ala.

PAGLLDLRQG MFAQLVAQNV LLIGGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:172), where X is any amino acid other than Tyr. In some cases, X is Ala.

PAGLLDLRQG MFAQLVAQNV LLIGGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:173), where X is any amino acid other than Ser. In some cases, X is Ala.

PAGLLDLRQG MFAQLVAQNV LLIGGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:174), where X is any amino acid other than Asp. In some cases, X is Ala.

PAGLLDLRQG MFAQLVAQNV LLIGGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:175), where X is any amino acid other than Pro. In some cases, X is Ala.

PAGLLDLRQG MFAQLVAQNV LLIGGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:176), where X is any amino acid other than Gly. In some cases, X is Ala.

PAGLLDLRQG MFAQLVAQNV LLIGGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:177), where X is any amino acid other than Leu. In some cases, X is Ala.

PAGLLDLRQG MFAQLVAQNV LLIGGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:178), where X is any amino acid other than Gly. In some cases, X is Ala.

PAGLLDLRQG MFAQLVAQNV LLIGGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:179), where X is any amino acid other than Val. In some cases, X is Ala.

PAGLLDLRQG MFAQLVAQNV LLIGGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:180), where X is any amino acid other than Ser. In some cases, X is Ala.

PAGLLDLRQG MFAQLVAQNV LLIGGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:181), where X is any amino acid other than Leu. In some cases, X is Ala.

PAGLLDLRQG MFAQLVAQNV LLIGGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:182), where X is any amino acid other than Thr. In some cases, X is Ala.

PAGLLDLRQG MFAQLVAQNV LLIGGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:183), where X is any amino acid other than Gly. In some cases, X is Ala.

PAGLLDLRQG MFAQLVAQNV LLIGGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:184), where X is any amino acid other than Gly. In some cases, X is Ala.

PAGLLDLRQG MFAQLVAQNV LLIGGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:185), where X is any amino acid other than Leu. In some cases, X is Ala.

PAGLLDLRQG MFAQLVAQNV LLIGGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:186), where X is any amino acid other than Ser. In some cases, X is Ala.

PAGLLDLRQG MFAQLVAQNV LLIGGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:187), where X is any amino acid other than Tyr. In some cases, X is Ala.

PAGLLDLRQG MFAQLVAQNV LLIGGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:188), where X is any amino acid other than Glu. In some cases, X is Ala.

PAGLLDLRQG MFAQLVAQNV LLIGGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:189), where X is any amino acid other than Asp. In some cases, X is Ala.

PAGLLDLRQG MFAQLVAQNV LLIGGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:190), where X is any amino acid other than Thr. In some cases, X is Ala.

PAGLLDLRQG MFAQLVAQNV LLIGGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:191), where X is any amino acid other than Lys. In some cases, X is Ala.

PAGLLDLRQG MFAQLVAQNV LLIGGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:192), where X is any amino acid other than Glu. In some cases, X is Ala.

PAGLLDLRQG MFAQLVAQNV LLIGGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:193), where X is any amino acid other than Phe. In some cases, X is Ala.

PAGLLDLRQG MFAQLVAQNV LLIGGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:194), where X is any amino acid other than Phe. In some cases, X is Ala.

PAGLLDLRQG MFAQLVAQNV LLIGGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:195), where X is any amino acid other than Gln. In some cases, X is Ala.

PAGLLDLRQG MFAQLVAQNV LLIGGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:196), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:197), where X is any amino acid other than Glu. In some cases, X is Ala.

PAGLLDLRQG MFAQLVAQNV LLIGGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:198), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:199), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:200), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:201), where X is any amino acid other than Val. In some cases, X is Ala.

PAGLLDLRQG MFAQLVAQNV LLIGGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:202), where X is any amino acid other than Val. In some cases, X is Ala.

PAGLLDLRQG MFAQLVAQNV LLIGGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:203), where X is any amino acid other than Gly. In some cases, X is Ala.

PAGLLDLRQG MFAQLVAQNV LLIGGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:204), where X is any amino acid other than Glu. In some cases, X is Ala.

PAGLLDLRQG MFAQLVAQNV LLIGGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:205), where X is any amino acid other than Gly. In some cases, X is Ala.

PAGLLDLRQG MFAQLVAQNV LLIGGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:206), where X is any amino acid other than Ser. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO:207), where X is any amino acid other than Asp. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO:208), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO:209), where X is any amino acid other than Pro. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO:210), where X is any amino acid other than Ser. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO:211), where X is any amino acid other than Ser. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO:212), where X is any amino acid other than Glu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO:213), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO:214), where X is any amino acid other than Asn. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO:215), where X is any amino acid other than Ser. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO:216), where X is any amino acid other than Phe. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO:217), where X is any amino acid other than Gln. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO:218), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO:219), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO:220), where X is any amino acid other than Gly. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO:221), where X is any amino acid other than Val. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO:222), where X is any amino acid other than His. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO:223), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO:224), where X is any amino acid other than His. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO:225), where X is any amino acid other than Thr. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO:226), where X is any amino acid other than Glu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO:227), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO:228), where X is any amino acid other than Arg. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO:229), where X is any amino acid other than His. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO:230), where X is any amino acid other than Trp. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO:231), where X is any amino acid other than Leu. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO:232), where X is any amino acid other than Thr. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO:233), where X is any amino acid other than Gln. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO:234), where X is any amino acid other than Gly. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO:235), where X is any amino acid other than Thr. In some cases, X is Ala.

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO:236), where X is any amino acid other than Val. In some cases, X is Ala.

e. IL-2 Variants

In some cases, a variant MOD polypeptide present in a T-Cell-MMP of the present disclosure is a variant IL-2 polypeptide. Wild-type IL-2 binds to IL-2 receptor (IL-2R), i.e., a heterotrimeric polypeptide comprising IL-2Rα, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (UniProt, P60568, SEQ ID NO:237).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122), and a gamma chain (IL-2Rγ; also referred to as CD132). Amino acid sequences of human IL-2Rα, IL2Rβ, and IL-2Rγ can be as follows.

Human IL-2Rα:

(SEQ ID NO: 238)

ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:

(SEQ ID NO: 239)

VNG TSQFTCFYNS RANISCVWSQ DGALQDTSCQ VHAWPDRRRW
NQTCELLPVS QASWACNLIL GAPDSQKLTT VDIVTLRVLC
REGVRWRVMA IQDFKPFENL RLMAPISLQV VHVETHRCNI
SWEISQASHY FERHLEFEAR TLSPGHTWEE APLLTLKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL
V.
Human IL-2Rγ:

(SEQ ID NO: 240)

LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a T-Cell-MMP of the present disclosure comprises a variant IL-2 polypeptide, a Co-MOD is an IL-2R comprising polypeptides comprising the amino acid sequences of SEQ ID NO:238, 239, and 240.

In some cases, a variant IL-2 polypeptide exhibits reduced binding affinity to IL-2R, compared to the binding affinity of an IL-2 polypeptide comprising the amino acid sequence set forth in SEQ ID NO:237. For example, in some cases, a variant IL-2 polypeptide binds IL-2R with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of an IL-2 polypeptide comprising the amino acid sequence set forth in SEQ ID NO:237 for an IL-2R (e.g., an IL-2R comprising polypeptides comprising the amino acid sequences set forth in SEQ ID NOs: 238, 239, and 240), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequences set forth in SEQ ID NOs: 238, 239, and 240) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution compared to the IL-2 amino acid sequence set forth in SEQ ID NO:237. In some cases, a variant IL-2 polypeptide has from 2 to 10 amino acid substitutions compared to the IL-2 amino acid sequence set forth in SEQ ID NO:237. In some cases, a variant IL-2 polypeptide has 2, 3, 4, 5, 6, 7, 8, 9, or 10 amino acid substitutions compared to the IL-2 amino acid sequence set forth in SEQ ID NO:237.

Suitable IL-2 variant MOD polypeptides include a polypeptide that comprises an amino acid sequence having at least 90%, at least 95%, at least 98%, at least 99%, or 100% amino acid sequence identity to any one of the amino acid sequences that follow.

APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:241), where X is any amino acid other than Phe. In some cases, X is Ala In some cases, X is Met. In some cases, X is Pro. In some cases, X is Ser. In some cases, X is Thr. In some cases, X is Trp. In some cases, X is Tyr. In some cases, X is Val. In some cases, X is His.

APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:242), where X is any amino acid other than Asp. In some cases, X is Ala.

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:243), where X is any amino acid other than Glu. In some cases, X is Ala.

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:244), where X is any amino acid other than His. In some cases, X is Ala. In some cases, X is Thr. In some cases, X is Asn. In some cases, X is Cys. In some cases, X is Gln. In some cases, X is Met. In some cases, X is Val. In some cases, X is Trp.

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:245), where X is any amino acid other than His. In some cases, X is Ala, Asn, Arg, Asp, Cys, Glu, Gln, Gly, Ile, Lys, Leu, Met, Phe, Pro, Ser, Thr, Tyr, Trp, or Val.

APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:246), where X is any amino acid other than Tyr. In some cases, X is Ala.

APTSSSTKKT QLQLEHLLLD LQMILNGINN YKNPKLTRML TFKFYMPKKA TELKHLQCLE EELKPLEEVL NLAQSKNFHL RPRDLISXIN VIVLELKGSE TTFMCEYADE TATIVEFLNR WITFCQSIIS TLT (SEQ ID NO:247), where X (N88) is any amino acid other than Asn. In some cases, X is Ala; in some cases, X is Arg.

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:248), where X is any amino acid other than Gln. In some cases, X is Ala.

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:249), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala.

APTSSSTKKT QLQLEX1LLLD LQMILNGINN YKNPKLTRML TX2KFYMPKKA TELKHLQCLE EELKPLEEVL NLAQSKNFHL RPRDLISRIN VIVLELKGSE TTFMCEYADE TATIVEFLNR WITFCQSIIS TLT (SEQ ID NO:250), which comprises an additional N88R substitution, and where X1 (H16) is any amino acid other than His, and where X2 (F42) is any amino acid other than Phe. In some cases, X1 is Ala. In some cases, X2 is Ala. In some cases, X1 is Ala; and X2 is Ala. In some cases, X1 is Thr; and X2 is Ala. In some cases, X1 is Ala; and X2 is Thr. In some cases, X1 is Thr; and X2 is Thr.

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:251), where X₁ is any amino acid other than Asp; and where X₂ is any amino acid other than Phe. In some cases, X₁ is Ala. In some cases, X₂ is Ala. In some cases, X₁ is Ala; and X₂ is Ala.

APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:252), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala.

APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:253), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala.

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:254), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:255), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala.

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:256), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala.

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO:257), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala.

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:258), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala.

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:259), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

In any of the wild-type or variant IL-2 sequences provided herein, the cysteine at position 125 may be substituted with an alanine (a C125A substitution). In addition to any stability provided by the substitution, it may be employed where, for example, an epitope containing peptide or payload is to be conjugated to a cysteine residue elsewhere in a T-Cell-MMP first or second polypeptide, thereby avoiding competition from the C125 of the IL-2 MOD sequence.

9 Additional Polypeptides

A polypeptide chain of a T-Cell-MMP or its epitope conjugate can include one or more polypeptides in addition to those described above. Suitable additional polypeptides include epitope tags and affinity domains. The one or more additional polypeptide(s) can be included as part of a polypeptide translated by cell or cell free system at the N-terminus of a polypeptide chain of a multimeric polypeptide, at the C-terminus of a polypeptide chain of a multimeric polypeptide, or internally within a polypeptide chain of a multimeric polypeptide.

10 Epitope Tags

Suitable epitope tags include, but are not limited to, hemagglutinin (HA; e.g., YPYDVPDYA (SEQ ID NO:260)); FLAG (e.g., DYKDDDDK (SEQ ID NO261)); c-myc (e.g., EQKLISEEDL; SEQ ID NO:262)), and the like.

11 Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel SEPHAROSE®. Exemplary affinity domains include His5 (HHHHH) (SEQ ID NO263), HisX6 (HHHHHH) (SEQ ID NO:264), C-myc (EQKLISEEDL) (SEQ ID NO:26533), Flag (DYKDDDDK) (SEQ ID NO:266, StrepTag (WSHPQFEK) (SEQ ID NO:267), hemagglutinin, (e.g., HA Tag (YPYDVPDYA) (SEQ ID NO:268)), glutathione-S-transferase (GST), thioredoxin, cellulose binding domain, RYIRS (SEQ ID NO:269), Phe-His-His-Thr (SEQ ID NO:270), chitin binding domain, S-peptide, T7 peptide, SH2 domain, C-end RNA tag, WEAAAREACCRECCARA (SEQ ID NO:271), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, 5100 proteins, parvalbumin, calbindin D9K, calbindin D28K, and calretinin, inteins, biotin, streptavidin, MyoD, Id, leucine zipper sequences, and maltose binding protein.

12 Epitopes

The chemical conjugation sites and chemistries described herein permit the incorporation of an epitope-presenting peptide (e.g., phosphopeptide, lipopeptides or glycopeptide) that presents, for example, a cancer-associated epitope, an infectious disease-associated epitope (such as a virus or bacterial epitope), or a self-epitope (e.g., the peptide is a cancer-associated peptide), an infectious disease-associated peptide into a T-Cell-MMP to form a T-Cell-MMP-epitope conjugate. Epitopes of a T-Cell-MMP conjugate are not part of the first or second polypeptide as translated from mRNA, but are added to a T-Cell-MMP at a chemical conjugation site. Selection of candidate MHC allele and peptide (e.g., phosphopeptide, lipopeptides or glycopeptide) epitope combinations for effective presentation to a TCR by a T-Cell-MMP-epitope conjugate can be accomplished using any of a number of well-known methods to determine if the free peptide has affinity for the specific HLA allele used to construct the T-Cell-MMP in which it will be presented as part of the epitope conjugate. It is also possible to determine if the peptide in combination with the specific heavy chain allele and β2M can affect the T-Cell in the desired manner (e.g., induction of proliferation, anergy, or apoptosis). Applicable methods include binding assays and T-cell activation assays.

a. Cell-Based Binding Assays

As one example, cell-based peptide-induced stabilization assays can be used to determine if a candidate peptide binds an HLA class I allele intended for use in a T-Cell-MMP-epitope conjugate. The binding assay can be used in the selection of peptides for incorporation into a T-Cell-MMP-epitope conjugate using the intended allele. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174×CEM.T2; American Type Culture Collection (ATCC) No. CRL-1992)). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody, or directly where the peptide is fluorescently labeled. The stabilization and resultant increased life-span of peptide-HLA complexes on the cell surface by the addition of a peptide validates their identity. Accordingly, binding of candidate peptides for presentation by various Class I HLA heavy chain alleles can be tested by genetically modifying the T2 cells to express the HLA H allele of interest.

In a non-limiting example of use of a T2 assay to assess peptide binding to HLA A*0201, T2 cells are washed in cell culture medium, and suspended at 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 μL and final peptide concentrations of 100 μM, 50 μM, 10 μM and 1 μM. A HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. in 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

b. Biochemical Binding Assays

MHC Class I complexes comprising a β2M polypeptide complexed with an HLA heavy chain polypeptide of a specific allele intended for use in construction of a T-Cell-MMP can be tested for binding to a peptide of interest in a cell-free in vitro assay system. For example, a labeled reference peptide (e.g., fluorescently labeled) is allowed to bind the MHC-class I complex to form an MHC-reference peptide complex. The ability of a test peptide of interest to displace the labeled reference peptide from the complex is tested. The relative binding affinity is calculated as the amount of test peptide needed to displace the bound reference peptide. See, e.g., van der Burg et al. (1995) Human Immunol. 44:189.

As another example, a peptide of interest can be incubated with a MHC Class I complex (containing an HLA heavy chain peptide and β2M) and the stabilization of the MHC complex by bound peptide can be measured in an immunoassay format. The ability of a peptide of interest to stabilize the MHC complex is compared to that of a control peptide presenting a known T-cell epitope. Detection of stabilization is based on the presence or absence of the native conformation of the MHC complex bound to the peptide using an anti-HLA antibody. See, e.g., Westrop et al. (2009) J. Immunol. Methods 341:76; Steinitz et al. (2012) Blood 119:4073; and U.S. Pat. No. 9,205,144.

c. T-cell Activation Assays

Whether a given peptide binds a MHC Class I complex (comprising an HLA heavy chain and a β2M polypeptide), and, when bound to the HLA complex, can effectively present an epitope to a TCR, can be determined by assessing T-cell response to the peptide-HLA complex. T-cell responses that can be measured include, e.g., interferon-gamma (IFNγ) production, cytotoxic activity, and the like.

(i) ELISPOT Assays

Suitable assays include, e.g., an enzyme linked immunospot (ELISPOT) assay where production of a product by target cells (e.g., IFNγ production by target CD8⁺ T) is measured following contact of the target with an antigen-presenting cell (APC) that presents a peptide of interest complexed with a class I MHC (e.g., HLA). Antibody to IFNγ is immobilized on wells of a multi-well plate. APCs are added to the wells, and the plates are incubated for a period of time with a peptide of interest, such that the peptide binds HLA class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA heavy chain alleles can be used to determine whether a peptide of interest effectively binds to a HLA class I molecule comprising a particular HLA H chain.

(ii) Cytotoxicity Assays

Whether a given peptide binds to a particular MHC class I heavy chain allele complexed with β2M and, when bound, can effectively present an epitope to a TCR, can also be determined using a cytotoxicity assay. A cytotoxicity assay involves incubation of a target cell with a cytotoxic CD8⁺ T cell. The target cell displays on its surface a MHC class I complex comprising β2M, and an epitope-peptide and MHC heavy chain allele combination to be tested. The target cells can be radioactively labeled, e.g., with ⁵¹Cr. Whether the target cell effectively presents the epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

(iii) Detection of Antigen-Specific T Cells with Peptide-HLA Tetramers

As another example, multimers (e.g., tetramers) of peptide-MHC complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

d. Peptides Presenting Epitopes

A T-Cell-MMP-epitope conjugate of the present disclosure comprises any of a variety of peptide epitopes. A T-Cell-MMP-epitope conjugate may comprise a peptide that, when in an MHC/peptide complex (e.g., an HLA/peptide complex), presents an epitope to a T cell. A peptide, present in a T-Cell-MMP-epitope conjugate of the present disclosure, that, when in an MHC/peptide complex (e.g., an HLA/peptide complex), presents an epitope to a T cell, may be referred to herein as a “peptide epitope,” “T-Cell-MMP-epitope conjugate,” or, simply, an “epitope.” An epitope peptide in a T-Cell-MMP-epitope conjugate may present, for example, a cancer-associated epitope, an infectious disease-associated epitope (such as a virus or bacterial epitope), or a self-epitope (e.g., the peptide is a cancer-associated peptide, an infectious disease-associated peptide (e.g., a virus- or bacteria-encoded peptide or a peptide of a self-antigen). Peptide epitopes from post-translational modified polypeptides/proteins may also serve as epitopes, including phosphopeptides, glycopeptides and lipopeptides (e.g., peptides modified with fatty acids, isoprenoids, sterols, phospholipids, or glycosylphosphatidyl inositol).

In some cases, the epitope peptide present in a T-Cell-MMP-epitope conjugate of the present disclosure presents an epitope specific to an HLA-A, -B, -C, -E, -F or -G allele. In an embodiment, the epitope peptide present in a T-Cell-MMP-epitope conjugate presents an epitope restricted to HLA-A*0101, A*0201, A*0301, A*1101, A*2301, A*2402, A*2407, A*3303, and/or A*3401. In an embodiment, the epitope peptide present in a T-Cell-MMP-epitope conjugate presents an epitope restricted to HLA-B*0702, B*0801, B*1502, B*3802, B*4001, B*4601, and/or B*5301. In an embodiment, the epitope peptide present in a T-Cell-MMP-epitope conjugate presents an epitope restricted to C*0102, C*0303, C*0304, C*0401, C*0602, C*0701, C*702, C*0801, and/or C*1502.

An epitope (or the epitope presenting sequence of the peptide) present in a T-Cell-MMP-epitope conjugate can be a peptide of from 4 to 25 contiguous aas (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, or from 7 to 25 aa, from 7 to 12, from 7 to 25, from 10 aa to 15 aa, from 15 aa to 20 aa, or from 20 aa to 25 aa).

In an embodiment, an epitope present in a T-Cell-MMP-epitope conjugate of the present disclosure is a peptide specifically bound by a T-cell, i.e., the epitope is specifically bound by a T-cell with a T-cell receptor specific for that epitope. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸M, at least 10⁻⁹ M, or at least 10⁻¹⁰ M.

(i) Epitopes Present in a Cancer-Associated Antigens

Suitable epitopes include, but are not limited to, epitopes present in a cancer-associated antigen. Cancer-associated antigens are known in the art; see, e.g., Cheever et al. (2009) Clin. Cancer Res. 15:5323. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A 1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2). See, e.g., Vigneron et al. (2013) Cancer Immunity 13:15; and Vigneron (2015) BioMed Res. Int'l Article ID 948501; and epidermal growth factor receptor (EGFR) vIII polypeptide (see, e.g., Wong et al. (1992) Proc. Natl. Acad. Sci. USA 89:2965; and Miao et al. (2014) PLoSOne 9:e94281).

In some cases, a suitable peptide epitope is a peptide fragment of from about 4 amino acids (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, or a Fos-related antigen-1 (FOSL) polypeptide. In some cases, a human papilloma virus (HPV) antigen is specifically excluded. In some cases, an alpha-feto protein (AFP) antigen is specifically excluded. In some cases, a Wilms tumor-1 (WT1) antigen is specifically excluded.

Amino acid sequences of cancer-associated antigens are known in the art; see, e.g., MUC1 (GenBank CAA56734); LMP2 (GenBank CAA47024); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CAO99273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFβ (GenBank NP_002600); MAD-CT-2 (GenBank NP_001138574); and FOSL (GenBank NP_005429). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

(ii) Epitopes Associated with Infectious Disease Agents

Suitable epitopes include, but are not limited to, epitopes of an infectious disease agent (e.g., an epitope presented by a bacteria, fungi, mycoplasma (Mycoplasma pneumonia) or viral agent) such as a virus-encoded polypeptide. Examples of viral infectious disease agents include, e.g., Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)-Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus. Antigens encoded by such viruses are known in the art; a peptide epitope suitable for use in a T-Cell MMP-epitope conjugate of the present disclosure can include a peptide from any known viral antigen. In some cases, an HPV antigen is specifically excluded. In some cases, an HBV antigen is specifically excluded.

(iii) Epitopes Associated with HBV

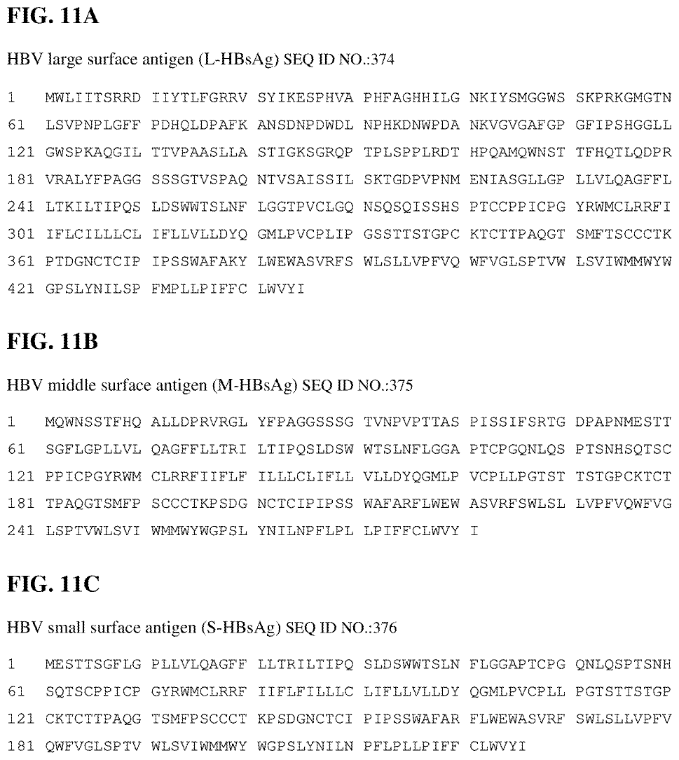

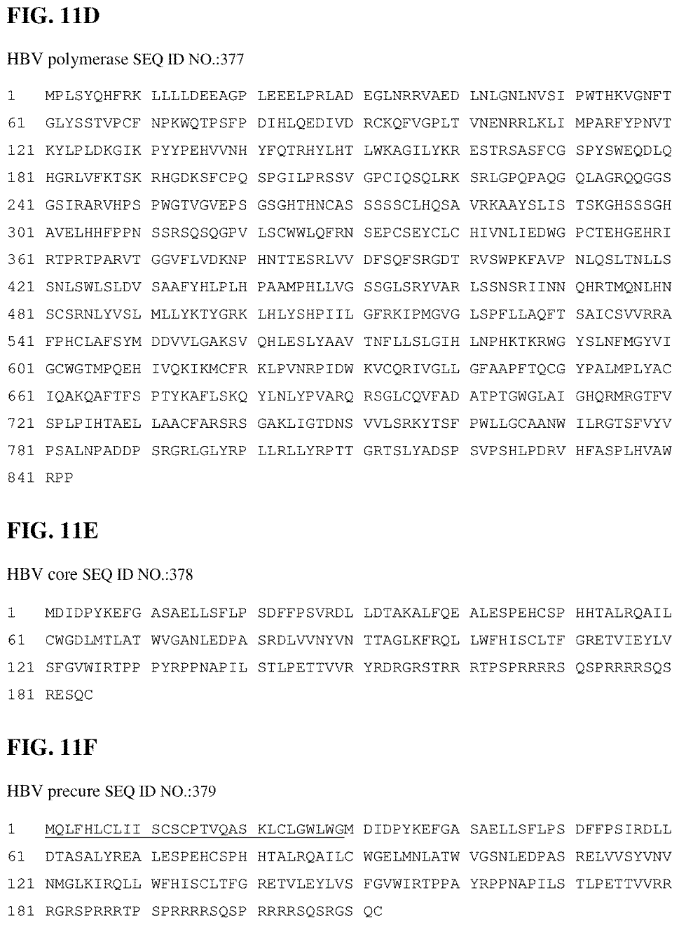

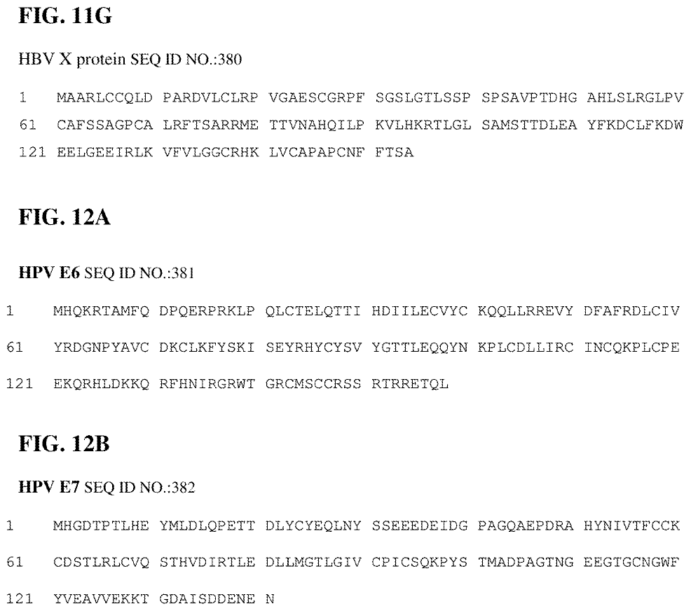

An epitope (a peptide presenting one or more epitopes) present in a T-Cell-MMP-epitope conjugate of the present disclosure is a HBV peptide (e.g., a HBV peptide that, together with a MHC, presents an epitope to a TCR). Amino acid sequences of HBV proteins are presented in FIGS. 11A-11G. A portion of a HBV protein that presents one or more epitopes is referred to herein as a “HBV peptide” or a “HBV epitope.” In some cases, a HBV peptide presenting an epitope (or the epitope presenting portion of the peptide) present in a T-Cell-MMP-epitope conjugate of the present disclosure can comprise a peptide of from 4 to 25 contiguous aas (e.g., 4 aa, 5 aa, 6 aa, 7 aa, 8 aa, 9 aa, 10 aa, 11 aa, 12 aa, 6-15 aa, 7-15 aa, 10-15 aa, 15-20 aa, or 20-25 aa) of an aa sequence having at least 90%, at least 95%, at least 98%, at least 99%, or 100% aa sequence identity to any of the aa sequences depicted in FIGS. 11A to 11G. In some cases, a HBV epitope present in a T-Cell-MMP-epitope conjugate of the present disclosure can be a peptide of from 7 to 25 contiguous aas (e.g., 6 aa, 7 aa, 8 aa, 9 aa, 10-15 aa, 15-20 aa, or 20-25 aa) of an aa sequence having at least 90%, at least 95%, at least 98%, at least 99%, or 100% aa sequence identity to any one of the HBV aa sequences depicted in FIGS. 11A-11G.

A suitable HBV peptide can be a HBV peptide from any HBV genotype, e.g., genotype A, genotype B, genotype C, genotype D, genotype E, genotype F, genotype G, genotype H, genotype I, or genotype J. The reference sequence of the viral genome may be retrieved from the NCBI GenBank or calculated from sequences obtained from clinical specimens. For example, reference sequences for HBV genotype A may be retrieved from NCBI GenBank (https://www(dot)ncbi(dot)nlm(dot)nih(dot)gov/genbank/) Accession No. AP007263, HE974383 or HE974381; reference sequences for HBV genotype B may be retrieved from GenBank Accession No. AB981581, AB602818, or AB554017; reference sequences for HBV genotype C may be retrieved from GenBank Accession No. LC360507, AB644287 or AB113879; reference sequences for HBV genotype D may be retrieved from GenBank Accession No. HE815465, HE974382 or AB554024; reference sequences for HBV genotype E may be retrieved from GenBank Accession No. HE974380, HE974384, AP007262; reference sequences for HBV genotype F may be retrieved from GenBank Accession No. DQ823095, AB036909 or AB036920; reference sequences for HBV genotype G may be retrieved from GenBank Accession No. AB625342, HE981176 or GU563559; reference sequences for HBV genotype H may be retrieved from GenBank Accession No. AB298362, AB846650, AB516395; reference sequences for HBV genotype I may be retrieved from GenBank Accession No. EU833891, KF214680 or KU950741; and reference sequences for HBV genotype J may be retrieved from GenBank Accession No. AB486012. The global geographic distribution of HBV genotypes has been reported; see, e.g., Sunbul (2014) World J. Gastroenterol. 20:5427.

In an embodiment, a HBV epitope present in a T-Cell-MMP-epitope conjugate of the present disclosure is a peptide specifically bound by a T-cell, i.e., the epitope is specifically bound by a HBV epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸M, at least 10⁻⁹ M, or at least 10⁻¹⁰ M.

A suitable HBV peptide epitope can be an HBV peptide epitope derived from HBV polymerase, HBV envelope, HBV precore, HBV large surface antigen (L-HBsAg), HBV middle surface antigen (M-HBsAg), HBV small surface antigen (S-HBsAg), or HBV X-protein. See, e.g., Venkatakrishnan and Zlotnick (2016) Ann. Rev. Virol. 3:429.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an amino acid sequence having at least 90%, at least 95%, at least 98%, at least 99%, or 100%, aa sequence identity to the L-HBsAg amino acid sequence depicted in FIG. 11A.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an amino acid sequence having at least 90%, at least 95%, at least 98%, at least 99%, or 100%, aa sequence identity to the M-HBsAg amino acid sequence depicted in FIG. 11B.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an amino acid sequence having at least 90%, at least 95%, at least 98%, at least 99%, or 100%, aa sequence identity to the S-HBsAg amino acid sequence depicted in FIG. 11C.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an aa sequence having at least 90%, at least 95%, at least 98%, at least 99%, or 100%, aa sequence identity to the HBV polymerase amino acid sequence depicted in FIG. 11D.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an amino acid sequence having at least 90%, at least 95%, at least 98%, at least 99%, or 100%, aa sequence identity to the HBV core amino acid sequence depicted in FIG. 3E.

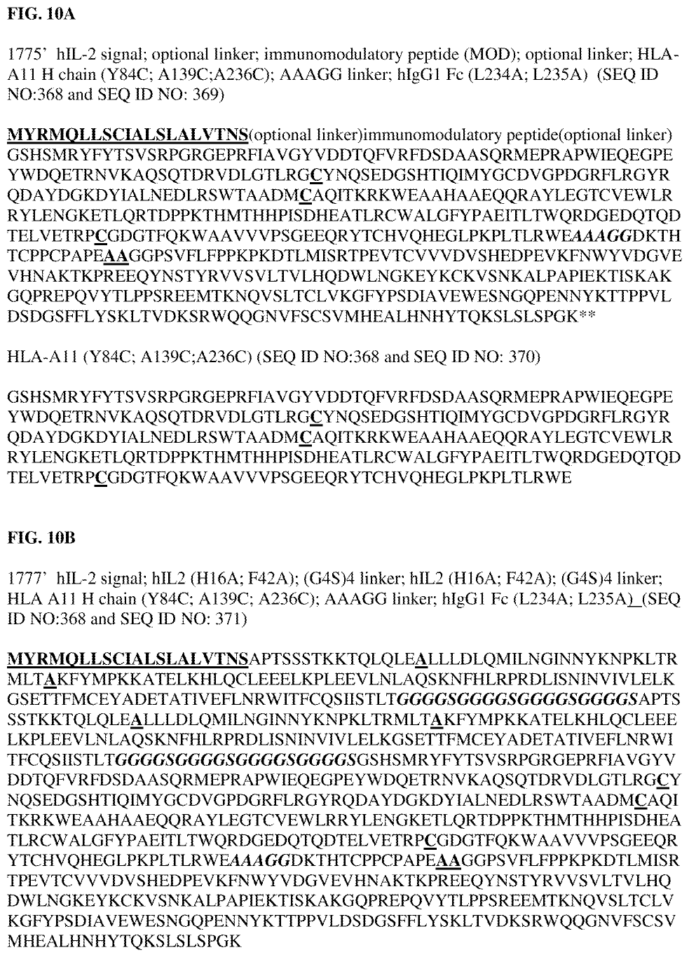

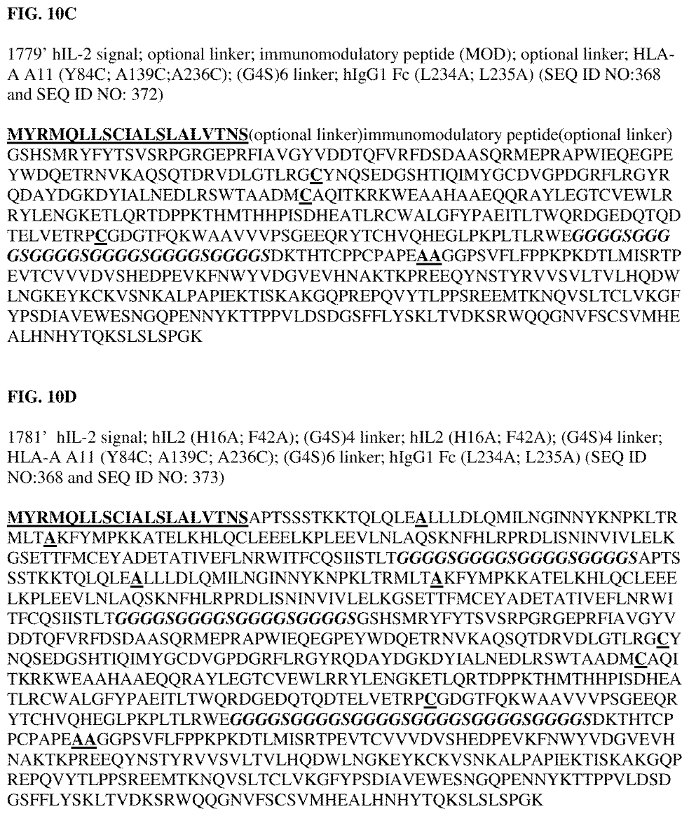

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an amino acid sequence having at least 90%, at least 95%, at least 98%, at least 99%, or 100%, aa sequence identity to the HBV precore amino acid sequence depicted in FIG. 10F.

In some cases, an HBV epitope present in a T-Cell-MMP-epitope conjugate of the present disclosure is a peptide of from 4 to 25 contiguous aas (e.g., 4 aa, 5 aa, 6 aa, 7 aa, 8 aa, 9 aa, 10-15 aa, 15-20 aa, or 20-25 aa) of an aa sequence having at least 90%, at least 95%, at least 98%, at least 99%, or 100%, aa sequence identity to the HBV X protein amino acid sequence depicted in FIG. 10G.

In some cases, the HBV epitope is an HBV Core peptide. For example, an HBV Core peptide can have the amino acid sequence: FLPSDFFPSV (SEQ ID NO:272). In some cases, the HBV epitope is an HBV polymerase (Pol) peptide. Suitable HBV Pol peptides include, e.g., GLSRYVARLG (SEQ ID NO:273), KLHLYSHPI (SEQ ID NO:274), FLLSLGIHL (SEQ ID NO:275), ALMPLYACI (SEQ ID NO:276), and SLYADSPSV (SEQ ID NO:277).

Suitable HBV peptides include: FLPSDFFPSV (SEQ ID NO:272), GLSRYVARLG (SEQ ID NO:273), KLHLYSHPI (SEQ ID NO:274), FLLSLGIHL (SEQ ID NO:275), ALMPLYACI (SEQ ID NO:276), SLYADSPSV (SEQ ID NO:277), STLPETTVV (SEQ ID NO:278), LIMPARFYPK (SEQ ID NO:279), AIMPARFYPK (SEQ ID NO:280), YVNVNMGLK (SEQ ID NO:281), MQWNSTALHQALQDP (from HBV large S protein) (SEQ ID NO:282), LLDPRVRGL (SEQ ID NO:283), SILSKTGDPV (SEQ ID NO:284), VLQAGFFLL (SEQ ID NO:285), FLLTRILTI (SEQ ID NO:286), FLGGTPVCL (SEQ ID NO:287), LLCLIFLLV (SEQ ID NO:288), LVLLDYQGML (SEQ ID NO:289), LLDYQGMLPV (SEQ ID NO:290), IPIPSSWAF (SEQ ID NO:291), WLSLLVPFV (SEQ ID NO:292), GLSPTVWLSV (SEQ ID NO:293), SIVSPFIPLL (SEQ ID NO:294), ILSPFLPLL (SEQ ID NO:295), ATVELLSFLPSDFFPSV (SEQ ID NO:296), LPSDFFPSV (SEQ ID NO:297), CLTFGRETV (SEQ ID NO:298), VLEYLVSFGV (SEQ ID NO:299), EYLVSFGVW (SEQ ID NO:300), ILSTLPETTV (SEQ ID NO:301), STLPETTVVRR (SEQ ID NO:302), NVSIPWTHK (SEQ ID NO:303), KVGNFTGLY (SEQ ID NO:304), GLYSSTVPV (SEQ ID NO:305), TLWKAGILYK (SEQ ID NO:306), TPARVTGGVF (SEQ ID NO:307), LVVDFSQFSR (SEQ ID NO:308), GLSRYVARL (SEQ ID NO:309), SAICSVVRR (SEQ ID NO:310), YMDDVVLGA (SEQ ID NO:311), PLGFFPDH (SEQ ID NO:312), QAFTFSPTYK (SEQ ID NO:313), KYTSFPWLL (SEQ ID NO:314), ILRGTSFVYV (SEQ ID NO:315), HLSLRGLFV (SEQ ID NO:316), VLHKRTLGL (SEQ ID NO:317), GLSAMSTTDL (SEQ ID NO318), CLFKDWEEL (SEQ ID NO:319), and VLGGCRHKL (SEQ ID NO:320), where the peptide has a length of from 9 amino acids to 19 amino acids (e.g., 9, 10, 11, 12, 13, 14, 15, 16, 17, 18, or 19 amino acids).

In some cases, the HBV epitope comprises all or part of a peptide depicted in Table 2.

TABLE 2

| | | Length | SEQ | | | Length | SEQ |
|---|---|---|---|---|---|---|---|
| | Sequence; | in aa | ID | | | in aa | ID |
| No | source | residues | NO. | No. | Sequence | residues | NO. |
| 1 | FLPSDFFPSV; | 10-12 | 272 | 26 | | | |
| | HBV core protein | | | | | | |
| 2 | GLSRYVARLG; | 10-12 | 273 | 27 | LPSDFFPSV | 9-11 | 297 |
| | HBV polymerase | | | | | | |
| 3 | KLHLYSHPI; | 9-11 | 274 | 28 | CLTFGRETV | 9-11 | 298 |
| | HBV polymerase | | | | | | |
| 4 | FLLSLGIHL; | 9-11 | 275 | 29 | VLEYLVSFGV | 10-12 | 299 |
| | HBV polymerase | | | | | | |
| 5 | ALMPLYACI; | 9-11 | 276 | 30 | EYLVSFGVW | 9-11 | 300 |
| | HBV polymerase | | | | | | |
| 6 | SLYADSPSV; | 9-11 | 277 | 31 | ILSTLPETTV; | 10-12 | 301 |
| | HBV polymerase | | | | HBV precore | | |
| 7 | STLPETTVV; | 9-11 | 278 | 32 | STLPETTVVRR; | 11-13 | 302 |
| | HBV core | | | | HBV precore | | |
| 8 | LIMPARFYPK; | 10-12 | 279 | 33 | NVSIPWTHK; | 9-11 | 303 |
| | HB polymerase | | | | HBV polymerase | | |
| 9 | AIMPARFYPK; | 10-12 | 280 | 34 | KVGNFTGLY; | 9-11 | 304 |
| | HBV polymerase | | | | HBV polymerase | | |
| 10 | YVNVNMGLK; | 9-11 | 281 | 35 | GLYSSTVPV; | 9-11 | 305 |
| | HBV precore | | | | HBV polymerase | | |
| 11 | MQWNSTALHQALQDP; | 15-17 | 282 | 36 | TLWKAGILYK; | 10-12 | 306 |
| | HBV L-HBsAg | | | | HBV polymerase | | |
| 12 | LLDPRVRGL; | 9-11 | 283 | 37 | TPARVTGGVF; | 10-12 | 307 |
| | HBV M-HBsAg | | | | HBV polymerase | | |
| 13 | SILSKTGDPV; | 10-12 | 284 | 38 | LVVDFSQFSR; | 10-12 | 308 |
| | HBV L-HBsAg | | | | HBV polymerase | | |
| 14 | VLQAGFFLL; | 9-11 | 285 | 39 | GLSRYVARL; | 9-11 | 309 |
| | HBV L-HBsAg | | | | HBV polymerase | | |
| 15 | FLLTRILTI; | 9-11 | 286 | 40 | SAICSVVRR; | 9-11 | 310 |
| | HBV M-HBsAg | | | | HBV polymerase | | |
| 16 | FLGGTPVCL; | 9-11 | 287 | 41 | YMDDVVLGA; | 9-11 | 311 |
| | HBV L-HBsAg | | | | HBV polymerase | | |
| 17 | LLCLIFLLV; | 9-11 | 288 | 42 | PLGFFPDH; | 8-10 | 312 |
| | HBV M-HBsAg | | | | HBV L-HBsAg | | |
| 18 | LVLLDYQGML; | 10-11 | 289 | 43 | QAFTFSPTYK; | 9-11 | 313 |
| | HBV S-HBsAg | | | | HBV polymerase | | |
| 19 | LLDYQGMLPV; | 10-12 | 290 | 44 | KYTSFPWLL; | 9-11 | 314 |
| | HBV S-HBsAg | | | | HBV polymerase | | |
| 20 | IPIPSSWAF; | 9-11 | 291 | 45 | ILRGTSFVYV; | 10-12 | 315 |
| | HBV M-HBsAg | | | | HBV polymerase | | |
| 21 | WLSLLVPFV; | 9-11 | 292 | 46 | HLSLRGLFV; | 9-11 | 316 |
| | HBV S-HBsAg | | | | HBV X protein | | |
| 22 | GLSPTVWLSV; | 10-12 | 293 | 47 | VLHKRTLGL; | 9-11 | 317 |
| | HBV S-HBsAg | | | | HBV X protein | | |
| 23 | SIVSPFIPLL; | 9-11 | 294 | 48 | GLSAMSTTDL; | 10-12 | 318 |
| | HBV L-HBsAg | | | | HBV X protein | | |
| 24 | ILSPFLPLL; | 9-11 | 295 | 49 | CLFKDWEEL; | 9-11 | 319 |
| | HBV L-HBsAg | | | | HBV X protein | | |
| 25 | ATVELLSFLPSDFFPSV | 17-19 | 296 | 50 | VLGGCRHKL; | 9-11 | 320 |
| | | | | | HBV X protein | | |

(iv) Epitopes Associated with HPV

An epitope (a peptide presenting one or more epitopes) present in a T-Cell-MMP-epitope conjugate of the present disclosure is a HPV peptide (e.g., a HPV peptide that, together with a MHC, presents an epitope to a TCR). Amino acid sequences of HPV proteins are presented in FIGS. 11A-11B. A portion of a HPV protein that presents one or more epitopes is referred to herein as a “HPV peptide” or a “HPV epitope.” An HPV peptide suitable for inclusion in a T-Cell-MMP-epitope conjugate of the present disclosure can be a peptide of an HPV E1 polypeptide, an HPV E2 polypeptide, an HPV E3 polypeptide, an HPV E4 polypeptide, an HPV E5 polypeptide, an HPV E6 polypeptide, an HPV E7 polypeptide, or an HPV L2 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. In some cases, the epitope is an HPV E6 epitope. In some cases, the epitope is an HPV E7 epitope. In some cases, a HPV peptide presenting an epitope (or the epitope presenting portion of the peptide) present in a T-Cell-MMP-epitope conjugate of the present disclosure can comprise a peptide of from 4 to 25 contiguous aas (e.g., 4 aa, 5 aa, 6 aa, 7 aa, 8 aa, 9 aa, 10 aa, 11 aa, 12 aa, 6-15 aa, 7-15 aa, 10-15 aa, 15-20 aa, or 20-25 aa) of an amino acid sequence having at least 90%, at least 95%, at least 98%, at least 99%, or 100% aa sequence identity to the HPV E6 amino acid depicted in FIG. 11A, or the HPV E7 amino acid sequence depicted in FIG. 11B. In some cases, a HPV epitope present in a T-Cell-MMP-epitope conjugate of the present disclosure can be a peptide of from 4 to 25 contiguous aas (e.g., 6 aa, 7 aa, 8 aa, 9 aa, 7-25 aa, 10-15 aa, 15-20 aa, or 20-25 aa) of an amino acid sequence having at least 90%, at least 95%, at least 98%, at least 99%, or 100% aa sequence identity to the HPV amino acid sequence depicted in FIG. 11A. In some cases, a HPV epitope present in a T-Cell-MMP-epitope conjugate of the present disclosure can be a peptide of from 4 to 25 contiguous aas (e.g., 6 aa, 7 aa, 8 aa, 9 aa, 10-15 aa, 15-20 aa, or 20-25 aa) of an amino acid sequence having at least 90%, at least 95%, at least 98%, at least 99%, or 100% aa sequence identity to the HPV amino acid sequence depicted in FIG. 11B.

In an embodiment, an HPV epitope present in a T-Cell-MMP-epitope conjugate of the present disclosure is a peptide specifically bound by a T-cell, i.e., the epitope is specifically bound by a HPV epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷M, at least 10⁻⁸M, at least 10⁻⁹ M, or at least 10⁻¹⁰ M.

Examples of HPV E6 peptides suitable for inclusion in a T-Cell-MMP of the present disclosure include, but are not limited to, E6 18-26, KLPQLCTEL (SEQ ID NO:321); E6 26-34, LQTTIHDII (SEQ ID NO:322); E6 49-57, VYDFAFRDL (SEQ ID NO:323); E6 52-60, FAFRDLCIV (SEQ ID NO:324); E6 75-83, KFYSKISEY (SEQ ID NO:325); and E6 80-88, ISEYRHYCY (SEQ ID NO:326);

Examples of HPV E7 peptides suitable for inclusion in a T-Cell-MMP of the present disclosure include, but are not limited to E7 7-15, TLHEYMLDL; (SEQ ID NO:327); E7 11-19, YMLDLQPET (SEQ ID NO:328); E7 44-52, QAEPDRAHY (SEQ ID NO:329); E7 49-57, RAHYNIVTF (SEQ ID NO:330); E7 61-69, CDSTLRLCV (SEQ ID NO:331); E7 67-76, LCVQSTHVDI (SEQ ID NO:332); E7 82-90, LLMGTLGIV (SEQ ID NO:333); E7 86-93, TLGIVCPI (SEQ ID NO:334); and E7 92-93, LLMGTLGIVCPI (SEQ ID NO:335).

In some cases, a suitable HPV peptide is an HPV E6 peptide that binds HLA-A24 (e.g., is an HLA-A2401-restricted epitope). Non-limiting examples include: KLPQLCTEL (SEQ ID NO:321); VYDFAFRDL (SEQ ID NO:323); CYSLYGTTL (SEQ ID NO:336); EYRHYCYSL (SEQ ID NO:337); DPQERPRKL (SEQ ID NO:338); HYCYSLYGT (SEQ ID NO:339); DFAFRDLCI (SEQ ID NO:340); LYGTTLEQQY (SEQ ID NO:341); HYCYSLYGTT (SEQ ID NO:342); EVYDFAFRDL (SEQ ID NO:343); EYRHYCYSLY (SEQ ID NO:344); VYDFAFRDLC (SEQ ID NO:345); YCYSIYGTTL (SEQ ID NO:346); VYCKTVLEL (SEQ ID NO:347); VYGDTLEKL (SEQ ID NO:348); and LTNTGLYNLL (SEQ ID NO:349).

In some cases, a suitable HPV peptide is selected from the group consisting of: TLHEYMLDL (SEQ ID NO:327); RAHYNIVTF (SEQ ID NO:330); DLQPETTDL (SEQ ID NO:350); TPTLHEYML (SEQ ID NO:351); GTLGIVCPI (SEQ ID NO:352); EPDRAHYNI (SEQ ID NO:353); QLFLNTLSF (SEQ ID NO:354); FQQLFLNTL (SEQ ID NO:355); and AFQQLFLNTL (SEQ ID NO:356).

In some cases, a suitable HPV peptide presents an HLA-A*2401-restricted epitope. Non-limiting examples of HPV peptides presenting an HLA-A*2401-restricted epitope are: VYDFAFRDL (SEQ ID NO:323); RAHYNIVTF (SEQ ID NO:330); CDSTLRLCV (SEQ ID NO:331); and LCVQSTHVDI (SEQ ID NO:332). In some cases, an HPV peptide suitable for inclusion in a T-Cell-MMP-epitope conjugate of the present disclosure is VYDFAFRDL (SEQ ID NO:323). In some cases, an HPV peptide suitable for inclusion in a T-Cell-MMP-epitope conjugate of the present disclosure is RAHYNIVTF (SEQ ID NO:330). In some cases, an HPV peptide suitable for inclusion in a T-Cell-MMP-epitope conjugate of the present disclosure is CDSTLRLCV (SEQ ID NO:331). In some cases, an HPV peptide suitable for inclusion in a T-Cell-MMP-epitope conjugate of the present disclosure is LCVQSTHVDI (SEQ ID NO:332).

13 Payloads

A broad variety of payloads may be associated with T-Cell-MMPs and T-Cell-MMP-epitope conjugates, which may incorporate more than one type of payload in addition to epitopes conjugated (covalently) to the T-Cell-MMPs at a first or second chemical conjugation site. In addition, where the T-Cell-MMP molecules or their epitope conjugates multimerize, it may be possible to incorporate monomers labeled with different payloads into a multimer. Accordingly, it is possible to introduce one or more payloads selected, for example, from the group consisting of: therapeutic agents, chemotherapeutic agents, diagnostic agents, labels and the like. It will be apparent that some payloads may fall into more than one category (e.g., a radio label may be useful as a diagnostic and as a therapeutic for selectively irradiating specific tissue or cell type).

As noted above, T-Cell-MMP polypeptides (e.g., a scaffold or Fc polypeptide) can be modified with crosslinking reagents to conjugate payloads and/or epitopes to chemical conjugation sites attached to or in the first or second polypeptide of the T-Cell-MMPs (e.g., at a chemical conjugation site such as an engineered cysteine or lysine). Such crosslinking agents include succinimidyl 4-(N-maleimidomethyl)-cyclohexane-1-carboxylate (SMCC), sulfo-SMCC, maleimidobenzoyl-N-hydroxysuccinimide ester (MB S), sulfo-MBS or succinimidyl-iodoacetate. Introducing payloads using an excess of such crosslinking agents can result in multiple molecules of payload being incorporated into the T-Cell-MMP. Some bifunctional linkers for introducing payloads into T-Cell-MMPs and their epitope conjugates include cleavable linkers and non-cleavable linkers. In some cases, the payload linker is a protease-cleavable linker. Suitable payload linkers include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting examples of suitable linkers are: N-succinimidyl-RN-maleimidopropionamido)-tetraethyleneglycoflester (NHS-PEG4-maleimide); N-succinimidyl 4-(2-pyridyldithio)butanoate (SPDB); disuccinimidyl suberate (DSS); disuccinimidyl glutarate (DGS); dimethyl adipimidate (DMA); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); succinimidyl 3-(2-pyridyldithio)propionate (SPDP); PEG4-SPDP (PEGylated, long-chain SPDP crosslinker); BS(PEG)₅ (PEGylated bis(sulfosuccinimidyl)suberate); BS(PEG)₉ (PEGylated bis(sulfosuccinimidyl)suberate); maleimide-PEG₆-succinimidyl ester; maleimide-PEG₈-succinimidyl ester; maleimide-PEG₁₂-succinimidyl ester; PEG₄-SPDP (PEGylated, long-chain SPDP crosslinker); PEG₁₂-SPDP (PEGylated, long-chain SPDP crosslinker); a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

Control of the stoichiometry of the reaction may result in some selective modification where engineered sites with chemistry orthogonal to all other groups in the molecule is not utilized. Reagents that display far more selectivity, such as the bis-thio linkers discussed above, tend to permit more precise control of the location and stoichiometry than reagents that react with single lysine, or cysteine residues.

Where a T-Cell-MMP of the present disclosure comprises a Fc polypeptide, the Fc polypeptide can comprise one or more covalently attached molecules of payload that are attached directly or indirectly through a linker. By way of example, where a T-Cell-MMP of the present disclosure comprises a Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a payload (e.g., a cytotoxic agent). (L), if present, links (A) to (C). In some cases, the polypeptide chain comprising the Fc polypeptide can comprise more than one molecule of payload (e.g., 2, 3, 4, 5, or more than 5 cytotoxic agent molecules).

In an embodiment, the payload is selected from the group consisting of: biologically active agents or drugs, diagnostic agents or labels, nucleotide or nucleoside analogs, nucleic acids or synthetic nucleic acids (e.g., antisense nucleic acids, small interfering RNA, double stranded (ds)DNA, single stranded (ss)DNA, ssRNA, dsRNA), toxins, liposomes (e.g., incorporating a chemotherapeutic such as 5-fluorodeoxyuridine), nanoparticles (e.g., gold or other metal bearing nucleic acids or other molecules, lipids, particle bearing nucleic acids or other molecules), and combinations thereof.

In an embodiment, the payload is selected from biologically active agents or drugs selected independently from the group consisting of: therapeutic agents (e.g., drugs or prodrugs), chemotherapeutic agents, cytotoxic agents, antibiotics, antivirals, cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or antigen binding fragments thereof, enzymes, proenzymes, hormones and combinations thereof.

In an embodiment, the payload is selected from biologically active agents or drugs selected independently from therapeutic diagnostic agents or labels, selected independently from the group consisting of photodetectable labels (e.g., dyes, fluorescent labels, phosphorescent labels, luminescent labels), contrast agents (e.g., iodine or barium containing materials), radiolabels, imaging agents, paramagnetic labels/imaging agents (gadolinium containing magnetic resonance imaging labels), ultrasound labels and combinations thereof.

a. Therapeutic Agents and Chemotherapeutic Agents

As discussed above, a polypeptide chain of a T-Cell-MMP or its epitope conjugate can comprise a payload including, but not limited to, a small molecule drug, such as a therapeutic or chemotherapeutic agent, linked (e.g., covalently attached) to the first or second polypeptide chain at chemical conjugation sites. The linkage between a payload and a first or second polypeptide chain of a T-Cell-MMP or its epitope conjugate may be a direct or indirect linkage. Direct linkage can involve linkage directly to an amino acid side chain. Indirect linkage can be linkage via a linker. A drug (e.g., a payload such as a cancer chemotherapeutic agent) can be linked to a polypeptide chain (e.g., a Fc polypeptide) of a T-Cell-MMP of the present disclosure via a thioether bond, an amide bond, a carbamate bond, a disulfide bond, or an ether bond.

Suitable therapeutic agents include, e.g., rapamycin, retinoids, such as all-trans retinoic acid (ATRA); vitamin D3; vitamin D3 analogs; and the like. As noted above, in some cases, a drug is a cytotoxic agent. Cytotoxic agents are known in the art. A suitable cytotoxic agent can be any compound that results in the death of a cell, induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatins and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin.

For example, in some cases, the cytotoxic agent is a compound that inhibits microtubule formation in eukaryotic cells. Such agents include, e.g., maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. Maytansinoid compounds include, e.g., N(2′)-deacetyl-N(2′)-(3-mercapto-1-oxopropyl)-maytansine (DM1); N(2′)-deacetyl-N(2′)-(4-mercapto-1-oxopentyl)-maytansine (DM3); and N(2)-deacetyl-N2-(4-mercapto-4-methyl-1-oxopentyl)-maytansine (DM4). Benzodiazepines include, e.g., indolinobenzodiazepines and oxazolidinobenzodiazepines.

Cytotoxic agents include taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; maytansine or an analog or derivative thereof; an auristatin or a functional peptide analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; 6 mercaptopurine; 6 thioguanine; cytarabine; fludarabin; 5 fluorouracil; decarbazine; hydroxyurea; asparaginase; gemcitabine; cladribine; an alkylating agent; a platinum derivative; duocarmycin A; duocarmycin SA; rachelmycin (CC-1065) or an analog or derivative thereof; an antibiotic; pyrrolo[2,1-c][1,4]-benzodiazepines (PDB); diphtheria toxin; ricin toxin; cholera toxin; a Shiga-like toxin; LT toxin; C3 toxin; Shiga toxin; pertussis toxin; tetanus toxin; soybean Bowman-Birk protease inhibitor; Pseudomonas exotoxin; alorin; saporin; modeccin; gelanin; abrin A chain; modeccin A chain; alpha-sarcin; Aleurites fordii proteins; dianthin proteins; Phytolacca americana proteins; Momordica charantia inhibitor; curcin; crotin; Sapaonaria officinalis inhibitor; gelonin; mitogellin; restrictocin; phenomycin; enomycin toxins; ribonuclease (RNase); DNase I; Staphylococcal enterotoxin A; pokeweed antiviral protein; diphtherin toxin; and Pseudomonas endotoxin.

b. Diagnostic Agents and Labels

The first and/or second polypeptide chains of a T-Cell-MMP can comprise one or more molecules of payload of photodetectable labels (e.g., dyes, fluorescent labels, phosphorescent labels, luminescent labels), contrast agents (e.g., iodine or barium containing materials), radiolabels, imaging agents, spin labels, Forster Resonance Energy Transfer (FRET)-type labels, paramagnetic labels/imaging agents (e.g., gadolinium containing magnetic resonance imaging labels), ultrasound labels and combinations thereof.

In some embodiments, the conjugate moiety comprises a label that is or includes a radioisotope. Examples of radioisotopes or other labels include, but are not limited to, ³H, ¹¹C, ¹⁴C, ¹⁵N, ³⁵S, ¹⁸F, ³²P, ³³P, ⁶⁴Cu, ⁶⁸Ga, ⁸⁹Zr, ⁹⁰Y, ⁹⁹Tc, ¹²³I, ¹²⁴I, ¹²⁵I, ¹³¹I, ¹¹¹In, ¹³¹In, ¹⁵³Sm, ¹⁸⁶Re, ¹⁸⁸Re, ²¹¹At, ²¹²Bi, and ¹⁵³Pb.

### II. Methods of Generating T-Cell-MMP Polypeptides

The present disclosure provides a method of obtaining T-Cell-MMPs and/or T-Cell-MMP-epitope conjugates, including those comprising one or more variant MODs that exhibit lower affinity for a Co-MOD compared to the affinity of the corresponding parental wild-type MOD for the Co-MOD, the method comprising:
A) generating a T-Cell-MMP by introducing nucleic acids encoding a first polypeptide and a second polypeptide of the T-Cell-MMP in cells or cell free systems, wherein:
   a) the first polypeptide comprises i) a first MHC Class I polypeptide (e.g., a β2M polypeptide); and
   b) the second polypeptide comprises i) a second MHC polypeptide (e.g., a MHC Class I heavy chain polypeptide), and ii) optionally an Ig Fc polypeptide or a non-Ig scaffold;
wherein the first polypeptide comprises a first chemical conjugation site and/or the second polypeptide comprises a second chemical conjugation site, and at least one of the first polypeptide or second polypeptide comprises one or more independently selected MODs (e.g., 1, 2, 3 or more wild-type and/or variant MODs); and
B) contacting the first polypeptide and second polypeptide (if co-expressed in the same cell or cell-free system the polypeptides may come into contact as they are translated) to form a T-Cell-MMP;
wherein when the T-Cell-MMP comprises one or more nascent (e.g., unactivated) chemical conjugation sites, the nascent chemical conjugation site may be optionally activated to produce a T-Cell-MMP with the first and/or second chemical conjugation site (e.g., reacting sulfatase motifs with a formyl glycine generating enzyme if the cells expressing the T-Cell-MMP do not express a formylglycine generating enzyme).
   The method may be stopped at this point and the T-Cell-MMP obtained by purification; alternatively, where a T-Cell-MMP-epitope conjugate is desired, the method may be continued with the reaction of the T-Cell-MMP with an epitope presenting molecule:
C) providing an epitope (e.g., an epitope presenting peptide) suitable for conjugation with the first and/or second chemical conjugation site (e.g., a hydrazinyl or hydrazinyl indole modified peptide for reaction with a formyl glycine of a sulfatase motif) and contacting the epitope with the T-Cell-MMP (e.g., under suitable reaction conditions) to produce a T-Cell-MMP-epitope conjugate.

Where it is desirable for a T-Cell-MMP to contain a payload (e.g., a small molecule drug, radiolabel, etc.), the payload may be reacted with the T-Cell-MMP in place of the epitope conjugate as described above. Where it is desirable for a T-Cell-MMP-epitope conjugate to contain a payload, the payload may be reacted with the chemical conjugation site(s) either before or after the epitope is contacted and reacted with its chemical reaction site(s). The selectivity of the epitope and the payload for different conjugation sites (e.g., first and second chemical conjugation sites) may be controlled through the use of orthogonal chemistries and/or control of stoichiometry in the conjugation reactions. In embodiments, linkers (e.g., polypeptides or other bifunctional chemical linkers) may be used to attach the epitope and/or payloads to their conjugation sites.

The present disclosure provides a method of obtaining a T-Cell-MMP and/or T-Cell-MMP-epitope conjugate comprising one or more variant MODs that exhibit lower affinity for a Co-MOD compared to the affinity of the corresponding parental wild-type MOD for the Co-MOD, the method comprising:
A) generating a library of T-Cell-MMP-epitope conjugates comprising a plurality of members, wherein each member comprises: a) a first polypeptide comprising: i) an epitope; and ii) a first MHC polypeptide (e.g., a β2M polypeptide); and b) a second polypeptide comprising: i) a second MHC polypeptide (e.g., a MHC Class I heavy chain polypeptide); and ii) optionally an Ig Fc polypeptide or a non-Ig scaffold, wherein each member comprises a different variant MOD on the first polypeptide, the second polypeptide, or both the first and the second polypeptide;
B) determining the affinity of each member of the library for a Co-MOD; and
C) selecting a library member that exhibits reduced affinity for the Co-MOD.
   In some cases, the affinity is determined by BLI using purified T-Cell-MMP or T-Cell-MMP-epitope conjugate library members and the Co-MOD. BLI methods are well known to those skilled in the art. A BLI assay is described above. See, e.g., Lad et al. (2015) J. Biomol. Screen. 20(4): 498-507; and Shah and Duncan (2014) J. Vis. Exp. 18:e51383.

The present disclosure provides a method of obtaining a T-Cell-MMP-epitope conjugate that exhibits selective binding to a T-cell, the method comprising:
A) generating a library of T-Cell-MMP-epitope conjugates comprising a plurality of members, wherein each member comprises:
   a) a first polypeptide comprising i) a first MHC polypeptide; and
   b) a second polypeptide comprising i) a second MHC polypeptide, and ii) optionally an immunoglobulin (Ig) Fc polypeptide or a non-Ig scaffold;
   wherein each member comprises a different variant MOD on the first polypeptide, the second polypeptide, or both the first and the second polypeptide, wherein the variant MOD differs in amino acid sequence by from 1 aa to 10 aa from a parental wild-type MOD,
   wherein the T-Cell-MMP-epitope conjugate library members further comprise an epitope tag or a fluorescent label), and
   wherein one of the first or second polypeptides comprises an epitope covalently bound through a chemical conjugation site, either directly or indirectly through a linker, to the first and/or second polypeptide;
B) contacting a T-Cell-MMP-epitope conjugate library member with a target T-cell expressing on its surface with: i) a Co-MOD that binds the parental wild-type MOD; and ii) a TCR that binds to the epitope;
C) when the T-Cell-MMP-epitope conjugate comprises an epitope tag, contacting the T-Cell-MMP-epitope conjugate library member bound to the target T-cell with a fluorescently labeled binding agent that binds to the epitope tag (which is unnecessary with a fluorescently labeled T-Cell-MMP-epitope conjugate), generating a library member/target T-cell/binding agent complex;
D) measuring the mean fluorescence intensity (MFI) of the T-Cell-MMP-epitope conjugate library member/target T-cell/binding agent complex using flow cytometry, wherein the MFI measured over a range of concentrations of the T-Cell-MMP-epitope conjugate library member provides a measure of the affinity and apparent avidity; and
E) selecting a T-Cell-MMP-epitope conjugate library member that selectively binds the target T-cell, compared to binding of the T-Cell-MMP-epitope conjugate library member to a control T-cell that comprises: i) the Co-MOD that binds the parental wild-type MOD; and ii) a TCR that binds to an epitope other than the epitope present in the T-Cell-MMP library member.

In some cases, a T-Cell-MMP library member that is identified as selectively binding to a target T-cell is isolated from the library. In some cases, parental wild-type MOD and Co-MOD pairs are selected from: IL-2 and IL-2 receptor; 4-1BBL and 4-1BB; PD-L1 and PD-1; FasL and Fas; TGF-β and TGF-β receptor; CD80 and CD28; CD86 and CD28; OX40L and OX40; ICOS-L and ICOS; ICAM and LFA-1; JAG1 and Notch; JAG1 and CD46; CD70 and CD27; CD80 and CTLA4; and CD86 and CTLA4.

The present disclosure provides a method of obtaining a T-Cell-MMP-epitope conjugate comprising one or more variant MODs that exhibit reduced affinity for a Co-MOD compared to the affinity of the corresponding parental wild-type MOD for the Co-MOD, the method comprising selecting, from a library of T-Cell-MMP-epitope conjugates comprising a plurality of members, a member that exhibits reduced affinity for the Co-MOD, wherein each of the plurality of members comprises: a) a first polypeptide comprising: i) an epitope covalently bound to a chemical conjugation site; and ii) a first MHC polypeptide; and b) a second polypeptide comprising: i) a second MHC polypeptide; and ii) optionally an Ig Fc polypeptide or a non-Ig scaffold, wherein the members of the library comprise a plurality of variant MODs present in the first polypeptide, the second polypeptide, or both the first and the second polypeptide. In some cases, the selecting step comprises determining the affinity, using BLI, of binding between T-Cell-MMP-epitope conjugate library members and the Co-MOD. In some cases, the T-Cell-MMP-epitope conjugate is as described above.

In some cases, the method of obtaining T-Cell-MMP-epitope conjugates comprising one or more variant MODs that exhibit reduced affinity for a Co-MOD compared to the affinity of the corresponding parental wild-type MODs for the Co-MOD further comprises: a) contacting the selected T-Cell-MMP-epitope conjugate library member with a target T-cell expressing on its surface: i) a Co-MOD that binds the parental wild-type MOD; and ii) a TCR that binds to the epitope, wherein the T-Cell-MMP-epitope conjugate library member comprises an epitope tag, such that the T-Cell-MMP-epitope conjugate library member binds to the target T-cell; b) contacting the selected T-Cell-MMP-epitope conjugate library member bound to the target T-cell with a fluorescently labeled binding agent that binds to the epitope tag, generating a selected T-Cell-MMP-epitope conjugate library member/target T-cell/binding agent complex; and c) measuring the MFI of the selected T-Cell-MMP-epitope conjugate library member/target T-cell/binding agent complex using flow cytometry, wherein the MFI measured over a range of concentrations of the selected T-Cell-MMP-epitope conjugate library member provides a measure of the affinity and apparent avidity. A selected T-Cell-MMP-epitope conjugate library member that selectively binds the target T-cell, compared to binding of the T-Cell-MMP-epitope conjugate library member to a control T-cell that comprises: i) the Co-MOD that binds the parental wild-type MOD; and ii) a TCR that binds to an epitope other than the epitope present in the T-Cell-MMP-epitope conjugate library member, is identified as selectively binding to the target T-cell. In some cases, the binding agent is an antibody specific for the epitope tag. In some cases, the variant MOD comprises from 1 to 20 aa substitutions (e.g., 1, 2, 3, 4, 5, 6, 7, 8, 9, 10, 11, 12, 13, 14, 15, 16, 17, 18, 19, or 20 aa substitutions) compared to the corresponding parental wild-type MOD. In some cases, the T-Cell-MMP-epitope conjugate comprises two variant MODs. In some cases, the two variant MODs comprise the same amino acid sequence. In some cases, the first polypeptide comprises one of the two variant MODs and the second polypeptide comprises the second of the two variant MODs. In some cases, the two variant MODs are on the same polypeptide chain of the T-Cell-MMP-epitope conjugate. In some cases, the two variant MODs are on the first polypeptide of the T-Cell-MMP-epitope conjugate. In some cases, the two variant MODs are on the second polypeptide of the T-Cell-MMP-epitope conjugate.

In some cases, the method of obtaining a T-Cell-MMP-epitope conjugate comprising one or more variant MODs that exhibit reduced affinity for a Co-MOD compared to the affinity of the corresponding parental wild-type MOD for the Co-MOD further comprises isolating the selected T-Cell-MMP-epitope conjugate library member from the library. In some cases, the method further comprises providing a nucleic acid comprising a nucleotide sequence encoding a T-Cell-MMP with at least one chemical conjugation site used to prepare the selected library member. In some cases, the nucleic acid is present in a recombinant expression vector. In some cases, the nucleotide sequence is operably linked to a transcriptional control element that is functional in a eukaryotic cell. In some cases, the method further comprises introducing the nucleic acid into a eukaryotic host cell, and culturing the cell in a liquid medium to synthesize the encoded T-Cell-MMP with at least one chemical conjugation site in the cell, isolating the synthesized T-Cell-MMP with at least one chemical conjugation site from the cell or from liquid culture medium, and conjugating it to at least one epitope to form the selected T-Cell-MMP-epitope conjugate. In some cases, the selected T-Cell-MMP with at least one chemical conjugation site comprises an Ig Fc polypeptide. In some cases, the method further comprises conjugating a drug to the Ig Fc polypeptide. In some cases, the drug is a cytotoxic agent that is selected from maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. In some cases, the drug is a retinoid. In some cases, the parental wild-type MOD and the Co-MODs are selected from: IL-2 and IL-2 receptor; 4-1BBL and 4-1BB; PD-L1 and PD-1; FasL and Fas; TGF-β and TGF-β receptor; CD70 and CD27; CD80 and CD28; CD86 and CD28; OX40L and OX40; FasL and Fas; ICOS-L and ICOS; ICAM and LFA-1; and JAG1 and Notch; JAG1 and CD46; CD80 and CTLA4; and CD86 and CTLA4.

The present disclosure provides a method of obtaining a T-Cell-MMP-epitope conjugate comprising one or more variant MODs that exhibit reduced affinity for a Co-MOD compared to the affinity of the corresponding parental wild-type MOD for the Co-MOD, the method comprising: A) providing a library of T-Cell-MMP-epitope conjugates comprising a plurality of members, wherein the plurality of members comprise: a) a first polypeptide comprising: i) an epitope covalently bound at a chemical conjugation site; and ii) a first MHC polypeptide; and b) a second polypeptide comprising: i) a second MHC polypeptide; and ii) optionally an Ig Fc polypeptide or a non-Ig scaffold, wherein the members of the library comprise a plurality of variant MODs present in the first polypeptide, the second polypeptide, or both the first and the second polypeptide; and B) selecting from the library a member that exhibits reduced affinity for the Co-MOD. In some cases, the selecting step comprises determining the affinity, using BLI, of binding between T-Cell-MMP-epitope conjugate library members and the Co-MOD. In some cases, the T-Cell-MMP-epitope conjugate is as described above.

In some cases, the method further comprises: a) contacting the selected T-Cell-MMP-epitope conjugate library member with a target T-cell expressing on its surface: i) a Co-MOD that binds the parental wild-type MOD; and ii) a T-cell receptor that binds to the epitope, wherein the T-Cell-MMP-epitope conjugate library member comprises an epitope tag, such that the T-Cell-MMP-epitope conjugate library member binds to the target T-cell; b) contacting the selected T-Cell-MMP-epitope conjugate library member bound to the target T-cell with a fluorescently labeled binding agent that binds to the epitope tag, generating a selected T-Cell-MMP-epitope conjugate library member/target T-cell/binding agent complex; and c) measuring the MFI of the selected T-Cell-MMP-epitope conjugate library member/target T-cell/binding agent complex using flow cytometry, wherein the MFI measured over a range of concentrations of the selected T-Cell-MMP-epitope conjugate library member provides a measure of the affinity and apparent avidity. A selected T-Cell-MMP-epitope conjugate library member that selectively binds the target T-cell, compared to binding of the T-Cell-MMP-epitope conjugate library member to a control T-cell that comprises: i) the Co-MOD that binds the parental wild-type MOD; and ii) a T-cell receptor that binds to an epitope other than the epitope present in the T-Cell-MMP-epitope conjugate library member, is identified as selectively binding to the target T-cell. In some cases, the binding agent is an antibody specific for the epitope tag. In some cases, the variant MOD comprises from 1 to 20 aa substitutions (e.g., 1, 2, 3, 4, 5, 6, 7, 8, 9, 10, 11, 12, 13, 14, 15, 16, 17, 18, 19 or 20 aa substitutions) compared to the corresponding parental wild-type MOD. In some cases, the T-Cell-MMP-epitope conjugate comprises two variant MODs. In some cases, the two variant MODs comprise the same amino acid sequence. In some cases, the first polypeptide comprises one of the two variant MODs and the second polypeptide comprises the second of the two variant MODs. In some cases, the two variant MODs are on the same polypeptide chain of the T-Cell-MMP-epitope conjugate. In some cases, the two variant MODs are on the first polypeptide of the T-Cell-MMP-epitope conjugate. In some cases, the two variant MODs are on the second polypeptide of the T-Cell-MMP-epitope conjugate.

In some cases, the method further comprises isolating the selected T-Cell-MMP-epitope conjugate library member from the library. In some cases, the method further comprises providing a nucleic acid comprising a nucleotide sequence encoding a T-Cell-MMP with at least one chemical conjugation site used to prepare the selected library member. In some cases, the nucleic acid is present in a recombinant expression vector. In some cases, the nucleotide sequence is operably linked to a transcriptional control element that is functional in a eukaryotic cell. In some cases, the method further comprises introducing the nucleic acid into a eukaryotic host cell, and culturing the cell in a liquid medium to synthesize the encoded T-Cell-MMP with at least one chemical conjugation site in the cell, isolating the synthesized selected T-Cell-MMP with at least one chemical conjugation site from the cell or from the liquid culture medium, and conjugating it to at least one epitope to form the selected T-Cell-MMP-epitope conjugate. In some cases, the selected T-Cell-MMP library member comprises an Ig Fc polypeptide. In some cases, the method further comprises conjugating a drug to the Ig Fc polypeptide. In some cases, the drug is a cytotoxic agent selected from maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. In some cases, the drug is a retinoid. In some cases, the parental wild-type MODs and the cognate co-MODs are selected from: IL-2 and IL-2 receptor; 4-1BBL and 4-1BB; PD-L1 and PD-1; FasL and Fas; TGF-β and TGF-β receptor; CD70 and CD27; CD80 and CD28; CD86 and CD28; OX40L and OX40; FasL and Fas; ICOS-L and ICOS; ICAM and LFA-1; and JAG1 and Notch; JAG1 and CD46; CD80 and CTLA4; and CD86 and CTLA4.

### III. Nucleic Acids

The present disclosure provides a nucleic acid comprising a nucleotide sequence encoding a T-Cell-MMP of the present disclosure. The present disclosure provides a nucleic acid comprising a nucleotide sequence encoding a T-Cell-MMP of the present disclosure including chemical conjugation sites that are engineered into the polypeptides of the T-Cell-MMP.

The present disclosure provides nucleic acids comprising nucleotide sequences encoding the T-Cell-MMPs described herein. In some cases, the individual polypeptide chains of a T-Cell-MMP of the present disclosure are encoded in separate nucleic acids. In some cases, all polypeptide chains of a T-Cell-MMP of the present disclosure are encoded in a single nucleic acid. In some cases, a first nucleic acid comprises a nucleotide sequence encoding a first polypeptide of a T-Cell-MMP of the present disclosure; and a second nucleic acid comprises a nucleotide sequence encoding a second polypeptide of a T-Cell-MMP of the present disclosure. In some cases, a single nucleic acid comprises a nucleotide sequence encoding a first polypeptide of a T-Cell-MMP of the present disclosure and a second polypeptide of a T-Cell-MMP of the present disclosure.

A. Separate Nucleic Acids Encoding Individual Polypeptide Chains of a Multimeric Polypeptide

The present disclosure provides nucleic acids comprising nucleotide sequences encoding a T-Cell-MMP. As noted above, in some cases, the individual polypeptide chains of a T-Cell-MMP are encoded in separate nucleic acids. In some cases, nucleotide sequences encoding the separate polypeptide chains of a T-Cell-MMP are operably linked to transcriptional control elements, e.g., promoters, such as promoters that are functional in a eukaryotic cell, where the promoter can be a constitutive promoter or an inducible promoter.

The present disclosure provides a first nucleic acid and a second nucleic acid, where the first nucleic acid comprises a nucleotide sequence encoding a first polypeptide of a T-Cell-MMP of the present disclosure, where the first polypeptide comprises, in order from N-terminus to C-terminus: a) a first MHC polypeptide; and b) a MOD (e.g., a reduced-affinity variant MOD polypeptide as described above); and where the second nucleic acid comprises a nucleotide sequence encoding a second polypeptide of a T-Cell-MMP, where the second polypeptide comprises, in order from N-terminus to C-terminus: a) a second MHC polypeptide; and b) an Ig Fc polypeptide. Suitable epitopes, MHC polypeptides, MODs, and Ig Fc polypeptides are described above. At least one of the first and second polypeptides comprises a chemical conjugation site (or a nascent site that can be converted to a chemical conjugation site). In some cases, the nucleotide sequences encoding the first and second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a T-Cell-MMP, where the first polypeptide comprises a first MHC polypeptide; and where the second nucleic acid comprises a nucleotide sequence encoding a second polypeptide of a T-Cell-MMP, where the second polypeptide comprises, in order from N-terminus to C-terminus: a) a MOD (e.g., a reduced-affinity variant MOD polypeptide as described above); b) a second MHC polypeptide; and c) an Ig Fc polypeptide. Suitable MHC polypeptides, MODs, and Ig Fc polypeptides are described above. At least one of the first and second polypeptides comprises a chemical conjugation site. In some cases, the nucleotide sequences encoding the first and second polypeptides are operably linked to transcriptional control elements. In some cases, the transcriptional control element is a promoter that is functional in a eukaryotic cell. In some cases, the nucleic acids are present in separate expression vectors.

B. Nucleic Acid Encoding Two or More Polypeptides Present in a T-Cell-MMP

The present disclosure provides a nucleic acid comprising nucleotide sequences encoding at least the first polypeptide and the second polypeptide of a T-Cell-MMP. In some cases, where a T-Cell-MMP of the present disclosure includes a first, second, and third polypeptide, the nucleic acid includes a nucleotide sequence encoding the first, second, and third polypeptides. In some cases, the nucleotide sequences encoding the first polypeptide and the second polypeptide of a T-Cell-MMP include a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a T-Cell-MMP include an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a T-Cell-MMP include a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a T-Cell-MMP; in any of these embodiments, an IRES or a ribosome skipping signal can be used in place of the nucleotide sequence encoding the proteolytically cleavable linker.

In some cases provided for herein, a first nucleic acid (e.g., a recombinant expression vector, an mRNA, a viral RNA, etc.) comprises a nucleotide sequence encoding a first polypeptide chain of a T-Cell-MMP; and a second nucleic acid (e.g., a recombinant expression vector, an mRNA, a viral RNA, etc.) comprises a nucleotide sequence encoding a second polypeptide chain of the T-Cell-MMP. In some cases, the nucleotide sequence encoding the first polypeptide and the nucleotide sequence encoding the second polypeptide are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus the elements: a) a first MHC polypeptide; b) a MOD (e.g., a reduced-affinity variant as described above); c) a proteolytically cleavable linker; d) a second MHC polypeptide; and e) an immunoglobulin (Ig) Fc polypeptide; wherein at least one of the elements comprises a chemical conjugation site that is not removed during cellular processing. The present disclosure provides a nucleic acid comprising a nucleotide sequence encoding a recombinant polypeptide, where the recombinant polypeptide comprises, in order from N-terminus to C-terminus the elements: a) a first leader peptide; b) a first MHC polypeptide; c) a MOD (e.g., a reduced-affinity variant as described above); d) a proteolytically cleavable linker; e) a second leader peptide; f) a second MHC polypeptide; and g) an Ig Fc polypeptide; wherein at least one of the elements comprises a chemical conjugation site that is not removed during cellular processing. The present disclosure provides a nucleic acid comprising a nucleotide sequence encoding a recombinant polypeptide, where the recombinant polypeptide comprises, in order from N-terminus to C-terminus, the elements: a) a first MHC polypeptide; b) a proteolytically cleavable linker; c) a MOD (e.g., a reduced-affinity variant as described above); d) a second MHC polypeptide; and e) an Ig Fc polypeptide; wherein at least one of the elements comprises a chemical conjugation site that is not removed during cellular processing. In some cases, the first leader peptide and the second leader peptide are β2M leader peptides. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide comprises a β2-microglobulin (β2M) polypeptide; and the second MHC polypeptide comprises a MHC Class I heavy chain polypeptide. In some cases, the β2M polypeptide comprises an amino acid sequence having at least about 85% (e.g., at lease about 90%, 95%, 98%, 99%, or even 100%) aa sequence identity to a β2M amino acid sequence depicted in FIG. 4. In some cases, the MHC Class I heavy chain polypeptide is a HLA-A, HLA-B, HLA-C, HLA-E, HLA-F, HLA-G, HLA-K, or HLA-L heavy chain. In some cases, the MHC Class I heavy chain polypeptide comprises an amino acid sequence having at least 85% aa sequence identity to the amino acid sequence depicted in any one of FIGS. 3A-3D. In such an embodiment the MHC Class I heavy chain polypeptide may not comprise a transmembrane anchoring domain (e.g., the heavy chain polypeptide comprises a sequence in FIG. 3D).

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% aa sequence identity to an amino acid sequence depicted in FIGS. 2A-2G.

Suitable immunomodulatory polypeptides (MODs) are described above.

In addition to any other proteolytically cleavable linkers, in some cases, the proteolytically cleavable linker comprises an amino acid sequence selected from the group consisting of: a) LEVLFQGP (SEQ ID NO:357); b) ENLYTQS (SEQ ID NO:358); c) DDDDK (SEQ ID NO:359); d) LVPR (SEQ ID NO:360); and e) GSGATNFSLLKQAGDVEENPGP (SEQ ID NO:361).

In some cases, a linker comprising a first Cys residue attached to the first MHC polypeptide is provided, and the second MHC polypeptide comprises an aa substitution to provide a second (engineered) Cys residue, such that the first and second Cys residues provide for a disulfide linkage between the linker and the second MHC polypeptide. In some cases, the first MHC polypeptide comprises an aa substitution to provide a first engineered Cys residue, and the second MHC polypeptide comprises an aa substitution to provide a second engineered Cys residue, such that the first Cys residue and the second Cys residue provide for a disulfide linkage between the first MHC polypeptide and the second MHC polypeptide. As discussed above, where disulfide bridges are provided, it is possible to use either thiol reactive agents or bis-thiol linkers to incorporate payloads or epitopes.

C. Recombinant Expression Vectors

The present disclosure provides recombinant expression vectors comprising nucleic acids of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example for eukaryotic host cells: pXT1, pSG5 (Stratagene®), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc., may be used in the expression vector (see, e.g., Bitter et al. (1987), Methods in Enzymology, 153:516-544).

In some embodiments, a nucleotide sequence encoding a DNA-targeting RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a DNA-targeting RNA and/or a site-directed modifying polypeptide is operably linked to multiple control elements that allow expression of the nucleotide sequence encoding the DNA-targeting RNA and/or site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### IV. Genetically Modified Host Cells

The present disclosure provides a genetically modified host cell, where the host cell is genetically modified with a nucleic acid of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2M and/or such that it does not synthesize endogenous MHC Class I heavy chains (MHC-H). In addition to the foregoing, host cells expressing formylglycine generating enzyme (FGE) activity are discussed above for use with T-Cell-MMPs comprising a sulfatase motif, and such cells may advantageously be modified such that they do not express at least one, if not both, of the endogenous MHC β2M and MHC-H proteins.

### V. Compositions

The present disclosure provides compositions, including pharmaceutical compositions, comprising one or more T-Cell-MMPs and/or T-Cell-MMP-epitope conjugates, wherein the pharmaceutical compositions may comprise one or more pharmaceutically acceptable excipients as provided below. The present disclosure also provides compositions, including pharmaceutical compositions, comprising a nucleic acid or a recombinant expression vector of the present disclosure.

A. Compositions Comprising T-Cell-MMPs

A composition of the present disclosure can comprise, in addition to a T-Cell-MMP or its epitope conjugate of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

A composition comprising a T-Cell-MMP or its epitope conjugate may further comprise a pharmaceutically acceptable excipient, a variety of which are known in the art and need not be discussed in detail herein. Pharmaceutically acceptable excipients have been amply described in a variety of publications including, for example, “Remington: The Science and Practice of Pharmacy”, 19^{th} Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a T-Cell-MMP of the present disclosure, and a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include (e.g., be in the form of) aqueous or other solutions, powders, granules, tablets, pills, suppositories, capsules, suspensions, sprays, and the like. The composition may be formulated according to the various routes of administration described below.

Where a T-Cell-MMP of the present disclosure is administered as an injectable (e.g., subcutaneously, intraperitoneally, intramuscularly, and/or intravenously) directly into a tissue, a formulation can be provided as a ready-to-use dosage form, a non-aqueous form (e.g., a reconstitutable storage-stable powder) or an aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a T-Cell-MMP and/or T-Cell-MMP-epitope conjugate in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

The present disclosure provides compositions, including pharmaceutical compositions, comprising a T-Cell-MMP or its epitope conjugate. A composition can comprise: a) a T-Cell-MMP and/or a T-Cell-MMP-epitope conjugate; and b) an excipient, as described above for the T-Cell-MMPs and their epitope conjugates. In some cases, the excipient is a pharmaceutically acceptable excipient.

In some cases, a T-Cell-MMP and/or T-Cell-MMP-epitope conjugate is present in a liquid composition. Thus, the present disclosure provides compositions (e.g., liquid compositions, including pharmaceutical compositions) comprising a T-Cell-MMP and/or T-Cell-MMP-epitope conjugate of the present disclosure. In some cases, a composition of the present disclosure comprises: a) a T-Cell-MMP and/or T-Cell-MMP-epitope conjugate of the present disclosure; and b) saline (e.g., 0.9% or about 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a T-Cell-MMP and/or T-Cell-MMP-epitope conjugate; and b) saline (e.g., 0.9% or about 0.9% NaCl), where the composition is sterile and is free of detectable pyrogens and/or other toxins.

B. Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a T-Cell-MMP; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (for example) N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some embodiments, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in one or more spherical bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers, surfactants and chelators. Suitable surfactants include, but are not limited to, fatty acids and/or esters or salts thereof, bile acids, and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

### VI. Methods of Modulating T-Cell Activity

The present disclosure provides a method of selectively modulating the activity of a T cell, the method comprising contacting the T cell with a MODs on a T-Cell-MMP-epitope conjugate, and in some instances the payload the T-Cell-MMP-epitope conjugate may be carrying. Where the T-Cell-MMP has been conjugated to an epitope (i.e. it is a T-Cell-MMP-epitope conjugate), contacting the conjugate to a T-cell can result in epitope-specific T-cell modulation. In some cases, the contacting occurs in vivo (e.g., in a mammal such as a human, rat, mouse, dog, cat, pig, horse, or primate). In some cases, the contacting occurs in vitro. In some cases, the contacting occurs ex vivo. In some cases, the T-cell is a CD8+ T-cell, CD4+ T-cell, a NK-T-cell, or a Treg cell as described below under Treatment Methods. In some cases, the T-cell is a CD8+ T-cell as described below under Treatment Methods.

The present disclosure provides a method of selectively modulating the activity of an epitope-specific T-cell, the method comprising contacting the T-Cell with a T-Cell-MMP-epitope conjugate of the present disclosure bearing the epitope recognized by the epitope-specific T-Cell, where contacting the T-Cell with a T-Cell-MMP-epitope conjugate of the present disclosure selectively modulates the activity of the epitope-specific T-Cell. In some cases, the contacting occurs in vitro. In some cases, the contacting occurs in vivo. In some cases, the contacting occurs ex vivo.

In some cases, e.g., where the target T-cell is a CD8⁺ T-cell, the T-Cell-MMP-epitope conjugate comprises Class I MHC polypeptides (e.g., β2-microglobulin and Class I MHC heavy chain).

Where a T-Cell-MMP-epitope conjugate of the present disclosure includes a MOD that is an activating polypeptide, contacting the T-cell with the T-Cell-MMP-epitope conjugate activates the epitope-specific T-cell. In some instances, the epitope-specific T-cell is a T-cell that is specific for an epitope present on a cancer cell, and contacting the epitope-specific T-cell with the T-Cell-MMP-epitope conjugate increases cytotoxic activity of the T-cell toward the cancer cell. In some instances, the epitope-specific T-cell is a T-cell that is specific for an epitope present on a cancer cell, and contacting the epitope-specific T-cell with the T-Cell-MMP-epitope conjugate increases the number of the epitope-specific T-cells.

In some instances, the epitope-specific T-cell is a T-cell that is specific for an epitope present on a virus-infected cell, and contacting the epitope-specific T-cell with the T-Cell-MMP-epitope conjugate increases cytotoxic activity of the T-cell toward the virus-infected cell. In some instances, the epitope-specific T-cell is a T-cell that is specific for an epitope present on a virus-infected cell, and contacting the epitope-specific T-cell with the T-Cell-MMP-epitope conjugate increases the number of the epitope-specific T-cells.

Where a T-Cell-MMP-epitope conjugate of the present disclosure includes a MOD that is an inhibiting polypeptide, contacting the T-cell with the multimer inhibits the epitope-specific T-cell. In some instances, the epitope-specific T-cell is a self-reactive T-cell that is specific for an epitope present in a self-antigen, and the contacting reduces the number of the self-reactive T-cells.

The present disclosure provides a method of modulating an immune response in an individual, the method comprising administering to the individual an effective amount of a T-Cell-MMP-epitope conjugate of the present disclosure. Administering the T-Cell-MMP-epitope conjugate induces an epitope-specific T cell response (e.g., an cancer epitope-specific T-cell response) and an epitope-non-specific T cell response, where the ratio of the epitope-specific T cell response to the epitope-non-specific T cell response is at least 2:1. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1. In some cases, the individual is a human. In some cases, the modulating increases a cytotoxic T-cell response to a cancer cell, e.g., a cancer cell expressing an antigen that displays the same epitope displayed by the peptide epitope present in the T-Cell-MMP-epitope conjugate. In some cases, the administering is intravenous, subcutaneous, intramuscular, systemic, intralymphatic, distal to a treatment site, local, or at or near a treatment site.

The present disclosure also provides a method of detecting, in a mixed population of cells (e.g., a mixed population of T cells) obtained from an individual, the presence of a target T cells that binds an epitope of interest (e.g., a cancer, pathogen, or self epitope), the method comprising: a) contacting in vitro the mixed population of cell (e.g., mixed population of T cells) with a T-Cell-MMP-epitope conjugate of the present disclosure, wherein the T-Cell-MMP-epitope conjugate comprises the epitope of interest (e.g., the cancer, pathogen, or self epitope); and b) detecting activation and/or proliferation of T cells in response to said contacting, wherein activated and/or proliferated T cells indicates the presence of the target T cell.

### VII. Methods of Selectively Delivering a Costimulatory Polypeptide (MOD)

The present disclosure provides a method of delivering one or more independently selected MODs and/or a reduced-affinity variant of a naturally occurring MODs (such as a variant disclosed herein) to a selected T-cell or a selected T-cell population, e.g., in a manner such that a TCR specific for a given epitope is targeted (e.g., a cancer, pathogen, or self epitope). The present disclosure provides a method of delivering a MOD or a reduced-affinity variant of a naturally occurring MOD disclosed herein, selectively to a target T-cell bearing a TCR specific for the epitope (e.g., a cancer, pathogen, or self epitope) present in a T-Cell-MMP-epitope conjugate of the present disclosure. The method comprises contacting a population of T-cells with a T-Cell-MMP-epitope conjugate of the present disclosure. The population of T-cells can be a mixed population that comprises: i) the target T-cell; and ii) non-target T-cells that are not specific for the epitope (e.g., T-cells that are specific for an epitope(s) other than the epitope to which the epitope-specific T-cell binds). The epitope-specific T-cell is specific for the epitope-presenting peptide (e.g., a peptide presenting a cancer, pathogen, or self epitope) present in the T-Cell-MMP-epitope conjugate and binds to the peptide HLA complex or peptide MHC complex provided by the T-Cell-MMP-epitope conjugate. Accordingly, contacting the population of T-cells with the T-Cell-MMP-epitope conjugate delivers the costimulatory polypeptide (e.g., a wild-type MOD or a reduced-affinity variant of the wild-type MOD, as described herein) selectively to the T-cell(s) that are specific for the epitope present in the T-Cell-MMP-epitope conjugate. In some cases, the population of T cells is in vitro. In some cases, the population of T cells is in vivo in an individual. In some cases, the method comprises administering the T-Cell-MMP-epitope conjugate to the individual. In some case, the T cell is a cytotoxic T cell. In some cases, the mixed population of T cells is an in vitro population of mixed T cells obtained from an individual, and the contacting step results in activation and/or proliferation of the target T cell(s), generating a population of activated and/or proliferated target T cells; in some of these instances, the method further comprises administering the population of activated and/or proliferated target T cells to the individual.

Thus, the present disclosure provides a method of delivering a MOD (such as IL-2), or a reduced-affinity variant of a naturally occurring MOD (such as an IL-2 variant) disclosed herein, or a combination of both, selectively to a target T-cell, the method comprising contacting a mixed population of T-cells with a T-Cell-MMP-epitope conjugate of the present disclosure. The mixed population of T-cells comprises the target T-cell and non-target T-cells. The target T-cell is specific for the epitope present within the T-Cell-MMP-epitope conjugate. Contacting the mixed population of T-cells with a T-Cell-MMP-epitope conjugate of the present disclosure delivers the MOD(s) present within the T-Cell-MMP-epitope conjugate to the target T-cell.

### VIII. Treatment Methods

The present disclosure provides a method of selectively modulating the activity of an epitope-specific T-cell in an individual (e.g., treat an individual), the method comprising administering to the individual an amount of a T-Cell-MMP and/or T-Cell-MMP-epitope conjugate of the present disclosure, or one or more nucleic acids encoding a T-Cell-MMP. Also provided is a T-Cell-MMP-epitope conjugate of the present disclosure for use in a method of treatment of the human or animal body. In some cases, a treatment method of the present disclosure comprises administering to an individual in need thereof one or more recombinant expression vectors comprising nucleotide sequences encoding a T-Cell-MMP of the present disclosure. In some cases, a treatment method of the present disclosure comprises administering to an individual in need thereof one or more mRNA molecules comprising nucleotide sequences encoding a T-Cell-MMP of the present disclosure. In some cases, a treatment method of the present disclosure comprises administering to an individual in need thereof a T-Cell-MMP-epitope conjugate of the present disclosure. Conditions that can be treated include, infections, cancer, and autoimmune disorders, examples of some of which are described below.

In some cases, a T-cell-MMP-epitope conjugate of the present disclosure, when administered to an individual in need thereof, induces both an epitope-specific T-cell response and an epitope non-specific T-cell response. In other words, in some cases, a T-cell-MMP-epitope conjugate of the present disclosure, when administered to an individual in need thereof, induces an epitope-specific T-cell response by modulating the activity of a first T-cell that displays both: i) a TCR specific for the epitope present in the T-Cell-MMP; and ii) a Co-MOD that binds to the MOD present in the T-Cell-MMP-epitope conjugate; and induces an epitope non-specific T-cell response by modulating the activity of a second T-cell that displays: i) a TCR specific for an epitope other than the epitope present in the T-Cell-MMP; and ii) a Co-MOD that binds to the MOD present in the T-Cell-MMP. The ratio of the epitope-specific T-cell response to the epitope-non-specific T-cell response is at least 2:1, at least 5:1, at least 10:1, at least 15:1, at least 20:1, at least 25:1, at least 50:1, or at least 100:1. The ratio of the epitope-specific T-cell response to the epitope-non-specific T-cell response is from about 2:1 to about 5:1, from about 5:1 to about 10:1, from about 10:1 to about 15:1, from about 15:1 to about 20:1, from about 20:1 to about 25:1, from about 25:1 to about 50:1, from about 50:1 to about 100:1, or more than 100:1. “Modulating the activity” of a T-cell can include one or more of: i) activating a cytotoxic (e.g., CD8⁺) T-cell; ii) inducing cytotoxic activity of a cytotoxic (e.g., CD8⁺) T-cell; iii) inducing production and release of a cytotoxin (e.g., a perforin; a granzyme; a granulysin) by a cytotoxic (e.g., CD8⁺) T-cell; iv) inhibiting activity of an autoreactive T-cell; and the like.

The combination of the reduced affinity of the MOD for its Co-MOD, and the affinity of the epitope for a TCR, provides for enhanced selectivity of a T-Cell-MMP-epitope conjugate of the present disclosure. Thus, for example, a T-Cell-MMP-epitope conjugate of the present disclosure binds with higher avidity to a first T-cell that displays both: i) a TCR specific for the epitope present in the T-Cell-MMP-epitope conjugate; and ii) a Co-MOD that binds to the MOD present in the T-Cell-MMP-epitope conjugate, compared to the avidity to which it binds to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate.

The present disclosure provides a method of selectively modulating the activity of an epitope-specific T-cell in an individual, the method comprising administering to the individual an effective amount of a T-Cell-MMP-epitope conjugate of the present disclosure, where the T-Cell-MMP-epitope conjugate selectively modulates the activity of the epitope-specific T-cell in the individual. Selectively modulating the activity of an epitope-specific T-cell can treat a disease or disorder in the individual. Thus, the present disclosure provides a treatment method comprising administering to an individual in need thereof an effective amount of a T-Cell-MMP-epitope conjugate.

In some cases, the MOD is an activating polypeptide, and the T-Cell-MMP-epitope conjugate activates the epitope-specific T cell. In some cases, the epitope is a cancer-associated epitope, and the T-Cell-MMP-epitope conjugate increases the activity of a T cell specific for the cancer-associate epitope.

In some cases, the MOD is an activating polypeptide, and the T-Cell-MMP-epitope conjugate activates an epitope-specific T-cell that recognizes a cancer or pathogen specific antigen (e.g., a cancer-associated antigen or a viral or bacterial antigen). In some cases, the T cells are T-helper cells (CD4⁺ cells), cytotoxic T-cells (CD8⁺ cells), or NK-T-cells. In some cases T-Cell-MMP-epitope conjugate increases the activity of a T-cell specific for a cancer cell expressing the epitope (e.g., T-helper cells (CD4⁺ cells), cytotoxic T-cells (CD8⁺ cells), and/or NK-T-cells). Activation of CD4⁺ T cells can include increasing proliferation of CD4⁺ T cells and/or inducing or enhancing release cytokines by CD4⁺ T cells. Activation of NK-T-cells and/or CD8+ cells can include: increasing proliferation of NK-T-cells and/or CD8+ cells; and/or inducing release of cytokines such as interferon γ by NK-T-cells and/or CD8+ cells.

In some cases, a T-Cell-MMP-epitope conjugate of the present disclosure reduces proliferation and/or activity of a regulatory T (Treg) cell. Tregs are FoxP3⁺, CD4⁺ T cells. In some cases, e.g., where a T-Cell-MMP-epitope conjugate of the present disclosure comprises an inhibitory MOD (e.g., PD-L1, FasL, and the like), the T-Cell-MMP-epitope conjugate reduces the proliferation and/or activity of a Treg.

Where a T-Cell-MMP-epitope conjugate of the present disclosure comprises an infectious disease-associated epitope (such as a viral or bacterial lipo-, glyco-. or phosphopeptide epitope), the T-Cell-MMP-epitope conjugate can be administered to an individual in need thereof to treat an infection in the individual, where the infectious agent expresses the peptide epitope present in the T-Cell-MMP-epitope conjugate. The present disclosure provides a method of treating an infection an individual, the method comprising administering to the individual an effective amount of a T-Cell-MMP-epitope conjugate of the present disclosure.

Where a T-Cell-MMP-epitope conjugate of the present disclosure comprises a cancer peptide epitope, the T-Cell-MMP-epitope conjugate can be administered to an individual in need thereof to treat a cancer in the individual, where the cancer expresses the cancer peptide epitope present in the T-Cell-MMP-epitope conjugate. The present disclosure provides a method of treating cancer in an individual, the method comprising administering to the individual an effective amount of a T-Cell-MMP-epitope conjugate of the present disclosure. In such a method of treatment the T-Cell-MMP-epitope conjugate may comprises a stimulatory MOD. In some cases, an “effective amount” of a T-Cell-MMP-epitope conjugate is an amount that, when administered in one or more doses to an individual in need thereof, reduces the number of cancer cells in the individual. For example, in some cases, an “effective amount” of a T-Cell-MMP-epitope conjugate of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, at least 95%, or to undetectable levels compared to the number of cancer cells in the individual before administration of the T-Cell-MMP-epitope conjugate, or in the absence of administration with the T-Cell-MMP-epitope conjugate. In some cases, an “effective amount” of a T-Cell-MMP-epitope conjugate of the present disclosure is an amount that, when administered in one or more doses to an individual in need thereof (an individual having a tumor), reduces either the number of cancer cells in the individual compared to the number of cancer cells in the individual before administration of the T-Cell-MMP-epitope conjugate, or in the absence of administration with the T-Cell-MMP-epitope conjugate. In some cases, an “effective amount” of a T-Cell MMP-epitope conjugate of the present disclosure is an amount that, when administered in one or more doses to an individual in need thereof, reduces the number of cancer cells in the individual to undetectable levels.

In some cases, an “effective amount” of a T-Cell-MMP-epitope conjugate of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, at least 95%, or to an undetectable level compared to the total tumor mass in the individual before administration of the T-Cell-MMP-epitope conjugate, or in the absence of administration of the T-Cell-MMP-epitope conjugate.

In another embodiment, the “effective amount” of a T-Cell-MMP-epitope conjugate of the present disclosure is an amount that, when administered in one or more doses to an individual in need thereof (an individual having a tumor), reduces the tumor volume of at least one tumor in the individual. For example, in some cases, an “effective amount” of a multimeric polypeptide of the present disclosure is an amount that, when administered in one or more doses to an individual in need thereof (an individual having a tumor), reduces the tumor volume by at least 10%, at least 15%, at least 20%, at least 25%, at least 30%, at least 40%, at least 50%, at least 60%, at least 70%, at least 80%, at least 90%, at least 95%, or to undetectable levels (volume) compared to the tumor volume in the individual before administration of the T-Cell-MMP-epitope conjugate, or in the absence of administration of the T-Cell-MMP-epitope conjugate. In such an embodiment the mass may be calculated based on tumor density and volume.

In some cases, an “effective amount” of a T-Cell-MMP-epitope conjugate of the present disclosure is an amount that, when administered in one or more doses to an individual in need thereof, increases survival time of the individual. For example, in some cases, an “effective amount” of a T-Cell-MMP-epitope conjugate of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the T-Cell-MMP-epitope conjugate.

In some cases, an “effective amount” of a T-Cell-MMP-epitope conjugate of the present disclosure is an amount that, when administered in one or more doses to individuals in a population of individuals in need thereof, increases average survival time of the population. For example, in some cases, an “effective amount” of a T-Cell-MMP-epitope conjugate of the present disclosure is an amount that, when administered in one or more doses to individuals in a population of individuals suffering from a specified disease (e.g., type of cancer) in need thereof, increases the average survival time of the population of individuals receiving the T-Cell-MMP-epitope conjugate by at least 1 month, at least 2 months, at least 3 months, from 3 months to 6 months, from 6 months to 1 year, from 1 year to 2 years, from 2 years to 5 years, from 5 years to 10 years, or more than 10 years, compared to the average survival time of the individuals suffering from the specified disease not receiving the T-Cell-MMP-epitope conjugate; wherein the population is an age, gender, weight, and/or disease state (disease and degree of progression) matched population.

In some instances, the epitope-specific T-cell is a T-cell that is specific for an epitope present on a virus-infected cell, and contacting the epitope-specific T-cell with the T-Cell-MMP-epitope conjugate increases cytotoxic activity of the T-cell toward the virus-infected cell. In some instances, the epitope-specific T-cell is a T-cell that is specific for an epitope present on a virus-infected cell, and contacting the epitope-specific T-cell with the T-Cell-MMP-epitope conjugate increases the number of the epitope-specific T-cells. Accordingly, the present disclosure provides a method of treating a virus infection in an individual, the method comprising administering to the individual an effective amount of a T-Cell-MMP-epitope conjugate of the present disclosure, where the T-Cell-MMP-epitope conjugate comprises a T-cell epitope that is a viral epitope, and where the T-Cell-MMP-epitope conjugate comprises a stimulatory MOD. In some cases, an “effective amount” of a T-Cell-MMP-epitope conjugate is an amount that, when administered in one or more doses to an individual in need thereof, reduces the number of virus-infected cells in the individual. For example, in some cases, an “effective amount” of a T-Cell-MMP-epitope conjugate of the present disclosure is an amount that, when administered in one or more doses to an individual in need thereof, reduces the number of virus-infected cells in the individual by at least 10%, at least 15%, at least 20%, at least 25%, at least 30%, at least 40%, at least 50%, at least 60%, at least 70%, at least 80%, at least 90%, or at least 95%, compared to the number of virus-infected cells in the individual before administration of the T-Cell-MMP-epitope conjugate, or in the absence of administration with the T-Cell-MMP-epitope conjugate. In some cases, an “effective amount” of a T-Cell-MMP-epitope conjugate of the present disclosure is an amount that, when administered in one or more doses to an individual in need thereof, reduces the number of virus-infected cells in the individual to undetectable levels.

The present disclosure also provides a method of treating an infection in an individual, the method comprising administering to the individual an effective amount of a T-Cell-MMP and/or T-Cell-MMP-epitope conjugate of the present disclosure, where the T-Cell-MMP-epitope conjugate comprises a T-cell epitope that is a pathogen-associated epitope, and where the T-Cell-MMP and/or T-Cell-MMP-epitope conjugate comprises a stimulatory MOD. In some cases, an “effective amount” of a T-Cell-MMP is an amount that, when administered in one or more doses to an individual in need thereof, reduces the number of pathogens in the individual. For example, in some cases, an “effective amount” of a T-Cell-MMP and/or T-Cell-MMP-epitope conjugate of the present disclosure is an amount that, when administered in one or more doses to an individual in need thereof, reduces the number of pathogens in the individual by at least 10%, at least 15%, at least 20%, at least 25%, at least 30%, at least 40%, at least 50%, at least 60%, at least 70%, at least 80%, at least 90%, or at least 95%, compared to the number of pathogens in the individual before administration of the T-Cell-MMP and/or T-Cell-MMP-epitope conjugate, or in the absence of administration with the T-Cell-MMP and/or T-Cell-MMP-epitope conjugate. In some cases, an “effective amount” of a T-Cell-MMP and/or T-Cell-MMP-epitope conjugate of the present disclosure is an amount that, when administered in one or more doses to an individual in need thereof, reduces the number of pathogens in the individual to undetectable levels. Pathogens include viruses, bacteria, protozoans, and the like.

In some cases, the MOD is an inhibitory polypeptide, and the T-Cell-MMP-epitope conjugate inhibits activity of the epitope-specific T-cell. In some cases, the epitope is a self-epitope, and the T-Cell-MMP-epitope conjugate selectively inhibits the activity of a T-cell specific for the self-epitope.

The present disclosure provides a method of treating an autoimmune disorder in an individual, the method comprising administering to the individual an effective amount of a T-Cell-MMP (or one or more nucleic acids comprising nucleotide sequences encoding the T-Cell-MMP) and/or T-Cell-MMP-epitope conjugate comprising a self-epitope, where the T-Cell-MMP and/or T-Cell-MMP-epitope conjugate comprises an inhibitory MOD. In such cases, an “effective amount” of a T-Cell-MMP and/or T-Cell-MMP-epitope conjugate is an amount that, when administered in one or more doses to an individual in need thereof, reduces the number of self-reactive T-cells by at least 10%, at least 15%, at least 20%, at least 25%, at least 30%, at least 40%, at least 50%, at least 60%, at least 70%, at least 80%, at least 90%, or at least 95%, compared to the number of self-reactive T-cells in the individual before administration of the T-Cell-MMP and/or T-Cell-MMP-epitope conjugate, or in the absence of administration of the T-Cell-MMP and/or T-Cell-MMP-epitope conjugate. In some cases, an “effective amount” of such a T-Cell-MMP and/or T-Cell-MMP-epitope conjugate is an amount that, when administered in one or more doses to an individual in need thereof, reduces production of Th2 cytokines (e.g., by at least 10%, at least 15%, at least 20%, at least 25%, at least 30%, at least 40%, at least 50%, at least 60%, at least 70%, at least 80%, at least 90%, or at least 95%) in the individual. In some cases, an “effective amount” of such a T-Cell-MMP and/or T-Cell-MMP-epitope conjugate is an amount that, when administered in one or more doses to an individual in need thereof, ameliorates one or more symptoms associated with an autoimmune disease in the individual.

As noted above, in some cases, in carrying out a subject treatment method, a T-Cell-MMP and/or T-Cell-MMP-epitope conjugate of the present disclosure is administered to an individual in need thereof, as the polypeptide per se. In other instances, in carrying out a subject treatment method, one or more nucleic acids comprising nucleotide sequences encoding a T-Cell-MMP of the present disclosure is/are administered to an individual in need thereof. Thus, in other instances, one or more nucleic acids of the present disclosure, e.g., one or more recombinant expression vectors of the present disclosure encoding a T-Cell-MMP, is/are administered to an individual in need thereof.

### IX. Formulations

Suitable formulations are described above, where suitable formulations include a pharmaceutically acceptable excipient. In some cases, a suitable formulation comprises: a) a T-Cell-MMP and/or T-Cell-MMP-epitope conjugate (e.g., comprising a peptide presenting a cancer-associated epitope, an infectious disease-associated epitope, or a self-epitope) of the present disclosure; and b) a pharmaceutically acceptable excipient. In some cases, a suitable formulation comprises: a) a nucleic acid comprising a nucleotide sequence encoding a T-Cell-MMP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a T-Cell-MMP of the present disclosure; b) a second nucleic acid comprising a nucleotide sequence encoding the second polypeptide of a T-Cell-MMP of the present disclosure; and c) a pharmaceutically acceptable excipient. In some cases, a suitable formulation comprises: a) a recombinant expression vector comprising a nucleotide sequence encoding a T-Cell-MMP of the present disclosure; and b) a pharmaceutically acceptable excipient. In some cases, a suitable formulation comprises: a) a first recombinant expression vector comprising a nucleotide sequence encoding the first polypeptide of a T-Cell-MMP of the present disclosure; b) a second recombinant expression vector comprising a nucleotide sequence encoding the second polypeptide of a T-Cell-MMP of the present disclosure; and c) a pharmaceutically acceptable excipient.

Suitable pharmaceutically acceptable excipients are described above.

A. Dosages

A suitable dosage can be determined by an attending physician, or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, route of administration, general health, and other drugs being administered concurrently. A T-Cell-MMP and/or T-Cell-MMP-epitope conjugate of the present disclosure may be administered in amounts between 1 ng/kg body weight and 20 mg/kg body weight per dose, e.g., between 0.1 mg/kg body weight to 10 mg/kg body weight, e.g., between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A T-Cell-MMP and/or T-Cell-MMP-epitope conjugate of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a T-Cell-MMP and/or T-Cell-MMP-epitope conjugate of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a T-Cell-MMP and/or T-Cell-MMP-epitope conjugate of the present disclosure is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific T-Cell-MMP (or its epitope conjugate), the severity of the symptoms and the susceptibility of the subject to side effects. Preferred dosages for a given compound are readily determinable by those of skill in the art by a variety of means.

In some embodiments, multiple doses of a T-Cell-MMP and/or T-Cell-MMP-epitope conjugate of the present disclosure, a nucleic acid of the present disclosure, or a recombinant expression vector of the present disclosure are administered. The frequency of administration of a T-Cell-MMP and/or T-Cell-MMP-epitope conjugate of the present disclosure, a nucleic acid of the present disclosure, or a recombinant expression vector of the present disclosure can vary depending on any of a variety of factors, e.g., severity of the symptoms, etc. For example, in some embodiments, a T-Cell-MMP and/or T-Cell-MMP-epitope conjugate of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a T-Cell-MMP and/or T-Cell-MMP-epitope conjugate of the present disclosure, a nucleic acid of the present disclosure, or a recombinant expression vector of the present disclosure, e.g., the period of time over which a T-Cell-MMP and/or T-Cell-MMP-epitope conjugate of the present disclosure, a nucleic acid of the present disclosure, or a recombinant expression vector of the present disclosure is administered can vary, depending on any of a variety of factors, e.g., patient response, etc. For example, a T-Cell-MMP and/or T-Cell-MMP-epitope conjugate of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

B. Routes of Administration

An active agent (a T-Cell-MMP and/or T-Cell-MMP-epitope conjugate of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intralymphatic, intratracheal, intracranial, subcutaneous, intradermal, topical, intravenous, intraarterial, rectal, nasal, oral, and other enteral and parenteral routes of administration. Routes of administration may be combined, if desired, or adjusted depending upon the T-Cell-MMP and/or T-Cell-MMP-epitope conjugate and/or the desired effect. A T-Cell-MMP and/or T-Cell-MMP-epitope conjugate of the present disclosure, or a nucleic acid or recombinant expression vector of the present disclosure, can be administered in a single dose or in multiple doses.

In some embodiments, a T-Cell-MMP and/or T-Cell-MMP-epitope conjugate of the present disclosure, a nucleic acid of the present disclosure, or a recombinant expression vector of the present disclosure is administered intravenously. In some embodiments, a T-Cell-MMP and/or T-Cell-MMP-epitope conjugate of the present disclosure, a nucleic acid of the present disclosure, or a recombinant expression vector of the present disclosure is administered intramuscularly. In some embodiments, a T-Cell-MMP and/or T-Cell-MMP-epitope conjugate of the present disclosure, a nucleic acid of the present disclosure, or a recombinant expression vector of the present disclosure is administered intralymphatically. In some embodiments, a T-Cell-MMP and/or T-Cell-MMP-epitope conjugate, a nucleic acid of the present disclosure, or a recombinant expression vector of the present disclosure is administered locally. In some embodiments, a T-Cell-MMP and/or T-Cell-MMP-epitope conjugate of the present disclosure, a nucleic acid of the present disclosure, or a recombinant expression vector of the present disclosure is administered intratumorally. In some embodiments, a T-Cell-MMP and/or T-Cell-MMP-epitope conjugate of the present disclosure, a nucleic acid of the present disclosure, or a recombinant expression vector of the present disclosure is administered peritumorally. In some embodiments, a T-Cell-MMP and/or T-Cell-MMP-epitope conjugate of the present disclosure, a nucleic acid of the present disclosure, or a recombinant expression vector of the present disclosure is administered intracranially. In some embodiments, a T-Cell-MMP and/or T-Cell-MMP-epitope conjugate of the present disclosure, a nucleic acid of the present disclosure, or a recombinant expression vector of the present disclosure is administered subcutaneously.

In some embodiments, a T-Cell-MMP and/or T-Cell-MMP-epitope conjugate of the present disclosure is administered intravenously. In some embodiments, a T-Cell-MMP and/or T-Cell-MMP-epitope conjugate of the present disclosure is administered intramuscularly. In some embodiments, a T-Cell-MMP and/or T-Cell-MMP-epitope conjugate of the present disclosure is administered locally. In some embodiments, a T-Cell-MMP and/or T-Cell-MMP-epitope conjugate of the present disclosure is administered intratumorally. In some embodiments, a T-Cell-MMP and/or T-Cell-MMP-epitope conjugate of the present disclosure is administered peritumorally. In some embodiments, a T-Cell-MMP and/or T-Cell-MMP-epitope conjugate of the present disclosure is administered intracranially. In some embodiments, a T-Cell-MMP and/or T-Cell-MMP-epitope conjugate is administered subcutaneously. In some embodiments, a T-Cell-MMP and/or T-Cell-MMP-epitope conjugate is administered intralymphatically. In some embodiments, a T-Cell-MMP and/or T-Cell-MMP-epitope conjugate is administered intralymphatically.

A T-Cell-MMP and/or T-Cell-MMP-epitope conjugate of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried out to effect systemic or local delivery of a T-Cell-MMP and/or T-Cell-MMP-epitope conjugate of the present disclosure, a nucleic acid of the present disclosure, or a recombinant expression vector of the present disclosure. Where systemic delivery is desired, administration typically involves invasive or systemically absorbed topical or mucosal administration of pharmaceutical preparations.

C. Subjects Suitable for Treatment

Subjects suitable for treatment with a method or T-Cell-MMP-epitope conjugate of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment with a method of the present disclosure include individuals who have an infection (e.g., an infection with a pathogen such as a bacterium, a virus, a protozoan, etc.), including individuals who have been diagnosed as having an infection, and individuals who have been treated for an infection but who failed to respond to the treatment. Subjects suitable for treatment with a method of the present disclosure include individuals who have bacterial infection, including individuals who have been diagnosed as having a bacterial infection, and individuals who have been treated for a bacterial infection but who failed to respond to the treatment. Subjects suitable for treatment with a method of the present disclosure include individuals who have a viral infection, including individuals who have been diagnosed as having a viral infection, and individuals who have been treated for a viral infection but who failed to respond to the treatment. Subjects suitable for treatment with a method of the present disclosure include individuals who have an autoimmune disease, including individuals who have been diagnosed as having an autoimmune disease, and individuals who have been treated for an autoimmune disease but who failed to respond to the treatment.

### X. Certain Embodiments

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, and/or process step or steps, to the objective, spirit and scope of the present invention. All such modifications are intended to be within the scope of the claims appended hereto.

1. A T-Cell-MMP comprising:
a) a first polypeptide having an N-terminus and a C-terminus, the first polypeptide comprising,
   i) a first major histocompatibility complex (MHC) polypeptide having an N-terminus and a C-terminus, and an optional linker at its N-terminus or C-terminus;
b) a second polypeptide having an N-terminus and a C-terminus, the second polypeptide comprising (e.g., in order from N-terminus to C-terminus),
   i) a second MHC polypeptide;
   ii) optionally an immunoglobulin (Ig) Fc polypeptide or a non-Ig polypeptide scaffold, and
   iii) an optional linker at the N-terminus or the C-terminus of the second polypeptide;
c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide; and
d) one or more (e.g., two or more) immunomodulatory polypeptides (MODs), wherein at least one of the one or more MODs is
   A) at the C-terminus of the first polypeptide,
   B) at the N-terminus of the second polypeptide,
   C) at the C-terminus of the second polypeptide,
   D) at the C-terminus of the first polypeptide and at the N-terminus of the second polypeptide or
   E) within the first or second polypeptide;
      wherein each of the one or more MODs is an independently selected wild-type or variant MOD. The T-Cell-MMP of embodiment 1, which does not contain an epitope peptide as part of the translated sequence or chemically conjugated (covalently linked) to it, may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP of embodiment 1 may also be subject to the proviso that it does not include a T-Cell-MMP and/or T-Cell-MMP-epitope conjugate disclosed in International Appln. PCT/US2018/049803.
      2. A T-Cell-MMP comprising:
a) a first polypeptide having an N-terminus and a C-terminus, the first polypeptide comprising,
   i) a first major histocompatibility complex (MHC) polypeptide having an N-terminus and a C-terminus, and an optional linker at its N-terminus or C-terminus;
b) a second polypeptide having an N-terminus and a C-terminus, the second polypeptide comprising (e.g., in order from N-terminus to C-terminus),
   i) a second MHC polypeptide;
   ii) optionally an immunoglobulin (Ig) Fc polypeptide or a non-Ig polypeptide scaffold, and
   (iii) an optional linker at the N-terminus or the C-terminus of the second polypeptide;
c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide; and
d) one or more (e.g., two or more) immunomodulatory polypeptides (MODs), wherein at least one of the one or more MODs is
   A) at the C-terminus of the first polypeptide,
   B) at the N-terminus of the second polypeptide,
   C) at the C-terminus of the second polypeptide,
   D) at the C-terminus of the first polypeptide and at the N-terminus of the second polypeptide or
   E) within the first or second polypeptide;
wherein each of the one or more MODs is an independently selected wild-type or variant MOD; and
wherein the first or second polypeptide comprises an MHC-H polypeptide sequence having at least 85% (e.g., at least 90%, at least 95%, at least 97%, at least 98%, at least 99%, or 100%) sequence identity to at least a portion (e.g., 20-250, 20-100, 30-100, 40-120, 50-150, 70-170, 100-150, 100-200, 150-200, 200-250 contiguous aas, more than 250 contiguous aas, or all) of an MHC-H chain polypeptide selected from the group consisting of: HLA-A*0301 (SEQ ID NO:31), HLA-A*2407 (SEQ ID NO:33), HLA-A*3401 (SEQ ID NO:34), HLA-B*0801 (SEQ ID NO:37), HLA-B*1502 (SEQ ID NO:38), HLA-B*3802 (SEQ ID NO:39), HLA-B*4001 (SEQ ID NO:40), HLA-B*4601 (SEQ ID NO:41), HLA-B*5301 (SEQ ID NO:42), HLA-C*0102 (SEQ ID NO:44), HLA-C*0303 (SEQ ID NO:45), HLA-C*0304 (SEQ ID NO:46), HLA-C*0401 (SEQ ID NO:47), HLA-C*0602 (SEQ ID NO:48), HLA-C*0701 (SEQ ID NO:49), HLA-C*0702 (SEQ ID NO:50), HLA-C*0801 (SEQ ID NO:51), HLA-C*1502 (SEQ ID NO:52), an HLA-E polypeptide (SEQ ID NO: 54), an HLA-F polypeptide (SEQ ID NO: 55), and an HLA-G polypeptide (SEQ ID NO:56).
   The T-Cell-MMP of embodiment 2, which does not contain an epitope peptide as part of the translated sequence or chemically conjugated (covalently linked) to it, may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP of embodiment 2, may also be subject to the proviso that it does not include a T-Cell-MMP and/or T-Cell-MMP-epitope conjugate disclosed in International Appln. PCT/US2018/049803.
   3. A T-Cell-MMP comprising:
a) a first polypeptide having an N-terminus and a C-terminus, the first polypeptide comprising,
   i) a first major histocompatibility complex (MHC) polypeptide having an N-terminus and a C-terminus, and an optional linker at its N-terminus or C-terminus;
b) a second polypeptide having an N-terminus and a C-terminus, the second polypeptide comprising (e.g., in order from N-terminus to C-terminus),
   i) a second MHC polypeptide;
   ii) optionally an immunoglobulin (Ig) Fc polypeptide or a non-Ig polypeptide scaffold, and
   (iii) an optional linker at the N-terminus or the C-terminus of the second polypeptide;
c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide; and
d) one or more (e.g., two or more) immunomodulatory polypeptides (MODs), wherein at least one of the one or more MODs is
   A) at the C-terminus of the first polypeptide,
   B) at the N-terminus of the second polypeptide,
   C) at the C-terminus of the second polypeptide,
   D) at the C-terminus of the first polypeptide and at the N-terminus of the second polypeptide or
   E) within the first or second polypeptide;
wherein each of the one or more MODs is an independently selected wild-type or variant MOD; and
   wherein the first or second polypeptide comprises an MHC-H polypeptide sequence having at least 85% (e.g., at least 90%, at least 95%, at least 97%, at least 98% at least 99% or a 100%) sequence identity to at least a portion (e.g., 20-250, 20-100, 30-100, 40-120, 50-150, 70-170, 100-150, 100-200, 150-200, 200-250 contiguous aas, more than 250 contiguous aas, or all) of an MHC-H chain polypeptide selected from the group consisting of: an HLA-A polypeptide of SEQ ID NO:35, an HLA-B polypeptide of SEQ ID NO: 43, an HLA-C polypeptide of SEQ ID NO 53, an HLA-E polypeptide of SEQ ID NO: 54, an HLA-F polypeptide of SEQ ID NO: 55, and an HLA-G polypeptide of SEQ ID NO:56. The T-Cell-MMP of embodiment 3, which does not contain an epitope peptide as part of the translated sequence or chemically conjugated (covalently linked) to it, may be subject to the proviso that neither the first nor the second polypeptide comprises an MHC-H polypeptide explicitly disclosed (e.g., as a sequence) in International Appln. PCT/US2018/049803, which published as WO 2019/051127.
   The T-Cell-MMP of embodiment 3 may also be subject to the proviso that it does not include a T-Cell-MMP and/or T-Cell-MMP-epitope conjugate disclosed in International Appln. PCT/US2018/049803.
   4. The T-Cell-MMP of any of embodiments 1-3, wherein the first polypeptide comprises:
a first MHC polypeptide without a linker (e.g., a polypeptide linker) on its N-terminus and C-terminus,
a first MHC polypeptide bearing a linker (e.g., a polypeptide linker) on its N-terminus,
a first MHC polypeptide bearing a linker (e.g., a polypeptide linker) on its C-terminus, or
a first MHC polypeptide bearing a linker (e.g., a polypeptide linker) on its N-terminus and C-terminus.
   5. The T-Cell-MMP of any one of embodiments 1 to 4, wherein at least one of the one or more first polypeptide chemical conjugation sites is:
a) attached to (e.g., at the N- or C-terminus), or within, the sequence of the first MHC polypeptide, where the first MHC polypeptide is without a linker on its N- and C-termini;
b) attached to (e.g., at the N- or C-terminus), or within, the sequence of the first MHC polypeptide where the first MHC polypeptide comprises a linker on its N- and C-termini;
c) attached to (e.g., at the N- or C-terminus) or within, the sequence of the linker on the N-terminus of the first MHC polypeptide; and/or
d) attached to (e.g., at the N- or C-terminus) or within, the sequence of the linker on the C-terminus of the first MHC polypeptide.
   6. The T-Cell-MMP of any one of embodiments 1 to 5, wherein the first and second MHC polypeptides are Class I MHC polypeptides, and the first MHC polypeptide comprises:
a beta-2-microglobulin (“β2M”) polypeptide (see, e.g., FIG. 4) having an N-terminus and a C-terminus with or without a linker on its N- and/or C-termini,
a β2M polypeptide bearing a linker on its N-terminus,
a β2M polypeptide bearing a linker on its C-terminus, or
a β2M polypeptide bearing a linker on its N-terminus and C-terminus.
   7. The T-Cell-MMP of embodiment 6, wherein at least one of the one or more first polypeptide chemical conjugation sites is:
a) attached to (e.g., at the N- or C-terminus) or within the sequence of the β2M polypeptide without a linker on its N- or C-terminus;
b) attached to (e.g., at the N- or C-terminus) or within the sequence of the β2M polypeptide where the β2M polypeptide comprises a linker on its N- and C-termini;
c) attached to (e.g., at the N- or C-terminus) or within the sequence of the linker on the N-terminus of the β2M polypeptide; and/or
d) attached to (e.g., at the N- or C-terminus) or within the sequence of the linker on the C-terminus of the β2M polypeptide.
   8. The T-Cell-MMP of any one of embodiments 1 to 7, comprising:
a second MHC polypeptide without a linker on its N-terminus and C-terminus,
a second MHC polypeptide bearing a linker on its N-terminus,
a second MHC polypeptide bearing a linker on its C-terminus, or
a second MHC polypeptide bearing a linker on its N-terminus and C-terminus.
   9. The T-Cell-MMP of embodiment 8, wherein the second polypeptide further comprises an immunoglobulin (Ig) Fc polypeptide or a non-Ig polypeptide scaffold.
   10. The T-Cell-MMP of embodiment 9, wherein the second polypeptide comprises, in order from N-terminus to C-terminus:
a second MHC polypeptide bearing a linker on its C-terminus followed by an immunoglobulin (Ig) Fc polypeptide or a non-Ig polypeptide scaffold; or
a second MHC polypeptide bearing a linker on its N-terminus and/or C-terminus followed by an immunoglobulin (Ig) Fc polypeptide or a non-Ig polypeptide scaffold.
   11. The T-Cell-MMP of any one of embodiments 1 to 10, wherein at least one of the one or more second polypeptide chemical conjugation sites is:
a) attached to (e.g., at the N- or C-terminus) or within the sequence of the second MHC polypeptide, wherein the second MHC polypeptide is without a linker on its N- and C-termini;
b) attached to (e.g., at the N- or C-terminus) or within the sequence of the second MHC polypeptide, wherein the second MHC polypeptide comprises a linker on its N- and/or C-terminus;
c) attached to (e.g., at the N- or C-terminus) or within the sequence of the linker on the N-terminus of the second MHC polypeptide;
d) attached to (e.g., at the N- or C-terminus) or within the sequence of the linker on the C-terminus of the second MHC polypeptide; and/or
e) attached to (e.g., at the N- or C-terminus) or within the sequence of an immunoglobulin (Ig) Fc polypeptide or a non-Ig polypeptide scaffold when the second MHC polypeptide is followed by an immunoglobulin (Ig) Fc polypeptide or a non-Ig polypeptide scaffold.
   12. The T-Cell-MMP of any one of embodiments 1 to 11, wherein the second MHC polypeptide comprises: a MHC Class I heavy chain (“MHC-H” see, e.g., FIGS. 3A-3I) polypeptide having an N-terminus and a C-terminus without a linker on its N- and C-termini, a MHC-H polypeptide bearing a linker on its N-terminus, a MHC-H polypeptide bearing a linker on its C-terminus, or a MHC-H polypeptide bearing a linker on its N-terminus and C-terminus.
   13. The T-Cell-MMP of any one of embodiments 6-12, wherein at least one of the one or more first polypeptide chemical conjugation sites is:
a) attached to (e.g., at the N- or C-terminus), or within, the sequence of the β2M polypeptide without a linker on its N- or C-terminus;
b) attached to (e.g., at the N- or C-terminus), or within, the sequence of the β2M polypeptide where the β2M polypeptide comprises a linker on its N- and C-termini;
c) attached to (e.g., at the N- or C-terminus), or within, the sequence of the linker on the N-terminus of the β2M polypeptide; and/or
d) attached to (e.g., at the N- or C-terminus), or within, the sequence of the linker on the C-terminus of the β2M polypeptide.
   14. The T-Cell-MMP of any one of embodiments 6-12, wherein at least one of the one or more first polypeptide chemical conjugation sites replaces and/or is inserted between any of the amino terminal 15 amino acids of a mature β2M polypeptide sequence lacking its signal sequence (e.g., a β2M polypeptide sequence shown in FIG. 4).
   15. The T-Cell-MMP of any one of embodiments 1 to 14, wherein the second polypeptide comprises an Ig Fc polypeptide.
   16. The T-Cell-MMP of embodiment 15, wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide (e.g., an immunoglobulin sequence in any of FIGS. 2A to 2G).
   17. The T-Cell-MMP of embodiment 16, wherein the Ig Fc polypeptide comprises an amino acid sequence having at least 85% amino acid sequence identity (e.g., at least 90%, 95%, 98% or 99% identity, or even 100% identity) to an amino acid sequence depicted in one of FIGS. 2A-2D, or a portion of a sequence (at least about 50, 75, 100, 125 or 150 amino acids in length) in one of FIGS. 2A-2D corresponding to the IgFc polypeptide.
   18. The T-Cell-MMP of embodiment 17, wherein the IgFc polypeptide is an IgG1 Fc polypeptide.
   19. The T-Cell-MMP of embodiment 18, wherein the IgG1 Fc polypeptide comprises one or more amino acid substitutions selected from N297A, L234A, L235A, L234F, L235E, and P331S.
   20. The T-Cell-MMP of embodiment 19, wherein the IgG1 Fc polypeptide comprises L234A and L235A substitutions.
   21. The T-Cell-MMP of any one of embodiments 1 to 20, wherein at least one (e.g., at least two, or each) of the one or more MODs is a wild-type or variant MOD selected independently from the group consisting of CD7, CD80, CD86, PD-L1, PD-L2, 4-1BBL, OX40L, Fas ligand (FasL), ICOS-L, ICAM, TGF-β, CD30L, CD40, CD70, CD83, HVEM, lymphotoxin beta receptor, 3/TR6, ILT3, ILT4, HVEM, ILCD70, JAG1, and TGF-β.
   22. The T-Cell-MMP of any one of embodiments 1 to 21, wherein at least one (e.g., at least two) of the one or more MODs is a wild-type or variant MOD selected independently from the group consisting of: IL-2, 4-1BBL, PD-L1, CD80, and CD86.
   23. The T-Cell-MMP of any one of embodiments 1 to 21, wherein the T-Cell-MMP comprises one or more independently selected wild type or variant IL-2 MODs.
   24. The T-Cell-MMP of any one of embodiments 1 to 21, wherein the T-Cell-MMP comprises one or more independently selected wild type or variant 4-1BBL MODs.
   25. The T-Cell-MMP of any one of embodiments 1 to 21, wherein the T-Cell-MMP comprises one or more independently selected wild type or variant PD-L1 MODs.
   26. The T-Cell-MMP of any one of embodiments 1 to 21, wherein the T-Cell-MMP comprises one or more independently selected wild type or variant CD80 MODs.
   27. The T-Cell-MMP of any one of embodiments 1 to 21, wherein the T-Cell-MMP comprises one or more independently selected wild type or variant CD86 MODs.
   28. The T-Cell-MMP of any one of embodiments 1 to 27, wherein the T-Cell-MMP comprises one or more independently selected wild-type and/or variant MOD polypeptides; wherein at least one of the one or more variant MOD polypeptides exhibits a reduced affinity to a Co-MOD (its Co-MOD) compared to the affinity of a corresponding wild-type MOD for the Co-MOD (e.g., the ratio of i) the binding affinity of a control T-Cell-MMP (where the control comprises a wild-type MOD) to a Co-MOD to ii) the binding affinity of a T-Cell-MMP of the present disclosure comprising a variant of the wild-type MOD to the Co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 102:1, at least 5×102:1, at least 103:1, at least 5×103:1, at least 104:1, at least 105:1, or at least 106:1).
   29. The T-Cell-MMP of embodiment 28, wherein the variant MOD polypeptides comprise from 1, 2, 3, 4, 5, 6, 7, 8, 9, or 10 amino acid substitutions, insertions, or deletions relative to a corresponding wild-type immunomodulatory polypeptide, or comprises an amino acid sequence having at least 85% amino acid sequence identity (e.g., at least 90%, 95%, 98% or 99% identity, or even 100% identity) to an amino acid sequence of the corresponding wild-type MOD, or a portion of the sequence of a wild-type MOD (e.g., at least about 50, 75, 100, 125 or 150 contiguous amino acids of the wild-type MOD in length).
   30. The T-Cell-MMP of any one of embodiments 1 to 29, wherein the first MHC polypeptide comprises a β2M polypeptide; and wherein the second MHC polypeptide comprises a MHC Class I heavy chain polypeptide.
   31. The T-Cell-MMP of any one of embodiments 6 to 30, wherein the β2M polypeptide comprises an amino acid sequence having at least 85% amino acid sequence identity (e.g., at least 90%, 95%, 98% or 99% identity, or even 100% identity) to one of the amino acid sequences set forth in FIG. 4, or at least 60 contiguous amino acids of a mature sequence β2M polypeptide in FIG. 4 (e.g., at least about 60, 70, 80, or 90 amino acids).
   32. The T-Cell-MMP of any one of embodiments 6 to 31, wherein the β2M polypeptide comprises, consists essentially of, or consists of a sequence of at least 20, 30, 40, 50, 60, 70, 80, 90 or 99 contiguous amino acids having identity with at least a portion of one of the amino acid sequences set forth in FIG. 4 (e.g., a sequence having 20-99, 20-40, 30-50, 40-60, 40-90, 50-70, 60 to 80, 60-99, 70-90, or 79-99 contiguous amino acids with identity to a sequence of mature β2M polypeptide lacking its signal sequence set forth in FIG. 4).
   33. The T-Cell-MMP of any one of embodiments 12 to 32, wherein the MHC Class I heavy chain polypeptide comprises all or part of a HLA-A, HLA-B, HLA-C, HLA-E, HLA-F or HLA-G heavy chain (e.g., from FIGS. 3A to 3I).
   34. The T-Cell-MMP of embodiment 33, wherein the MHC Class I heavy chain polypeptide sequence comprises an amino acid sequence having at least 85% amino acid sequence identity (e.g., at least 90%, 95%, 98% or 99% identity, or even 100% identity) to all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of the amino acid sequences set forth in one of FIGS. 3D-3I,
   35. The T-Cell-MMP of embodiment 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-A polypeptide sequence having greater than 85%, 90%, 95%, or 98% sequence identity to an HLA-A allele sequence set forth in FIG. 3E.
   36. The T-Cell-MMP of embodiment 35, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-A polypeptide sequence having greater than 90%, 95%, 98% or 99% sequence identity to the HLA-A allele consensus sequence set forth in FIG. 3E (excluding positions X1 to X45 with defined amino acid variations in the consensus sequence).
   37. The T-Cell-MMP of embodiment 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-B polypeptide sequence having greater than 90%, 95%, 98% or 99% sequence identity to an HLA-B allele sequence set forth in FIG. 3F.
   38. The T-Cell-MMP of embodiment 37, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-B polypeptide sequence having greater than 90%, 95%, 98% or 99% sequence identity to the HLA-B allele consensus sequence set forth in FIG. 3F (excluding positions X1 to X34 with defined amino acid variations in the consensus sequence)
   39. The T-Cell-MMP of embodiment 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-C polypeptide sequence having greater than 85%, 90%, 95%, or 98% sequence identity to an HLA-C allele sequence set forth in FIG. 3G.
   40. The T-Cell-MMP of embodiment 39, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-C polypeptide sequence having greater than 90%, 95%, 98% or 99% sequence identity to the HLA-C allele consensus sequence set forth in FIG. 3G (excluding positions X1 to X37 with defined amino acid variations in the consensus sequence).
   41. The T-Cell-MMP of embodiment 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-E, F, or G polypeptide sequence having greater than 85%, 90%, 95%, or 98% sequence identity to an HLA-E, F, or G allele sequence set forth in FIG. 3H.
   42. The T-Cell-MMP of embodiment 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-E polypeptide sequence having greater than 90%, 95%, 98% or 99% sequence identity to an HLA-E allele consensus sequence set forth in FIG. 3H (excluding positions indicated by an “X” with defined amino acid variations in the consensus sequences).
   43. The T-Cell-MMP of embodiment 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-F polypeptide sequence having greater than 90%, 95%, 98% or 99% sequence identity to an HLA-F allele consensus sequence set forth in FIG. 3H (excluding positions indicated by an “X” with defined amino acid variations in the consensus sequences).
   44. The T-Cell-MMP of embodiment 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-G polypeptide sequence having greater than 90%, 95%, 98% or 99% sequence identity to an HLA-G allele consensus sequence set forth in FIG. 3H (excluding positions indicated by an “X” with defined amino acid variations in the consensus sequences).
   45. The T-Cell-MMP of any one of embodiments 12 to 44, wherein the MHC Class I heavy chain polypeptide sequence comprises a disulfide bond between a cysteine at the carboxyl end portion of the MHC heavy chain α1 helix and a cysteine in the amino end portion of the MHC heavy chain α2-1 helix, and/or a cysteine or a cysteine substitution at any one or more (two, three, four, etc.) of amino acid residues 2, 5, 7, 59, 84, 116, 139, 167, 168, 170 or 171.
   46. The T-Cell-MMP of embodiment 45, wherein the carboxyl end portion of the MHC heavy chain α1 helix is from about amino acid position 79 to about amino acid position 89 and the amino end portion of the MHC heavy chain α2-1 helix is from about amino acid position 134 to amino acid position 144 of the MHC Class I heavy chain, wherein the amino acid positions are determined based on the sequence of the heavy chains without their leader sequence (see, e.g., FIGS. 3D to 3I).
   47. The T-Cell-MMP of any one of embodiments 45 to 46, wherein the disulfide bond is between a cysteine located at positions 83, 84, or 85 and a cysteine located at position 138, 139 or 140 (e.g., from position 83 to position 138, 139 or 140, from position 84 to position 138, 139 or 140, or from position 85 to position 138, 139 or 140).
   48. The T-Cell-MMP of any one of embodiments 45 to 47, wherein the disulfide bond is between a cysteine located at positions 84 and a cysteine located at position 139.
   49. The T-Cell-MMP of any of embodiments 45 to 48, wherein the MHC Class I heavy chain sequence may have insertions, deletions and/or substitutions of 1 to 5 aas (e.g., 1, 2, 3 or 4 aas) preceding and/or following either or both cysteines forming the disulfide bond between the carboxyl end portion of the α1 helix and the amino end portion of the α2-1 helix.
   50. The T-Cell-MMP of embodiment 49, wherein, when substitutions and/or insertions are present, the amino acids may be selected from any naturally occurring amino acid, or any naturally occurring amino acid except glycine and/or proline.
   51. The T-Cell-MMP of any one of embodiments 33 to 50, wherein the MHC Class I heavy chain polypeptide amino acid sequence at positions 1 to 79 has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, 6, 7, 8, 9 or 10 amino acid insertions, deletions, or substitutions relative to a sequence in FIGS. 3D to 3H).
   52. The T-Cell-MMP of any one of embodiments 33 to 50, wherein the MHC Class I heavy chain polypeptide amino acid sequence from position 89 to 134 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, or 6 amino acid insertions, deletions, or substitutions relative to a sequence in FIGS. 3D to 3H).
   53. The T-Cell-MMP of any one of embodiments 33 to 52, wherein the MHC Class I heavy chain polypeptide amino acid sequence from position 144 to 230 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, 6, 7, 8, 9, 10, 11, 12, or 13 amino acid insertions, deletions, or substitutions relative to a sequence in FIGS. 3D to 3H).
   54. The T-Cell-MMP of any one of embodiments 33 to 53, wherein the MHC Class I heavy chain polypeptide amino acid sequence from positions 242 to 274 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, or 4 amino acid insertions, deletions, or substitutions relative to a sequence in FIGS. 3D to 3H).
   55. The T-Cell-MMP of any one of embodiments 1 to 54, wherein the first polypeptide and the second polypeptide are non-covalently associated.
   56. The T-Cell-MMP of any one of embodiments 1 to 54, wherein the first polypeptide and the second polypeptide are covalently linked to one another.
   57. The T-Cell-MMP of embodiment 56, wherein the covalent linkage is via a disulfide bond.
   58. The T-Cell-MMP of any one of embodiments 1 to 57, comprising two or more, three or more, or four or more independently selected MODs.
   59. The T-Cell-MMP of any one of embodiments 1 to 58, comprising a peptide linker between any two or more, three or more, or four or more of the two or more (e.g., two, three or four) wild-type and/or variant MODs.
   60. The T-Cell-MMP of any one of embodiments 1 to 59, wherein the first polypeptide comprises a peptide linker between the first MHC polypeptide and at least one wild-type or variant MOD.
   61. The T-Cell-MMP of any one of embodiments 1 to 58, wherein the second polypeptide comprises a peptide linker between the second MHC polypeptide and at least one wild-type or variant MOD.
   62. The T-Cell-MMP of any one of embodiments 1 to 61, comprising at least one peptide linker in the first and/or second polypeptide chain, wherein the linker has a length of from 5 amino acids to 30 amino acids (e.g., 5-10, 10-20, or 20-30 amino acids).
   63. The T-Cell-MMP of embodiment 62, wherein the linker comprises a peptide of the formula AAAGG or GGGGS, which may be repeated from one to ten times (e.g., from 1 to 4, 3 to 6, or 4 to 8 times).
   64. The T-Cell-MMP of any one of embodiments 1 to 63, wherein the first and second chemical conjugation sites are independently selected from:
a) peptide sequences that act as enzymatic modification sequences (e.g., a sulfatase motif);
b) non-natural amino acids and/or selenocysteines;
c) engineered amino acid chemical conjugation sites;
d) carbohydrate or oligosaccharide moieties; and/or
e) IgG nucleotide binding sites.
   65. The T-Cell-MMP of any one of embodiments 1 to 64, wherein at least one of the one or more first and second chemical conjugation sites comprises an enzymatic modification sequence.
   66. The T-Cell-MMP of any of embodiments 1 to 65, wherein at least one of the one or more first or second chemical conjugation site is a sulfatase motif.
   67. The T-Cell-MMP of embodiment 66, wherein the sulfatase motif comprises the sequence X1Z1X2Z2X3Z3, wherein
Z1 is cysteine or serine;
Z2 is either a proline or alanine residue;
Z3 is a basic amino acid (arginine, lysine, or histidine, usually lysine), or an aliphatic amino acid (alanine, glycine, leucine, valine, isoleucine, or proline, usually A, G, L, V, or I);
X1 is present or absent and, when present, can be any amino acid, though usually an aliphatic amino acid, a sulfur-containing amino acid, or a polar, uncharged amino acid (i.e., other than an aromatic amino acid or a charged amino acid), usually L, M, V, S or T, more usually L, M, S or V, with the proviso that, when the sulfatase motif is at the N-terminus of the target polypeptide, X1 is present; and
X2 and X3 independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.
   68. The T-Cell-MMP of embodiment 67, comprising one or more fGly amino acid residues in the amino acid sequence of the first polypeptide or the second polypeptide.
   69. The T-Cell-MMP of any one of embodiments 1 to 68, wherein at least one of the one or more first or second chemical conjugation sites is a Sortase A enzyme site comprising the amino acid sequence LP(X5)TG, LP(X5)TA, or LPETGG positioned at the C-terminus of the first and/or second polypeptide and wherein X5 is any amino acid.
   70. The T-Cell-MMP of any one of embodiments 1 to 69, wherein at least one of the one or more first or second chemical conjugation sites is a Sortase A enzyme site comprising at least one oligoglycine (e.g., (G)_{2, 3, 4, or 5}) at the amino terminus of the first and/or second polypeptides, and/or at least one oligo alanine (e.g., (A)_{2, 3, 4, or 5}) at the amino terminus of the first and/or second polypeptides.
   71. The T-Cell-MMP of any one of embodiments 1 to 70, wherein at least one of the one or more first or second chemical conjugation sites is a transglutaminase site.
   72. The T-Cell-MMP of embodiment 71, wherein at least one of the one or more transglutaminase sites is selected from the group consisting of: LQG, LLQGG, LLQG, LSLSQG, and LLQLQG.
   73. The T-Cell-MMP of any one of embodiments 1 to 72, wherein at least one of the one or more first and second chemical conjugation sites comprises a selenocysteine or an amino acid sequence containing one or more independently selected non-natural amino acids.
   74. The T-Cell-MMP of embodiment 73, wherein at least one of the one or more non-natural amino acids is selected from the group consisting of para-acetylphenylalanine, para-azido phenylalanine and propynyl-tyrosine (e.g., a cysteine engineered into the first or second polypeptide).
   75. The T-Cell-MMP of any one of embodiments 1 to 74, wherein at least one of the one or more first and second chemical conjugation sites comprises an engineered amino acid site.
   76. The T-Cell-MMP of any one of embodiments 1 to 75, wherein at least one of the one or more first and second chemical conjugation sites comprises one or more sulfhydryl or amine groups (e.g., a cysteine substitution at any one or more (two, three, four, etc.) of amino acid residues 2, 5, 7, 59, 84, 116, 139, 167, 168, 170, or 171).
   77. The T-Cell-MMP of embodiment 76, wherein at least one of the one or more sulfhydryl or amine groups results from the presence of a lysine or cysteine in the first and or second polypeptide.
   78. The T-Cell-MMP of any one of embodiments 1 to 77, wherein at least one of the one or more first and second chemical conjugation sites comprises an independently selected carbohydrate, monosaccharide, disaccharide and/or oligosaccharide.
   79. The T-Cell-MMP of any one of embodiments 1 to 78, wherein at least one of the one or more first and second chemical conjugation sites comprises one or more IgG nucleotide antibody binding sites.
   80. The T-Cell-MMP of any of embodiments 1 to 79, wherein:
a) the first polypeptide comprises a β2M polypeptide sequence,
b) the second polypeptide comprises (e.g., in order from N-terminus to C-terminus),
   i) a MHC-H polypeptide and an immunoglobulin (Ig) Fc polypeptide;
c) one or more first polypeptide chemical conjugation sites within the β2M polypeptide or on a peptide linker are attached (located) at the N-terminal to the β2M polypeptide sequence; and
d) one or more MODs, wherein at least one of the one or more MODs is
   A) at the C-terminus of the first polypeptide,
   B) at the N-terminus of the second polypeptide,
   C) at the C-terminus of the second polypeptide,
   D) at the C-terminus of the first polypeptide and at the N-terminus of the second polypeptide and/or
   E) within the first or second polypeptide;
      wherein each of the one or more MODs is an independently selected wild-type or variant MOD; and wherein the first and second polypeptides are optionally joined by an interpeptide covalent bond. The T-Cell-MMP of embodiment 80, which does not contain an epitope peptide as part of the translated sequence or chemically conjugated (covalently linked) to it, may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP of embodiment 80 may also be subject to the proviso that it does not include a T-Cell-MMP and/or T-Cell-MMP-epitope conjugate disclosed in International Appln. PCT/US2018/049803.
      81. The T-Cell-MMP of embodiment 80, comprising at least one MOD at:
A) the C-terminus of the first polypeptide;
B) the N-terminus of the second polypeptide; and/or
C) the C-terminus of the second polypeptide.
   82. The T-Cell-MMP of embodiment 80,
wherein the first and second polypeptides are joined by a disulfide bond between the MHC-H polypeptide and the β2M polypeptide; or
wherein the first and second polypeptides are joined by a disulfide bond between the MHC-H polypeptide and a peptide linker attached at the N-terminal to the β2M polypeptide sequence.
   83. The T-Cell-MMP of any of embodiments 80 to 82, wherein the disulfide bond between the MHC-H polypeptide and the β2M polypeptide is between an aa of the MHC-H polypeptide at about position 236 (e.g., A236C) and an aa of the β2M polypeptide at about position 12 (e.g., R12C).
   84. The T-Cell-MMP of any of embodiments 80 to 83, wherein at least one chemical conjugation site comprises a cysteine engineered into the first or second polypeptide.
   85. The T-Cell-MMP of any of embodiments 80 to 84, wherein at least one chemical conjugation site is a cysteine engineered into the β2M polypeptide sequence at position 2, 44, 50, 77, 85, 88, 91 or 98 (e.g., a Q2C, E44C, E50C, E77C, V85V, S88C, K91C, or D98C substitution) in a mature β2M polypeptide.
   86. The T-Cell-MMP of any of embodiments 80 to 85, wherein the chemical conjugation site is a cysteine engineered into the β2M polypeptide sequence as a Q2C or E44C substitution of a mature β2M polypeptide (e.g., the polypeptide of SEQ ID NOs: 57-60, which lacks the signal sequence).
   87. The T-Cell-MMP of any of embodiments 80 to 86, wherein the MHC-H polypeptide comprises a cysteine at positions 84 and 139 (e.g., Y84C and A139C substitutions) that form an intrachain disulfide bond (e.g., stabilizing the protein for expression).
   88. A T-Cell-MMP-epitope conjugate comprising a T-Cell-MMP of any one of embodiments 1 to 79, having an epitope covalently bound to the first or second polypeptide at a chemical conjugation site. The epitope covalently bound in the T-Cell-MMP-epitope conjugate is not part of the first or second polypeptide of the T-Cell-MMP, but rather is added (conjugated) to the first or second polypeptide after those peptides have been prepared (e.g., by cellular expression through transcription/translation).
   89. The T-Cell-MMP-epitope conjugate of embodiment 88, wherein the epitope is conjugated to a T-Cell-MMP of embodiment 80 or 81.
   90. The T-Cell-MMP-epitope conjugate of embodiment 88, wherein the epitope is conjugated to a T-Cell-MMP of embodiment 82 or 83.
   91. The T-Cell-MMP-epitope conjugate of embodiment 88, wherein the epitope is conjugated to a T-Cell-MMP of embodiment 84.
   92. The T-Cell-MMP-epitope conjugate of embodiment 88, wherein the epitope is conjugated to a T-Cell-MMP of embodiment 85 or 86.
   93. The T-Cell-MMP-epitope conjugate of embodiment 88, wherein the epitope is conjugated to a T-Cell-MMP of embodiment 87.
   94. The T-Cell-MMP-epitope conjugate of embodiment 88, wherein the T-Cell-MMP-epitope conjugate comprises a structure selected from structures A, B, C, D, E, F, G, H, I, J, K, or L of FIG. 6, wherein the first polypeptide and the second polypeptide are each organized from N-terminus to C-terminus as in the selected structure, and the first and second polypeptides are optionally linked by a covalent bond (e.g., a disulfide bond).
   95. The T-Cell-MMP-epitope conjugate of any one of embodiments 88 to 94, wherein the epitope is indirectly covalently bound by a linker or spacer to the first or second peptide chemical conjugation site.
   96. The T-Cell-MMP-epitope conjugate of any one of embodiments 88 to 94, wherein the epitope is conjugated to a chemical conjugation site through a linker, selected from a peptide or non-peptide polymer.
   97. The T-Cell-MMP-epitope conjugate of embodiment 94, wherein the epitope is conjugated through a linker that comprises a peptide having a length of from 4 amino acids to 30 amino acids (e.g., 4-10, 10-20 or 20-30 amino acids), including, but not limited to, polypeptides comprising from 1-10 repeating units of: glycine (polyG); glycine-serine polymer repeating units (including, for example, (GS), (GSGGS), (GGGS), (GGSG), (GGSGG), (GSGSG), (GSGGG), (GGGSG), (GSSSG), and (GGGGS)n); glycine-alanine polymer repeating units such as (AAAGG); alanine-serine polymers; cysteine containing linkers (e.g., GCGASGGGGSGGGGS, GCGGSGGGGSGGGGSGGGGS, GCGGSGGGGSGGGGS, or GCGGS(G4S)n, where n is an integer of at least one (e.g., 1, 2, 3, 4, 5, 6, 7, 8, 9, or 10).
   98. The T-Cell-MMP-epitope conjugate of embodiment 97, wherein the epitope is conjugated through a linker that comprises a peptide of the formula (AAAGG) or (GGGGS), which may be repeated from 1 to 8 times (e.g., 1, 2, 3, 4, 5, 6, 7, or 8 time, or in a range selected from 1 to 4, 3 to 6, or 4 to 8 times).
   99. The T-Cell-MMP-epitope conjugate of any of embodiments 88 to 98, wherein at least one MOD is a variant MOD.
   100. The T-Cell-MMP-epitope conjugate of any one of embodiments 88 to 99, wherein:
(a) the T-Cell-MMP-epitope conjugate binds to a first T-cell with an affinity that is at least 25% higher (1.25 times higher) than the affinity with which the T-Cell-MMP binds to a second T-cell,
   wherein the first T-cell expresses on its surface a Co-MOD and a TCR that binds the epitope with an affinity of at least 10⁻⁷ M (e.g., 10⁻⁸ or 10⁻⁹ M), and
   wherein the second T-cell expresses on its surface the Co-MOD but does not express on its surface a TCR that binds the epitope with an affinity of at least 10⁻⁷ M (e.g., an affinity less than 10⁻⁷ M, such as 10⁻⁶ or 10⁻⁵ M); or
(b) wherein the T-Cell-MMP-epitope conjugate binds to a first T-cell with an affinity that is at least 10% (e.g., at least 15%, at least 20%, at least 25%, at least 30%, at least 40%, at least 50%, at least 60%, at least 70%, at least 80%, at least 90%), or at least 2-fold (e.g., at least 2.5-fold, at least 5-fold, at least 10-fold, at least 15-fold, at least 20-fold, at least 25-fold, at least 50-fold, at least 100-fold, or more than 100-fold) higher than the affinity to which it binds the second T-cell,
   wherein the first T-cell displays both i) a TCR specific for the epitope present in the T-Cell-MMP-epitope conjugate, and ii) a Co-MOD that binds to the MOD present in the T-Cell-MMP-epitope conjugate, and
wherein the second T-cell displays: i) a TCR specific for an epitope other than the epitope present in the T-Cell-MMP-epitope conjugate; and ii) a Co-MOD that binds to the MOD present in the T-Cell-MMP-epitope conjugate.
   101. The T-Cell-MMP-epitope conjugate of any one of embodiments 88 to 100, wherein the epitope is conjugated at a sortase, sulfatase, or transglutaminase site.
   102. The T-Cell-MMP-epitope conjugate of any one of embodiments 88 to 100, wherein the epitope is conjugated through a non-natural amino acid or selenocysteine.
   103. The T-Cell-MMP-epitope conjugate of any one of embodiments 92 to 100, wherein the epitope is conjugated through an engineered amino acid.
   104. The T-Cell-MMP-epitope conjugate of embodiment 103, wherein the epitope is conjugated through an engineered cysteine site.
   105. The T-Cell-MMP-epitope conjugate of any of embodiments 103 to 104, where the epitope is conjugated through an engineered cysteine by a maleimide group incorporated into the peptide or a linker attached to the peptide bearing a maleimide group.
   106. The T-Cell-MMP-epitope conjugate of embodiment 105, wherein the maleimide group is attached to the epitope through a linker comprising a peptide (e.g., a GGGGS sequence repeated from 1 to 8 times) and a maleimide group.
   107. The T-Cell-MMP-epitope conjugate of embodiment 106, wherein the epitope conjugated to the T-Cell-MMP has the structure, from N-terminus to C-terminus, (epitope)-(peptide linker)-(optional alkyl linker)-(maleimide) or (epitope)-(peptide linker)-(lysine)-(alkyl linker bound to the epsilon amino group of the lysine)-(maleimide).
   108. The T-Cell-MMP-epitope conjugate of any of embodiments 88 to 107, wherein the epitope is a peptide that is not post-translationally modified (e.g., it is not a glycopeptide, phosphopeptide, or lipopeptide).
   109. The T-Cell-MMP-epitope conjugate of any of embodiments 88 to 107, wherein the epitope is a peptide that has been post-translationally modified (e.g., it is a glycopeptide, phosphopeptide, or lipopeptide).
   110. The T-Cell-MMP-epitope conjugate of any one of embodiments 88 to 109, wherein the epitope is a peptide that comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12 (entitled “Epitopes”).
   111. The T-Cell-MMP-epitope conjugate of embodiment 88, wherein the epitope is a peptide that comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12 (entitled “Epitopes”).
   112. The T-Cell-MMP-epitope conjugate of embodiment 89 or 90, wherein the epitope is a peptide that comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12 (entitled “Epitopes”).
   113. The T-Cell-MMP-epitope conjugate of embodiment 91 or 92, wherein the epitope is a peptide that comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12 (entitled “Epitopes”).
   114. The T-Cell-MMP-epitope conjugate of embodiment 93, wherein the epitope is a peptide that comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12 (entitled “Epitopes”).
   115. The T-Cell-MMP-epitope conjugate of any one of embodiments 88 to 114, wherein the epitope is a cancer-associated antigen comprising from 4 to 25 aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 aas).
   116. The T-Cell-MMP-epitope conjugate of any one of embodiments 88 to 114, wherein the epitope is a cancer-associated antigen comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12.d.(i) (Epitopes present in a cancer-associated antigens”).
   117. The T-Cell-MMP-epitope conjugate of any one of embodiments 88 to 114, wherein the epitope is an epitope associated with an infectious disease agent (e.g., an epitope of the infectious disease agent) comprising from 4 to 25 aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 aas).
   118. The T-Cell-MMP-epitope conjugate of any one of embodiments 88 to 114, wherein the epitope is an epitope associated with an infectious disease agent (e.g., an epitope of the infectious disease agent) comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12.d(ii) (entitled “Epitopes associated with infectious disease agents”).
   119. The T-Cell-MMP-epitope conjugate of any one of embodiments 88 to 114, wherein the epitope is an epitope associated with an HBV comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 aas) of an HBV protein.
   120. The T-Cell-MMP-epitope conjugate of any one of embodiments 88 to 114, wherein the epitope is an epitope associated with HBV comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12.d(iii) (entitled “Epitopes associated with HBV”).
   121. The T-Cell-MMP-epitope conjugate of embodiment 119, wherein the epitope is an epitope associated with HBV selected from the group consisting of: FLPSDFFPSV; GLSRYVARLG; KLHLYSHPI; FLLSLGIHL; ALMPLYACI; SLYADSPSV; STLPETTVV; LIMPARFYPK; AIMPARFYPK; YVNVNMGLK; MQWNSTALHQALQDP; LLDPRVRGL; SILSKTGDPV; VLQAGFFLL; FLLTRILTI; FLGGTPVCL; LLCLIFLLV; LVLLDYQGML; LLDYQGMLPV; IPIPSSWAF; WLSLLVPFV; GLSPTVWLSV; SIVSPFIPLL; ILSPFLPLL; ATVELLSFLPSDFFPSV; LPSDFFPSV; CLTFGRETV; VLEYLVSFGV; EYLVSFGVW; ILSTLPETTV; STLPETTVVRR; NVSIPWTHK; KVGNFTGLY; GLYSSTVPV; TLWKAGILYK; TPARVTGGVF; LVVDFSQFSR; GLSRYVARL; SAICSVVRR; YMDDVVLGA; PLGFFPDH; QAFTFSPTYK; KYTSFPWLL; ILRGTSFVYV; HLSLRGLFV; VLHKRTLGL; GLSAMSTTDL; CLFKDWEEL; and VLGGCRHKL.
   122. The T-Cell-MMP-epitope conjugate of any one of embodiments 88-114, wherein the epitope is an epitope associated with an HPV comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 aas) of an HPV protein.
   123. The T-Cell-MMP-epitope conjugate of any one of embodiments 88-114, wherein the epitope is an epitope associated with HPV comprising from 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in section I.A.12.d(iv) (entitled “Epitopes associated with HPV”).
   124. The T-Cell-MMP-epitope conjugate of embodiment 122, wherein the epitope is an epitope associated with HPV selected from the group consisting of: KLPQLCTEL; LQTTIHDII; VYDFAFRDL; FAFRDLCIV; KFYSKISEY; ISEYRHYCY; TLHEYMLDL; YMLDLQPET; QAEPDRAHY; RAHYNIVTF; CDSTLRLCV; LCVQSTHVDI; LLMGTLGIV; TLGIVCPI; LLMGTLGIVCPI.
   125. The T-Cell-MMP-epitope conjugate of embodiment 122, wherein the epitope is an epitope associated with HPV selected from the group consisting of: VYDFAFRDL RAHYNIVTF and CDSTLRLCV; and the MHC-H polypeptide comprises an HLA-A*2401 polypeptide or a fragment thereof.
   126. A dimer of a T-Cell-MMP or a dimer of a T-Cell-MMP-epitope conjugate of any of embodiments 1-125.
   127. The dimer of a T-Cell-MMP or the dimer of a T-Cell-MMP-epitope conjugate of embodiment 126, wherein each of the T-Cell-MMPs and T-Cell-MMP-epitope conjugates to be dimerized comprises an immunoglobulin heavy chain through which the T-Cell-MMP or T-Cell-MMP-epitope conjugate dimerizes, and wherein the dimer is optionally stabilized by one or two disulfide bonds between the immunoglobulin heavy chains.
   128. The T-Cell-MMP or T-Cell-MMP-epitope conjugate of any one of embodiments 1 to 127 (e.g., embodiments 109-114), further comprising one or more independently selected payloads covalently bound to one or more first and/or second chemical conjugation sites either directly or indirectly through a spacer or linker, wherein the spacer or linker is optionally cleavable (e.g., in an endosome of a mammalian cell).
   129. The T-Cell-MMP or T-Cell-MMP-epitope conjugate of embodiment 128, wherein the payload comprises one or more independently selected biologically active agents or drugs, diagnostic agents or labels, nucleotide or nucleoside analogs, nucleic acids or synthetic nucleic acids, toxins, a liposomes (e.g., incorporating a drugs such as 5-fluorodeoxyuridine), nanoparticles, or a combination thereof.
   130. The T-Cell-MMP or T-Cell-MMP-epitope conjugate of embodiment 128, comprising a payload selected from one or more biologically active agents or drug selected independently from the group consisting of: therapeutic agents (e.g., drug or prodrug), chemotherapeutic agents, cytotoxic agents, antibiotics, antivirals, cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or an antigen binding fragment thereof, enzymes, proenzymes, hormones, or combinations thereof.
   131. The T-Cell-MMP or T-Cell-MMP-epitope conjugate of embodiment 128, comprising a payload selected from one or more diagnostic agents or labels, selected independently from the group consisting of: photodetectable labels (e.g., dyes, fluorescent labels, phosphorescent labels, luminescent labels), radiolabels, imaging agents, contrast agents, paramagnetic labels, ultrasound labels, and combinations thereof.
   132. The T-Cell-MMP or T-Cell-MMP-epitope conjugate of embodiment 128, comprising a payload selected from one or more nucleotides or nucleosides, nucleoside analogs, nucleic acids, or synthetic nucleic acids selected from the group consisting of: single or double stranded DNA, single or double stranded RNA, DNA/RNA hybrids, ribozymes, siRNA, antisense RNA, cDNA, spherical nucleic acids, and plasmids.
   133. The T-Cell-MMP or T-Cell-MMP-epitope conjugate of embodiment 128, comprising a payload selected from one or more liposomes and/or nanoparticles selected independently from the group consisting of: micelles, metal nanoparticles (e.g., gold nanoparticles), and non-metal nanoparticles, any or all of which may be conjugated to nucleic acids and or proteins.
   134. The T-Cell-MMP or T-Cell-MMP-epitope conjugate of embodiment 128, wherein the payload is conjugated via a linker having from 1 to 20, (e.g., 1-2, 2-4, 5-10 or 10-20) independently selected alpha, beta, delta, gamma amino acids, or a combination thereof; or wherein the linker is a peptide of the formula poly-glycine poly-alanine, a random poly glycine/alanine copolymer, or poly(GGGGS)n where n is 1, 2, 3, 4, 5, 6, 7, or 8.
   135. The T-Cell-MMP or T-Cell-MMP-epitope conjugate of embodiment 128, wherein the payload is attached to a chemical conjugation site by a spacer, wherein the spacer comprises two or more carbon atoms joined by a single or double bond, a disulfide bond, a carbon-oxygen bond, a carbon nitrogen bond, or a combination thereof.
   136. The T-Cell-MMP or T-Cell-MMP-epitope conjugate of embodiment 128, wherein the payload is attached to a chemical conjugation site by a spacer, wherein the spacer results from the action of a homofunctional (e.g., homobifunctional) crosslinker or a heterofunctional (e.g., heterobifunctional) crosslinker.
   137. The T-Cell-MMP or T-Cell-MMP-epitope conjugate of any one of embodiments 128 to 136, wherein the payload is bound by a linker and can be removed from the T-Cell-MMP or its epitope conjugate by cleavage of the linker or spacer within a human T-cell endosome, or by reduction with an excess of thiol reducing agent (e.g., dithiothreitol, DTT).
   138. A composition comprising a T-Cell-MMP of any of embodiments 1 to 87 or 128 to 137, or a T-Cell-MMP-epitope conjugate of any one of embodiments 88 to 137.
   139. A composition comprising:
a) the T-Cell-MMP-epitope conjugate of any one of embodiments 88 to 137; and
b) a pharmaceutically acceptable excipient.
   140. A method of delivering an immunomodulatory polypeptide (MOD) to a target T-cell (e.g., a regulatory T-cell or cytotoxic T-cell) in an epitope-selective or epitope-selective/specific manner in vitro, or to an individual in vivo, comprising:
contacting a T-Cell-MMP-epitope conjugate of any one of embodiments 88 to 137 (e.g., embodiments 109-114) with the target T-cell or a mixed population of T-cells comprising the target T-cell in vitro; or
administering the T-Cell-MMP-epitope conjugate of any one of embodiments 88 to 137 or a composition comprising the T-Cell-MMP-epitope conjugate of any one of embodiments 138 to 139 to the individual;
   wherein the target T-cells are specific for the epitope present in the T-Cell-MMP-epitope conjugate.
   141. A method of modulating the activity (e.g., activation or proliferation) of a target T-cell (e.g., a regulatory T-cell or cytotoxic T-cell) in an epitope-selective or epitope-selective/specific manner in vitro, or in an individual in vivo, comprising:
contacting a T-Cell-MMP-epitope conjugate of any one of embodiments 88 to 137 (e.g., embodiments 109-114) with the T-cell in vitro, or
administering the T-Cell-MMP-epitope conjugate of any one of embodiments 88 to 137 or a composition comprising the T-Cell-MMP-epitope conjugate of any one of embodiments 138 to 139 to the individual;
   wherein the target T-cells are specific for the epitope present in the T-Cell-MMP-epitope conjugate.
   142. The method of embodiment 141, wherein said modulating comprises increasing a cytotoxic T-cell response to a cancer cell.
   143. A method of treating a patient having a cancer, the method comprising administering to the patient an effective amount of a composition according to any one of embodiments 138 to 139.
   144. The method of embodiment 142, wherein the cancer is hepatocellular carcinoma, pancreatic cancer, stomach cancer, colorectal cancer, hepatoblastoma, or an ovarian yolk sac tumor.
   145. The method of embodiment 141, wherein said modulating comprises increasing a T-cell response to an infectious agent.
   146. A method of treating a patient having a viral, a fungal, and/or a bacterial infection, the method comprising administering to the patient an effective amount of composition according to any one of embodiments 138 to 139.
   147. The method of embodiment 146, wherein the infectious agent is HPV or HBV.
   148. The method of any one of embodiments 140 to 147, wherein the individual is a human.
   149. The method of any one of embodiments 140 to 148, wherein said administering is rectal, nasal, oral, and other enteral and/or parenteral routes of administration.
   150. The method of any one of embodiments 140 to 149, wherein said administering is intratumoral, peritumoral, intramuscular, intratracheal, intracranial, subcutaneous, intralymphatic, intradermal, topical, intravenous, and/or intraarterial.
   151. The T-Cell-MMP or T-Cell-MMP-epitope conjugate of any of embodiments 1 to 137, wherein the chemical conjugation site (e.g., the site to which the epitope is covalently bound to prepare the T-Cell-MMP-epitope conjugate) is not located in or immediately adjacent to (within one amino acid) an amino acid sequence having 100% amino acid identity to:
the Fc polypeptide sequence in FIGS. 2A-2G;
the MHC Class I heavy chain polypeptide sequences in FIGS. 3A-3H; or
the β-2 microglobulin polypeptide sequences in FIG. 4.
   152. The T-Cell-MMP or T-Cell-MMP-epitope conjugate of any of embodiments 1 to 137, wherein the chemical conjugation site (e.g., the site to which the epitope is covalently bound to prepare the T-Cell-MMP-epitope conjugate) is not located in or immediately adjacent to (within one amino acid) of a 10, 20, 30, 40, or 50 amino acid long sequence having 100% amino acid identity to any portion of any one of:
the Fc polypeptide sequence in FIGS. 2A-2G;
the MHC Class I heavy chain polypeptide sequences in FIGS. 3A-3H; or
the β-2 microglobulin polypeptide sequences in FIG. 4.
   153. The T-Cell-MMP or T-Cell-MMP-epitope conjugate of any of embodiments 1 to 137, wherein the chemical conjugation site (e.g., the site to which the epitope is covalently bound to prepare the T-Cell-MMP-epitope conjugate) is not an amino acid appearing in a 5, 6, 7, 8, 9, 10, 20, 30, 40, 50, 60, or 70 amino acid long sequence having 100% amino acid identity to any portion of any one of:
the Fc polypeptides in FIGS. 2A-2G;
the MHC Class I heavy chain polypeptides in FIGS. 3A-3H; or
the β-2 microglobulin polypeptide sequences in FIG. 4.
   154. The T-Cell-MMP or T-Cell-MMP-epitope conjugate of any of embodiments 1 to 137, wherein the chemical conjugation site (e.g., the site to which the epitope is covalently bound to prepare the T-Cell-MMP-epitope conjugate) is not a lysine, cysteine, serine, threonine, arginine, aspartic acid, glutamic acid, asparagine, or glutamine located in a 10, 20, 30, 40, 50, 60, or 70 amino acid long sequence having 100% amino acid identity to any portion of any one of:
the Fc polypeptide sequence in FIGS. 2A-2G;
the MHC Class I heavy chain polypeptide sequences in FIGS. 3A-3H; or
the β-2 microglobulin polypeptide sequences in FIG. 4.
   155. A method of modulating an immune response in an individual, the method comprising administering to the individual an effective amount of the T-cell MMP-epitope conjugate of any one of embodiments 1 to 137 or a composition of embodiments 138 to 139, wherein said administering induces an epitope-specific T cell response (e.g., a T cell response specific for the epitope present in the T-cell-MMP-epitope conjugate) and an epitope-non-specific T cell response, wherein the ratio of the epitope-specific T cell response to the epitope-non-specific T cell response is at least 2:1 (e.g., at least 5:1, at least 10:1, at least 25:1, at least 50:1, or at least 100:1).
   156. The method of any of embodiments 140 to 155, wherein the individual is a human.
   157. The method of any one of embodiments 155 to 156, wherein said modulating comprises increasing a cytotoxic T-cell response to an epitope-expressing cancer cell).
   158. The method of any one of embodiments 155 to 157, wherein said administering is intravenous, subcutaneous, intramuscular, systemic, intralymphatic, distal to a treatment site, local, or at or near a treatment site.
   159. The method of any one of embodiments 155 to 158, wherein the epitope-non-specific T-cell response is less than the epitope non-specific T-cell response that would be induced by a control T-cell MMP-epitope conjugate comprising a corresponding wild-type immunomodulatory polypeptide.
   160. A method of detecting, in a mixed population of T cells obtained from an individual, the presence of a target T cell that binds an epitope of interest, the method comprising: a) contacting in vitro the mixed population of T cells with T-cell MMP-epitope conjugate of any one of embodiments 88 to 137, wherein the T-Cell-MMP-epitope conjugate comprises the epitope of interest; and b) detecting activation and/or proliferation of T cells in response to said contacting, wherein activated and/or proliferated T cells indicates the presence of the target T-cell.
   161. A polypeptide construct comprising a mature β2M polypeptide sequence (lacking its signal sequence) having an N-terminus and a C-terminus and an optional linker; and
   a peptide epitope that comprises 4 or more amino acids (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, or 4-25 aa, 7-25 aa, 10-15 aa, 15-20 aa, or 20-25 aa). For example, the epitope can be an epitope peptide of 4 or more amino acids from a protein set forth in section I.A.12 of this disclosure.
   162. The polypeptide of embodiment 161, further comprising one or more chemical conjugation sites within (e.g., at Q2C, E44C, E50C, E77C, V85V, S88C, K91C, and/or D98C) or at the ends of the sequence of the mature β2M polypeptide, or covalently attached to the mature β2M polypeptide via the optional linker, wherein the epitope is covalently bound, directly or indirectly, to at least one of the one or more chemical conjugation sites.
   163. The polypeptide of embodiment 161 or 162, wherein the mature β2M polypeptide has a sequence with at least 85%, (e.g., at least 90%, 95%, 98% or 99% identity, or even 100%) amino acid sequence identity to the sequence of a mature β2M provided in FIG. 4.
   164. The polypeptide of any of embodiments 161 to 163, wherein the β2M polypeptide sequence comprises, consists essentially of, or consists of a sequence of at least 20, 30, 40, 50, 60, 70, 80, 90 or 99 contiguous amino acids having identity with at least a portion of one of the amino acid sequences set forth in FIG. 4 (e.g., a sequence having 20-99, 20-40, 30-50, 40-60, 40-90, 50-70, 60 to 80, 60-99, 70-90, or 79-99 contiguous amino acids with identity to a sequence of a mature β2M polypeptide lacking its signal sequence set forth in FIG. 4).
   165. The polypeptide of any one of embodiments 161 to 164, wherein the β2M polypeptide sequence comprises a cysteine at one, two or more of amino acid positions 12, 44, 50, 77, 88, 91 and/or 98, of the mature β2M polypeptide sequence.
   166. The polypeptide of embodiment 165, wherein the first 12 amino acids of the β2M polypeptide sequence are IQRTPKIQVYSC.
   167. The polypeptide of any one of embodiments 161 to 164, comprising a sulfatase motif with the sequence X1Z1X2Z2X3Z3; wherein
Z1 is cysteine or serine;
Z2 is either a proline or alanine residue;
Z3 is a basic amino acid (arginine, lysine, or histidine, usually lysine), or an aliphatic amino acid (alanine, glycine, leucine, valine, isoleucine, or proline, usually A, G, L, V, or I);
X1 is present or absent and, when present, can be any amino acid, though usually an aliphatic amino acid, a sulfur-containing amino acid, or a polar, uncharged amino acid (i.e., other than an aromatic amino acid or a charged amino acid), usually L, M, V, S or T, more usually L, M, S or V, with the proviso that, when the sulfatase motif is at the N-terminus of the target polypeptide, X1 is present; and
X2 and X3 independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.
   168. The polypeptide of embodiment 167, wherein the sulfatase motif is linked directly, or indirectly via a linker, to the N-terminus of the β2M polypeptide sequence.
   169. The polypeptide of any one of embodiments 167 to 168, comprising a sulfatase motif, or a sulfatase motif wherein serine or cysteine of the sulfatase motif has been converted to an fGly (formylglycine) residue.
   170. The polypeptide of embodiment 169, wherein the epitope is covalently bound to the polypeptide through a chemical reaction with the fGly residue (e.g., the reaction of a thiosemicarbazide, aminooxy, hydrazide, or hydrazino modified epitope polypeptide with the aldehyde of the fGly).
   171. The polypeptide of embodiment 170 wherein the epitope comprises a hydrazinyl indole group for reaction with the aldehyde of the fGly residue.
   172. The polypeptide of any of embodiments 161 to 171, further comprising a signal sequence, or a signal sequence and a linker, wherein the signal sequence is the amino terminal-most element of the polypeptide.
   173. The polypeptide of any one of embodiments 161 to 172, wherein the one or more linkers comprise, consist essentially of, or consist of an independently selected polypeptide.
   174. The polypeptide of any of embodiments 161 to 170, comprising one or more optional linkers selected independently from a peptide of the formula (AAAGG)n or (GGGGS)n, where n is from 1 to 8 (e.g., 1, 2, 3, 4, 5, 6, 7, or 8, or in a range selected from 1 to 4, 3 to 6, or 4 to 8).
   175. The polypeptide of embodiment 165, wherein the β2M polypeptide comprises a mature β2M polypeptide comprising a cysteine substitution selected from: Q2C, E44C, E50C, E77C, V85V, S88C, K91C, and D98C to which the epitope is bound.
   176. A composition comprising a polypeptide of any one of embodiments 161 to 175.
   177. A composition comprising a polypeptide of any one of embodiments 161 to 175 and a pharmaceutically acceptable carrier.
   178. A polypeptide construct comprising a mature MHC Class I heavy chain polypeptide sequence (lacking its signal sequence) and an optional linker; and
an epitope polypeptide that comprises 4 or more amino acids (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, or 4-25 aa, 7-25 aa, 10-15 aa, 15-20 aa, or 20-25 aa). For example, the epitope can be an epitope peptide of 4 or more contiguous amino acids from a protein set forth in section I.A.12 of this disclosure;
and optionally an immunoglobulin (Ig) Fc polypeptide or a non-Ig polypeptide scaffold.
   179. The polypeptide of embodiment 178, further comprising one or more chemical conjugation sites within or at the ends of the sequence of the mature MHC Class I heavy chain polypeptide, or covalently attached to the mature MHC Class I heavy chain polypeptide via the optional linker, wherein the peptide epitope is covalently bound, directly or indirectly, to at least one of the one or more chemical conjugation sites.
   180. The polypeptide of embodiment 178 or 179, wherein the MHC Class I heavy chain polypeptide has a sequence with at least 85% (e.g., at least 90%, 95%, 98%, 99%, or even 100%) amino acid sequence identity to any of the sequences provided in FIGS. 3D-3H; wherein identity between the MHC Class I heavy chain polypeptide and the corresponding sequences in FIGS. 3D-3H is determined without consideration of the (Ig) Fc polypeptide and any optional linker present.
   181. The polypeptide of any of embodiments 178 to 180, wherein the MHC Class I heavy chain polypeptide comprises, consists essentially of, or consists of a sequence of at least 20, 30, 40, 50, 60, 70, 80, 90 or 100 contiguous amino acids having identity with at least a portion of one of the amino acid sequence set forth in any of FIGS. 3D-3H (e.g., a sequence having 20-100, 20-40, 30-50, 40-60, 40-90, 50-70, 60-80, 60-90, 70-90, or 80-100 contiguous amino acids with identity to a MHC Class I heavy chain polypeptide sequence set forth in any of FIGS. 3D-3H).
   182. The polypeptide of embodiment 181, wherein the MHC Class I heavy chain polypeptide comprises one, two or three sequences selected from the group consisting of:
i) a sequence from about amino acid position 79 to about amino acid position 89;
ii) a sequence from about amino acid position 134 to about amino acid position 144; and
iii) a sequence from about amino acid position 231 to about amino acid position 241 of the MHC Class I heavy chain sequences set forth in any of FIGS. 3D-3H.
   183. The polypeptide of embodiment 182, wherein the MHC Class I heavy chain polypeptide comprises:
i) the sequence from about amino acid position 79 to about amino acid position 89; and
ii) the sequence from about amino acid position 134 to about amino acid position 144;
wherein one of positions 83, 84, or 85 has been substituted with cysteine that forms an intrachain disulfide bond with a cysteine substituted at one of positions 138, 139, or 140.
   184. The polypeptide of any of embodiments 182 to 183, wherein the polypeptide comprises a MHC Class I heavy chain polypeptide sequence from about amino acid position 231 to about amino acid position 241 of the MHC Class I heavy chain sequences set forth in any of FIGS. 3D-3H wherein one of positions 235, 236, or 237 has been substituted by a cysteine.
   185. The polypeptide of any one of embodiments 178 to 184, wherein any one or more of the linkers is selected independently from peptides of formula (AAAGG)n or (GGGGS)n, where n is from 1 to 8 (e.g., 1, 2, 3, 4, 5, 6, 7, or 8, or in a range selected from 1 to 4, 3 to 6, or 4 to 8).
   186. A composition comprising a polypeptide of any one of embodiments 178 to 185.
   187. A composition comprising a polypeptide of any one of embodiments 178 to 185 and a pharmaceutically acceptable carrier.
   The subject matter of this disclosure and any of embodiments 1-187 may be subject to the proviso that neither the T-Cell-MMPs nor their epitope conjugates comprise an MHC-H polypeptide explicitly disclosed (e.g., as a sequence) in International Appln. PCT/US2018/049803, which published as WO 2019/051127. The subject matter of this disclosure and any of embodiments 1-187 may be subject to the proviso that neither the T-Cell-MMPs nor their epitope conjugates include T-Cell-MMP and/or T-Cell-MM-epitope conjugate disclosed in International Appln. PCT/US2018/049803.

### XI. Examples

### Example 1

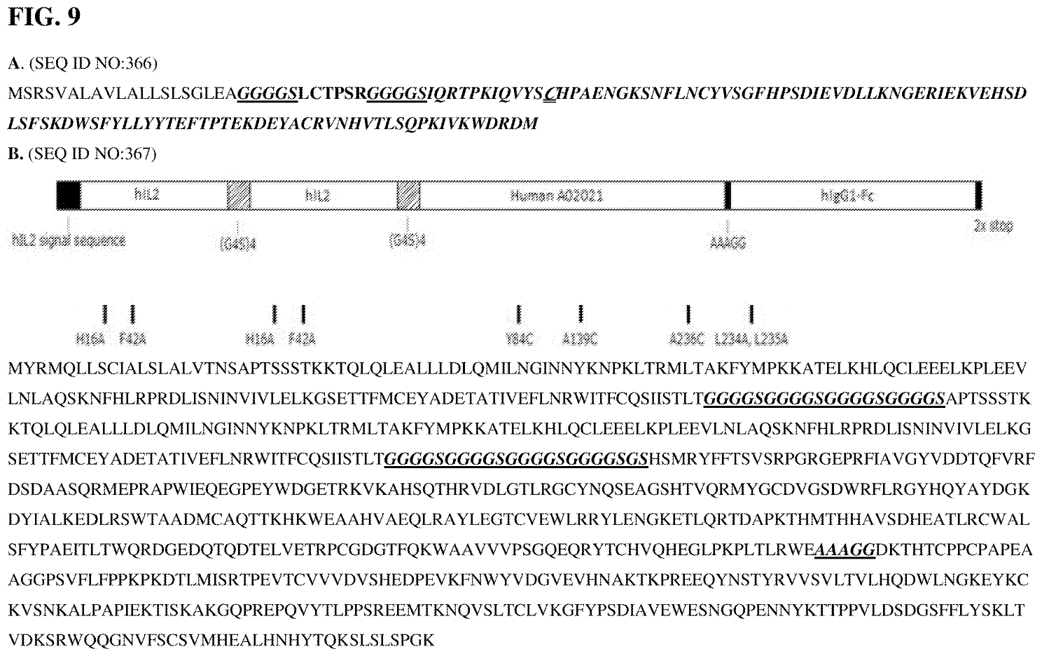

This example describes and provides for the preparation of a T-Cell-MMP having a first polypeptide (see FIG. 9 A) containing a sulfatase motif (bolded but not underlined) that can be acted on by an FGE to provide a fGly chemical conjugation site and a second polypeptide. The first and second polypeptides taken together form a T-Cell-MMP into which an epitope can be conjugated, and which can be dimerized through the IgFc regions. At B, FIG. 9 shows a second polypeptide of a T-Cell-MMP having tandem IL-2 MODs attached to the amino end of a human MHC Class I HLA-A heavy chain polypeptide followed by a human IgG1 Fc polypeptide.

The polypeptides are prepared by assembling the coding sequences of the first and second polypeptides in expression cassettes that include constitutive or inducible promoter elements for driving the expression of mRNA molecules encoding the first and second polypeptides along with polyadenylation and stop codons. The expression cassettes are assembled into separate vectors (plasmid, viral etc.), or a single vector, for transient expression from a suitable cell line (e.g., CHO, HEK, Vero, COS, yeast etc.). Alternatively, the assembled cassettes are stably integrated into such cells for constitutive or induced expression of the first and second polypeptides.

1A. First Polypeptides

The first polypeptide of this example comprises from the N-terminus to the C-terminus a) a leader sequence, b) a sulfatase motif to introduce an fGly chemical coupling site, c) an optional linker, and d) a β2M polypeptide. Following the action of a FGE, the first peptide has a cysteine in the motif converted to a formylglycine (fGly) residue.

Within the above-mentioned first peptide, the first 20 aas serve as the signal sequence and are removed during cellular processing during maturation of the polypeptide. The residues of the sulfatase motif (X1, Z1, X2, Z2, X3, and Z3), here LCTPSR, are described in Section I.A above flanked by the linker sequence GGGGS (SEQ ID NO:76) to emphasize that linkers may be placed before and/or after the motif. The map also indicates by double underlining the location of a potential amino acid substitution at position 12 in the β2M polypeptide changing an arginine to a cysteine (R12C).

1B. Second Polypeptides

The second polypeptide of this example comprises from N-terminus to C-terminus a) a leader sequence, b) a MOD polypeptide(s), c) an optional linker, d) a MHC Class 1 heavy chain polypeptide, e) an optional linker, and f) an immunoglobulin Fc region.

The mRNAs encode the second polypeptide polypeptides having the overall structure: signal sequence-linker-tandem IL-2 (IL2 polypeptide-optional linker-IL2 polypeptide)-linker-MHC Class 1 heavy chain polypeptide-linker-immunoglobulin heavy chain Fc polypeptide where the signal sequence is a 20 aa human IL2 signal sequence. The polypeptide also contains a human HLA-A polypeptide and a human IgG1 Fc polypeptide. Indicated below the map are the locations of potential amino acid substitutions including the location of the Y84C, A139C, and the A236C cysteine substitutions. The Y84C and A139C substitutions permit a stabilizing disulfide bond to form between the region near the carboxyl end of the HLA α1 helix and the region around the amino terminus of the HLA α2-1 helix. The cysteine resulting from the A236C substitution can form an interchain disulfide bond with a cysteine at, for example, position 12 of the β2M polypeptide in the first polypeptide. Below the map appears an exemplary peptide sequence for a second polypeptide including the leader sequence.

Additional polypeptides that could be used to prepare T-Cell-MMPs and their epitope conjugates are provided in FIG. 10. In addition, although exemplified principally with the A11 MHC Class I heavy chain HLA-A*1101, any of the other MHC heavy chains set forth in FIGS. 3A to 3D, or portions thereof, could have been employed, including, but not limited to, HLA-A*0201; HLA-A*2401; HLA-A*2402 (SEQ ID NO:136) and HLA-A*3303 (SEQ ID NO:137).

1C. Expression and Maturation of the First Second Polypeptides

As indicated above, first and second polypeptides are prepared by transient or stable expression in a suitable cell line (e.g., a eukaryotic or mammalian cell line). Processing in the cell removes the signal sequence and forms a fGly residue when the cells employed for polypeptide expression also express an FGE that is capable of converting a cysteine or serine of the sulfatase motif to a formylglycine (fGly) residue.

T-Cell-MMPs can be processed by cells as a complex that includes the first and second polypeptides and a bound (non-covalently associated) epitope or null polypeptide. The introduction of the disulfide bond in the HLA heavy chain polypeptide between the region at the carboxyl end of the α1 helix and the region at the amino terminus of the α2-1 helix permits expression in the absence of an epitope polypeptide associated with the first and second polypeptides. In addition, as the T-Cell-MMP complexes do not contain a membrane anchor region, the complex is released from the expressing cell in soluble form.

Cell culture media containing the expressed T-Cell-MMP is collected after suitable levels of the expressed T-Cell-MMP have been attained. Where the cells used for expression did not have FGE activity, the T-Cell-MMPs are treated with an FGE capable of forming the fGly residue at the sulfatase motif. Isolation and concentration of the T-Cell-MMP from the media (e.g., serum free media) is conducted using, for example, chromatographic methods to produce a purified T-Cell-MMP having a fGly chemical conjugation site at or near the amino terminus of the first polypeptide of the complex. The resulting T-Cell-MMP has the general structure shown in FIG. 5, part B, where the MHC-1 in the first polypeptide is the β2M polypeptide, the second polypeptide “MOD” is the pair of IL2 polypeptides, the MHC-2 is a HLA-A polypeptide, and Fc is a IGg1 heavy chain constant region. The disulfide bond between the first and second polypeptides results from the cysteines arising from the β2M polypeptide R12C and HLA-A A236C substitutions.

1.D. Preparation of T-Cell-MMP-Epitope Conjugates

Epitope polypeptides are conjugated to the fGly-containing T-Cell-MMP prepared above by forming on the epitope peptide a group capable of reacting with the fGly aldehyde. While thiosemicarbazide, aminooxy, hydrazide, or hydrazino aldehyde reactive groups can be utilized, this example is illustrated by the use of a hydrazinyl group (e.g., attached to an indole) where the epitope peptide is covalently bound, directly or indirectly, to the nitrogen of the indole ring. Contacting the epitope peptide with the fGly containing polypeptide of the T-Cell-MMP results in the T-Cell-MMP and epitope becoming covalently linked, thereby forming the T-Cell-MMP-epitope conjugates.

1.E. Epitopes for T-Cell-MMP Conjugates

Non-limiting examples of epitopes that can be used to form T-Cell-MMP-epitope conjugates include those recited in section I.A.12, including the cancer-associated epitopes in Section I.A.12.d(i), the epitopes associated with infectious disease agents in I.A.12.d(ii), epitopes associated with HBV in Section I.A.12.d(iii), and Epitopes associated with HPV in Section I.A.12.d(iv).

### Example 2

Example 2 illustrates the ability to produce T-Cell-MMPs and conjugate them to a peptide resulting in a protein that is not aggregated (a dimer of T-cell-MMPs), displays suitable stability for use at 37° C., and can be purified. The example is conducted with a CMV peptide; however, other epitope peptides can equally be utilized to form epitope conjugates providing similar results. In the Example two immunomodulatory proteins were prepared by cellular expression in Expi-CHO cells using transient transfection using an expression vector containing a nucleic acid construct encoding the proteins. The proteins were purified over Protein A (MabSelect SuRe™; GE), followed by further purification by size exclusion chromatography.

The first immunomodulatory protein, having structure A set forth in FIG. 13 (generically termed an IL-2 Control Construct), is not a T-Cell-MMP or an epitope conjugate thereof. The protein acts as a control for the T-Cell-MMP of the present disclosure. That control protein comprises a first polypeptide having a 9 aa cytomegalovirus (CMV) epitope at the N-terminus of a β2M polypeptide sequence:

(SEQ ID NO: 362)
NLVPMVATV [symbol] [symbol] IQRTPKIQVYSCHPAENGKSNFLNCY
VSGFHPSDIEVDLLKNGERIEKVEHSDLSFSKDWSFYLLYYTEFTPTEKD
EYACRVNHVTLSQPKIVKWDRDM.

Linkers are shown in bold and italics.

The second polypeptide of the control protein has, from N-terminus to C-terminus: two copies of an IL-2 immunomodulatory sequence (with H16A F42A substitutions) in tandem; a linker; a HLA-A*0201 polypeptide (with Y84A and A236C substitutions); a linker; and a human IgG1-Fc polypeptide having a LALA (L234A/L235A, see, e.g., FIG. 2G) substitution:

(SEQ ID NO: 363)
APTSSSTKKTQLQLEALLLDLQMILNGINNYKNPKLTRMLTAKFYMPKKA
TELKHLQCLEEELKPLEEVLNLAQSKNFHLRPRDLISNINVIVLELKGSE
TTFMCEYADETATIVEFLNRWITFCQSIISTLT [symbol] [symbol] [symbol]
APTSSSTKKTQLQLEALLLDLQMILNGINNYKNPKLTRMLTAKFYM
PKKATELKHLQCLEEELKPLEEVLNLAQSKNFHLRPRDLISNINVIVLEL
KGSETTFMCEYADETATIVEFLNRWITFCQSIISTLT [symbol] [symbol] [symbol]
GSHSMRYFFTSVSRPGRGEPRFIAVGYVDDTQFVRFDS
DAASQRMEPRAPWIEQEGPEYWDGETRKVKAHSQTHRVDLGTLRGAYNQS
EAGSHTVQRMYGCDVGSDWRFLRGYHQYAYDGKDYIALKEDLRSWTAADM
AAQTTKHKWEAAHVAEQLRAYLEGTCVEWLRRYLENGKETLQRTDAPKTH
MTHHAVSDHEATLRCWALSFYPAEITLTWQRDGEDQTQDTELVETRPCGD
GTFQKWAAVVVPSGQEQRYTCHVQHEGLPKPLTLRWE [symbol] DKTHTCPP
CPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK.

Linkers are shown in bold and italics.

The second immunomodulatory protein, having structure B set forth in FIG. 13 is an IL-2 containing T-Cell-MMP of the present disclosure having tandem IL-2 polypeptide sequences. The T-Cell-MMP comprises a first polypeptide having a linker bearing cysteine at aa 44 of the mature β2M polypeptide (E44C substitution marked as C*) that acts as a chemical conjugation site located at the N-terminus of the β2M polypeptide sequence:

(SEQ ID NO: 364)
IQRTPKIQVYSCHPAENGKSNFLNCYVSGFHPSDIEVDLLKNG [symbol] *RIEK
VEHSDLSFSKDWSFYLLYYTEFTPTEKDEYACRVNHVTLSQPKIVKWDR
DM.

The second polypeptide of the T-Cell-MMP has, from N-terminus to C-terminus: two copies of an IL-2 immunomodulatory sequence (with H16A F42A substitutions) in tandem; a linker; a HLA-A*0201 polypeptide (with Y84C, A139C, and A236C substitutions); a linker; and a human IgG1-Fc polypeptide having a LALA (L234A/L235A, see, e.g., FIG. 2G) substitution:

SEQ ID NO: 365)
APTSSSTKKTQLQLEALLLDLQMILNGINNYKNPKLTRMLTAKFYMPKKA
TELKHLQCLEEELKPLEEVLNLAQSKNFHLRPRDLISNINVIVLELKGSE
TTFMCEYADETATIVEFLNRWITFCQSIISTLT [symbol] [symbol] [symbol]
APTSSSTKKTQLQLEALLLDLQMILNGINNYKNPKLTRMLTAKFYM
PKKATELKHLQCLEEELKPLEEVLNLAQSKNFHLRPRDLISNINVIVLEL
KGSETTFMCEYADETATIVEFLNRWITFCQSIISTLT [symbol] [symbol] [symbol]
GSHSMRYFFTSVSRPGRGEPRFIAVGYVDDTQFVRFDS
DAASQRMEPRAPWIEQEGPEYWDGETRKVKAHSQTHRVDLGTLRGCYNQS
EAGSHTVQRMYGCDVGSDWRFLRGYHQYAYDGKDYIALKEDLRSWTAADM
CAQTTKHKWEAAHVAEQLRAYLEGTCVEWLRRYLENGKETLQRTDAPKTH
MTHHAVSDHEATLRCWALSFYPAEITLTWQRDGEDQTQDTELVETRPCGD
GTFQKWAAVVVPSGQEQRYTCHVQHEGLPKPLTLRWE [symbol] DKTHTCPP
CPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK.

Linkers are shown in bold and italics. The Y84C and A139C substitutions are shown as forming a disulfide bond between the α1 and α2 segments (helices) of the Class I heavy chain.

As shown in FIG. 13 at C, the expressed and purified proteins were subject to reducing SDS PAGE gel electrophoresis with both the control and T-Cell-MMP providing a light chain (first polypeptide) and heavy chain (second polypeptide).

### Example 3

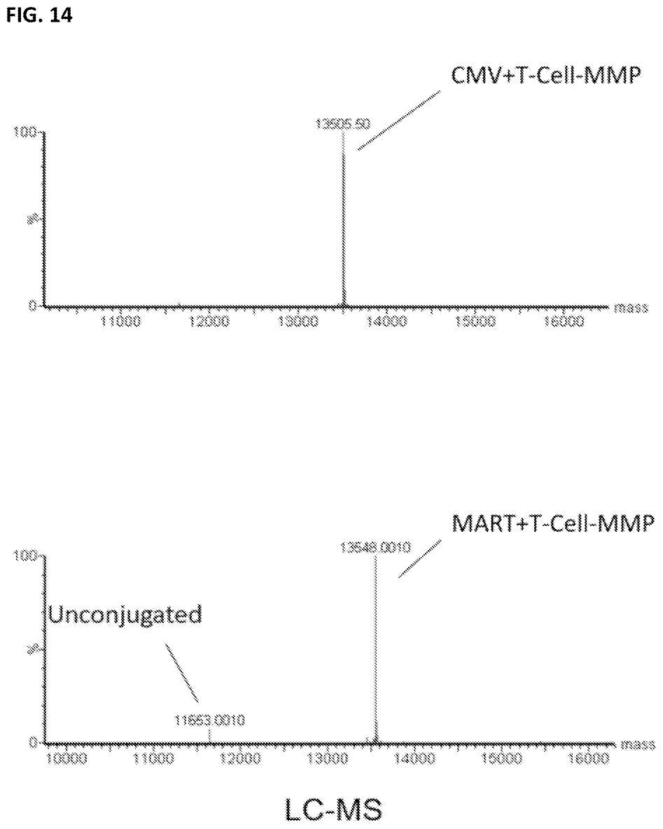

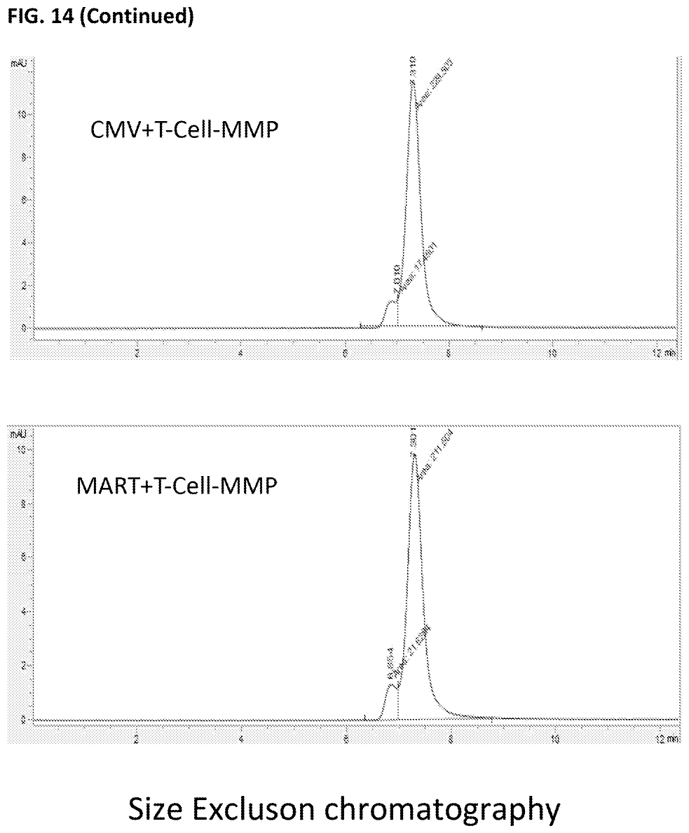

A T-Cell-MMP similar to Example 2 structure B was expressed and purified as described in Example 2. The purified T-Cell-MMP (labeled “IL-2 T-Cell-MMP”), which has an engineered cysteine residue as a chemical conjugation site, was conjugated to a CMV polypeptide (“CMV+T-Cell-MMP”) or a melanoma antigen MART-1 (“MART+T-Cell-MMP”) via a maleimide reactive linker attached to the peptide. The T-Cell-MMP-epitope conjugates were subjected to LCMS. The upper LCMS plot in FIG. 14 shows the IL-2 T-Cell-MMP+CMV light chain parent ion at 13,505.50 mass units with substantially complete conjugation. The lower LCMS plot in FIG. 14 shows the IL-2 T-Cell-MMP+MART light chain parent ion at 13,548.0010 mass units, with a minor amount of unconjugated T-Cell-MMP at 11,653.0010 mass units. Size exclusion chromatography indicates that at least 93% of the T-Cell-MMP conjugated to the CMV polypeptide and 91% of the T-Cell-MMP conjugated to the MART-1 polypeptide is in the form of an unaggregated dimer comprised of two first and two second polypeptides (see FIGS. 13 and 14 for reference).

### Example 4

Control constructs (see FIG. 12 structure A) comprising a first polypeptide having at the N-terminus of a β2M polypeptide sequence either a CMV epitope peptide as in Example 2 (“CMV+Cont-CON”) or a MART-1 epitope peptide (“MART+ContCON”) were prepared by cell expression and purification as described in Examples 2 and 3.

T-Cell-MMP-epitope-conjugates with tandem IL-2 MODs having a conjugated CMV peptide (“CMV+T-Cell-MMP”) or conjugated MART-1 peptide (“MART+T-Cell-MMP”) were prepared as in Example 3.

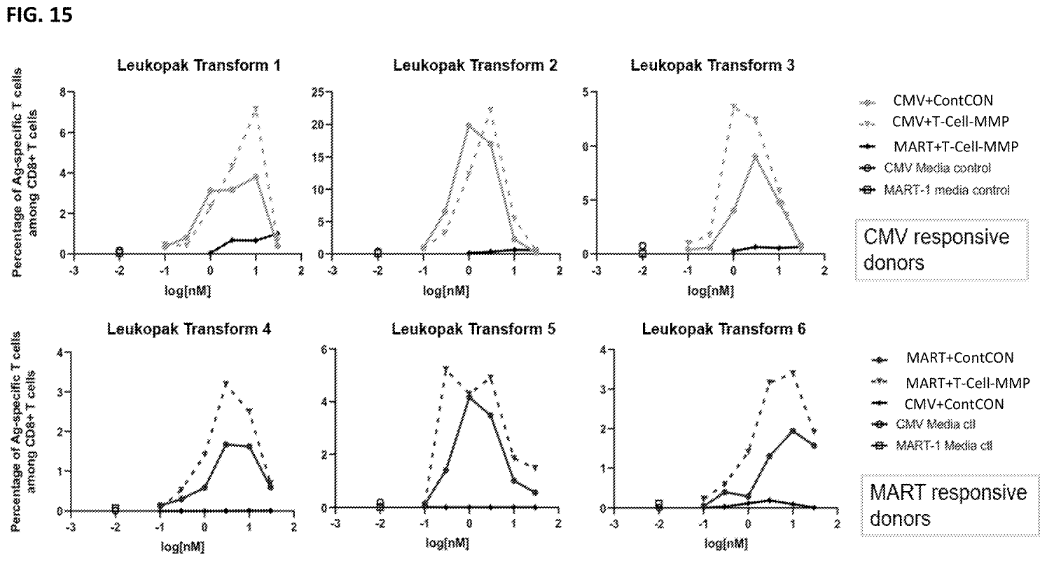

Ficoll-Paque® samples of leukocytes from three CMV responsive donors (Leukopak Transforms 1-3) and from three MART-1 (MART) responsive donors (Leukopak Transforms 4-6) were used. Responsiveness of the donor cells was determined based on the ability to expand CMV or MART-1 specific T-Cells upon CMV or MART-1 peptide stimulation in the presence of IL-2 as determined by flow cytometry. For the test shown in FIG. 15 the leukocytes were suspended at 2.5×10⁶ cells per ml in Immunocult media containing the indicated amounts of the control constructs or T-Cell-MMPs. As an additional control, cells grown without stimulation were stained with CMV or MART-1 tetramers purchased from MBL Internationl Corp. After 10 days in culture the number of cells responsive to CMV or MART-1 were assessed by Flowcytometry. The results indicate that both the CMV+ContCON (having a CMV epitope expressed as part of the protein) and CMV+IL-2 T-Cell-MMPs (having a conjugated CMV epitope peptide) stimulate the expansion of CMV responsive CD8+ T-Cells in CMV responsive donors in a concentration dependent manner. Similarly, both the MART-1 control construct (having a MART-1 epitope expressed as part of the protein) and MART-1 IL-2 T-Cell-MMPs (having a conjugated MART-1 epitope peptide) stimulate the expansion of MART-1 responsive CD8+ T-Cells in MART-1 responsive donors in a concentration dependent manner.

The antigen specificity in the responses are evidenced by the fact that CMV Control Construct and IL-2 T-Cell-MMP molecules did not stimulate expansion of MART-1 responsive CD8+ T-cells. Likewise, MART-1 Control Construct and IL-2 T-Cell-MMP molecules did not stimulate expansion of CMV responsive CD8+ T-cells. Accordingly, the presence of IL-2 polypeptide sequences present in each of the molecules were not responsible for nonspecific expansion of the leukocytes.

1. A T-cell modulatory multimeric polypeptide (T-Cell-MMP) comprising:
a) a first polypeptide having an N-terminus and a C-terminus, the first polypeptide comprising,
i) a first major histocompatibility complex (MHC) polypeptide having an N-terminus and a C-terminus, and an optional linker at its N-terminus or C-terminus;
b) a second polypeptide having an N-terminus and a C-terminus, the second polypeptide comprising,
i) a second MHC polypeptide;
ii) optionally an immunoglobulin (Ig) Fc polypeptide or a non-Ig polypeptide scaffold, and an optional linker at the N-terminus or the C-terminus of the second polypeptide;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
A) at the C-terminus of the first polypeptide,
B) at the N-terminus of the second polypeptide,
C) at the C-terminus of the second polypeptide,
D) at the C-terminus of the first polypeptide and at the N-terminus of the second polypeptide or
E) within the first or second polypeptide;
wherein the T-Cell-MMP does not contain an epitope peptide as part of its sequence or chemically conjugated (covalently linked) to it; and
wherein each of the one or more MODs is an independently selected wild-type or variant MOD.

2. A T-cell modulatory multimeric polypeptide (T-Cell-MMP) comprising:
a) a first polypeptide having an N-terminus and a C-terminus, the first polypeptide comprising,
i) a first major histocompatibility complex (MHC) polypeptide having an N-terminus and a C-terminus, and an optional linker at the N-terminus or the C-terminus;
b) a second polypeptide having an N-terminus and a C-terminus, the second polypeptide comprising,
i) a second MHC polypeptide;
ii) optionally an immunoglobulin (Ig) Fc polypeptide or a non-Ig polypeptide scaffold, and an optional linker at the N-terminus or the C-terminus of the second polypeptide;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
A) at the C-terminus of the first polypeptide,
B) at the N-terminus of the second polypeptide,
C) at the C-terminus of the second polypeptide,
D) at the C-terminus of the first polypeptide and at the N-terminus of the second polypeptide or
E) within the first or second polypeptide;
wherein the T-Cell-MMP does not contain an epitope peptide as part of its sequence or chemically conjugated (covalently linked) to it;
wherein each of the one or more MODs is an independently selected wild-type or variant MOD; and
wherein the first or second polypeptide comprises an MHC-H polypeptide sequence having at least 85% sequence identity to 200-250 aas of an MHC-H chain polypeptide selected from the group consisting of: HLA-A*0301 (SEQ ID NO:31), HLA-A*2407 (SEQ ID NO:33), HLA-A*3401 (SEQ ID NO:34), HLA-B*0801 (SEQ ID NO:37); HLA-B*1502 (SEQ ID NO:38), HLA-B*3802 (SEQ ID NO:39), HLA-B*4001 (SEQ ID NO:40), HLA-B*4601 (SEQ ID NO:41), HLA-B*5301 (SEQ ID NO:42), HLA-C*0102 (SEQ ID NO:44), HLA-C*0303 (SEQ ID NO:45), HLA-C*0304 (SEQ ID NO:46), HLA-C*0401 (SEQ ID NO:47), HLA-C*0602 (SEQ ID NO:48), HLA-C*0701 (SEQ ID NO:49), HLA-C*0702 (SEQ ID NO:50), HLA-C*0801 (SEQ ID NO:51), HLA-C*1502 (SEQ ID NO:52), an HLA-E polypeptide (SEQ ID NO: 54), an HLA-F polypeptide (SEQ ID NO: 55), and an HLA-G polypeptide (SEQ ID NO:56).

3. A T-cell modulatory multimeric polypeptide (T-Cell-MMP) comprising:
a) a first polypeptide having an N-terminus and a C-terminus, the first polypeptide comprising,
i) a first major histocompatibility complex (MHC) polypeptide having an N-terminus and a C-terminus, and an optional linker at the N-terminus or the C-terminus;
b) a second polypeptide having an N-terminus and a C-terminus, the second polypeptide comprising,
i) a second MHC polypeptide;
ii) optionally an immunoglobulin (Ig) Fc polypeptide or a non-Ig polypeptide scaffold, and an optional linker at the N-terminus or the C-terminus of the second polypeptide;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
A) at the C-terminus of the first polypeptide,
B) at the N-terminus of the second polypeptide,
C) at the C-terminus of the second polypeptide,
D) at the C-terminus of the first polypeptide and at the N-terminus of the second polypeptide or
E) within the first or second polypeptide;
wherein the T-Cell-MMP does not contain an epitope peptide as part of its sequence or chemically conjugated (covalently linked) to it;
wherein each of the one or more MODs is an independently selected wild-type or variant MOD; and
wherein the first or second polypeptide comprises an MHC-H polypeptide sequence having at least 85% sequence identity to 200-250 aas of an MHC-H chain polypeptide selected from the group consisting of: an HLA-A polypeptide of SEQ ID NO:35, an HLA-B polypeptide of SEQ ID NO: 43, an HLA-C polypeptide of SEQ ID NO 53, an HLA-E polypeptide of SEQ ID NO: 54, an HLA-F polypeptide of SEQ ID NO: 55, and an HLA-G polypeptide of SEQ ID NO:56.

4. A T-Cell-MMP-epitope conjugate comprising a T-Cell-MMP of claim 1, wherein the T-Cell-MMP is covalently bound, directly or indirectly through a peptide linker, to an epitope peptide through a covalent bond formed with one of the first polypeptide chemical conjugation site(s) or one of the second polypeptide chemical conjugation site(s) and wherein the epitope comprises four (4) or more amino acids.

5. The T-Cell-MMP-epitope conjugate of claim 4, wherein the first and second MHC polypeptides are Class I MHC polypeptides, and the first MHC polypeptide comprises:
a beta-2-microglobulin (“δ2M”) polypeptide having an N-terminus and a C-terminus without a linker on its N-terminus and C-terminus,
a β2M polypeptide bearing a linker on its N-terminus,
a β2M polypeptide bearing a linker on its C-terminus, or
a β2M polypeptide bearing a linker on its N-terminus and C-terminus.

6. The T-Cell-MMP-epitope conjugate of claim 5, wherein the second polypeptide comprises: a second MHC polypeptide comprising a MHC Class I heavy chain (“MHC-H”) polypeptide.

7. The T-Cell-MMP-epitope conjugate of claim 6, wherein the second polypeptide further comprises an immunoglobulin (Ig) Fc polypeptide or a non-Ig polypeptide scaffold.

8. The T-Cell-MMP-epitope conjugate of claim 7, wherein the T-Cell-MMP-epitope conjugate comprises one, two, or more independently selected wild-type and/or variant MOD polypeptides; wherein, if at least one variant MOD polypeptide is present, the variant MOD polypeptide exhibits a reduced affinity to a Co-MOD (its Co-MOD) compared to the affinity of a corresponding wild-type MOD for the Co-MOD.

9. The T-Cell-MMP-epitope conjugate of claim 8, wherein the wild-type MOD polypeptides are selected independently from the group consisting of IL-2, 4-1BBL, PD-L1, CD70, CD80, CD86, ICOS-L, OX-40L, FasL, JAG1, TGF-β, ICAM, and PD-L2, and the variant MOD polypeptides are variants thereof.

10. The T-Cell-MMP of claim 1, wherein the first and second chemical conjugation sites are independently selected from:
a) peptide sequences that act as enzymatic modification sequences;
b) non-natural amino acids and/or selenocysteines;
c) engineered amino acid chemical conjugation sites;
d) carbohydrate or oligosaccharide moieties; and/or
e) IgG nucleotide binding sites.

11. (canceled)

12. The T-Cell-MMP-epitope conjugate of claim 9, wherein the first or second chemical conjugation site to which the epitope is attached is a sulfhydryl group of a cysteine engineered into the β2M polypeptide or as an amino acid of a linker at the N-terminus of the α2M polypeptide.

13. The T-Cell-MMP-epitope conjugate of claim 12, wherein at least one chemical conjugation site to which the epitope is attached is a cysteine engineered into the β2M polypeptide sequence of the T-Cell-MMP-epitope conjugate as an aa substitution selected from Q2C, E44C, E50C, E77C, V85V, S88C, K91C, and/or D98C; and wherein the β2M polypeptide has a sequence with at least 85% sequence identity to at least 80 contiguous amino acids of a mature β2M polypeptide set forth in any of SEQ ID NOs: 57-61.

14. The T-Cell-MMP-epitope conjugate of claim 12, wherein the epitope is a peptide, glycopeptide, lipopeptide, or phosphopeptide.

15. The T-Cell-MMP-epitope conjugate of claim 14, wherein the MHC-H polypeptide comprises a polypeptide sequence having at least 85% sequence identity to 200-250 aas of an MHC-H chain polypeptide selected from the group consisting of: an HLA-A polypeptide of SEQ ID NO:35, an HLA-B polypeptide of SEQ ID NO:43, an HLA-C polypeptide of SEQ ID NO:53, an HLA-E polypeptide of SEQ ID NO:54, an HLA-F polypeptide of SEQ ID NO: 55, and an HLA-G polypeptide of SEQ ID NO:56, and
wherein the T-Cell-MMP comprises two copies of a variant IL-2 MOD, and wherein each variant IL-2 MOD has at least 95% sequence identity to SEQ ID NO:249, where X₁ is Ala or Thr, and X₂ is Ala.

16. The T-Cell-MMP-epitope conjugate of claim 15, wherein the epitope is an epitope present in a cancer associated antigen or an epitope associated with an infectious disease agent.

17. (canceled)

18. The T-Cell-MMP-epitope conjugate of claim 16, wherein the epitope is an HPV epitope or an HBV epitope.

19. (canceled)

20. The T-Cell-MMP-epitope conjugate of claim 14, wherein the epitope is conjugated via a linker peptide to the sulfhydryl of the cysteine engineered into the β2M polypeptide or the sulfhydryl of the cysteine present as an amino acid of a linker located at the N-terminus of the β2M polypeptide.

21. The T-Cell-MMP-epitope conjugate of claim 20, comprising a cysteine engineered into the β2M polypeptide sequence, wherein the linker peptide covalently bound to the sulfhydryl of a cysteine comprises a maleimide reacted with the sulfhydryl of the cysteine engineered into the β2M polypeptide, and wherein the β2M polypeptide has at least 85% sequence identity to at least 80 contiguous amino acids of a mature β2M polypeptide set forth in any of SEQ ID NOs: 57-61 as a Q2C, E44C, E50C, E77C, V85V, S88C, K91C, and/or D98C amino acid substitution.

22. (canceled)

23. A dimer comprising two T-Cell-MMP-epitope conjugates of claim 4, wherein in each T-Cell-MMP the second MHC polypeptide comprises an immunoglobulin (Ig) Fc polypeptide; and
wherein the dimer comprises one or more covalent bonds formed between the (Ig) Fc polypeptide of each T-Cell-MMP of the dimer.

24. A pharmaceutical composition comprising the dimer of claim 23.

25.-26. (canceled)

27. A method of treating a patient or individual, the method comprising administering to the patient or individual an effective amount of the pharmaceutical composition of claim 24.

28. The method of claim 27, wherein the epitope is an epitope present in a cancer associated antigen and the patient or individual is being treated for a cancer.

29. The method of claim 27, wherein the epitope is an epitope associated with an infectious disease agent and the patient or individual is being treated for an infectious disease.

30. The method of claim 29, wherein the patient or individual is being treated for an HPV or HBV infection.


SEQUENCE LISTING (382 sequences)

SEQ ID NO: 1 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 5 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 6 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 7 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 10 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 15 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 16 - protein, 365 aa, Homo sapiens [sequence omitted]

SEQ ID NO: 17 - protein, 365 aa, Homo sapiens [sequence omitted]

SEQ ID NO: 18 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 19 - protein, 365 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCK

SEQ ID NO: 20 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 21 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 22 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 23 - protein, 276 aa, Homo sapiens [sequence omitted]

SEQ ID NO: 24 - protein, 274 aa, Mouse
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 25 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 26 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 27 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 28 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 29 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 30 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 31 - protein, 276 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 32 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 33 - protein, 276 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 34 - protein, 276 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 35 - protein, 276 aa, Homo sapiens
  VARIANT (9)..(9): X1 is F, Y, S, or T
  VARIANT (44)..(44): X2 is K or R
  VARIANT (62)..(62): X3 is Q, G, E, or R
  VARIANT (63)..(63): X4 is N or E
  VARIANT (65)..(65): X5 is R or G
  VARIANT (66)..(66): X6 is N or K
  VARIANT (67)..(67): X7 is M or V
  VARIANT (70)..(70): X8 is H or Q
  VARIANT (73)..(73): X9 is T or I
  VARIANT (74)..(74): X10 is D or H
  VARIANT (76)..(76): X11 is A, V, or E
  VARIANT (77)..(77): X12 is N or D
  VARIANT (79)..(79): X13 is G or R
  VARIANT (80)..(80): X14 is T or I
  VARIANT (81)..(81): X15 is L or A
  VARIANT (82)..(82): X16 is R or L
  VARIANT (83)..(83): X17 is G or R
  VARIANT (90)..(90): X18 is A or D
  VARIANT (95)..(95): X19 is I, L, or V
  VARIANT (97)..(97): X20 is I, R or M
  VARIANT (99)..(99): X21 is F or Y
  VARIANT (105)..(105): X22 is S or P
  VARIANT (107)..(107): X23 is W or G
  VARIANT (114)..(114): X24 is R, H, or Q
  VARIANT (116)..(116): X25 is D or Y
  VARIANT (127)..(127): X26 is N or K
  VARIANT (142)..(142): X27 is T or I
  VARIANT (144)..(144): X28 is K or Q
  VARIANT (145)..(145): X29 is R or H
  VARIANT (149)..(149): X30 is A or T
  VARIANT (150)..(150): X31 is A or V
  VARIANT (151)..(151): X32 is H or R
  VARIANT (156)..(156): X33 is R, L, Q, or W
  VARIANT (158)..(158): X34 is V or A
  VARIANT (161)..(161): X35 is D or E
  VARIANT (163)..(163): X36 is R or T
  VARIANT (166)..(166): X37 is D or E
  VARIANT (167)..(167): X38 is W or G
  VARIANT (184)..(184): X39 is P or A
  VARIANT (193)..(193): X40 is P or A
  VARIANT (194)..(194): X41 is V or I
  VARIANT (207)..(207): X42 is S or G
  VARIANT (246)..(246): X43 is A or S
  VARIANT (253)..(253): X44 is Q or E
  VARIANT (276)..(276): X45 is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 36 - protein, 276 aa, Homo sapiens [sequence omitted]

SEQ ID NO: 37 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 276 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 39 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 40 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 41 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 42 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 43 - protein, 276 aa, Homo sapiens
  VARIANT (9)..(9): X1 is H, Y, or D
  VARIANT (11)..(11): X2 is A or S
  VARIANT (12)..(12): X3 is M or V
  VARIANT (24)..(24): X4 is A, S, or T
  VARIANT (32)..(32): X5 is Q or L
  VARIANT (41)..(41): X6 is A or T
  VARIANT (45)..(45): X7 is E, M K, or T
  VARIANT (46)..(46): X8 is A or T
  VARIANT (63)..(63): X9 is E or N
  VARIANT (66)..(66): X10 is I or K
  VARIANT (67)..(67): X11 is Y, F, S, or C
  VARIANT (70)..(70): X12 is N or Q
  VARIANT (71)..(71): X13 is A or T
  VARIANT (74)..(74): X14 is D or Y
  VARIANT (76)..(76): X15 is E or V
  VARIANT (77)..(77): X16 is S or N
  VARIANT (80)..(80): X17 is T, N, or I
  VARIANT (81)..(81): X18 is A or L
  VARIANT (82)..(82): X19 is L, or R
  VARIANT (83)..(83): X20 is R or G
  VARIANT (94)..(94): X21 is T or I
  VARIANT (95)..(95): X22 is L or I
  VARIANT (97)..(97): X23 is R or S
  VARIANT (131)..(131): X24 is R or S
  VARIANT (143)..(143): X25 is S or T
  VARIANT (147)..(147): X26 is L or W
  VARIANT (152)..(152): X27 is E OR V
  VARIANT (156)..(156): X28 is R, D, L or W
  VARIANT (158)..(158): X29 is A or T
  VARIANT (163)..(163): X30 is L, E or T
  VARIANT (177)..(177): X31 is E or D
  VARIANT (178)..(178): X32 is K or T
  VARIANT (180)..(180): X33 is E or Q
  VARIANT (194)..(194): X34 is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 44 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 45 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 46 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 47 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 48 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 49 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 50 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 51 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 52 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 53 - protein, 276 aa, Homo sapiens
  VARIANT (1)..(1): X1 is C or G
  VARIANT (6)..(6): X2 is R or K
  VARIANT (9)..(9): X3 is F, Y, S, or D
  VARIANT (14)..(14): X4 is R or W
  VARIANT (21)..(21): X5 is H or R
  VARIANT (24)..(24): X6 is A or S
  VARIANT (35)..(35): X7 is Q or R
  VARIANT (49)..(49): X8 is A or E
  VARIANT (66)..(66): X9 is N or K
  VARIANT (73)..(73): X10 is T or A
  VARIANT (77)..(77): X11 is S or N
  VARIANT (80)..(80): X12 is N or K
  VARIANT (90)..(90): X13 is A or D
  VARIANT (91)..(91): X14 is G or R
  VARIANT (94)..(94): X15 is T or I
  VARIANT (95)..(95): X16 is L or I
  VARIANT (97)..(97): X17 is W or R
  VARIANT (99)..(99): X18 is C, Y, F, or S
  VARIANT (103)..(103): X19 is L, or V
  VARIANT (113)..(113): X20 is Y or H
  VARIANT (114)..(114): X21 is D or N
  VARIANT (116)..(116): X22 is Y, F, S, or L
  VARIANT (147)..(147): X23 is L or W
  VARIANT (152)..(152): X24 is E, A, Or T
  VARIANT (156)..(156): X25 is R, L, or W
  VARIANT (163)..(163): X26 is L or T
  VARIANT (173)..(173): X27 is E OR K
  VARIANT (173)..(173): X27 is E or K
  VARIANT (177)..(177): X28 is E or K
  VARIANT (184)..(184): X29 is H or P
  VARIANT (194)..(194): X30 is R or V
  VARIANT (219)..(219): X31 is W or R
  VARIANT (248)..(248): X32 is V or M
  VARIANT (253)..(253): X33 is E or Q
  VARIANT (261)..(261): X34 is M or V
  VARIANT (267)..(267): X35 is P or Q
  VARIANT (273)..(273): X36 is R or S
  VARIANT (275)..(275): X37 is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 54 - protein, 284 aa, Homo sapiens
  VARIANT (89)..(89): X1= K or E
  VARIANT (107)..(107): X2= R or G
  VARIANT (157)..(157): X3= R or G
  VARIANT (158)..(158): X4= A or V
  VARIANT (255)..(255): X5= Q or P
  VARIANT (267)..(267): X6= P or S
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 55 - protein, 284 aa, Homo sapiens
  VARIANT (7)..(7): X1= Y or F
  VARIANT (50)..(50): X2= P or Q
  VARIANT (251)..(251): X3= S or P
  VARIANT (278)..(278): X4= P or L
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 56 - protein, 284 aa, Homo sapiens
  VARIANT (13)..(13): X1= S or F
  VARIANT (27)..(27): X2= Y or H
  VARIANT (31)..(31): X3= T, S, or M
  VARIANT (34)..(34): X4= L or V
  VARIANT (54)..(54): X5= Q or R
  VARIANT (81)..(81): X6= P or L
  VARIANT (100)..(100): X7= G or D
  VARIANT (104)..(104): X8= G or V
  VARIANT (105)..(105): X9= S or C
  VARIANT (110)..(110): X10= L or I
  VARIANT (159)..(159): X11= Y or H
  VARIANT (169)..(169): X12= H or R
  VARIANT (171)..(171): X13= Y or H
  VARIANT (178)..(178): X14= M or T
  VARIANT (185)..(185): X15= P or A
  VARIANT (219)..(219): X16= R, W, or Q
  VARIANT (258)..(258): X17= T or M
  VARIANT (275)..(275): X18= K or E
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 57 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 58 - protein, 119 aa, Pan troglodytes; identical to SEQ ID NO: 57

SEQ ID NO: 59 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 60 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 61 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 62 - protein, 6 aa, Artificial Sequence
  Sulfatase motifs
  VARIANT (1)..(1): Position 1 (X1) is present or absent and, when present, can be any amino acid, usually L, M, V, S or T, more usually L, M, S or V, with the proviso that, when the sulfatase motif is at the N-terminus of the target polypeptide, X1 is present.
  VARIANT (2)..(2): Position 2 (Z1) is cysteine or serine.
  VARIANT (3)..(3): Position 3 (X2) can be any amino acid, though usually an aliphatic aa, a polar, uncharged aa, or a sulfur containing amino acid (e.g., other than an aromatic aa or a charged aa), usually S, T, A, V, G or C, more usually S, T, A, V or G.
  VARIANT (4)..(4): Position 4 (Z2) is either a proline or alanine residue (which can also be represented by “P/A”).
  VARIANT (5)..(5): Position 5 (X3) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (6)..(6): Position 6 (Z3) is a basic amino acid (arginine, lysine, or histidine, usually lysine), or an aliphatic amino acid (alanine, glycine, leucine, valine, isoleucine, or proline, usually A, G, L, V, or I).
XXXXXX

SEQ ID NO: 63 - protein, 6 aa, Artificial Sequence
  Sulfatase motifs
  VARIANT (1)..(1): Position 1 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (3)..(3): Position 3 (X2) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (5)..(5): Position 5 (X3) can be any amino acid, though usually an aliphatic aa, a polar, uncharged amino acid, or a sulfur containing aa (e.g., other than an aromatic aa or a charged amino acid), usually S, T, A, V, G or C, more usually S, T, A, V or G.
  VARIANT (6)..(6): Position 6 (Z3) is a basic amino acid (arginine, lysine, or histidine, usually lysine), or an aliphatic aa (alanine, glycine, leucine, valine, isoleucine, or proline, usually A, G, L, V, or I).
XCXPXX

SEQ ID NO: 64 - protein, 5 aa, Artificial Sequence
  Sulfatase motif
  VARIANT (2)..(2): Position 2 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (4)..(4): Position 4 (X2) can be any aa, though usually an aliphatic aa, a polar, uncharged aa, or a sulfur containing aa (e.g., other than an aromatic aa or a charged aa), usually S, T, A, V, G or C, more usually S, T, A, V or G.
  VARIANT (5)..(5): Position 5 (Z3) is a basic amino acid (arginine, lysine, or histidine, usually lysine), or an aliphatic aa (alanine, glycine, leucine, valine, isoleucine, or proline, usually A, G, L, V, or I).
CXPXX

SEQ ID NO: 65 - protein, 5 aa, Artificial Sequence
  Sortase A Enzyme Site
  SITE (3)..(3): X5 can be any single amino acid.
  SITE (5)..(5): X can be a glycine or alanine.
LPXTX

SEQ ID NO: 66 - protein, 5 aa, Artificial Sequence
  Sortase enzyme conjugation partner sequence
GGGGG

SEQ ID NO: 67 - protein, 4 aa, Artificial Sequence
  Sortase enzyme conjugation partner sequence
GGGG

SEQ ID NO: 68 - protein, 5 aa, Artificial Sequence
  Sortase enzyme conjugation partner sequence
AAAAA

SEQ ID NO: 69 - protein, 4 aa, Artificial Sequence
  Sortase enzyme conjugation partner sequence
AAAA

SEQ ID NO: 70 - protein, 6 aa, Artificial Sequence
  Sortase A Enzyme Site
LPETGG

SEQ ID NO: 71 - protein, 6 aa, Artificial Sequence
  Sortase A Enzyme Site
LPETAA

SEQ ID NO: 72 - protein, 5 aa, Artificial Sequence
  Transglutaminase Enzyme Site
LLQGG

SEQ ID NO: 73 - protein, 4 aa, Artificial Sequence
  Transglutaminase Enzyme Site
LLQG

SEQ ID NO: 74 - protein, 6 aa, Artificial Sequence
  Transglutaminase Enzyme Site
LSLSQG

SEQ ID NO: 75 - protein, 6 aa, Artificial Sequence
  Transglutaminase Enzyme Site
LLQLQG

SEQ ID NO: 76 - protein, 276 aa, Homo sapiens
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 77 - protein, 99 aa, Homo sapiens
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 78 - protein, 276 aa, Homo sapiens
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
  VARIANT (231)..(235): Residues 231-235 each can be any amino acid or any amino acid other than glycine, in an embodiment it is the sequence VETRP
  DISULFID (236)..(236): Residue 236 can form a disulfide bond with beta-2M-containing polypeptide
  VARIANT (237)..(241): Residues 237-241 each can be any amino acid or any amino acid other than glycine, in an embodiment it is the sequence GDGTF
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELXXXXXCXXXXXQKWAAVVVPSGQEQRYTCHVQHEGLPKPLTLRWEP

SEQ ID NO: 79 - protein, 99 aa, Homo sapiens; identical to SEQ ID NO: 77

SEQ ID NO: 80 - protein, 5 aa, Artificial Sequence
  Scaffold polypeptide
  VARIANT (4)..(4): X is any amino acid other than proline.
VPGXG

SEQ ID NO: 81 - protein, 5 aa, Artificial Sequence
  Linker
GSGGS

SEQ ID NO: 82 - protein, 4 aa, Artificial Sequence
  Linker
GGGS

SEQ ID NO: 83 - protein, 4 aa, Artificial Sequence
  Linker
GGSG

SEQ ID NO: 84 - protein, 5 aa, Artificial Sequence
  Linker
GGSGG

SEQ ID NO: 85 - protein, 5 aa, Artificial Sequence
  Linker
GSGSG

SEQ ID NO: 86 - protein, 5 aa, Artificial Sequence
  Linker
GSGGG

SEQ ID NO: 87 - protein, 5 aa, Artificial Sequence
  Linker
GGGSG

SEQ ID NO: 88 - protein, 5 aa, Artificial Sequence
  Linker
GSSSG

SEQ ID NO: 89 - protein, 5 aa, Artificial Sequence
  Linker
GSSSS

SEQ ID NO: 90 - protein, 5 aa, Artificial Sequence
  Linker
GGGGS

SEQ ID NO: 91 - protein, 5 aa, Artificial Sequence
  Linker
AAAGG

SEQ ID NO: 92 - protein, 5 aa, Artificial Sequence
  Linker
GGGGS

SEQ ID NO: 93 - protein, 10 aa, Artificial Sequence
  Linker
GCGGSGGGGS

SEQ ID NO: 94 - protein, 15 aa, Artificial Sequence
  Linker
GCGASGGGGSGGGGS

SEQ ID NO: 95 - protein, 20 aa, Artificial Sequence
  Linker
GCGGSGGGGSGGGGSGGGGS

SEQ ID NO: 96 - protein, 15 aa, Artificial Sequence
  Linker
GCGGSGGGGSGGGGS

SEQ ID NO: 97 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 98 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 99 - protein, 124 aa, Homo sapiens
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 100 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 101 - protein, 208 aa, Homo sapiens [sequence omitted]

SEQ ID NO: 102 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 103 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 104 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 105 - protein, 208 aa, Homo sapiens
  VARIANT (19)..(19): X is any amino acid other than Asn.  In some cases, X is Ala.
VIHVTKEVKEVATLSCGHXVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 106 - protein, 208 aa, Homo sapiens
  VARIANT (63)..(63): X is any amino acid other than Asn.  In some cases, X is Ala.
VIHVTKEVKEVATLSCGHNVSVEELAQTRIYWQKEKKMVLTMMSGDMNIWPEYKNRTIFDITXNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 107 - protein, 208 aa, Homo sapiens
  VARIANT (67)..(67): X is any amino acid other than Ile.  In some cases, X is Ala.
VIHVTKEVKEVATLSCGHNVSVEELAQTRIYWQKEKKMVLTMMSGDMNIWPEYKNRTIFDITNNLSXVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 108 - protein, 208 aa, Homo sapiens
  VARIANT (86)..(86): X is any amino acid other than Lys.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLXYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 109 - protein, 208 aa, Homo sapiens
  VARIANT (157)..(157): X is any amino acid other than Gln.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSXDPETELYAVSSKLDFNMTTNHSFMCLIKYGHLRVNQTFNWNTTKQEHFPDN

SEQ ID NO: 110 - protein, 208 aa, Homo sapiens
  VARIANT (158)..(158): X is any amino acid other than Asp.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQXPETELYAVSSKLDFNMTTNHSFMCLIKYGHLRVNQTFNWNTTKQEHFPDN

SEQ ID NO: 111 - protein, 208 aa, Homo sapiens
  VARIANT (25)..(25): X is any amino acid other than Leu.  In some cases, X is Ala.
VIHVTKEVKEVATLSCGHNVSVEEXAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 112 - protein, 208 aa, Homo sapiens
  VARIANT (31)..(31): X is any amino acid other than Tyr.  In some cases, X is Ala.
VIHVTKEVKEVATLSCGHNVSVEELAQTRIXWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 113 - protein, 208 aa, Homo sapiens
  VARIANT (33)..(33): X is any amino acid other than Gln.  In some cases, X is Ala.
VIHVTKEVKEVATLSCGHNVSVEELAQTRIYWXKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 114 - protein, 208 aa, Homo sapiens
  VARIANT (38)..(38): X is any amino acid other than Met.  In some cases, X is Ala.
VIHVTKEVKEVATLSCGHNVSVEELAQTRIYWQKEKKXVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 115 - protein, 208 aa, Homo sapiens
  VARIANT (39)..(39): X is any amino acid other than Val.  In some cases, X is Ala.
VIHVTKEVKEVATLSCGHNVSVEELAQTRIYWQKEKKMXLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 116 - protein, 208 aa, Homo sapiens
  VARIANT (49)..(49): X is any amino acid other than Ile.  In some cases, X is Ala.
VIHVTKEVKEVATLSCGHNVSVEELAQTRIYWQKEKKMVLTMMSGDMNXWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 117 - protein, 208 aa, Homo sapiens
  VARIANT (53)..(53): X is any amino acid other than Tyr.  In some cases, X is Ala.
VIHVTKEVKEVATLSCGHNVSVEELAQTRIYWQKEKKMVLTMMSGDMNIWPEXKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 118 - protein, 208 aa, Homo sapiens
  VARIANT (60)..(60): X is any amino acid other than Asp.  In some cases, X is Ala.
VIHVTKEVKEVATLSCGHNVSVEELAQTRIYWQKEKKMVLTMMSGDMNIWPEYKNRTIFXITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 119 - protein, 208 aa, Homo sapiens
  VARIANT (108)..(108): X is any amino acid other than Phe.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADXPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 120 - protein, 208 aa, Homo sapiens
  VARIANT (156)..(156): X is any amino acid other than Ser.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVXQDPETELYAVSSKLDFNMTTNHSFMCLIKYGHLRVNQTFNWNTTKQEHFPDN

SEQ ID NO: 121 - protein, 208 aa, Homo sapiens
  VARIANT (111)..(111): X is any amino acid other than Pro.  In some cases, X is Ala.
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTXSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 122 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 123 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 124 - protein, 224 aa, Homo sapiens
  VARIANT (61)..(61): X is any amino acid other than Asn.  In some cases, X is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 125 - protein, 224 aa, Homo sapiens
  VARIANT (66)..(66): X is any amino acid other than Asp.  In some cases, X is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 126 - protein, 224 aa, Homo sapiens
  VARIANT (70)..(70): X is any amino acid other than Trp.  In some cases, X is Ala.
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 127 - protein, 224 aa, Homo sapiens
  VARIANT (91)..(91): X is any amino acid other than His.  In some cases, X is Ala.
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 128 - protein, 110 aa, Homo sapiens
  VARIANT (61)..(61): X is any amino acid other than Asn.  In some cases, X is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVL

SEQ ID NO: 129 - protein, 110 aa, Homo sapiens
  VARIANT (66)..(66): X is any amino acid other than Asp.  In some cases, X is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVL

SEQ ID NO: 130 - protein, 110 aa, Homo sapiens
  VARIANT (70)..(70): X is any amino acid other than Trp.  In some cases, X is Ala.
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVL

SEQ ID NO: 131 - protein, 110 aa, Homo sapiens
  VARIANT (91)..(91): X is any amino acid other than His.  In some cases, X is Ala.
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVL

SEQ ID NO: 132 - protein, 224 aa, Homo sapiens
  VARIANT (41)..(41): X is any amino acid other than Val.  In some cases, X is Ala.
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 133 - protein, 110 aa, Homo sapiens
  VARIANT (41)..(41): X is any amino acid other than Val.  In some cases, X is Ala.
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVL

SEQ ID NO: 134 - protein, 224 aa, Homo sapiens
  VARIANT (35)..(35): X is any amino acid other than Gln.  In some cases, X is Ala.
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 135 - protein, 110 aa, Homo sapiens
  VARIANT (35)..(35): X is any amino acid other than Gln.  In some cases, X is Ala.
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVL

SEQ ID NO: 136 - protein, 224 aa, Homo sapiens
  VARIANT (33)..(33): X is any amino acid other than Phe.  In some cases, X is Ala.
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 137 - protein, 110 aa, Homo sapiens
  VARIANT (33)..(33): X is any amino acid other than Phe.  In some cases, X is Ala.
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVL

SEQ ID NO: 138 - protein, 224 aa, Homo sapiens
  VARIANT (72)..(72): X is any amino acid other than Leu.  In some cases, X is Ala.
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 139 - protein, 110 aa, Homo sapiens
  VARIANT (72)..(72): X is any amino acid other than Leu.  In some cases, X is Ala.
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVL

SEQ ID NO: 140 - protein, 224 aa, Homo sapiens
  VARIANT (59)..(59): X is any amino acid other than Tyr.  In some cases, X is Ala.
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 141 - protein, 110 aa, Homo sapiens
  VARIANT (59)..(59): X is any amino acid other than Tyr.  In some cases, X is Ala.
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVL

SEQ ID NO: 142 - protein, 224 aa, Homo sapiens
  VARIANT (61)..(61): X is any amino acid other than Asn. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 143 - protein, 110 aa, Homo sapiens
  VARIANT (61)..(61): X is any amino acid other than Asn. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVL

SEQ ID NO: 144 - protein, 224 aa, Homo sapiens
  VARIANT (66)..(66): X is any amino acid other than Asp. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 145 - protein, 110 aa, Homo sapiens
  VARIANT (66)..(66): X is any amino acid other than Asn. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVL

SEQ ID NO: 146 - protein, 224 aa, Homo sapiens
  VARIANT (61)..(61): X is any amino acid other than Asn. In some cases, X is Ala.
  VARIANT (66)..(66): X is any amino acid other than Asp. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 147 - protein, 110 aa, Homo sapiens
  VARIANT (61)..(61): X is any amino acid other than Asn. In some cases, X is Ala.
  VARIANT (66)..(66): X is any amino acid other than Asp. In some cases, X is Ala.
  VARIANT (91)..(91): X is any amino acid other than His. In some cases, X is Ala.
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVL

SEQ ID NO: 148 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 149 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 150 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 151 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 152 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 153 - protein, 174 aa, Homo sapiens
  VARIANT (47)..(47): X is any amino acid other than Lys.  In some cases, X is Ala.
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 154 - protein, 174 aa, Homo sapiens
  VARIANT (147)..(147): X is any amino acid other than Gln.  In some cases, X is Ala.
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 155 - protein, 174 aa, Homo sapiens
  VARIANT (11)..(11): X is any amino acid other than Met.  In some cases, X is Ala.
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 156 - protein, 174 aa, Homo sapiens
  VARIANT (12)..(12): X is any amino acid other than Phe.  In some cases, X is Ala.
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 157 - protein, 174 aa, Homo sapiens
  VARIANT (14)..(14): X is any amino acid other than Gln.  In some cases, X is Ala.
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 158 - protein, 174 aa, Homo sapiens
  VARIANT (15)..(15): X is any amino acid other than Leu.  In some cases, X is Ala.
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 159 - protein, 174 aa, Homo sapiens
  VARIANT (16)..(16): X is any amino acid other than Val.  In some cases, X is Ala.
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 160 - protein, 174 aa, Homo sapiens
  VARIANT (18)..(18): X is any amino acid other than Gln.  In some cases, X is Ala.
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 161 - protein, 174 aa, Homo sapiens
  VARIANT (19)..(19): X is any amino acid other than Asn.  In some cases, X is Ala.
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 162 - protein, 174 aa, Homo sapiens
  VARIANT (20)..(20): X is any amino acid other than Val.  In some cases, X is Ala.
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 163 - protein, 174 aa, Homo sapiens
  VARIANT (21)..(21): X is any amino acid other than Leu.  In some cases, X is Ala.
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 164 - protein, 174 aa, Homo sapiens
  VARIANT (22)..(22): X is any amino acid other than Leu.  In some cases, X is Ala.
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 165 - protein, 174 aa, Homo sapiens
  VARIANT (23)..(23): X is any amino acid other than Ile.  In some cases, X is Ala.
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 166 - protein, 174 aa, Homo sapiens
  VARIANT (24)..(24): X is any amino acid other than Asp.  In some cases, X is Ala.
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 167 - protein, 174 aa, Homo sapiens
  VARIANT (25)..(25): X is any amino acid other than Gly.  In some cases, X is Ala.
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 168 - protein, 174 aa, Homo sapiens
  VARIANT (26)..(26): X is any amino acid other than Pro.  In some cases, X is Ala.
PAGLLDLRQGMFAQLVAQNVLLIGGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 169 - protein, 174 aa, Homo sapiens
  VARIANT (27)..(27): X is any amino acid other than Leu.  In some cases, X is Ala.
PAGLLDLRQGMFAQLVAQNVLLIGGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 170 - protein, 174 aa, Homo sapiens
  VARIANT (28)..(28): X is any amino acid other than Ser.  In some cases, X is Ala.
PAGLLDLRQGMFAQLVAQNVLLIGGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 171 - protein, 174 aa, Homo sapiens
  VARIANT (29)..(29): X is any amino acid other than Trp.  In some cases, X is Ala.
PAGLLDLRQGMFAQLVAQNVLLIGGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 172 - protein, 174 aa, Homo sapiens
  VARIANT (30)..(30): X is any amino acid other than Tyr.  In some cases, X is Ala.
PAGLLDLRQGMFAQLVAQNVLLIGGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 173 - protein, 174 aa, Homo sapiens
  VARIANT (31)..(31): X is any amino acid other than Ser.  In some cases, X is Ala.
PAGLLDLRQGMFAQLVAQNVLLIGGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 174 - protein, 174 aa, Homo sapiens
  VARIANT (32)..(32): X is any amino acid other than Asp.  In some cases, X is Ala.
PAGLLDLRQGMFAQLVAQNVLLIGGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 175 - protein, 174 aa, Homo sapiens
  VARIANT (33)..(33): X is any amino acid other than Pro.  In some cases, X is Ala.
PAGLLDLRQGMFAQLVAQNVLLIGGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 176 - protein, 174 aa, Homo sapiens
  VARIANT (34)..(34): X is any amino acid other than Gly.  In some cases, X is Ala.
PAGLLDLRQGMFAQLVAQNVLLIGGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 177 - protein, 174 aa, Homo sapiens
  VARIANT (35)..(35): X is any amino acid other than Leu.  In some cases, X is Ala.
PAGLLDLRQGMFAQLVAQNVLLIGGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 178 - protein, 174 aa, Homo sapiens
  VARIANT (37)..(37): X is any amino acid other than Gly.  In some cases, X is Ala.
PAGLLDLRQGMFAQLVAQNVLLIGGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 179 - protein, 174 aa, Homo sapiens
  VARIANT (38)..(38): X is any amino acid other than Val.  In some cases, X is Ala.
PAGLLDLRQGMFAQLVAQNVLLIGGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 180 - protein, 174 aa, Homo sapiens
  VARIANT (39)..(39): X is any amino acid other than Ser.  In some cases, X is Ala.
PAGLLDLRQGMFAQLVAQNVLLIGGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 181 - protein, 174 aa, Homo sapiens
  VARIANT (40)..(40): X is any amino acid other than Leu.  In some cases, X is Ala.
PAGLLDLRQGMFAQLVAQNVLLIGGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 182 - protein, 174 aa, Homo sapiens
  VARIANT (41)..(41): X is any amino acid other than Thr.  In some cases, X is Ala.
PAGLLDLRQGMFAQLVAQNVLLIGGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 183 - protein, 174 aa, Homo sapiens
  VARIANT (42)..(42): X is any amino acid other than Gly.  In some cases, X is Ala.
PAGLLDLRQGMFAQLVAQNVLLIGGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 184 - protein, 174 aa, Homo sapiens
  VARIANT (43)..(43): X is any amino acid other than Gly.  In some cases, X is Ala.
PAGLLDLRQGMFAQLVAQNVLLIGGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 185 - protein, 174 aa, Homo sapiens
  VARIANT (44)..(44): X is any amino acid other than Leu.  In some cases, X is Ala.
PAGLLDLRQGMFAQLVAQNVLLIGGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 186 - protein, 174 aa, Homo sapiens
  VARIANT (45)..(45): X is any amino acid other than Ser.  In some cases, X is Ala.
PAGLLDLRQGMFAQLVAQNVLLIGGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 187 - protein, 174 aa, Homo sapiens
  VARIANT (46)..(46): X is any amino acid other than Tyr.  In some cases, X is Ala.
PAGLLDLRQGMFAQLVAQNVLLIGGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 188 - protein, 174 aa, Homo sapiens
  VARIANT (48)..(48): X is any amino acid other than Glu.  In some cases, X is Ala.
PAGLLDLRQGMFAQLVAQNVLLIGGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 189 - protein, 174 aa, Homo sapiens
  VARIANT (49)..(49): X is any amino acid other than Asp.  In some cases, X is Ala.
PAGLLDLRQGMFAQLVAQNVLLIGGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 190 - protein, 174 aa, Homo sapiens
  VARIANT (50)..(50): X is any amino acid other than Thr.  In some cases, X is Ala.
PAGLLDLRQGMFAQLVAQNVLLIGGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 191 - protein, 174 aa, Homo sapiens
  VARIANT (51)..(51): X is any amino acid other than Lys.  In some cases, X is Ala.
PAGLLDLRQGMFAQLVAQNVLLIGGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 192 - protein, 174 aa, Homo sapiens
  VARIANT (52)..(52): X is any amino acid other than Glu.  In some cases, X is Ala.
PAGLLDLRQGMFAQLVAQNVLLIGGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 193 - protein, 174 aa, Homo sapiens
  VARIANT (64)..(64): X is any amino acid other than Phe.  In some cases, X is Ala.
PAGLLDLRQGMFAQLVAQNVLLIGGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 194 - protein, 174 aa, Homo sapiens
  VARIANT (65)..(65): X is any amino acid other than Phe.  In some cases, X is Ala.
PAGLLDLRQGMFAQLVAQNVLLIGGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 195 - protein, 174 aa, Homo sapiens
  VARIANT (66)..(66): X is any amino acid other than Gln.  In some cases, X is Ala.
PAGLLDLRQGMFAQLVAQNVLLIGGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 196 - protein, 174 aa, Homo sapiens
  VARIANT (67)..(67): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 197 - protein, 174 aa, Homo sapiens
  VARIANT (68)..(68): X is any amino acid other than Glu.  In some cases, X is Ala.
PAGLLDLRQGMFAQLVAQNVLLIGGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 198 - protein, 174 aa, Homo sapiens
  VARIANT (69)..(69): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 199 - protein, 174 aa, Homo sapiens
  VARIANT (70)..(70): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 200 - protein, 174 aa, Homo sapiens
  VARIANT (71)..(71): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 201 - protein, 174 aa, Homo sapiens
  VARIANT (72)..(72): X is any amino acid other than Val.  In some cases, X is Ala.
PAGLLDLRQGMFAQLVAQNVLLIGGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 202 - protein, 174 aa, Homo sapiens
  VARIANT (73)..(73): X is any amino acid other than Val.  In some cases, X is Ala.
PAGLLDLRQGMFAQLVAQNVLLIGGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 203 - protein, 174 aa, Homo sapiens
  VARIANT (75)..(75): X is any amino acid other than Gly.  In some cases, X is Ala.
PAGLLDLRQGMFAQLVAQNVLLIGGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 204 - protein, 174 aa, Homo sapiens
  VARIANT (76)..(76): X is any amino acid other than Glu.  In some cases, X is Ala.
PAGLLDLRQGMFAQLVAQNVLLIGGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 205 - protein, 174 aa, Homo sapiens
  VARIANT (77)..(77): X is any amino acid other than Gly.  In some cases, X is Ala.
PAGLLDLRQGMFAQLVAQNVLLIGGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 206 - protein, 174 aa, Homo sapiens
  VARIANT (78)..(78): X is any amino acid other than Ser.  In some cases, X is Ala.
PAGLLDLRQGMFAQLVAQNVLLIGGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 207 - protein, 174 aa, Homo sapiens
  VARIANT (104)..(104): X is any amino acid other than Asp.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 208 - protein, 174 aa, Homo sapiens
  VARIANT (105)..(105): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 209 - protein, 174 aa, Homo sapiens
  VARIANT (106)..(106): X is any amino acid other than Pro.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 210 - protein, 174 aa, Homo sapiens
  VARIANT (109)..(109): X is any amino acid other than Ser.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 211 - protein, 174 aa, Homo sapiens
  VARIANT (110)..(110): X is any amino acid other than Ser.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 212 - protein, 174 aa, Homo sapiens
  VARIANT (111)..(111): X is any amino acid other than Glu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 213 - protein, 174 aa, Homo sapiens
  VARIANT (113)..(113): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 214 - protein, 174 aa, Homo sapiens
  VARIANT (114)..(114): X is any amino acid other than Asn.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 215 - protein, 174 aa, Homo sapiens
  VARIANT (115)..(115): X is any amino acid other than Ser.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 216 - protein, 174 aa, Homo sapiens
  VARIANT (117)..(117): X is any amino acid other than Phe.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 217 - protein, 174 aa, Homo sapiens
  VARIANT (130)..(130): X is any amino acid other than Gln.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 218 - protein, 174 aa, Homo sapiens
  VARIANT (131)..(131): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 219 - protein, 174 aa, Homo sapiens
  VARIANT (132)..(132): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 220 - protein, 174 aa, Homo sapiens
  VARIANT (133)..(133): X is any amino acid other than Gly.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 221 - protein, 174 aa, Homo sapiens
  VARIANT (134)..(134): X is any amino acid other than Val.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 222 - protein, 174 aa, Homo sapiens
  VARIANT (135)..(135): X is any amino acid other than His.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 223 - protein, 174 aa, Homo sapiens
  VARIANT (136)..(136): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 224 - protein, 174 aa, Homo sapiens
  VARIANT (137)..(137): X is any amino acid other than His.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 225 - protein, 174 aa, Homo sapiens
  VARIANT (138)..(138): X is any amino acid other than Thr.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 226 - protein, 174 aa, Homo sapiens
  VARIANT (139)..(139): X is any amino acid other than Glu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 227 - protein, 174 aa, Homo sapiens
  VARIANT (141)..(141): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 228 - protein, 174 aa, Homo sapiens
  VARIANT (143)..(143): X is any amino acid other than Arg.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 229 - protein, 174 aa, Homo sapiens
  VARIANT (144)..(144): X is any amino acid other than His.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 230 - protein, 174 aa, Homo sapiens
  VARIANT (146)..(146): X is any amino acid other than Trp.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 231 - protein, 174 aa, Homo sapiens
  VARIANT (148)..(148): X is any amino acid other than Leu.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 232 - protein, 174 aa, Homo sapiens
  VARIANT (149)..(149): X is any amino acid other than Thr.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 233 - protein, 174 aa, Homo sapiens
  VARIANT (150)..(150): X is any amino acid other than Gln.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 234 - protein, 174 aa, Homo sapiens
  VARIANT (151)..(151): X is any amino acid other than Gly.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 235 - protein, 174 aa, Homo sapiens
  VARIANT (153)..(153): X is any amino acid other than Thr.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRSE

SEQ ID NO: 236 - protein, 174 aa, Homo sapiens
  VARIANT (154)..(154): X is any amino acid other than Val.  In some cases, X is Ala.
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 237 - protein, 133 aa, Homo sapiens [sequence omitted]

SEQ ID NO: 238 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 239 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 240 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 241 - protein, 133 aa, Homo sapiens
  VARIANT (42)..(42): X is any amino acid other than Phe.  In some cases, X is Ala, Met, Pro, Ser, Thr, Trp, Tyr, Val, or His.
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 242 - protein, 133 aa, Homo sapiens
  VARIANT (20)..(20): X is any amino acid other than Asp.  In some cases, X is Ala.
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 243 - protein, 133 aa, Homo sapiens
  VARIANT (15)..(15): X is any amino acid other than Glu.  In some cases, X is Ala.
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 244 - protein, 133 aa, Homo sapiens
  VARIANT (16)..(16): X is any amino acid other than His.  In some cases, X is Ala, Thr, Asn, Cys, Gln, Met, Val, or Trp.
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 245 - protein, 133 aa, Homo sapiens; identical to SEQ ID NO: 244
  VARIANT (16)..(16): X is any amino acid other than His.  In some cases, X is Ala, Asn, Arg, Asp, Cys, Glu, Gln, Gly, Ile, Lys, Leu, Met, Phe, Pro, Ser, Thr, Tyr, Trp, or Val.

SEQ ID NO: 246 - protein, 133 aa, Homo sapiens
  VARIANT (45)..(45): X is any amino acid other than Tyr.  In some cases, X is Ala.
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 247 - protein, 133 aa, Homo sapiens
  VARIANT (88)..(88): X is any amino acid other than Asn. In some cases, X is Ala or Arg.
APTSSSTKKTQLQLEHLLLDLQMILNGINNYKNPKLTRMLTFKFYMPKKATELKHLQCLEEELKPLEEVLNLAQSKNFHLRPRDLISXINVIVLELKGSETTFMCEYADETATIVEFLNRWITFCQSIISTLT

SEQ ID NO: 248 - protein, 133 aa, Homo sapiens
  VARIANT (126)..(126): X is any amino acid other than Gln.  In some cases, X is Ala.
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 249 - protein, 133 aa, Homo sapiens
  VARIANT (16)..(16): X is any amino acid other than His. In some cases, X is Ala or Thr.
  VARIANT (42)..(42): X is any amino acid other than Phe. In some cases, X is Ala.
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 250 - protein, 133 aa, Homo sapiens
  VARIANT (16)..(16): X is any amino acid other than His. In some cases, X is Ala or Thr.
  VARIANT (42)..(42): X is any amino acid other than Phe. In some cases, X is Ala or Thr.
APTSSSTKKTQLQLEXLLLDLQMILNGINNYKNPKLTRMLTXKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 251 - protein, 133 aa, Homo sapiens
  VARIANT (20)..(20): X is any amino acid other than Asp. In some cases, X is Ala.
  VARIANT (42)..(42): X is any amino acid other than Phe. In some cases, X is Ala.
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 252 - protein, 133 aa, Homo sapiens
  VARIANT (15)..(15): X is any amino acid other than Glu.  In some cases, X is Ala.
  VARIANT (20)..(20): X is any amino acid other than Asp.  In some cases, X is Ala.
  VARIANT (42)..(42): X is any amino acid other than Phe.  In some cases, X is Ala.
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 253 - protein, 133 aa, Homo sapiens
  VARIANT (16)..(16): X is any amino acid other than His. In some cases, X is Ala.
  VARIANT (20)..(20): X is any amino acid other than Asp. In some cases, X is Ala.
  VARIANT (42)..(42): X is any amino acid other than Phe. In some cases, X is Ala.
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 254 - protein, 133 aa, Homo sapiens
  VARIANT (20)..(20): X is any amino acid other than Asp. In some cases, X is Ala.
  VARIANT (42)..(42): X is any amino acid other than Phe. In some cases, X is Ala.
  VARIANT (126)..(126): X is any amino acid other than Gln. In some cases, X is Ala.
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 255 - protein, 133 aa, Homo sapiens
  VARIANT (20)..(20): X is any amino acid other than Asp. In some cases, X is Ala.
  VARIANT (42)..(42): X is any amino acid other than Phe. In some cases, X is Ala.
  VARIANT (45)..(45): X is any amino acid other than Tyr. In some cases, X is Ala.
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 256 - protein, 133 aa, Homo sapiens
  VARIANT (16)..(16): X is any amino acid other than His. In some cases, X is Ala.
  VARIANT (20)..(20): X is any amino acid other than Asp. In some cases, X is Ala.
  VARIANT (42)..(42): X is any amino acid other than Phe. In some cases, X is Ala.
  VARIANT (45)..(45): X is any amino acid other than Tyr. In some cases, X is Ala.
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 257 - protein, 133 aa, Homo sapiens
  VARIANT (20)..(20): X is any amino acid other than Asp. In some cases, X is Ala.
  VARIANT (42)..(42): X is any amino acid other than Phe. In some cases, X is Ala.
  VARIANT (45)..(45): X is any amino acid other than Tyr. In some cases, X is Ala.
  VARIANT (126)..(126): X is any amino acid other than Gln. In some cases, X is Ala.
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 258 - protein, 133 aa, Homo sapiens
  VARIANT (16)..(16): X is any amino acid other than His. In some cases, X is Ala.
  VARIANT (20)..(20): X is any amino acid other than Asp. In some cases, X is Ala.
  VARIANT (42)..(42): X is any amino acid other than Phe. In some cases, X is Ala.
  VARIANT (45)..(45): X is any amino acid other thanTyr. In some cases, X is Ala.
  VARIANT (126)..(126): X is any amino acid other than Gln. In some cases, X is Ala.
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 259 - protein, 133 aa, Homo sapiens
  VARIANT (16)..(16): X is any amino acid other than His.  In some cases, X is Ala.
  VARIANT (42)..(42): X is any amino acid other than Phe.  In some cases, X is Ala.
  VARIANT (126)..(126): X is any amino acid other than Gln.  In some cases, X is Ala.
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 260 - protein, 9 aa, Homo sapiens
YPYDVPDYA

SEQ ID NO: 261 - protein, 8 aa, Homo sapiens
DYKDDDDK

SEQ ID NO: 262 - protein, 10 aa, Homo sapiens
EQKLISEEDL

SEQ ID NO: 263 - protein, 5 aa, Homo sapiens
HHHHH

SEQ ID NO: 264 - protein, 6 aa, Homo sapiens
HHHHHH

SEQ ID NO: 265 - protein, 10 aa, Homo sapiens
EQKLISEEDL

SEQ ID NO: 266 - protein, 8 aa, Homo sapiens
DYKDDDDK

SEQ ID NO: 267 - protein, 8 aa, Homo sapiens
WSHPQFEK

SEQ ID NO: 268 - protein, 9 aa, Homo sapiens
YPYDVPDYA

SEQ ID NO: 269 - protein, 5 aa, Homo sapiens
RYIRS

SEQ ID NO: 270 - protein, 4 aa, Homo sapiens
FHHT

SEQ ID NO: 271 - protein, 17 aa, Homo sapiens
WEAAAREACCRECCARA

SEQ ID NO: 272 - protein, 10 aa, Hepatitis B virus
FLPSDFFPSV

SEQ ID NO: 273 - protein, 10 aa, Hepatitis B virus
GLSRYVARLG

SEQ ID NO: 274 - protein, 9 aa, Hepatitis B virus
KLHLYSHPI

SEQ ID NO: 275 - protein, 9 aa, Hepatitis B virus
FLLSLGIHL

SEQ ID NO: 276 - protein, 9 aa, Hepatitis B virus
ALMPLYACI

SEQ ID NO: 277 - protein, 9 aa, Hepatitis B virus
SLYADSPSV

SEQ ID NO: 278 - protein, 9 aa, Hepatitis B virus
STLPETTVV

SEQ ID NO: 279 - protein, 10 aa, Hepatitis B virus
LIMPARFYPK

SEQ ID NO: 280 - protein, 10 aa, Hepatitis B virus
AIMPARFYPK

SEQ ID NO: 281 - protein, 9 aa, Hepatitis B virus
YVNVNMGLK

SEQ ID NO: 282 - protein, 15 aa, Hepatitis B virus
MQWNSTALHQALQDP

SEQ ID NO: 283 - protein, 9 aa, Hepatitis B virus
LLDPRVRGL

SEQ ID NO: 284 - protein, 10 aa, Hepatitis B virus
SILSKTGDPV

SEQ ID NO: 285 - protein, 9 aa, Hepatitis B virus
VLQAGFFLL

SEQ ID NO: 286 - protein, 9 aa, Hepatitis B virus
FLLTRILTI

SEQ ID NO: 287 - protein, 9 aa, Hepatitis B virus
FLGGTPVCL

SEQ ID NO: 288 - protein, 9 aa, Hepatitis B virus
LLCLIFLLV

SEQ ID NO: 289 - protein, 10 aa, Hepatitis B virus
LVLLDYQGML

SEQ ID NO: 290 - protein, 10 aa, Hepatitis B virus
LLDYQGMLPV

SEQ ID NO: 291 - protein, 9 aa, Hepatitis B virus
IPIPSSWAF

SEQ ID NO: 292 - protein, 9 aa, Hepatitis B virus
WLSLLVPFV

SEQ ID NO: 293 - protein, 10 aa, Hepatitis B virus
GLSPTVWLSV

SEQ ID NO: 294 - protein, 10 aa, Hepatitis B virus
SIVSPFIPLL

SEQ ID NO: 295 - protein, 9 aa, Hepatitis B virus
ILSPFLPLL

SEQ ID NO: 296 - protein, 17 aa, Hepatitis B virus
ATVELLSFLPSDFFPSV

SEQ ID NO: 297 - protein, 9 aa, Hepatitis B virus
LPSDFFPSV

SEQ ID NO: 298 - protein, 9 aa, Hepatitis B virus
CLTFGRETV

SEQ ID NO: 299 - protein, 10 aa, Hepatitis B virus
VLEYLVSFGV

SEQ ID NO: 300 - protein, 9 aa, Hepatitis B virus
EYLVSFGVW

SEQ ID NO: 301 - protein, 10 aa, Hepatitis B virus
ILSTLPETTV

SEQ ID NO: 302 - protein, 11 aa, Hepatitis B virus
STLPETTVVRR

SEQ ID NO: 303 - protein, 9 aa, Hepatitis B virus
NVSIPWTHK

SEQ ID NO: 304 - protein, 9 aa, Hepatitis B virus
KVGNFTGLY

SEQ ID NO: 305 - protein, 9 aa, Hepatitis B virus
GLYSSTVPV

SEQ ID NO: 306 - protein, 10 aa, Hepatitis B virus
TLWKAGILYK

SEQ ID NO: 307 - protein, 10 aa, Hepatitis B virus
TPARVTGGVF

SEQ ID NO: 308 - protein, 10 aa, Hepatitis B virus
LVVDFSQFSR

SEQ ID NO: 309 - protein, 9 aa, Hepatitis B virus
GLSRYVARL

SEQ ID NO: 310 - protein, 9 aa, Hepatitis B virus
SAICSVVRR

SEQ ID NO: 311 - protein, 9 aa, Hepatitis B virus
YMDDVVLGA

SEQ ID NO: 312 - protein, 8 aa, Hepatitis B virus
PLGFFPDH

SEQ ID NO: 313 - protein, 10 aa, Hepatitis B virus
QAFTFSPTYK

SEQ ID NO: 314 - protein, 9 aa, Hepatitis B virus
KYTSFPWLL

SEQ ID NO: 315 - protein, 10 aa, Hepatitis B virus
ILRGTSFVYV

SEQ ID NO: 316 - protein, 9 aa, Hepatitis B virus
HLSLRGLFV

SEQ ID NO: 317 - protein, 9 aa, Hepatitis B virus
VLHKRTLGL

SEQ ID NO: 318 - protein, 10 aa, Hepatitis B virus
GLSAMSTTDL

SEQ ID NO: 319 - protein, 9 aa, Hepatitis B virus
CLFKDWEEL

SEQ ID NO: 320 - protein, 9 aa, Hepatitis B virus
VLGGCRHKL

SEQ ID NO: 321 - protein, 9 aa, Human papillomavirus
KLPQLCTEL

SEQ ID NO: 322 - protein, 9 aa, Human papillomavirus
LQTTIHDII

SEQ ID NO: 323 - protein, 9 aa, Human papillomavirus
VYDFAFRDL

SEQ ID NO: 324 - protein, 9 aa, Human papillomavirus
FAFRDLCIV

SEQ ID NO: 325 - protein, 9 aa, Human papillomavirus
KFYSKISEY

SEQ ID NO: 326 - protein, 9 aa, Human papillomavirus
ISEYRHYCY

SEQ ID NO: 327 - protein, 9 aa, Human papillomavirus
TLHEYMLDL

SEQ ID NO: 328 - protein, 9 aa, Human papillomavirus
YMLDLQPET

SEQ ID NO: 329 - protein, 9 aa, Human papillomavirus
QAEPDRAHY

SEQ ID NO: 330 - protein, 9 aa, Human papillomavirus
RAHYNIVTF

SEQ ID NO: 331 - protein, 9 aa, Human papillomavirus
CDSTLRLCV

SEQ ID NO: 332 - protein, 10 aa, Human papillomavirus
LCVQSTHVDI

SEQ ID NO: 333 - protein, 9 aa, Human papillomavirus
LLMGTLGIV

SEQ ID NO: 334 - protein, 8 aa, Human papillomavirus
TLGIVCPI

SEQ ID NO: 335 - protein, 12 aa, Human papillomavirus
LLMGTLGIVCPI

SEQ ID NO: 336 - protein, 9 aa, Human papillomavirus
CYSLYGTTL

SEQ ID NO: 337 - protein, 9 aa, Human papillomavirus
EYRHYCYSL

SEQ ID NO: 338 - protein, 9 aa, Human papillomavirus
DPQERPRKL

SEQ ID NO: 339 - protein, 9 aa, Human papillomavirus
HYCYSLYGT

SEQ ID NO: 340 - protein, 9 aa, Human papillomavirus
DFAFRDLCI

SEQ ID NO: 341 - protein, 10 aa, Human papillomavirus
LYGTTLEQQY

SEQ ID NO: 342 - protein, 10 aa, Human papillomavirus
HYCYSLYGTT

SEQ ID NO: 343 - protein, 10 aa, Human papillomavirus
EVYDFAFRDL

SEQ ID NO: 344 - protein, 10 aa, Human papillomavirus
EYRHYCYSLY

SEQ ID NO: 345 - protein, 10 aa, Human papillomavirus
VYDFAFRDLC

SEQ ID NO: 346 - protein, 10 aa, Human papillomavirus
YCYSIYGTTL

SEQ ID NO: 347 - protein, 9 aa, Human papillomavirus
VYCKTVLEL

SEQ ID NO: 348 - protein, 9 aa, Human papillomavirus
VYGDTLEKL

SEQ ID NO: 349 - protein, 10 aa, Human papillomavirus
LTNTGLYNLL

SEQ ID NO: 350 - protein, 9 aa, Human papillomavirus
DLQPETTDL

SEQ ID NO: 351 - protein, 9 aa, Human papillomavirus
TPTLHEYML

SEQ ID NO: 352 - protein, 9 aa, Human papillomavirus
GTLGIVCPI

SEQ ID NO: 353 - protein, 9 aa, Human papillomavirus
EPDRAHYNI

SEQ ID NO: 354 - protein, 9 aa, Human papillomavirus
QLFLNTLSF

SEQ ID NO: 355 - protein, 9 aa, Human papillomavirus
FQQLFLNTL

SEQ ID NO: 356 - protein, 10 aa, Human papillomavirus
AFQQLFLNTL

SEQ ID NO: 357 - protein, 8 aa, Artificial Sequence
  proteolytically cleavable linkers
LEVLFQGP

SEQ ID NO: 358 - protein, 7 aa, Artificial Sequence
  proteolytically cleavable linkers
ENLYTQS

SEQ ID NO: 359 - protein, 5 aa, Artificial Sequence
  proteolytically cleavable linkers
DDDDK

SEQ ID NO: 360 - protein, 4 aa, Artificial Sequence
  proteolytically cleavable linkers
LVPR

SEQ ID NO: 361 - protein, 22 aa, Artificial Sequence
  proteolytically cleavable linkers
GSGATNFSLLKQAGDVEENPGP

SEQ ID NO: 362 - protein, 123 aa, Artificial Sequence
  Protein/polypeptide construct
NLVPMVATVGGGASGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 363 - protein, 813 aa, Artificial Sequence
  Protein/polypeptide construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 99 aa, Artificial Sequence
  Protein/polypeptide construct
  SITE (7)..(7): cysteine that acts as a chemical conjugation site
  SITE (44)..(44): cysteine that acts as a chemical conjugation site
IQRTPKIQVYSCHPAENGKSNFLNCYVSGFHPSDIEVDLLKNGCRIEKVEHSDLSFSKDWSFYLLYYTEFTPTEKDEYACRVNHVTLSQPKIVKWDRDM

SEQ ID NO: 365 - protein, 813 aa, Artificial Sequence
  Protein/polypeptide construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 135 aa, Artificial Sequence
  Protein/polypeptide construct
MSRSVALAVLALLSLSGLEAGGGGSLCTPSRGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 367 - protein, 833 aa, Artificial Sequence
  Protein/polypeptide construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 20 aa, Artificial Sequence
  1st portion of a polypeptide/protein construct
MYRMQLLSCIALSLALVTNS

SEQ ID NO: 369 - protein, 507 aa, Artificial Sequence
  2nd portion of a polypeptide/protein construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 371 - protein, 813 aa, Artificial Sequence
  Polypeptide/protein construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 532 aa, Artificial Sequence
  Polypeptide/protein construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 838 aa, Artificial Sequence
  Polypeptide/protein construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 445 aa, Hepatitis B virus
MWLIITSRRDIIYTLFGRRVSYIKESPHVAPHFAGHHILGNKIYSMGGWSSKPRKGMGTNLSVPNPLGFFPDHQLDPAFKANSDNPDWDLNPHKDNWPDANKVGVGAFGPGFIPSHGGLLGWSPKAQGILTTVPAASLLASTIGKSGRQPTPLSPPLRDTHPQAMQWNSTTFHQTLQDPRVRALYFPAGGSSSGTVSPAQNTVSAISSILSKTGDPVPNMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFTSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 375 - protein, 281 aa, Hepatitis B virus
MQWNSSTFHQALLDPRVRGLYFPAGGSSSGTVNPVPTTASPISSIFSRTGDPAPNMESTTSGFLGPLLVLQAGFFLLTRILTIPQSLDSWWTSLNFLGGAPTCPGQNLQSPTSNHSQTSCPPICPGYRWMCLRRFIIFLFILLLCLIFLLVLLDYQGMLPVCPLLPGTSTTSTGPCKTCTTPAQGTSMFPSCCCTKPSDGNCTCIPIPSSWAFARFLWEWASVRFSWLSLLVPFVQWFVGLSPTVWLSVIWMMWYWGPSLYNILNPFLPLLPIFFCLWVYI

SEQ ID NO: 376 - protein, 226 aa, Hepatitis B virus
MESTTSGFLGPLLVLQAGFFLLTRILTIPQSLDSWWTSLNFLGGAPTCPGQNLQSPTSNHSQTSCPPICPGYRWMCLRRFIIFLFILLLCLIFLLVLLDYQGMLPVCPLLPGTSTTSTGPCKTCTTPAQGTSMFPSCCCTKPSDGNCTCIPIPSSWAFARFLWEWASVRFSWLSLLVPFVQWFVGLSPTVWLSVIWMMWYWGPSLYNILNPFLPLLPIFFCLWVYI

SEQ ID NO: 377 - protein, 843 aa, Hepatitis B virus
MPLSYQHFRKLLLLDEEAGPLEEELPRLADEGLNRRVAEDLNLGNLNVSIPWTHKVGNFTGLYSSTVPCFNPKWQTPSFPDIHLQEDIVDRCKQFVGPLTVNENRRLKLIMPARFYPNVTKYLPLDKGIKPYYPEHVVNHYFQTRHYLHTLWKAGILYKRESTRSASFCGSPYSWEQDLQHGRLVFKTSKRHGDKSFCPQSPGILPRSSVGPCIQSQLRKSRLGPQPAQGQLAGRQQGGSGSIRARVHPSPWGTVGVEPSGSGHTHNCASSSSSCLHQSAVRKAAYSLISTSKGHSSSGHAVELHHFPPNSSRSQSQGPVLSCWWLQFRNSEPCSEYCLCHIVNLIEDWGPCTEHGEHRIRTPRTPARVTGGVFLVDKNPHNTTESRLVVDFSQFSRGDTRVSWPKFAVPNLQSLTNLLSSNLSWLSLDVSAAFYHLPLHPAAMPHLLVGSSGLSRYVARLSSNSRIINNQHRTMQNLHNSCSRNLYVSLMLLYKTYGRKLHLYSHPIILGFRKIPMGVGLSPFLLAQFTSAICSVVRRAFPHCLAFSYMDDVVLGAKSVQHLESLYAAVTNFLLSLGIHLNPHKTKRWGYSLNFMGYVIGCWGTMPQEHIVQKIKMCFRKLPVNRPIDWKVCQRIVGLLGFAAPFTQCGYPALMPLYACIQAKQAFTFSPTYKAFLSKQYLNLYPVARQRSGLCQVFADATPTGWGLAIGHQRMRGTFVSPLPIHTAELLAACFARSRSGAKLIGTDNSVVLSRKYTSFPWLLGCAANWILRGTSFVYVPSALNPADDPSRGRLGLYRPLLRLLYRPTTGRTSLYADSPSVPSHLPDRVHFASPLHVAWRPP

SEQ ID NO: 378 - protein, 185 aa, Hepatitis B virus
MDIDPYKEFGASAELLSFLPSDFFPSVRDLLDTAKALFQEALESPEHCSPHHTALRQAILCWGDLMTLATWVGANLEDPASRDLVVNYVNTTAGLKFRQLLWFHISCLTFGRETVIEYLVSFGVWIRTPPPYRPPNAPILSTLPETTVVRYRDRGRSTRRRTPSPRRRRSQSPRRRRSQSRESQC

SEQ ID NO: 379 - protein, 212 aa, Hepatitis B virus
MQLFHLCLIISCSCPTVQASKLCLGWLWGMDIDPYKEFGASAELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRGSQC

SEQ ID NO: 380 - protein, 154 aa, Hepatitis B virus
MAARLCCQLDPARDVLCLRPVGAESCGRPFSGSLGTLSSPSPSAVPTDHGAHLSLRGLPVCAFSSAGPCALRFTSARRMETTVNAHQILPKVLHKRTLGLSAMSTTDLEAYFKDCLFKDWEELGEEIRLKVFVLGGCRHKLVCAPAPCNFFTSA

SEQ ID NO: 381 - protein, 158 aa, Hepatitis B virus
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 382 - protein, 141 aa, Hepatitis B virus
MHGDTPTLHEYMLDLQPETTDLYCYEQLNYSSEEEDEIDGPAGQAEPDRAHYNIVTFCCKCDSTLRLCVQSTHVDIRTLEDLLMGTLGIVCPICSQKPYSTMADPAGTNGEEGTGCNGWFYVEAVVEKKTGDAISDDENEN